(19) United States
(12) Patent Application Publication
(10) Pub. No.: US 2024/0186007 A1
(43) Pub. Date: Jun. 6, 2024

(54) COGNITIVE QUANTUM COMPUTER

(71) Applicant: Newton Howard, Potomac, MD (US)

(72) Inventor: Newton Howard, Potomac, MD (US)

(21) Appl. No.: 18/493,082

(22) Filed: Oct. 24, 2023

Related U.S. Application Data
(63) Continuation-in-part of application No. 18/474,227, filed on Sep. 26, 2023.
(60) Provisional application No. 63/418,960, filed on Oct. 24, 2022.

(51) Int. Cl. G16H 50/20; G06N 10/20

(52) U.S. Cl. CPC G16H 50/20; G06N 10/20

(57) ABSTRACT

For example, in an embodiment, a system may comprise a processor, memory, and program instructions and data, a plurality of stimulation devices, program instructions and data stored to control the stimulation devices to generate and transmit stimulation signals, a plurality of sensing devices, program instructions and data to receive sensed signals from the sensing devices, a communication device adapted, wherein the processor, memory, plurality of stimulation devices, plurality of sensing devices, and communication device are contained in an implantable device adapted to be implanted in a body of a person, and an external computer system comprising a processor, memory, and program instructions and data, to perform dynamic closed loop feedback of the stimulation signals based on the received sensed signals to provide self-guided, self-directed diagnostics and treatment of neural conditions, wherein the external computer system is adapted to be attached to a body of the person.

### CROSS-REFERENCE TO RELATED APPLICATIONS

This application claims the benefit of U.S. Provisional App. No. 63/418,960, filed Oct. 24, 2022, and is a continuation-in-part of U.S. application Ser. No. 18/474,227, filed Sep. 26, 2023, which claims the benefit of U.S. Provisional App. No. 63/410,004, filed Sep. 26, 2022, and is a continuation-in-part of U.S. application Ser. No. 18/209,447, filed Jun. 13, 2023, which claims the benefit of U.S. Provisional App. No. 63/351,957, filed Jun. 14, 2022, and is a continuation-in-part of U.S. application Ser. No. 18/197,099, filed May 14, 2023, which claims the benefit of U.S. Provisional App. No. 63/342,048, filed May 13, 2022, and is a continuation-in-part of U.S. application Ser. No. 17/961,976, filed Oct. 7, 2022, which claims the benefit of U.S. Provisional App. No. 63/321,650, filed Mar. 18, 2022, U.S. Provisional App. No. 63/325,973, filed Mar. 31, 2022, U.S. Provisional App. No. 63/341,089, filed May 12, 2022, and U.S. Provisional App. No. 63/341,152, filed May 12, 2022, and is a continuation-in-part of U.S. application Ser. No. 17/588,728, filed Jan. 31, 2022, which claims the benefit of U.S. Provisional App. No. 63/144,420, filed Feb. 1, 2021 and is a continuation-in-part of U.S. application Ser. No. 17/561,677, filed Dec. 23, 2021, which is a continuation-in-part of U.S. application Ser. No. 17/198,518, filed Mar. 11, 2021, which claims the benefit of U.S. Provisional App. No. 62/988,323, filed Mar. 11, 2020 and is a continuation in part of U.S. application Ser. No. 17/175,680, filed Feb. 14, 2021, which claims the benefit of U.S. Provisional App. No. 62/977,124, filed Feb. 14, 2020, U.S. Provisional App. No. 62/985,872, filed Mar. 5, 2020, U.S. Provisional App. No. 62/985,874, filed Mar. 5, 2020, and U.S. Provisional App. No. 62/985,889, filed Mar. 6, 2020, the contents of all of which are incorporated herein in their entirety.

### BACKGROUND

The present invention relates to techniques that may provide the capability to provide self-guided, self-directed diagnostics and treatment of neural conditions.

According to the United Nations, roughly one billion people, nearly ⅙th of the world's population, presently suffer from some form of neurological disorder, with some 6.8 million deaths each year. During the past decades, a large amount of work on several brain diseases were unsuccessful, because they take neither the initial state of the neuronal—brain region nor the initial neuronal interplay into consideration, greatly limiting the validity of conclusions made. Because the data recorded are only a snapshot of a precise situation, conclusions must be made based mainly on assumption about the properties of neurons and networks.

The UN estimates that one in every four people will suffer from a neurological or mental disorder in their lifetime and the vast majority of these cases will remain undiagnosed. Of those who are diagnosed, the World Health Organization claims two-thirds never seek treatment (reference). Conventional systems cannot quantitatively detect and track the progression of a neurological disease (or the efficacy of a treatment).

Accordingly, a need arises for techniques that may provide the capability to provide improved brain monitoring and stimulation devices.

### SUMMARY

Embodiments may provide techniques that may provide the capability to provide self-guided, self-directed diagnostics and treatment of neural conditions.

For example, in an embodiment, a system to provide self-guided, self-directed diagnostics and treatment of neural conditions may comprise a processor, memory accessible by the processor, and program instructions and data stored in the memory, a plurality of stimulation devices connected to signal output circuitry interfacing the processor with the stimulation devices program instructions and data stored in the memory to configure the processor to control the stimulation devices to generate and transmit stimulation signals, a plurality of sensing devices connected to signal input circuitry interfacing the processor with the sensing devices, program instructions and data stored in the memory to configure the processor to receive sensed signals from the sensing devices, a communication device adapted to wirelessly communicate with an external computer system, wherein the processor, memory, plurality of stimulation devices, plurality of sensing devices, and communication device are contained in an implantable device adapted to be implanted in a body of a person, and an external computer system comprising a processor, memory accessible by the processor, and program instructions and data stored in the memory, the program instructions and data stored in the memory to configure the processor to perform dynamic closed loop feedback of the stimulation signals based on the received sensed signals to provide self-guided, self-directed diagnostics and treatment of neural conditions using at least one recipe for a treatment strategy guided by artificial intelligence contained in the server computer system and communicated using the communication device, wherein the external computer system is adapted to be attached to a body of the person.

In embodiments, the plurality of stimulation devices may comprise at least one of stimulation devices in the implantable device providing electrical stimulation, stimulation devices in the implantable device providing optical stimulation, and stimulation devices in the implantable device providing both electrical and optical stimulation. The plurality of stimulation devices may further comprise stimulation devices not contained in the implantable device including at least one of a Transcranial Direct Current Stimulation (tDCS) device, a Transcranial Alternating Current Stimulation (tACS), and a Transcranial Photobiomodulation (tPBM). The plurality of sensing devices may comprise at least one of sensing devices contained in the implantable device providing electrical sensing, sensing devices contained in the implantable device providing optical sensing, and sensing devices contained in the implantable device providing both electrical and optical sensing. The plurality of sensing devices may further comprise sensing devices not contained in the implantable device including at least an Electro Encephalogram (EEG) device. The system may further comprise program instructions and data stored in the memory to configure the processor of the implant device to perform: applying stimulation based on a first version of stimulation parameters, sensing results of the applied stimulation to form patient treatment results data, transmitting the patient treatment results data to the external computer system, receiving a second version of stimulation parameters from the external computer system, and applying stimulation based on the second version of stimulation parameters; and may further comprise program instructions and data stored in the memory of the external computer system to configure the processor of the external computer system to perform: receiving the patient treatment results data, modifying the first version of stimulation parameters using the patient treatment results data to form a second version of stimulation parameters, and transmitting the second version of stimulation parameters to the implant device. The system may further comprise program instructions and data stored in the memory to configure the processors of the implant device and the external computer system to perform: repeating the applying, sensing, transmitting, receiving, and modifying using each modified version of stimulation parameters. The communication device may be adapted to communicate using WiFi wireless communications. The communication device may be adapted to communicate using Ultra Wide-Band wireless communications.

### BRIEF DESCRIPTION OF THE DRAWINGS

The details of the present invention, both as to its structure and operation, can best be understood by referring to the accompanying drawings, in which like reference numbers and designations refer to like elements.

FIG. 1 illustrates an exemplary embodiment of a Biological Co-Processor System (BCP).

FIG. 2 illustrates an exemplary embodiment of an implantable signal receiving, processing, and transmitting device, shown in FIG. 1.

FIG. 3 illustrates an exemplary embodiment of Brain Code Collection System earbud, shown in FIG. 1.

FIG. 4 illustrates an exemplary embodiment of a cloud platform, shown in FIG. 1.

FIG. 5 illustrates an exemplary embodiment of an inductive powering system, shown in FIG. 1.

FIG. 6 illustrates exemplary advantages of aspects of technologies that may be utilized by embodiments.

FIG. 7 illustrates exemplary advantages of aspects of technologies that may be utilized by embodiments.

FIG. 8 illustrates an exemplary embodiment of an implant device.

FIG. 9 illustrates an exemplary embodiment of an implant device.

FIG. 10 illustrates an exemplary embodiment of a tile design for an implant device.

FIG. 11 illustrates an exemplary embodiment of a tile arrangement for an implant device.

FIG. 12 is an exemplary illustration of an approximate representation of how the optrode array could fit over a dense neural network.

FIG. 13 illustrates an exemplary embodiment of an implant device.

FIG. 14 illustrates an exemplary embodiment of an implant device.

FIG. 15 illustrates an exemplary embodiment of CNT connection for an implant device.

FIG. 16 illustrates an example of fast-scan cyclic voltammetry.

FIG. 17 illustrates an example of how carbon nanotube color changes with chiral index.

FIG. 18 illustrates an exemplary embodiment of a nanoengineered electroporation microelectrodes (NEM).

FIG. 19 illustrates an exemplary embodiment of an electrophysiological recording pipeline.

FIG. 20 illustrates an exemplary embodiment of an optical recording pipeline.

FIG. 21 illustrates an exemplary embodiment of an optical recording pipeline.

FIG. 22 illustrates an example of cyclically applied potential for cyclic voltammetry.

FIG. 23 illustrates an exemplary embodiment of recording pipelines and data processing circuitry.

FIG. 24 illustrates an example of spike trains of ChR2 and NpHR expressing neurons when subjected to light beams of different wavelengths.

FIG. 25 illustrates an example of Poisson trains of spikes elicited by pulses of blue light (dashes), in two different neurons.

FIG. 26 illustrates examples of a light-driven spike blockade for different neurons.

FIG. 27 illustrates examples of reaction events for different neurons.

FIG. 28 examples of the correlation between wavelengths (nm) and normalized cumulative charge for different Channel rhodopsins neurons.

FIG. 29 illustrates an exemplary embodiment of an optical stimulation pipeline.

FIG. 30 illustrates an exemplary embodiment of an optical stimulation pipeline.

FIG. 31 illustrates an exemplary embodiment of an optical stimulation pipeline.

FIG. 32 illustrates an exemplary embodiment of optical stimulation pipelines.

FIG. 33 illustrates an exemplary embodiment of an implant device.

FIG. 34 illustrates an exemplary embodiment of pseudocode for a process of data recording.

FIG. 35 illustrates an exemplary embodiment of pseudocode for a process of stimulation requests.

FIG. 36 illustrates an exemplary embodiment of a closed loop control system.

FIG. 37 illustrates an exemplary embodiment of pseudocode for a closed loop control system.

FIG. 38 illustrates an exemplary embodiment of pseudocode for a PID algorithm.

FIG. 39 illustrates exemplary data flow block diagram of a spike sorting technique.

FIG. 40a illustrates a portion of an exemplary embodiment of pseudocode for performing an SPC method.

FIG. 40b illustrates a portion of an exemplary embodiment of pseudocode for performing an SPC method.

FIG. 41 illustrates an exemplary embodiment of pseudocode for a Spike Sorting technique.

FIG. 42 illustrates an exemplary embodiment of pseudocode for bit encoding techniques.

FIG. 43a illustrates a portion of an exemplary embodiment of code for bit encoding techniques.

FIG. 43b illustrates a portion of an exemplary embodiment of code for bit encoding techniques.

FIG. 44 illustrates an exemplary embodiment of pseudocode for a Startup Procedure.

FIG. 45 illustrates an exemplary embodiment of pseudocode for a Provisioning Procedure.

FIG. 46 illustrates an exemplary embodiment of pseudocode for a Configuration Interface.

FIG. 47 illustrates an exemplary embodiment of pseudocode for a Stimulation Interface.

FIG. 48 illustrates an exemplary embodiment of pseudocode for a Recording Interface.

FIG. 49 illustrates an exemplary embodiment of pseudocode for a Status Interface.

FIG. 50 illustrates an exemplary embodiment of pseudocode for a temperature and power monitoring module.

FIG. 51 illustrates an exemplary embodiment of pseudocode for a Startup Procedure.

FIG. 52 illustrates an exemplary embodiment of pseudocode for a Provisioning Procedure.

FIG. 53a illustrates a portion of an exemplary embodiment of pseudocode for a command execution procedure.

FIG. 53b illustrates a portion of an exemplary embodiment of pseudocode for a command execution procedure.

FIG. 53c illustrates a portion of an exemplary embodiment of pseudocode for a command execution procedure.

FIG. 54 illustrates an exemplary embodiment of pseudocode for a data streaming procedure.

FIG. 55 illustrates an exemplary block diagram of a Gateway.

FIG. 56 illustrates an exemplary block diagram of the Cloud.

FIG. 57 illustrates an exemplary embodiment of pseudocode for a command message.

FIG. 58 illustrates an exemplary embodiment of pseudocode for a Configuration Command.

FIG. 59 illustrates an exemplary embodiment of pseudocode for a Stimulation Command.

FIG. 60 illustrates an exemplary embodiment of pseudocode for an Activation Command.

FIG. 61 illustrates an exemplary embodiment of pseudocode for an OTA Command.

FIG. 62 illustrates an exemplary embodiment of pseudocode for a Recording Control Command.

FIG. 63 illustrates an exemplary embodiment of pseudocode for a Status Command.

FIG. 64 illustrates an exemplary embodiment of pseudocode for a command message.

FIG. 65 illustrates an exemplary embodiment of pseudocode for a command message.

FIG. 66 illustrates an exemplary embodiment of pseudocode for a data message.

FIG. 67 illustrates an exemplary block diagram of an architecture for data ingestion and data processing.

FIG. 68 illustrates an exemplary embodiment of pseudocode for an API that may be used to specify the input for real time processing.

FIG. 69 illustrates an exemplary embodiment of pseudocode for an API that may be used to specify the pre-processing for real time processing.

FIG. 70 illustrates an exemplary embodiment of pseudocode for an API that may be used to specify the machine learning processing for real time processing.

FIG. 71a illustrates a portion of an exemplary embodiment of pseudocode for an API that may be used to specify the output for real time processing.

FIG. 71b illustrates a portion of an exemplary embodiment of pseudocode for an API that may be used to specify the output for real time processing.

FIG. 72 illustrates an exemplary embodiment of pseudocode for an API that may be used to specify the input for batch processing.

FIG. 73 illustrates an exemplary embodiment of pseudocode for an API that may be used to specify the machine learning for training new models for batch processing.

FIG. 74 illustrates an exemplary embodiment of pseudocode for an API that may be used to specify custom blocks for batch processing.

FIG. 75 illustrates an exemplary embodiment of pseudocode for an API that may be used for output from batch processing.

FIG. 76 illustrates an exemplary block diagram of an automatic pipeline.

FIG. 77 illustrates an exemplary embodiment of a module for autonomous processes.

FIG. 78 illustrates an exemplary embodiment of a cascading module for workflows.

FIG. 79 illustrates an exemplary embodiment of a pipeline for processing.

FIG. 80 illustrates an exemplary embodiment of a Machine Learning (ML) Toolbox.

FIG. 81 illustrates an exemplary embodiment of a pipeline for processing.

FIG. 82 illustrates an exemplary embodiment of a portion of a process of fabrication of CNT implant devices.

FIG. 83 illustrates an exemplary embodiment of a portion of a process of fabrication of CNT implant devices.

FIG. 84 illustrates an exemplary embodiment of a recording and stimulation signal and data flow on an implant device.

FIG. 85 illustrates an exemplary embodiment of a recording and stimulation signal and data flow on the Gateway and Cloud.

FIG. 86 illustrates an exemplary block diagram of an embodiment of an implant device electrical system.

FIG. 87 illustrates an exemplary embodiment of a portion of an implant device electrical system.

FIG. 88 illustrates an exemplary embodiment of a portion of an implant device electrode connection and firing distribution.

FIG. 89 illustrates an exemplary embodiment of a portion of triggering of the first ADC and the quantization of the action potential.

FIG. 90 illustrates an exemplary block diagram of multiplexer connections.

FIG. 91 illustrates an exemplary block diagram of a Gain Block.

FIG. 92 illustrates an exemplary block diagram of a Gain Block.

FIG. 93 illustrates an exemplary block diagram of an ADC.

FIG. 94 illustrates an exemplary block diagram of a DAC Block.

FIG. 95 illustrates an example of light scattering effects with wavelength.

FIG. 96 illustrates an exemplary block diagram of a computing device in which embodiments of the present systems and method may be implemented.

FIG. 97 illustrates an exemplary embodiment of an implant device.

FIG. 98 illustrates an exemplary embodiment of a Brain-Machine Interface system.

FIG. 99 illustrates an exemplary high-level system block diagram of an embodiment of a Brain-Machine Interface system.

FIG. 100 illustrates an exemplary high-level system block diagram of an embodiment of a Brain-Machine Interface system.

FIG. 101 illustrates an exemplary high-level system block diagram of an embodiment of a Brain-Machine Interface system.

FIG. 102 illustrates an exemplary embodiment of an integrated sliding support brace.

FIG. 103 illustrates an exemplary embodiment of a sign-in interface of a no-code drag-and-drop AI user interface.

FIG. 104 illustrates an exemplary embodiment of a project name definition interface of a no-code drag-and-drop AI user interface.

FIG. 105 illustrates an exemplary embodiment of a data upload interface of a no-code drag-and-drop AI user interface.

FIG. 106 illustrates an exemplary embodiment of a directed project definition interface of a no-code drag-and-drop AI user interface.

FIG. 107 illustrates an exemplary embodiment of an experiment definition interface of a no-code drag-and-drop AI user interface.

FIG. 108 illustrates an exemplary embodiment of an experiment data analysis interface of a no-code drag-and-drop AI user interface.

FIG. 109 illustrates an exemplary embodiment of a projects overview interface of a no-code drag-and-drop AI user interface.

FIG. 110 illustrates an exemplary embodiment of a data analysis interface of a no-code drag-and-drop AI user interface.

FIG. 111 illustrates an exemplary embodiment of a data analysis interface of a no-code drag-and-drop AI user interface.

FIG. 112 illustrates an exemplary embodiment of a results analysis interface of a no-code drag-and-drop AI user interface.

FIG. 113 illustrates an exemplary embodiment of a results analysis interface of a no-code drag-and-drop AI user interface.

FIG. 114 illustrates an exemplary embodiment of a BCCS earbud.

FIG. 115 illustrates an exemplary embodiment of a BCCS earbud.

FIG. 116 illustrates an exemplary embodiment of a BCCS earbud.

FIG. 117 illustrates an exemplary embodiment of a results analysis interface of a no-code drag-and-drop AI user interface.

FIG. 118 illustrates an exemplary embodiment of a BCCS earbud.

FIG. 119 illustrates an exemplary embodiment of a BCCS earbud.

FIG. 120 illustrates an exemplary embodiment of a KIWI system.

FIG. 121 illustrates an exemplary embodiment of a processing pipeline.

FIG. 122 illustrates an exemplary embodiment of a sensor frame.

FIG. 123 illustrates an exemplary embodiment of a KIWI/BCCS system.

FIG. 124 illustrates an exemplary embodiment of a nasal cannula of a KIWI/BCCS system.

FIG. 125 illustrates an exemplary embodiment of an interior cap a KIWI/BCCS system.

FIG. 126 illustrates an exemplary block diagram of an embodiment of a KIWI/BCCS system.

FIG. 127 illustrates an exemplary block diagram of an embodiment of a Gateway.

FIG. 128 illustrates an exemplary block diagram of an embodiment of a KIWI/BCCS firmware.

FIG. 129 illustrates an exemplary block diagram of an embodiment of a KIWI/BCCS system.

FIG. 130 illustrates an example of an embodiment of LED/electrode placement on a person.

FIG. 131 illustrates an exemplary block diagram of an embodiment of a tKiwi architecture.

FIG. 132 illustrates an exemplary block diagram of an embodiment of a tKiwi system overview.

FIG. 133 illustrates an exemplary block diagram of an embodiment of a tKiwi system data flow.

FIG. 134 shows exemplary views of an embodiment of a tKiwi headset.

FIG. 135 illustrates an exemplary embodiment of a tKiwi system.

FIG. 136 illustrates an exemplary embodiment of personalized treatment using a tKiwi system.

FIG. 136 illustrates an exemplary embodiment of a process of personalized treatment using recipes.

FIG. 137 illustrates an exemplary embodiment of a process of personalized treatment using recipes.

FIG. 138 illustrates an exemplary embodiment of a process of personalized treatment using recipes.

FIG. 139 illustrates an exemplary processing module.

FIG. 140 illustrates an exemplary multiplexer.

FIG. 141 illustrates an exemplary multifunctional pixel or cell of a CMOS matrix or array.

FIG. 142 illustrates an exemplary multifunctional CMOS matrix or array.

FIG. 143 illustrates an exemplary software functionalities for embodiments of an implant system.

FIG. 144 illustrates exemplary files for embodiments of an implant system.

FIG. 145 illustrates an exemplary process of adding a command for embodiments of an implant system.

FIG. 146 illustrates an exemplary process of adding a command for embodiments of an implant system.

FIG. 147 illustrates exemplary range checks for a command for embodiments of an implant system.

FIG. 148 illustrates exemplary process of adding a handler function for embodiments of an implant system.

FIG. 149 illustrates an exemplary process of adding a command ID for embodiments of an implant system.

FIG. 150 illustrates an exemplary process of adding a command response for embodiments of an implant system.

FIG. 151 illustrates an exemplary process of adding a topic for embodiments of an implant system.

FIG. 152 is an exemplary block diagram of an embodiment of a Kiwi brain monitoring and stimulation system.

FIG. 153 illustrates an exemplary process of operation of the system shown in FIG. 152.

FIG. 154 illustrates an exemplary process of set up of the system shown in FIG. 152.

FIG. 155 illustrates an exemplary embodiment of a headstage of a Kiwi brain monitoring and stimulation system.

FIG. 156 illustrates an exemplary embodiment of a headstage of a Kiwi brain monitoring and stimulation system.

FIG. 157 illustrates an exemplary embodiment of a gateway of a Kiwi brain monitoring and stimulation system.

FIG. 158 illustrates an exemplary embodiment of a gateway of a Kiwi brain monitoring and stimulation system.

FIG. 159 illustrates an exemplary embodiment of an initial screen display of a software suite of a Kiwi brain monitoring and stimulation system.

FIG. 160 illustrates an exemplary embodiment of an RSL dialog of a software suite of a Kiwi brain monitoring and stimulation system.

FIG. 161 illustrates an exemplary embodiment of an RSL dialog of a software suite of a Kiwi brain monitoring and stimulation system.

FIG. 162 illustrates an exemplary embodiment of a dialog of a software suite of a Kiwi brain monitoring and stimulation system.

FIG. 163 illustrates an exemplary embodiment of a dialog of a software suite of a Kiwi brain monitoring and stimulation system.

FIG. 164 illustrates an exemplary embodiment of a dialog of a software suite of a Kiwi brain monitoring and stimulation system.

FIG. 165 illustrates an exemplary embodiment of a dialog of a software suite of a Kiwi brain monitoring and stimulation system.

FIG. 166 illustrates an exemplary embodiment of a dialog of a software suite of a Kiwi brain monitoring and stimulation system.

FIG. 167 illustrates an exemplary embodiment of a dialog of a software suite of a Kiwi brain monitoring and stimulation system.

FIG. 168 illustrates an exemplary embodiment of a dialog of a software suite of a Kiwi brain monitoring and stimulation system.

FIG. 169 illustrates an exemplary embodiment of a dialog of a software suite of a Kiwi brain monitoring and stimulation system.

FIG. 170 illustrates an exemplary embodiment of a dialog of a software suite of a Kiwi brain monitoring and stimulation system.

FIG. 171 illustrates an exemplary embodiment of a dialog of a software suite of a Kiwi brain monitoring and stimulation system.

FIG. 172 illustrates an exemplary embodiment of diagram on how the AI toolbox interacts with the rest of the components to achieve real time or near real time closed loop stimulation in a Kiwi brain monitoring and stimulation system.

FIG. 173 illustrates a 3-D view of an exemplary embodiment of a Kiwi brain monitoring and stimulation system.

FIG. 174 illustrates a 3-D view of an exemplary embodiment of a Kiwi brain monitoring and stimulation system.

FIG. 175 illustrates a 3-D view of an exemplary embodiment of a Kiwi brain monitoring and stimulation system.

FIG. 176 illustrates an overview of an exemplary MOSFET device.

FIG. 177 illustrates an example of the superposition principle: isolated electrical recording procedure.

FIG. 178 illustrates an example of the superposition principle: isolated stimulation.

FIG. 179 illustrates an example of a twisted nematic device architecture.

FIG. 180 illustrates an example of numbers of pixels vs Frequency of recording for an exemplary embodiment.

FIG. 181 illustrates an example of an individual pixel circuit.

FIG. 182 illustrates an example of stimulation simulation in SPICE.

FIG. 183 illustrates an example of recording power consumption estimation per cycle and continuous operation on different process nodes using SPICE.

FIG. 184 illustrates an example of a microcontroller that may be used in embodiments.

FIG. 185 illustrates an example of a Chip 3-D model for an exemplary embodiment.

FIG. 186 illustrates an example of Numbers of pixels vs Frequency of recording for exemplary embodiments.

FIG. 187 illustrates an example of conversion, transmission and geometrical characteristics for an exemplary embodiment.

FIG. 188 illustrates an exemplary features overview for an exemplary embodiment.

FIG. 189 illustrates an exemplary embodiment.

FIG. 190 illustrates an example of Numbers of pixels vs Frequency of recording for exemplary embodiments.

FIG. 191 illustrates an example of numbers of pixels vs Frequency of recording for an exemplary embodiment.

FIG. 192 illustrates an example of conversion, transmission and geometrical characteristics for an exemplary embodiment.

FIG. 193 illustrates an example of numbers of pixels vs Frequency of recording for an exemplary embodiment.

FIG. 194 illustrates an example of speed, size, and power parameters for exemplary embodiments.

FIG. 195 illustrates an exemplary block diagram of an ADC.

FIG. 196 illustrates an example of numbers of pixels vs Frequency of recording for an exemplary embodiment.

FIG. 197 illustrates an example of numbers of pixels vs Frequency of recording for exemplary embodiments.

FIG. 198 illustrates exemplary waveforms of Transcranial Current Stimulation (tCS) including Transcranial Direct Current Stimulation (tDCS) and Transcranial Alternating Current Stimulation (tACS).

FIG. 199 illustrates an exemplary embodiment of circuitry implementing simultaneous tDCS or tACS stimulation and EEG monitoring.

FIG. 200 illustrates an exemplary embodiment of circuitry implementing simultaneous tDCS or tACS stimulation and EEG monitoring.

FIG. 201 illustrates an exemplary embodiment of an artificial vision system.

FIG. 202 illustrates an exemplary embodiment of an artificial vision system.

FIG. 203 illustrates an exemplary embodiment of a perception robotics system.

FIG. 204 illustrates an exemplary embodiment of a perception robotics system.

FIG. 205 illustrates an exemplary embodiment of a process of perception robotics.

FIG. 206 illustrates an exemplary embodiment of a process of a sense of touch perception robotics.

FIG. 207 illustrates an exemplary embodiment of a process of a sense of sight and sound perception robotics.

FIG. 208 illustrates an exemplary embodiment of a process of a sense of smell perception robotics.

FIG. 209 illustrates an exemplary embodiment of a process of a sense of taste perception robotics.

### DETAILED DESCRIPTION

The following patent applications are incorporated herein in their entirety: U.S. Provisional App. No. 62/988,323, filed Mar. 11, 2020, U.S. Provisional App. No. 62/985,889, filed Mar. 6, 2020, U.S. Provisional App. No. 62/977,124, filed Feb. 14, 2020, U.S. Provisional App. No. 62/985,872, filed Mar. 5, 2020, U.S. Provisional App. No. 62/985,874, filed Mar. 5, 2020, U.S. application Ser. No. 16/842,964, filed Apr. 8, 2020, U.S. Provisional App. No. 62/831,575, filed Apr. 9, 2019, U.S. patent application Ser. No. 15/257,019, filed Sep. 6, 2016, U.S. patent application Ser. No. 15/431,283, filed Feb. 13, 2017, U.S. patent application Ser. No. 15/431,550, filed Feb. 13, 2017, U.S. patent application Ser. No. 15/458,179, filed Mar. 14, 2017, U.S. patent application Ser. No. 15/495,959, U.S. application Ser. No. 17/175,680, filed Feb. 14, 2021, U.S. Provisional App. No. 62/214,443, filed Sep. 4, 2015, U.S. Provisional App. No. 62/294,435, filed Feb. 12, 2016, U.S. Provisional App. No. 62/294,485, filed Feb. 12, 2016, U.S. Provisional App. No. 62/308,212, filed Mar. 14, 2016, U.S. Provisional App. No. 62/326,007, filed Apr. 22, 2016, U.S. Provisional App. No. 62/353,343, filed Jun. 22, 2016, U.S. Provisional App. No. 62/397,474, filed Sep. 21, 2016, U.S. Provisional App. No. 62/510,498, filed May 24, 2017, U.S. Provisional App. No. 62/510,519, filed May 24, 2017, U.S. Provisional App. No. 62/511,532, filed May 26, 2017, U.S. Provisional App. No. 62/515,133, filed Jun. 5, 2017, U.S. application Ser. No. 15/988,315, filed May 24, 2018, U.S. Provisional App. No. 62/534,671, filed Jul. 19, 2017, U.S. Provisional App. No. 62/560,750, filed Sep. 20, 2017, U.S. Provisional App. No. 62/588,210, filed Nov. 17, 2017, U.S. Provisional App. No. 62/658,764, filed Apr. 17, 2018, and U.S. Provisional App. No. 62/665,611, filed May 2, 2018.

Embodiments may provide the capability to provide brain monitoring and stimulation devices using parameter sets that are automatically refined during operation. The most effective way to improve a system is to increase its flexibility in relevant areas. In terms of implantable brain monitoring and stimulation devices, the challenges to the technology are achieving perfect biocompatibility and maximizing the number of channels and transition speed. The speed of the slowest component defines the speed of the system. Embodiments may provide sensing techniques, such as electrical sensing techniques, chemical sensing techniques, and optical sensing techniques. Such techniques may be invasive or non-invasive. For example, non-invasive electrical techniques may include an Electro Encephalogram (EEG), while invasive electrical techniques may include electrodes implanted in the body, such as in brain tissue. For example, chemical sensing techniques may include structures to detect neurotransmitters in vivo using, for example, fast-scan cyclic voltammetry (FSCV). For example, optical sensing techniques may include optical fibers to detect optical emissions from neural tissue. Further, optrodes may detect some or all of electrical, chemical, and optical conditions.

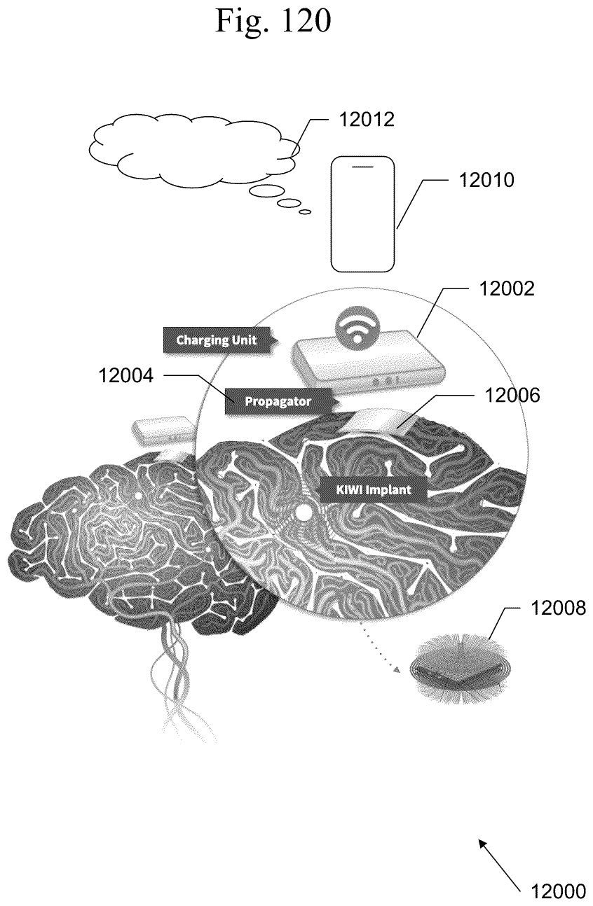

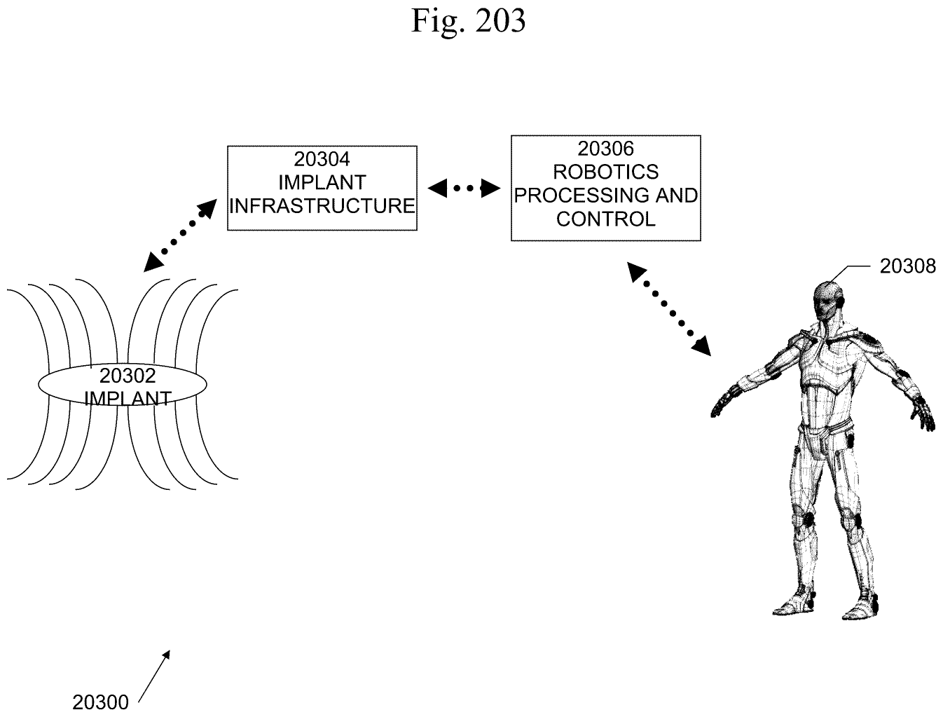

An example of a perception robotics system 20300 is shown in FIG. 203. In this example, perception robotics system 20300 includes one or more implant devices 20302, implant infrastructure 20304, perception robotics processing and control 20306, and robotic system 20308. Implant device 20302 and implant infrastructure may include the implant device and associated components described herein and shown at, for example, FIG. 120, etc. Robotics processing and control 20306 may include processors and other circuitry to interface with implant infrastructure 20304, interface with robotic system 20308, and to convert and translate between the two. Robotic system 20308 may include components such as a main body structure, actuators, sensors, local controllers, local CPU, memory, and software, that together implement a functional robotic system. Although a humanoid robotic system is shown in this example, the present techniques are applicable to any type of robotic device, such as industrial robots, surgical robots, transport robots and vehicles, etc.

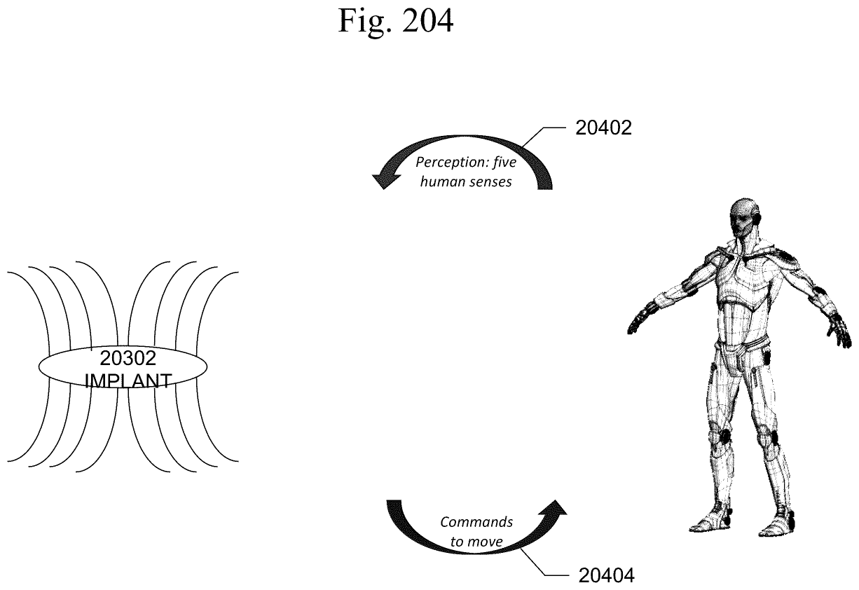

As shown in FIG. 204, datastreams representing, for example, sensor data 20402 and movement commands 20404, may be communicated by, through, and among the components as well. For example, data representing all five human senses, as well as other artificial senses, may be communicated from the robot to the implant device, as communicated and processed by the implant infrastructure and the robotics processing and control components. Likewise, movement commands may be communicated from the implant device to the robot, as communicated and processed by the implant infrastructure and the robotics processing and control components. The implant device and robotic system may operate as a closed-loop system to achieve improved precision and accuracy.

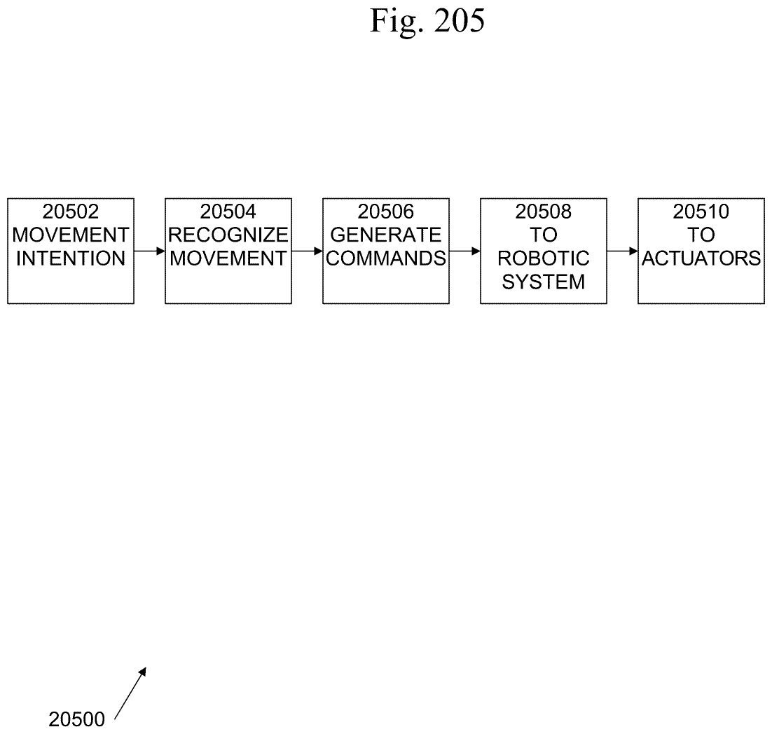

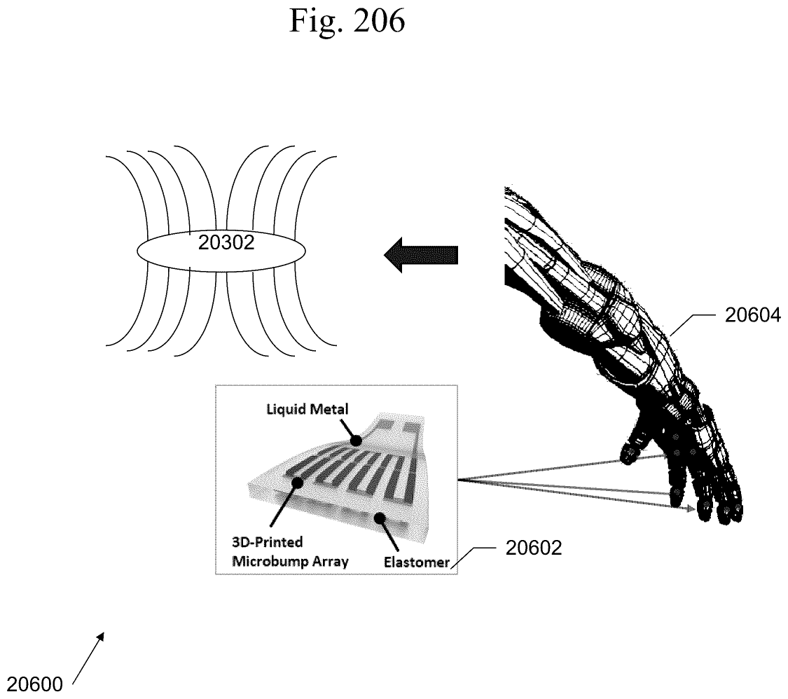

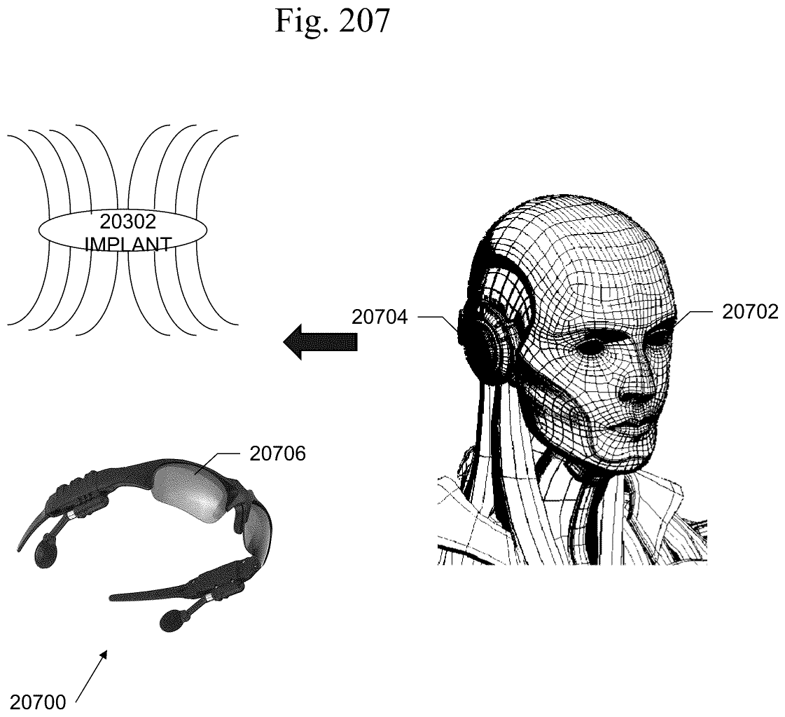

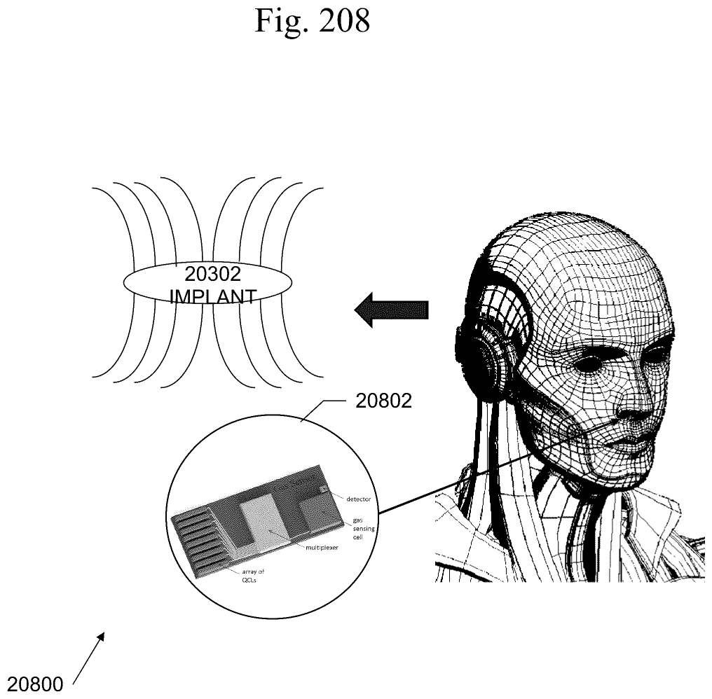

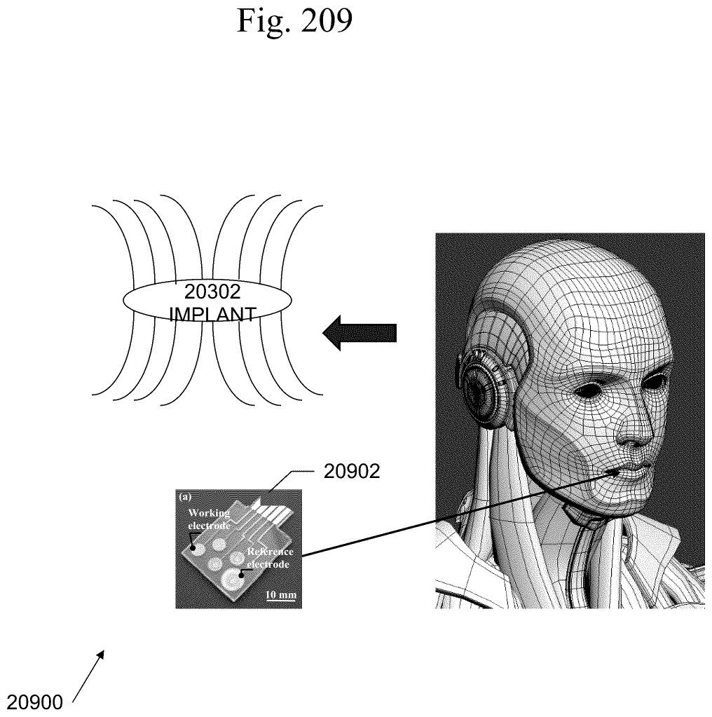

A process of operation 20500 of perception robotics system 20300 that may provide mobility of the robotic system 20308 is shown in FIG. 205. At 20502, the user's intention of the movement is recorded by the implant device and, at 20504, recognized at the implant infrastructure 20304 and/or the robotics processing and control 20306. At 20506, robotics processing and control 20306 generates movement commands for robotic system 20308. At 20508, the generated movement commands are sent to robotic system 20308, where they are decoded into control signals for actuators. At 20510, the control signals are sent to the actuators to effect movement of components of robotic system 20308. The actuators may include artificial muscles that may provide much more fluent movement than other actuator technologies, which is advantageous for a humanoid robot.

For example, the sense of touch 20600 may be provided by deploying pressure sensors 20602 at pertinent locations of robotics system 20300. For example, pressure sensors 20602 may be deployed on fingertips and palm of a robotic human hand and arm 20604. Data from pressure sensors 20602 may be communicated by, through, and among the components of system 20300, as well as processed to form stimulus signals for application to the brain of the user by the implant device 20302. The stimulus signal, when applied to the brain of the user, provide the perception of actual touch to the user.

For example, the sense of sight 20700 may be provided by deploying cameras 20702 and microphones 20704 at pertinent locations of robotics system 20300. Visual and audial information may be recorded by cameras 20702 and microphones 20704 deployed on robotics system 20300 and may be communicated by, through, and among the components of system 20300, as well as processed to form stimulus signals for application to the brain of the user by the implant device 20302. The applied stimulus signals may cause the user to directly perceive the visual information, as described herein.

Likewise, visual information may be transmitted from the robot 20300 to a headset 20704. Transmitted live video may be streamed and displayed to a user using a headset 20704 or other display device. Further, audio information may be transmitted from the robot 20300 to a headset 20704. Transmitted live audio may be streamed and displayed to a user using a headset 20704 or other audio device.

For example, the sense of smell 20800 may be provided by deploying gas detectors, such as optical gas sensors 20802, installed in a “nasal” area of robotics system 20300. Each gas has a unique response. The measured pattern of gases may be recorded by robotics system 20300 and processed to recognize the smell of the gas. An indicator of the smell may be communicated by, through, and among the components of system 20300, as well as processed to form stimulus signals for application to the brain of the user by the implant device 20302. The applied stimulus signals may cause the user to directly perceive the detected smell.

For example, the sense of taste 20900 may be provided by deploying electronic tongue technology sensors at pertinent locations of robotics system 20300. Each taste has a unique response pattern. An electronic tongue may include an array of chemical sensors with partial specificity to different chemicals and pattern recognition processing, which is capable of recognizing quantitative and qualitative compositions of simple and complex chemicals. The measured pattern of taste may be recorded by robotics system 20300 and processed to recognize the taste. An indicator of the taste may be communicated by, through, and among the components of system 20300, as well as processed to form stimulus signals for application to the brain of the user by the implant device 20302. The applied stimulus signals may cause the user to directly perceive the detected taste.

Embodiments may provide the capability for a total remote perception experience, as well as remote control of any type of robot and machinery with thoughts. Further, intellectual enhancement may be provided via brain stimulation from the implant device. Advantages may include improved accuracy and precision of robotics and machinery, reduction of job losses, remote working for all professions, minimization of casualties, development of more accurate prosthetics, capabilities to resolve the highest-difficulty tasks remotely, and reduction of reliance on AI.

Embodiments may provide the capability to provide brain monitoring and stimulation devices using parameter sets that are automatically refined during operation. The most effective way to improve a system is to increase its flexibility in relevant areas. In terms of implantable brain monitoring and stimulation devices, the challenges to the technology are achieving perfect biocompatibility and maximizing the number of channels and transition speed. The speed of the slowest component defines the speed of the system. Embodiments may provide sensing techniques, such as electrical sensing techniques, chemical sensing techniques, and optical sensing techniques. Such techniques may be invasive or non-invasive. For example, non-invasive electrical techniques may include an Electro Encephalogram (EEG), while invasive electrical techniques may include electrodes implanted in the body, such as in brain tissue. For example, chemical sensing techniques may include structures to detect neurotransmitters in vivo using, for example, fast-scan cyclic voltammetry (FSCV). For example, optical sensing techniques may include optical fibers to detect optical emissions from neural tissue. Further, optrodes may detect some or all of electrical, chemical, and optical conditions.

In embodiments, the processing module may play an essential role in the digital design of the device. For example, in an embodiment, the processor may be responsible for transferring data to Brain Code Collection System (BCCS) earbuds via a low power Bluetooth protocol and, accordingly, for reading neural electrical activity and performing electrical stimulation. Also, the processor may share the control load with an optical FPGA to successfully perform optical reading/stimulation, as described below.

For example, in an embodiment without an optical FPGA, the processor may bear a full control load over all reading/writing processes due to the absence of the optical FPGA.

Embodiments may include a Software Suite that uses, for example, the MQTT communication protocol to communicate with a Machine Learning (ML) engine. In order to successfully communicate over the MQTT protocol, a MQTT broker needs to be previously installed. For example, the Mosquito MQTT Broker may be used. In order to compile, run or modify the Software Suite, the developer needs to install, for example, the Microsoft Visual Studio Community 2017 IDE. All the extra libraries required are provided with the application. After the IDE installation, the developer can open the solution project and modify the application.

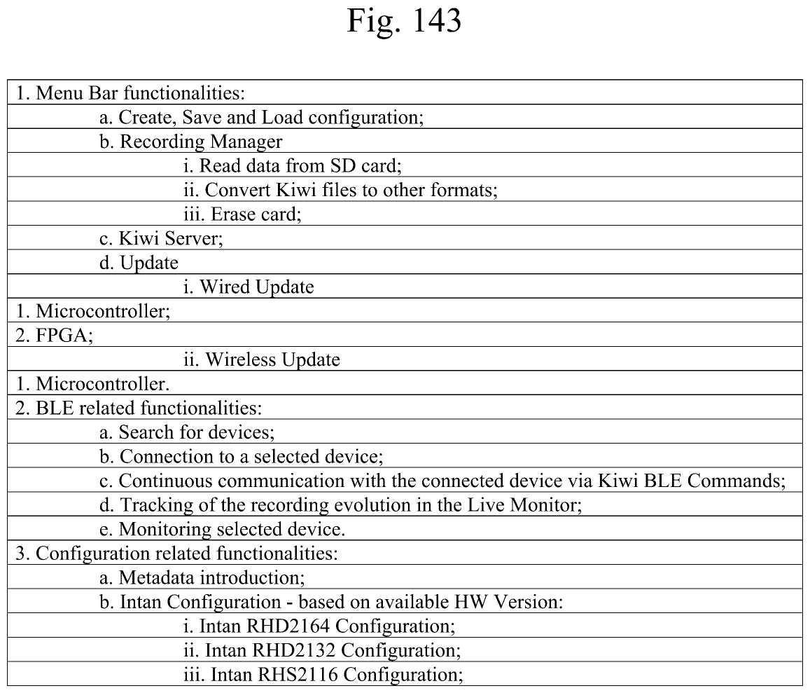

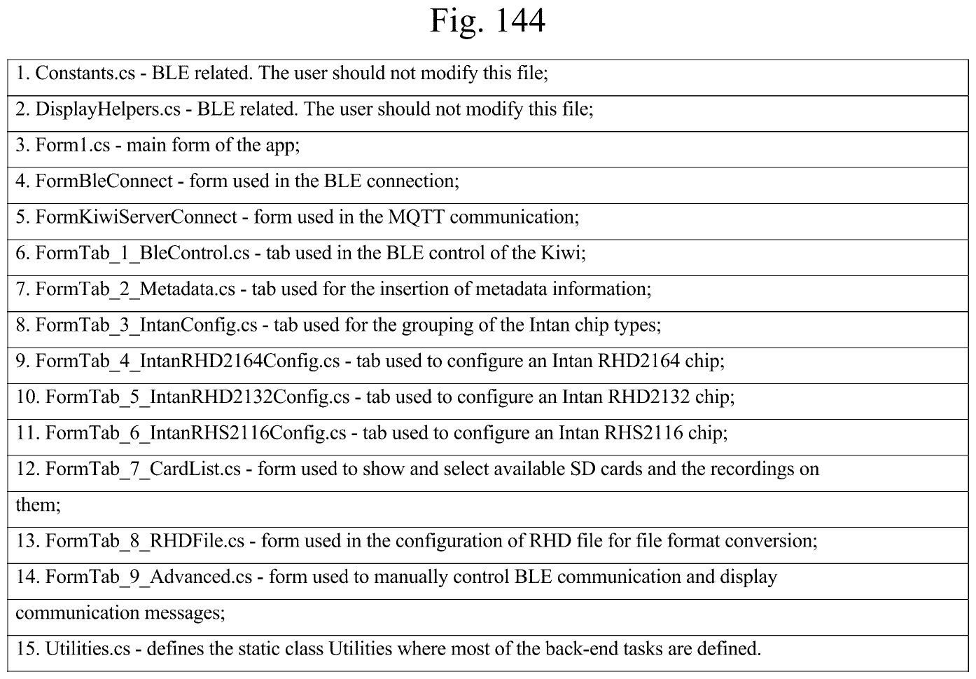

The Signal Acquisition functionality may be used to connect to a Kiwi Gateway via BLE, configure the circuitry and record biopotentials, and save raw format data on an SD card. The application allows the user to preview the signals in a user friendly way, in order to find out if the probe insertion area has significant neural activity. The Signal Acquisition functionality allows the user to easily start and track a recording experiment as well as a stimulation experiment. The application can be decomposed in 2 main functions+the menu bar functionalities, as shown in FIG. 143. The application may include the files such as those shown in FIG. 144.

The application may be multi-threaded and runs on, for example, three threads: The Main Window Thread—which takes care of all the user input and front-end interaction, the BLE Thread—which handles the BLE communication, and the MQTT Thread—which handles the MQTT communication. In order to easily manage the communication, the commands sent to the gateway (PC=>Gateway) are sent by the Main Window Thread while the responses (Gateway=>PC) are managed by the BLE Thread. All other tasks (Intan configuration, Metadata configuration, Recording Manager) are handled by the Main Window Thread. The MQTT communication per se (publish/subscribe) is handled by the MQTT Thread. Any message display from the MQTT Thread to a log for example, is made through the Invoke mechanism, because all the display elements are created on Main Window Thread.

Two types of commands can be added:—4 byte command—252 bytes command To avoid any issues with ID numbering, it is recommended that any new command is added after all the defined commands, that is, at the end of the list, using an incremental ID. For example, if the last command is 0x19, the newly added command should have the command ID 0x20; the ID should be kept the same in the firmware (see Firmware Technical Manual). The following files need to be modified to add a command: 1. FormTab_9_Advanced.Designer.cs—the Designer file associated with this form; 2. FormTab_9_Advanced.cs; 3. FormTab_1_BleControl.cs. In the file FormTab_9_Advanced.Designer.cs the developer needs to add an item to the Commands Combobox. The definition of items for this Combobox can be found in the InitializeComponent method, defined in this file, where all the UI components are defined.

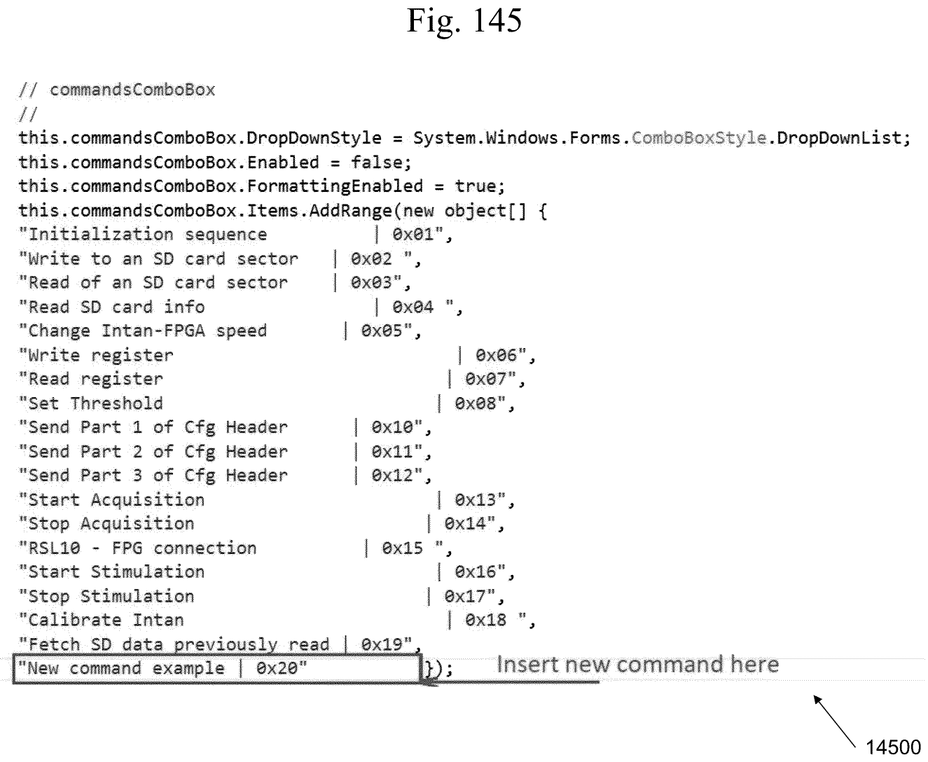

FIG. 145 illustrates an example 14500 of adding of a command to the list of available commands. The name of the command is up to the developer, but the command must be enclosed in double-quotes (“ ”), and must be separated by the previous command by a comma (,).

In the file FormTab_9_Advanced.cs, the developer needs to modify the following member functions:
1. commandsComboBox_SelectedlndexChanged;
2. parameterOTextBox_TextChanged;
3. parameter1TextBox_TextChanged;
4. loadCommandButton_Click.

In the first member function (commandsComboBox_SelectedlndexChanged), the developer needs to add a new switch case for the command and the number of parameters and their description. The number of parameters is added by toggling the Enable property of the fields for parameters. The description of the parameters is a string that describes the parameter to be entered.

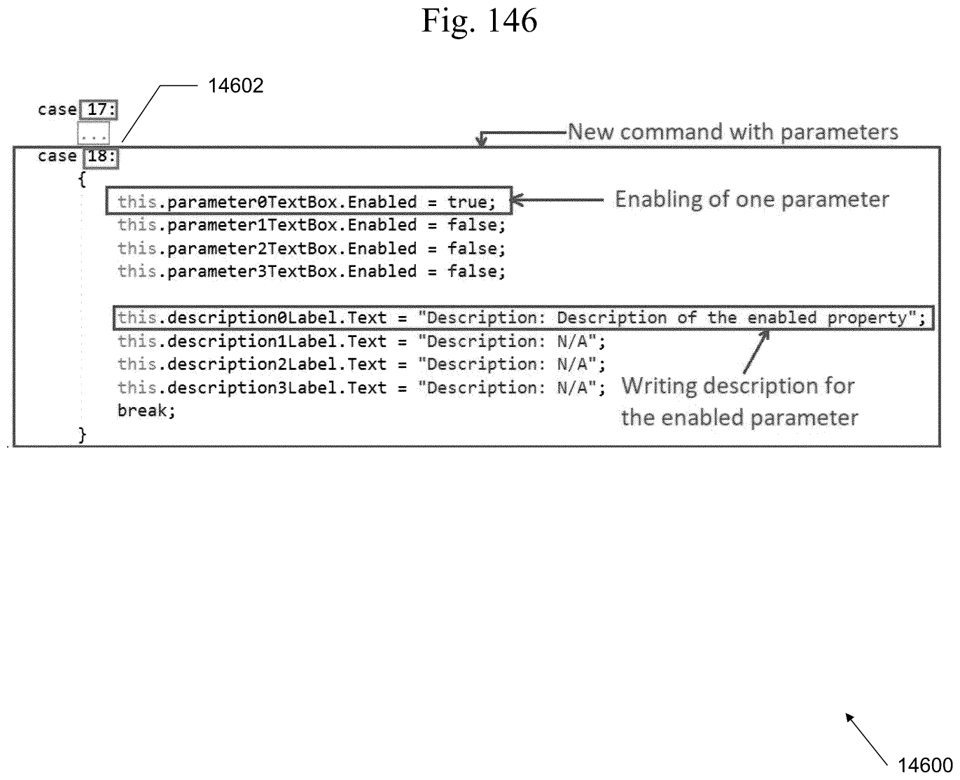

FIG. 146 illustrates an example 14600 of this process. First, observe the squares 14602, where the switch case of the new command has a value increased by one with respect to the last command. Second, observe how the enabling of parameters is made, by simply replacing the false value with true, for the selected parameter. Since a parameter is enabled, it should also have a description.

In the second and third member functions, the developer can add range conditions for the parameter 0 and 1 fields. For the moment, only one of the commands needs to check the parameters, the command 0x05. The range conditions for the commands are implemented using a switch-case construction. The developer should be aware of the fact that the switch case number value is the number of the selected index in the Commands ComboBox, with numbering starting from 0. To add new range checks for the same parameter field, the developer needs to add a switch case for the corresponding command index. To add range checks for other parameters also, the developer must define similar handler functions, below the existing ones, for the other parameter fields, i.e. parameter2TextBox_TextChanged and parameter3TextBox_TextChanged. The functions arguments remain the same. In addition, the developer must add these functions to the event handlers of the respective parameter field, in the InitializeComponent method from the FormTab_1_BleControl.Designer.cs file. Adding the handler function and the event handler is done once.

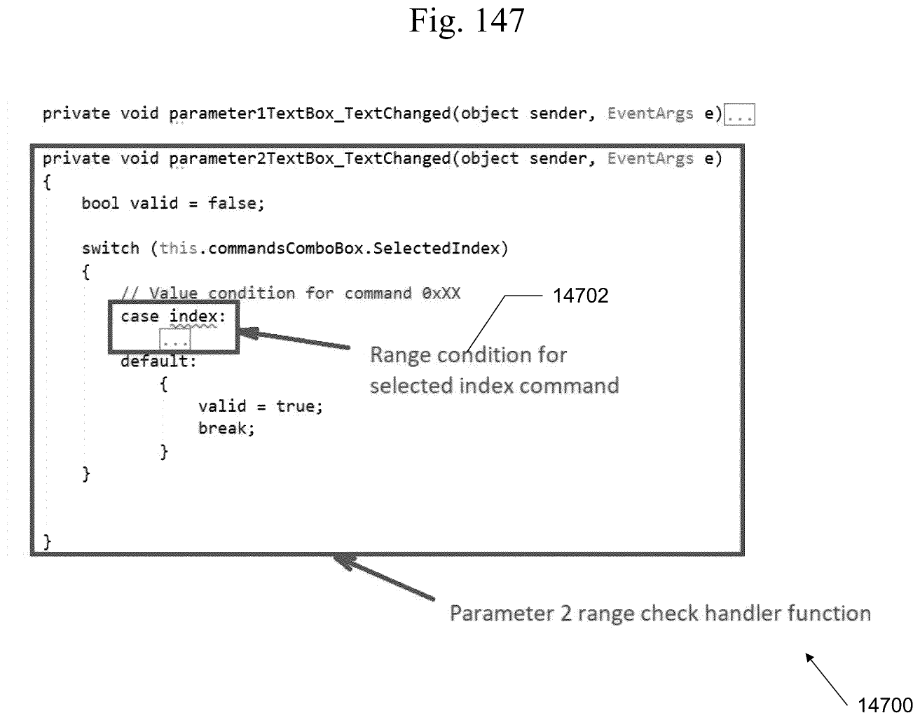

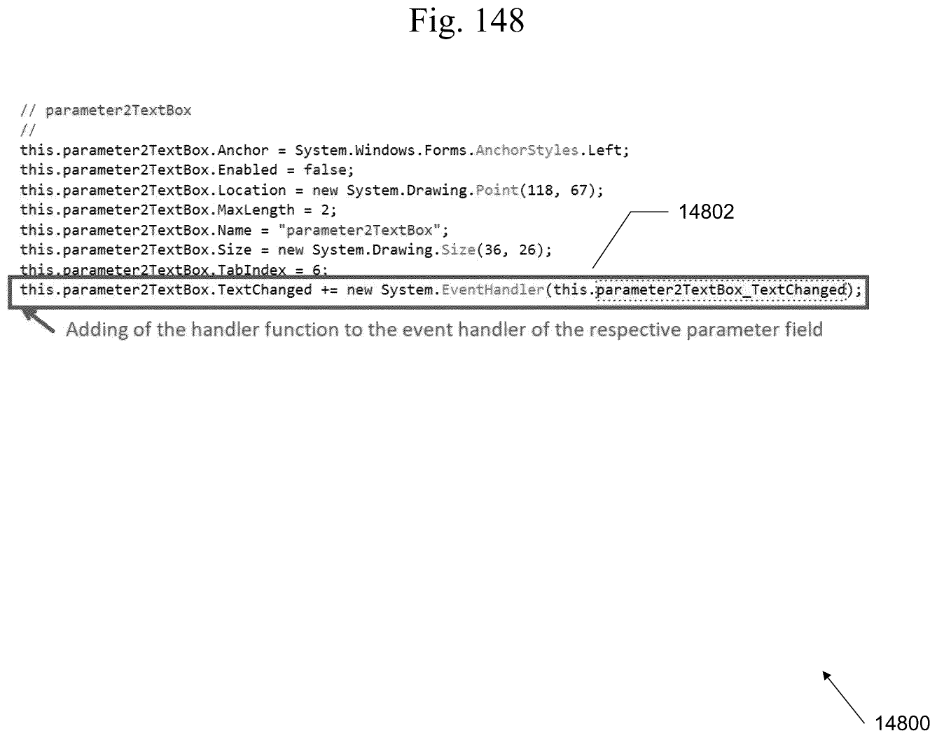

If new range checks are needed, they are added to the switch case construction. FIG. 147 illustrates an example for the case of range check for the parameter 2 field 14700. In the file FormTab_9_Advanced.cs the developer adds the handler functions with the range check conditions 14702. FIG. 148 illustrates an example 14800 of, in the file FormTab_9_Advanced.Designer.cs, the developer adds the handler function to the event handler of the respective parameter field 14802.

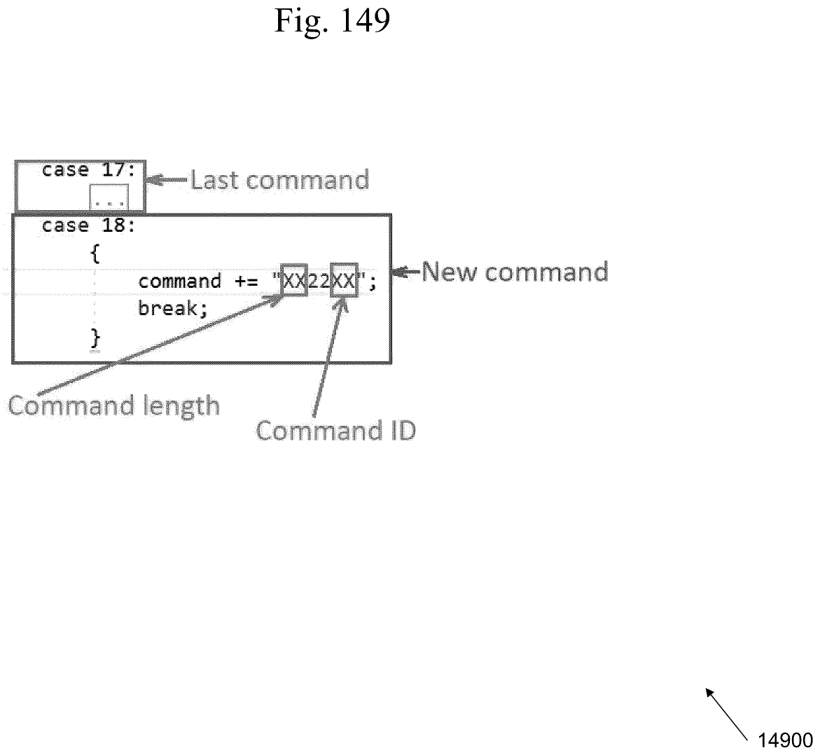

In the fourth member function (loadCommandButton_Click) the user needs to add another switch case for the command and add the command header. The command header is comprised of:—Command length, in bytes, which includes the total command length (header+parameters); —Command Group, which is always 22; —Command ID, which is the command ID added by the developer. For example, if the developer adds a new command, say 0x20, with 4 parameters, then the command header would be: 072220. Here, 07 stands for the total length (3 bytes for header and 4 bytes for parameters). FIG. 149 illustrates an example of this process 14900.

These four member functions are concerned with the input, that is, how the command is assembled and how it's parameters are enabled/disabled and checked. In the file FormTab_1_BleControl.cs the developer needs to address how the response is handled for a given command. This is done in the Process_Group_0x22 member function. Specific messages can be displayed in the LogBox. For each command, there is a corresponding response in the communication protocol. If a new command is added using the previous examples, a response should also be added. This is done in the Process_Group_0x22 member function. The developer needs to add a switch case for the command. The difference here is that the switch case number value is exactly the Command ID in hex, e.g. 0x20. The response is structured as follows: Command length, in bytes, which includes the total command length (header+parameters); Command Group, which is always 22; Command ID, which is the command ID of the command to whom the response belongs; Payload, which can take the following values: 55—command successfully sent; aa—error—any type of error; variable payload—which can be the set of parameters read (as in the case of reading sectors).

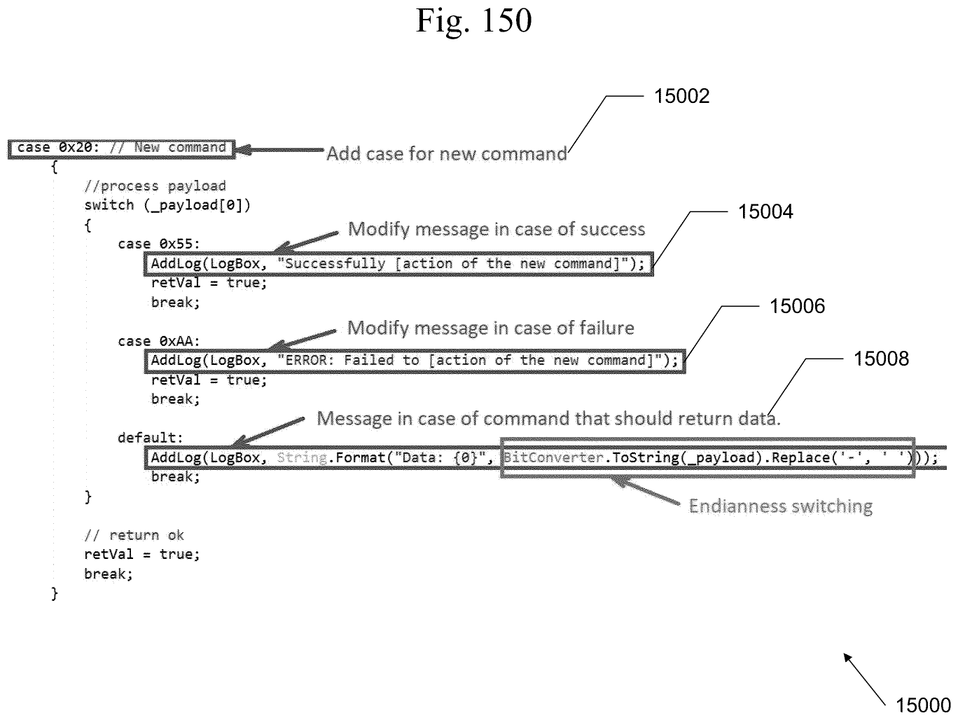

For a new command, the response should include all these 3 cases. FIG. 150 illustrates an example of this process 15000. Here, the user should add the whole switch case 15002, and modify only the switch case number, the strings for the success 1504 or failure 15006 and switch endianness if response has incorrect endianness. Nothing else should be modified. There are also commands that include more strict conditions on the response such as response length 15008 but the mechanism is similar. This is how a new command should be added. First, it is added in the list of available functions, then it's assembly, number of parameters and parameters checks are added, and finally, it's response is added.

The Recording Manager is a module that handles different file operations and SD card handling. For the moment, 3 functionalities are defined: 1. Read SD Card—a functionality that reads a recording from the SD card to a .ksd file; 2. Convert→To .rhd file—a functionality that converts a recording file (.ksd file format) to a .rhd file format; 3. Erase Card—a functionality that sends a command to the Gateway to erase the SD card without removing it from the Gateway. This section should not be modified by the developer. If the developer needs more information about the operation of this section, the PCSoftwareArchitecture document presents the mode of operation of each sub-module.

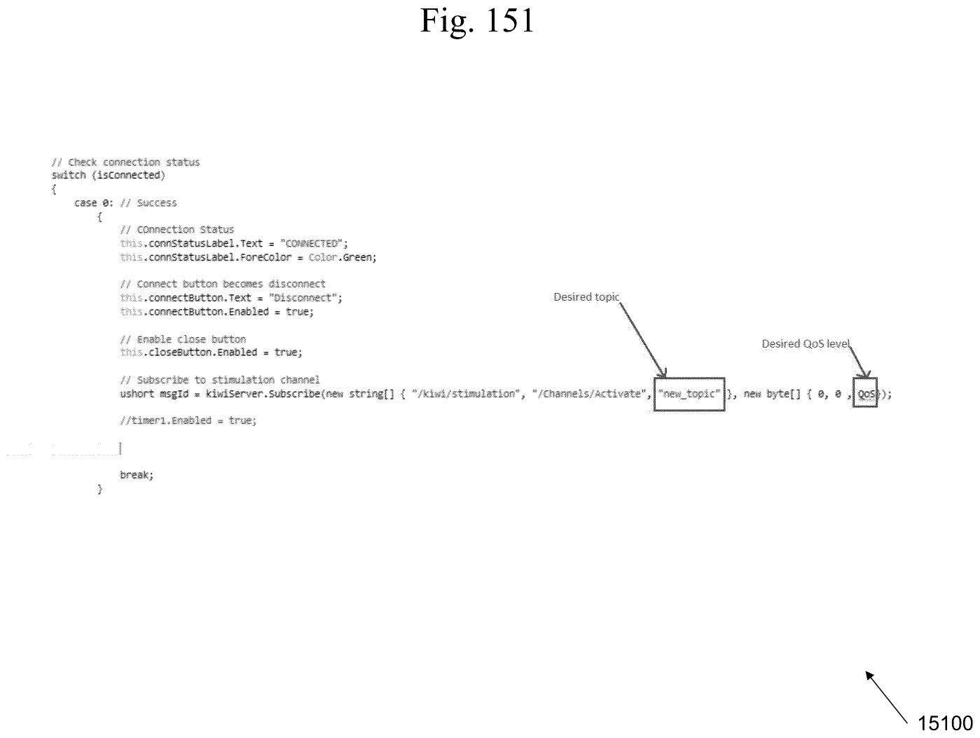

The Kiwi Server is a module that implements the MQTT communication with a broker. In this module, the developer can change the topics to which the PC Application subscribes or publishes messages. To add a topic for subscription or publishing, the developer needs to modify the FormKiwiServerConnect.cs file. To add a topic for subscription, the developer should add the desired topic inside the connectButton_Click member function. Here, inside the switch-case construction for the connection status, inside case 0 the developer can add another topic by simply adding the topic name as a string, and its QoS (Quality of Service). FIG. 151 illustrates an example of this process 15100. In order for the Application to handle a message published on a subscribed topic, the developer needs to add an if-else construction in the kiwiServer_MqttMsgPublishReceived member function. The developer can use the already implemented handlers as an example to add a handler for the newly subscribed topic. To add a new topic for publishing, the developer needs only to use the publishing function with the desired topic. This can be done in a member function for example, as in the encodeBuffer member function. Here, a buffer is processed and finally published in a topic.

The update module is used to update the code running in the Gateway, both Microcontroller and FPGA. There are 2 ways to update the Microcontroller code, wired and wireless. The wireless update is not currently implemented. The FPGA code can only be updated using a wired connection. The developer should not modify this section. For any question related to the operation of this section, the developer should consult the PCSoftwareArchitecture document.

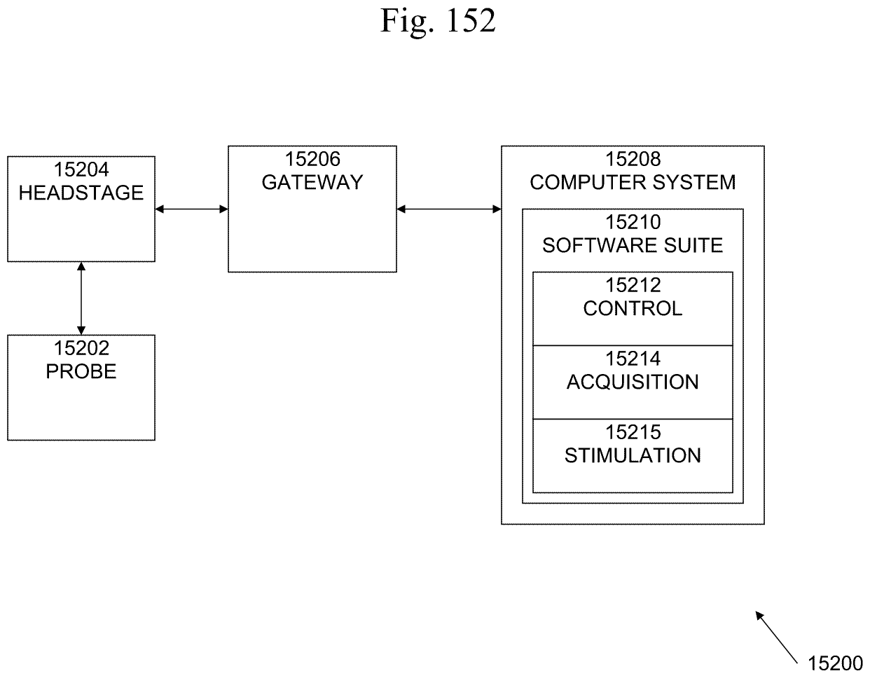

An exemplary block diagram of an embodiment of a Kiwi brain monitoring and stimulation system 15200 is shown in FIG. 152. System 15200 may include a probe 15202, a headstage 15204, a gateway 15206, and a computer system 15208. Probe 15202 may include a plurality of electrodes, optical fibers, optrodes, etc., as further described below, to acquire electrical and optical signals from neural tissue and to transmit electrical and optical signals to neural tissue. Headstage 15204 may include circuitry to provide an interface between probe 15202 and gateway 15206, as further described below. Gateway 15206 may include circuitry to provide an interface between headstage 15204 and computer system 15208, as further described below. Computer system 15208 may include software to provide an functionality such as control 15212 of system 15200, acquisition 15214 and analysis of data representing electrical and optical signals acquired from neural tissue, and generation of stimulation 15215 data to transmit electrical and optical signals to neural tissue, as further described herein.

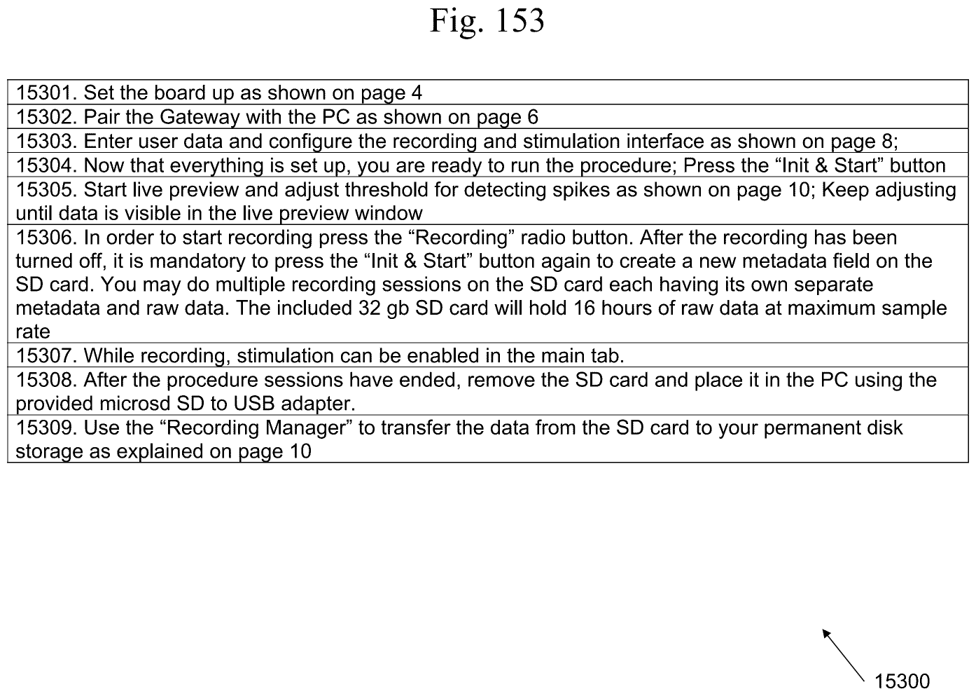

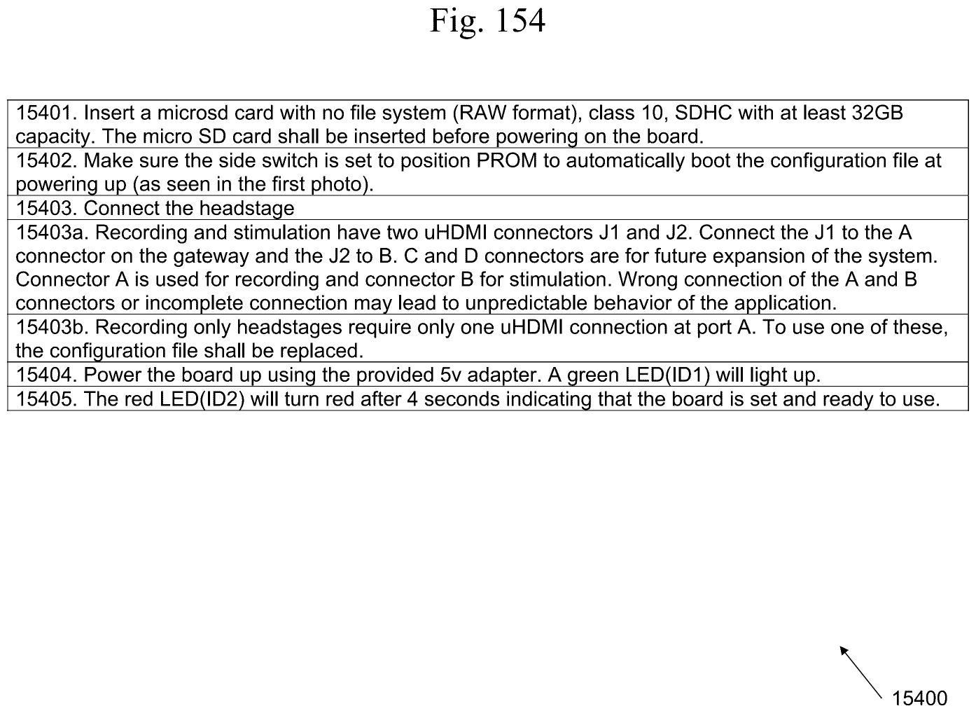

An exemplary process 15300 of operation of system 15200 is shown in FIG. 153. It is best viewed in conjunction with Process 15300 begins with 15301, in which the system is setup as shown in FIG. 154. At 15302, the Gateway may be paired with a computer, and at 15303, the software is configured. At 15304, operation of the procedure begins. At 15305, the threshold for detecting spikes may be adjusted. At 15306, recording may be started, while at 15307, stimulation may be entered. At 15308, when the procedure has ended, the memory card may be removed from system 15200 and placed in the PC and at 15309, data may be transferred from the memory card to the PC.

An exemplary process 15400 of setup of system 15200 is shown in FIG. 154.

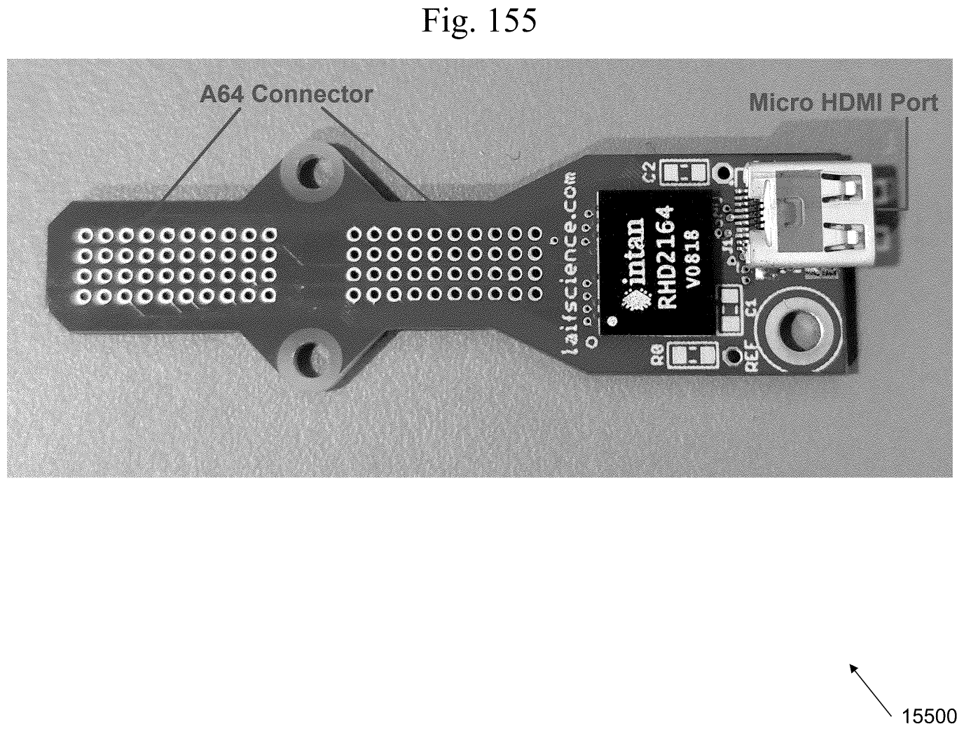

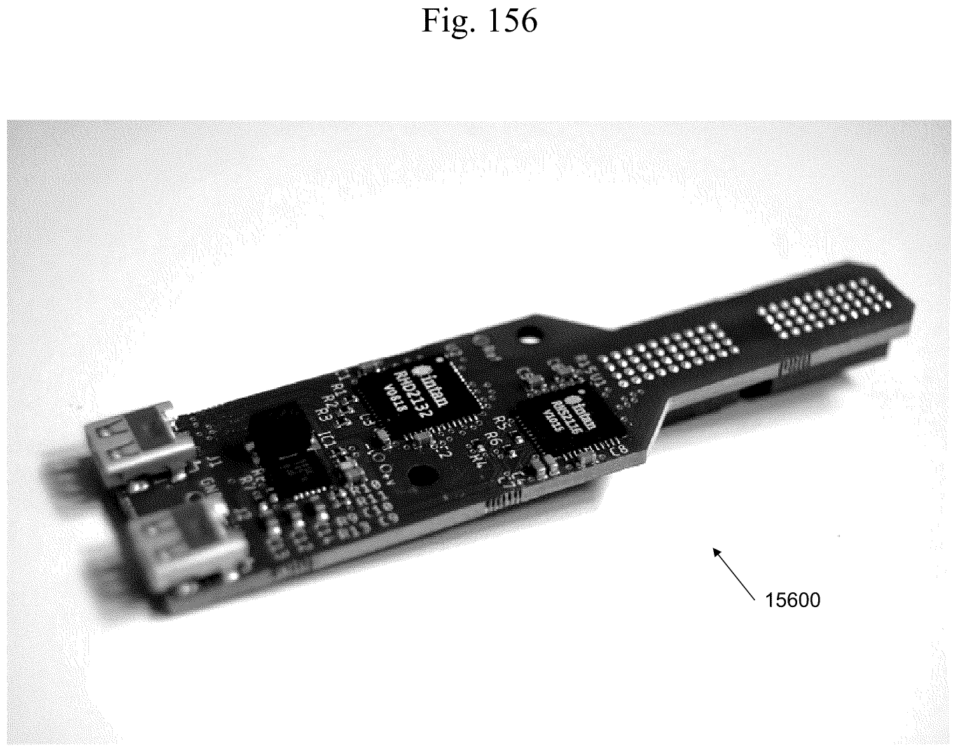

An exemplary embodiment of a headstage 15500 is shown in FIG. 155. This example illustrates a 64 channel acquisition only headstage. An exemplary embodiment of a headstage 15600 is shown in FIG. 156. This example illustrates a 32 channel acquisition and 16 channels stimulation headstage. Both headstages support 64 acute probes. After implantation of an acute probe is carried out, the acute probe may be connected using the A64 connector. Depending on the used headstage, one or two micro hdmi cables may be connected to the headstage.

Depending on the location of the electrodes on the probe, a different configuration can be implemented. This is visible in the BLE Client tab. Configurations are saved in the “settings.conf” file.

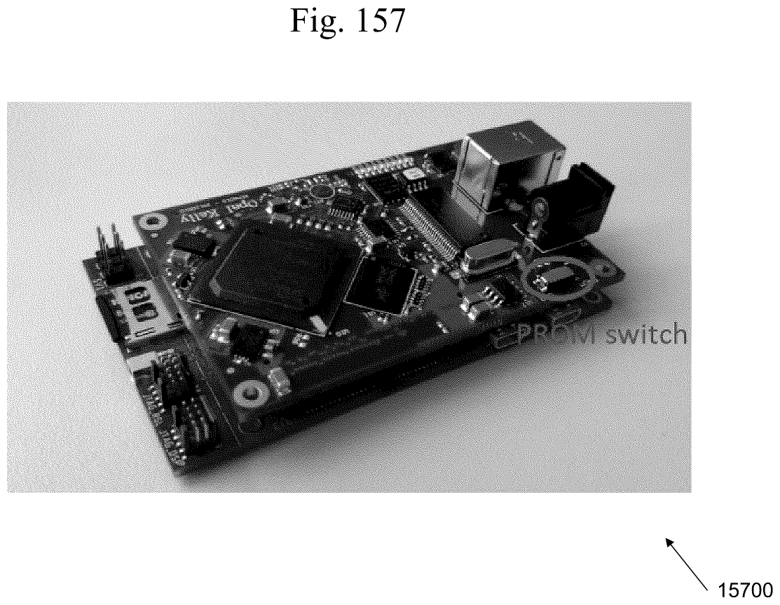

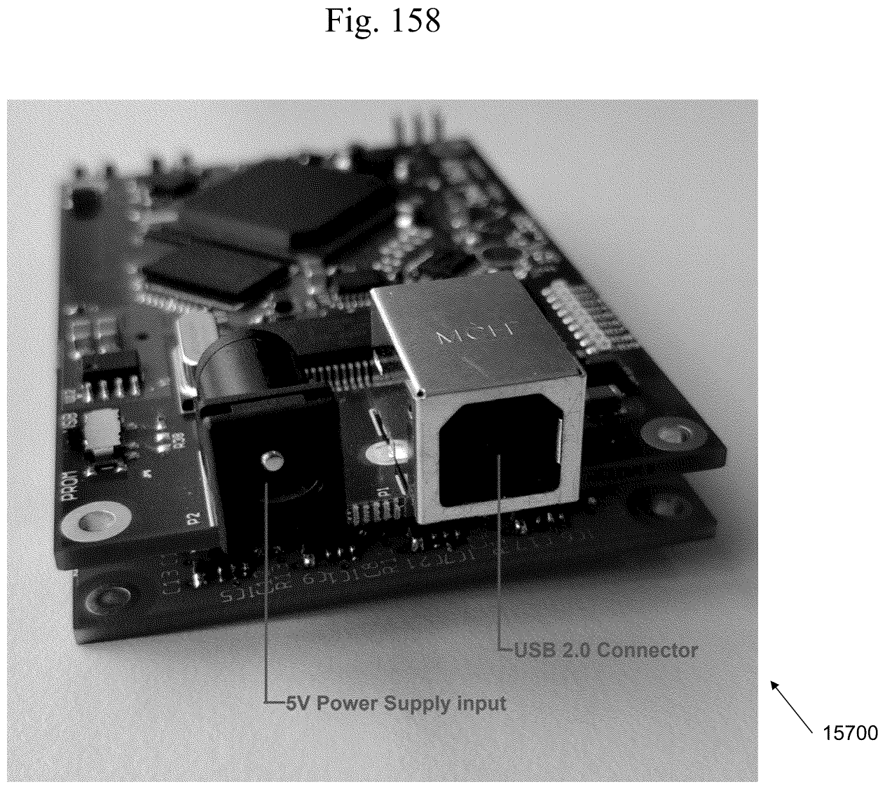

An exemplary embodiment of a gateway 15700 is shown in FIGS. 157 and 158. The gateway may be set up using process 15400, shown in FIG. 154.

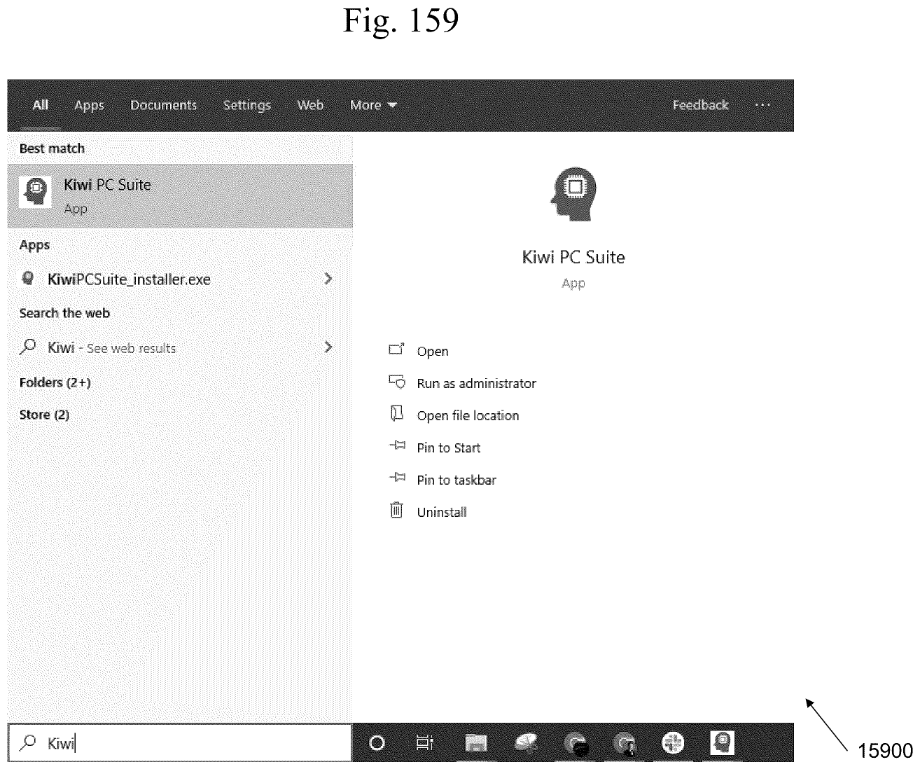

An exemplary embodiment of an initial screen display 15900 of software suite 15210, shown in FIG. 152, is shown in FIG. 159. The Kiwi Software Suite allows the user to control the Gateway wirelessly, starting and stopping acquisition, watching a live preview of the action potentials and controlling electric stimulation. In order to successfully run the Kiwi Software Suite the following prerequisites need to be installed:—MQTT Broker—the Kiwi Software Suite uses an MQTT Broker to send thresholded data to the ML Engine. For example, the Mosquitto MQTT Broker may be used.

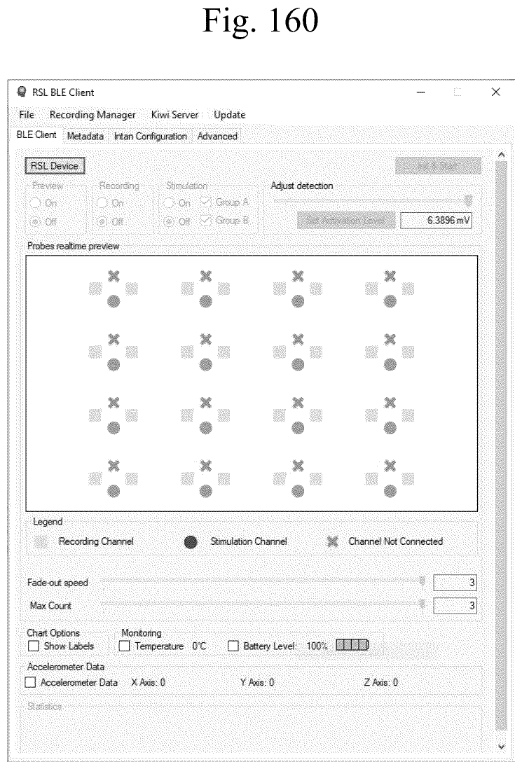

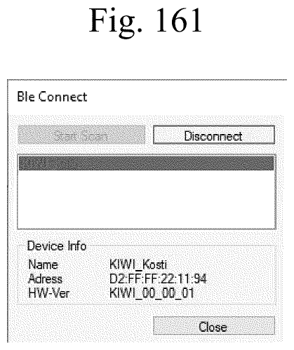

In order to connect to the gateway, the “RSL Device” button must be clicked. This opens a new dialog, which allows the discovery of nearby Bluetooth devices, as shown in FIG. 160. The name field is set in the firmware, and the address is unique to each device. The Gateway's ID may start with “KIWI_”, as shown in FIG. 161. After pressing “Connect”, the hardware version of the board should read, for example, “KIWI_XX_XX_XX”.

The Kiwi PC Software Suite receives monitoring data from the Gateway, such as temperature, battery voltage and accelerometer data. The user can select what monitoring information to receive, after a BLE Connection is made by checking the appropriate checkbox. The user can choose only one information to receive and the BLE Command decoder handles the responses and updates information only where it is available.

The Kiwi Software Suite uses an MQTT broker to send data to a ML Engine and to receive stimulation information from it. In order to use the MQTT Service (Kiwi Server), the BLE Connection between the Gateway and the PC Software must be established and the Configuration completed (see next step). After BLE Connection is established and Configuration completed, before starting recording and stimulation, the user has to connect to the Kiwi Server. For this task, the user should press the “Kiwi Server” menu item, then press the “Connect” item. A window containing the connection input (host name) and a log console is shown. Here, the user should enter the host name in the “Host Name” field, then press the “Connect” button. After the Kiwi Server connection is established, the user can see the messages from the communication in the log console. The publishing/subscribing tasks are out of reach for the user. The PC Software Suite automatically transfers data to the ML Engine and handles messages received from it. The Kiwi Server window can be closed to take other actions.

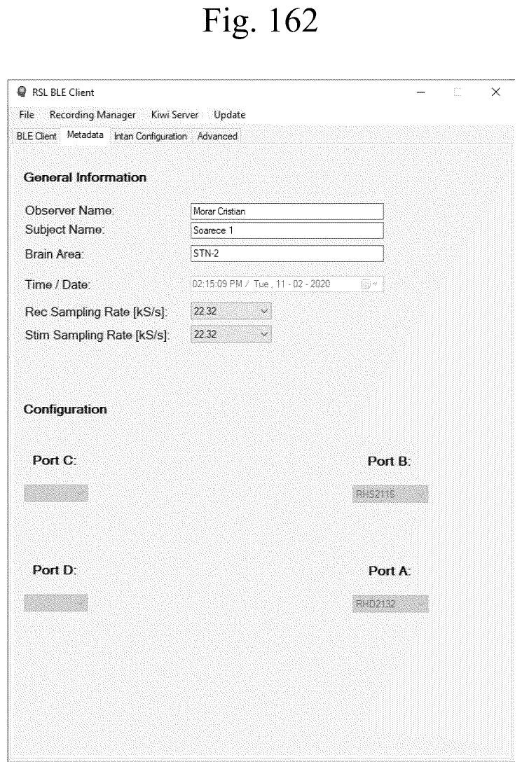

After a connection has been made, the user will notice the “Init & start” button greyed out. It will remain greyed out until procedure data (metadata and intan chip configuration) is filled and the acquisition devices are properly configured. The metadata tab, shown in FIG. 162, contains information pertaining to the current procedure. The fields which are greyed out are this way because of the current hardware configuration. The user cannot modify the connected headstages. They are automatically retrieved from the hardware version.

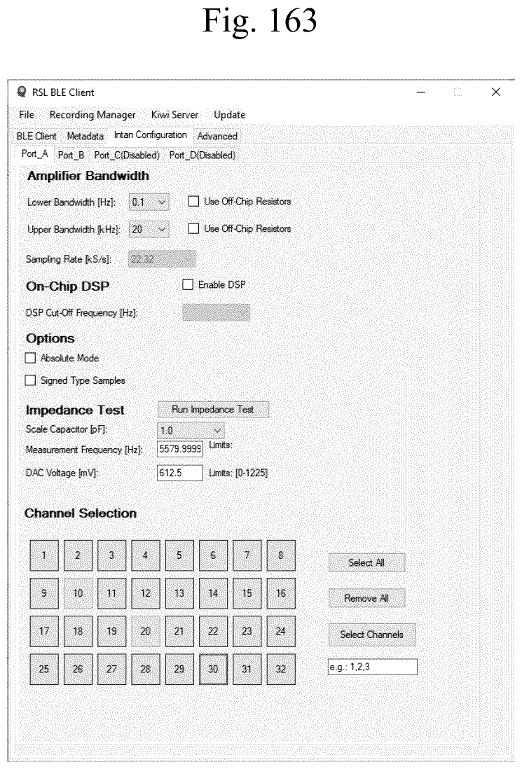

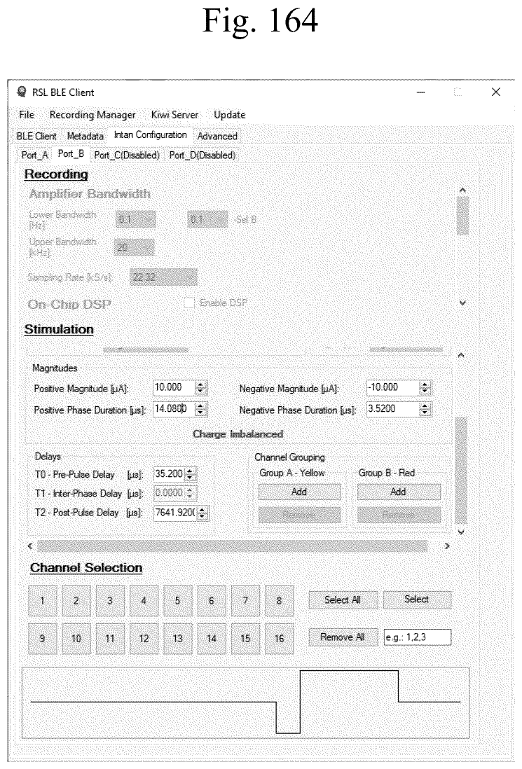

In the Intan Configuration tab, the user can configure the Intan acquisition and stimulation devices connected to the gateway. In this example, an RHD2132 is connected to Port_A, the tab for Port_A is shown in FIG. 163, and an RHS2116 is connected to Port_B, the tab for Port_B is shown in FIG. 164. While configuring the stimulation on Port_B, two groups can be defined. After selecting the relevant channels, press the Add button in the desired group. The channels will be added in the group and their respective buttons will have the color specified in the group label (Yellow for Group A and Red for Group B). Stimulation parameters are limited to obtain a total stimulation signal between 70 Hz and 130 Hz. Therefore, the Pre-Pulse Delay is limited 98.5 microseconds, as well as the Positive and Negative Phase Durations, while the Post-Pulse Delay has a range calculated such that the overall signal frequency is in the range of 70-130 Hz.

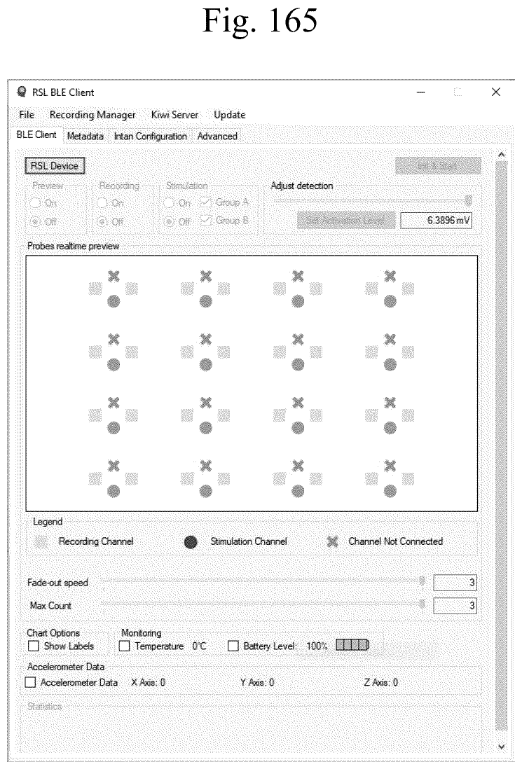

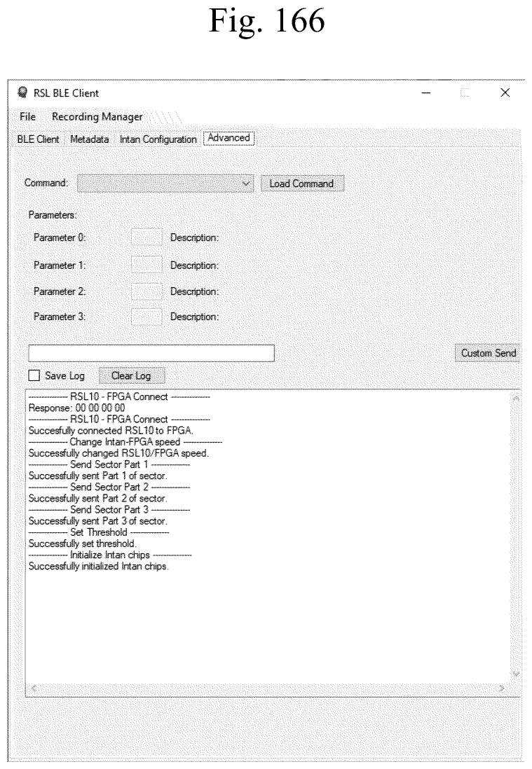

After the configuration is complete, the Init & Start button is enabled, as shown in FIGS. 165 and 166. This connects the FPGA to the microcontroller, sets the configuration of the Intan acquisition and stimulation chips, writes the configuration and metadata to the card, and sets the detection threshold. In order to check if all operations were successful, the responses are logged in the Advanced tab and, if the Save Log check box is ticked, saved to a timestamped file in the “Documents\Kiwi PC Suite\Application Log Data” folder.

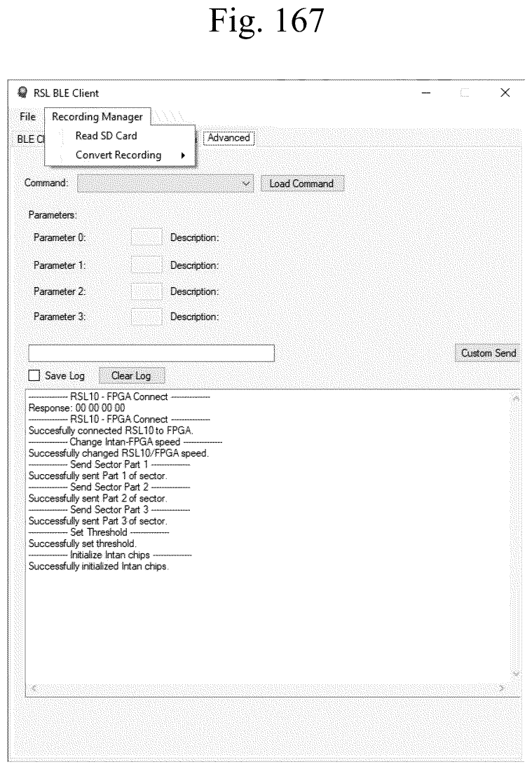

If the previous steps were executed correctly, the Recording radio button is enabled. Switching it starts a new recording on the card, as shown in FIG. 167.

The preview functionality is detecting peaks in the signal and outputting them to the Probes real time preview panel. By moving the Adjust detection slider, a new threshold value is set. The Fade-out speed slider sets the amount of time a peak is shown post detection, while the Max count slider sets a hard limit on the channel, saturating the output. This prevents the peak counter to go over a certain value. In order to display the channels and their values, the user should tick the Show Labels checkbox.

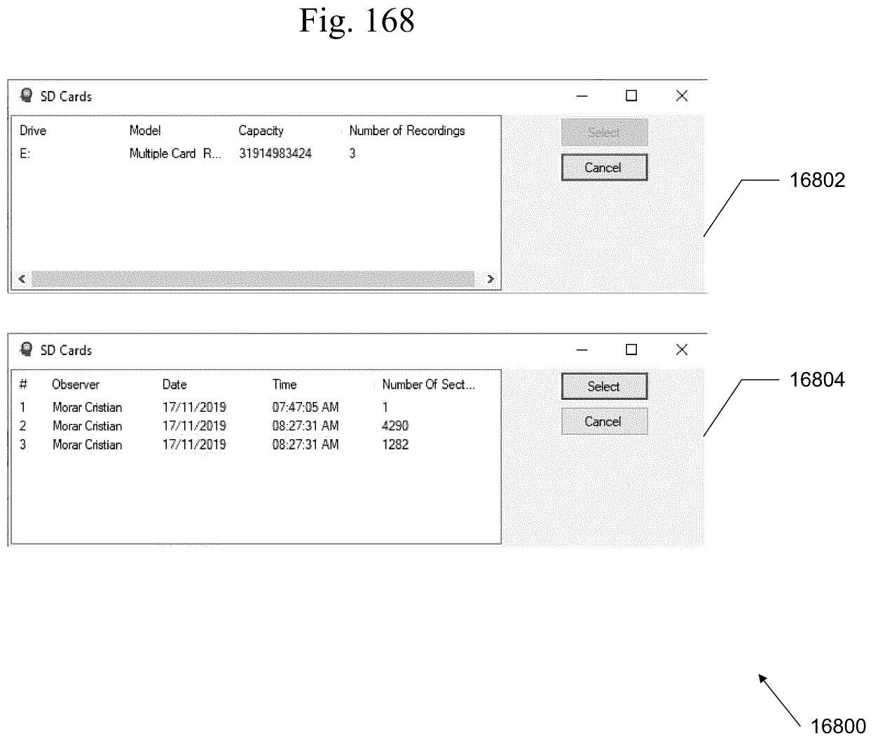

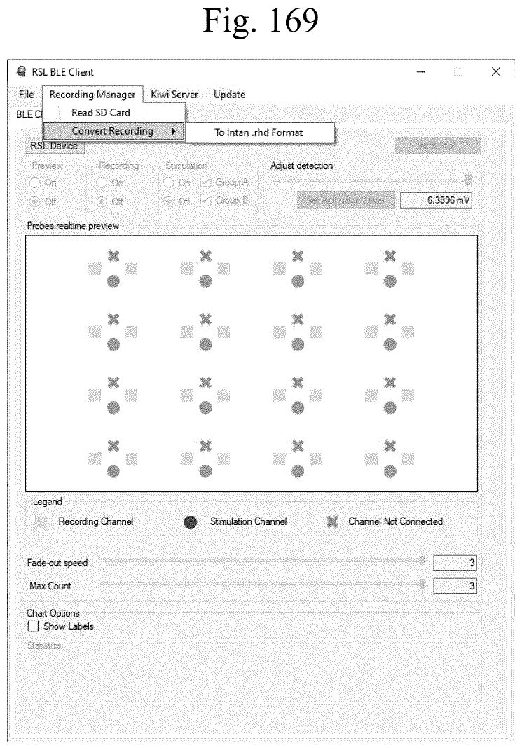

After a number of procedures are complete, the micro SD card can be removed from the Gateway and inserted into a computer. To download the experiment and recording data, use the Read SD Card functionality in the Recording Manager. An example of reading recordings 16800 is shown in FIG. 168. An exemplary display 16802 of available SD cards is shown. Selection of the SD card causes display 16804 of available recordings. In this example, 3 recordings can be seen in the list. Only the second and third recordings contain valid data, as the first recording's size (Number of Sectors) is 1. After selecting a recording from the list, press the Select button. This saves the recording to a file on the computer. As shown in FIG. 169, the recordings may be converted to various formats.

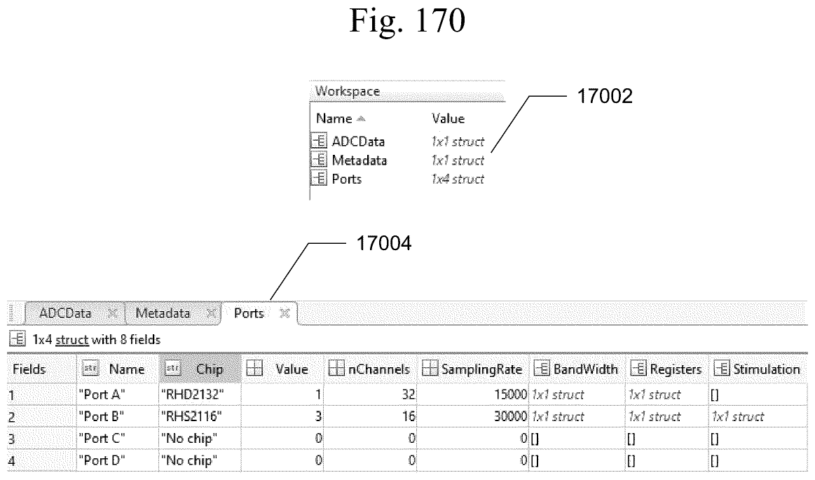

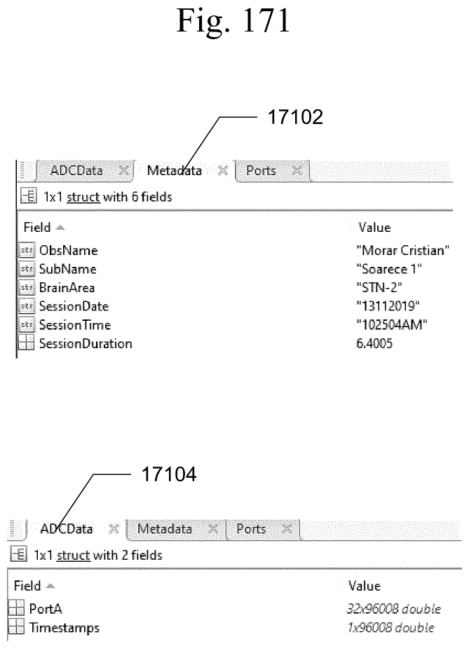

In embodiments, the obtained data may be analyzed using mathematical analysis software such as Matlab. Information used in such analyses may include Ports—a structure containing information about the ports used, number of channels for each port, bandwidth, sampling rate, etc.; Metadata—a structure containing all the metadata used in the loaded session, and ADCData—a structure containing an array for each port used for recording plus one for the timestamps, as shown at 17002 of FIG. 170. Each array contains the amplifier data from the corresponding port, with each channel on a line. Each entry in 17002 may be opened to reveal the underlying information, such as the Ports 17004, Metadata 17102, shown in FIG. 171, and ADCData 17104. Each of the underlying structures may also be opened to reveal additional information. The function also plots a figure for each port found.

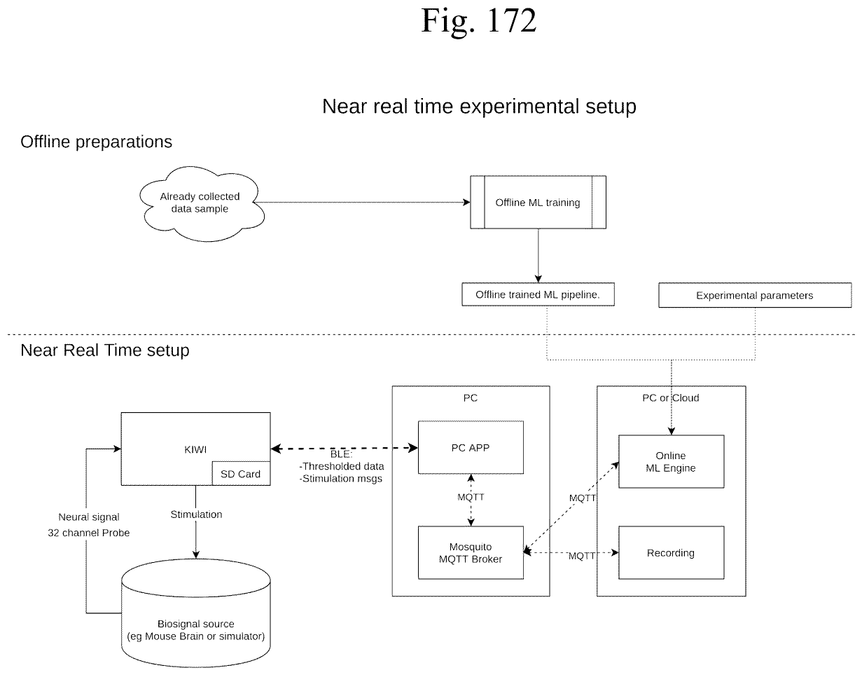

In embodiments, an Artificial Intelligence (AI) toolbox may be provided. The main goal of the library is to enable non-ML savvy neurology researcher to use ML tools and port the processing chain to the physical device. This goal is achieved by constructing a data pipeline formed by chaining preprocessing algorithms, machine learning algos and postprocessing routines. In summary, the Kiwi-ai toolbox allows the following features: Load data from a variety of sources, as long as the loader can be coded in Python. Construct a data processing pipeline to detect a biosignal. Train the data pipeline and estimate its performance. Plug this trained pipeline in a near real time experiment that, in conjunction with PC Software Suite will control the Kiwi device. The Kiwi AI toolbox can be run on the same PC as the Software Suite, on another PC or from the cloud. While the development of a new pipeline is recommended to be done under Linux, the toolbox is fairly OS agnostic and solutions are provided for non-Linux OS-es. FIG. 172 is an exemplary diagram on how the AI toolbox interacts with the rest of the components to achieve real time or near real time closed loop stimulation.

The device firmware and/or the FPGA may be updated with revised software code and configuration data, respectively.

Additional features that may be supported may include:

Update firmware to use the real time OS (Free RTOS) and be fully wireless. Battery life optimizations will be major focus.

Wireless Gateway (BLE to PC) with OTA updates and compression of data from gateway to PC.

PC to Gateway to use a publish/subscribe message protocol MQTT-sn using BLE RX/TX and MQTT using TCP/IP in PC app. A gateway/broker sets in between to convert transports and broker the subscribers with the publishers.

Infrastructure to support adaptive closed loop stimulation for battery level, temperature increase at probe, significant change in erratic motion such as at rest or sleep.

Some restructuring of FPGA to allow the addition of blocks for filtering signals, compression of raw data, power/battery management, and more structured commands that supports MQTT hierarchy, general extensibility and multiple probes (electrical & LED) types. 1

Sample at 16 bits to SD but use 10 bits plus compression for more raw data to PC app to enable analysis and real time or near real time simulation changes.

Cloud and PC data filtering and ML services to provide stimulation recipe support. Ideally some of the data filtering modules can be moved to the gateway over time.

BLE highest level of security and support for ACL with privilege levels for various user functions.

The PC app will in future releases need to support other devices beyond the gateway such as watch health data, motion, audio, etc. so a header device identifier field will be important. Likewise, we will need a method to sync time stamping to allow data correlation from multiple devices.

PC control and monitoring GUI app that is generalized to support multiple types of above probes and even other input devices such as accelerometer/gyro for motion. This app will have a consistent look and feel with the PC. App used for recorded data cleanup, tagging, and the machine learning and data correlation.

Shrink gateway to 25 mm square sized single board or smaller with piggy back battery 2032 (ideal multiple stacked in parallel for longer record/stimulate periods) with inductive charging thru skull bone.

Support for optical probes such as LED for this release.

Support for implantable probe and its associated analog to digital head stage with a focus on Parkinson's DBS.

Adaptive closed loop stimulation to automatically change based on predefined recipes in the stimulation table for the following events and include user alert messages when changed. Battery status 1. Too Low to stimulate (<xx % battery life shortened) then turn stimulation off 2. Low battery-xxx minutes of stimulation left (<yy %) then change to alternate stimulation recipe yyy per table

Probe temperature increase >z (typically 1 degree C.) then change to alternate stimulation per table or off until the temperature decreases to prior state

Motion indicates Rest or Sleep state detected then reduce to alternate stimulation recipe zz per table or turn off to save battery power.

Sufficient flash memory and persistent memory for adaptive stimulation recipes, data filtering, compression. Some of the algorithms maybe in the FPGA or RSL 10 memory.

Level 4 BLE security with Kiwi PC app using password protection. Both the PC and the Gateway support the following user privilege levels: (see attached chart page 3 of Architecture slides)

Support for 1 OOx compression of raw 10 bit data (step to Noah with no SD card)

Shrink gateway to 2032 battery diameter 25 mm×25 mm or smaller.

Include at least 3-axis low power accelerometer that can optionally be turned on and off via commands and used for significant change in motion or rest state.

Capable of supporting optical (LED) probe for stimulation with concurrent reads.

Support for implantable probe and its associated analog to digital needs focused on Parkinson's use

Regarding probes and their functions: 1. Test up to 3 probes 32 or greater channel read with 1 16/32 write capable probe (lntan compatible) −128 channels total 2. Simultaneous read on one probe while electrical stimulation on another probe further away 3. LED probe (to be specified) 4. Simultaneous read while optical stimulation 5. 1 16-32 channel write or read (lntan compatible) minimal size and connector (see lntan single chip read/write headstage minimal size) or An implantable probe with analog support typically used for DBS

Embodiments may include Record Capability per channel or groups of channels (groups may be defined in PC app) such as:

Sample at 16 bits per sample and write to SO but use 10 bits per sample with compression (100× per Noah) to send a few raw channels or selectable filtered channels to the PC over BLE for analysis. This is intended to allow for feedback changes to stimulation recipes in near real time (ideally <1 Oms per Noah requirements). This capability is the foundation for later stage full closed loop feedback.

Raw data (16 bit) stored on SO but readable from PC during recharging of battery. (10 bit compressed ok)

Spike with timestamp sent to PC over BLE. (may need to be compressed) all channels or groups of channels as selected in PC app. This likely should be shown to app user as count or calculated frequency data to allow determination of impact of a given stimulation recipe.

Pipeable filters Table in Flash (Note: filters must be pipeable and same as C.Vicas API) These are applied to raw data sent to the PC for analysis or near real time simulation changes. The table would include identifier and handle to filter for execution. Several may be selected prior to and in line with the compression algorithm. The compression algorithm is likely multiple stages of different algorithms so they probably should be treated as just another filter(s). All of these are user selectable with commands to select. The filters probable start as RSL 10 code but over time and for thru put get moved to the FPGA. We need a very extensible design for this.

Embodiments may provide Stimulation variables and ranges (TBD) such as: Voltage; Current; Frequency range; Variable pulse range; Stop and Start over defined period; Persistent Stimulation Table entries in Flash but user up-dateable: a. Default Recipe b. User selected Alternate #1 c. User selected Alternate #2 d. User selected Alternative #3 e. Low battery level recipe (may need two levels) f. Rest or Sleep level recipe (may need multiple levels) g. Reduced probe temp alternative (may be off) h. Chainable recipes i. End of table marker.

Embodiments may provide Adaptive stimulation using stored recipes. It is intended this will form the foundation for closed loop stimulation to be more fully implemented in a later release.

Adaptive closed loop stimulation changes per Stimulation table values for Battery status, Temp at probe >X degree C., Rest or Sleep detected. a. This should assume an accelerometer on the gateway or could update Motion status register from phone or watch using MQTT. b. The Temp status capability in lntan chip may be all you have now with some modeling. When we implant the gateway with the probe this will get a truer probe temp increase. c. This will need power management and battery status support.

Externally automatically turn on or off stimulation recipe on a per channel or group of specified channels basis. This will require the gateway to know which channels are active with stimulation and which table recipe is being used. Examples of how this might be used: a. PC app could control experiment by letting the stimulation run for 30 minutes and then turn off stimulation. b. PC app could pulse turning on and off for 10 sec periods. Both periods may be different. c. A person with implant and small simple control device could turn off to save power if feeling ok and then on when needing stimulation, d. A person wearing a watch which detects sleep could automatically turn off when sleeping and back on when awake. This would be a small phone or watch app using MQTT

Use external feedback supplied by control commands or motion determined by the accelerometer to modify above. This could be in the form of a PC scripting language that provides chains of commands with conditional control and looping. a. Start with motion and use persistent table entries. b. Other behaviors such as speech could use this.

Monitor spike amplitude and frequency of a group of channels and change stimulation recipe per recipe table with command changes automatically supplied by PC app or ML. As an example: a. Use table recipe 1, if frequency less than X ms over a period of time, otherwise use table recipe 2 b. Use signal data and ML and recalculate recipe and update table recipes.

Embodiments may provide Bidirectional Publish/Subscribe Messaging Using MQTT standard version 5.0 (Eclipse.org) On Gateway use MQTT-sn publish and subscribe clients (Eclipse/paho/embedded MQTT-SN C-C++ client) using BLE UART TX/RX to send data and receive commands between the PC the MQTT-sn gateway/MQTT broker (Eclipse Mosquitto broker and Eclipse gateway).

PC apps to use MQTT client libraries (Eclipse/paho/MQTT C-C++ client)

PC hosted MQTT broker will be used to handle multiple Kiwi gateways and other devices such as health data provided by phones or smart watches and will provide message routing to PC GUI apps, Cloud services, and commands to the Ver 3 Gateways. MQTT to use TCP/IP with security.

This may require structuring of commands to the gateway to better fit the MQTT hierarchy. I.e. /device/subdevice/command/ . . . MQTT-sn shortens this to a 2 or 4 octal hex value. Noah had used a 16 bit value for the command but that maybe too limiting since we want to support more probe types and chained filtering and stimulation recipes as well as other types of input devices such as watch or phone sensors.

MQTT and appropriate command support for: 1. Error handling and alert messages 2. Battery and temp status 3. Probe channels not functioning 4. OTA BLE updates to firmware or FPGA code 5. Commands (Stimulation recipes, table updates, read from SO, . . . ) 6. Buffering if BLE connection is lost for up to xx seconds or at least use the Last Will and Testament of MQTT to notify and handle. 7. Selecting 10 bit Raw signal data on given channel(s). 8. Pipeable Filter(S) and Compression selection per above. 9. Monitoring spike data 10. Gateway and PC authentication with support for user authentication modes: monitor data and alerts, change stimulation recipe(s) and appropriate tables, update firmware such as new data filters, update FPGA. Windows app may be able to use Access Control Lists provided by the OS but you will need to add enforcement on the Kiwi gateway. 11. BLE level 4 Security between BLE and gateway, SSUTSL PC to Broker or PC to cloud services. Don't wait until the end to start using this. Lots of things will likely break and using it is the best way to learn of problems. 12. Extensible to multiple types of devices (includes device code in packet header) 13. Handles command chaining or way of sending blocks of commands

Embodiments may provide a GUI app for Controlling and Monitoring experiment for Neurologist or Researcher 1. Set up experiment, pair devices, and write experiment Meta data to SO on gateway, start/stop experiment sessions (Continue to improve user experience of what you have now) 2. User can group channels to apply stimulation commands to be applied to the group instead of one channel at a time. Reading raw data would still be per channel. Example: Simulation Group A: channels 1, 3, 5, 7, 9, 11 on probe 1; Read Group channels 2, 4, 6, 8, 10, 12 on probe 1 and all channels on probe 2. 3. Per channel or group of channel spike and frequency graphics a. Sliding window ranges with coordinates b. Shows stimulation signals along with recorded data 4. Use recipe provided by Neurologist to graphically view recipe, edit, update gateway stimulation table and begin stimulation/record session 5. Use recipe provided from M L to allow Neurologist to graphically view recipe, edit and begin stimulation/record session 6. Control the stimulation based on battery status and change preconfigured closed loop cutoff points and corresponding stimulation values from the table 7. ERROR and ALERT codes and appropriate messages displayed with action taken in closed loop mode. 8. Control the motion stimulation recipe based on accelerometer or external input such as heart rate from external devices such as a watch or phone 9. Allow Neurologist to set bounds on stimulation or monitored data and if exceeded behave appropriately (this could use chaining of the stimulation recipes stored in the table on the gateway). 10. Provide graphical support for visually monitoring the experiment.

Embodiments may provide a GUI app for Data cleanup, marking and storage on PC or Cloud; Provides syncing of data from multiple devices 1. Electrical signals, 2. Accelerometer signals 3. General watch and phone health data 4. Video (future but would need time synchronization service which could be in this version due to acceleration support) 5. Speech (same as above).

Embodiments may provide a GUI app for controlling ML on data (to be expanded as C. Vicas provides more input and APIs) 1. Analysis via chained filters and ML 2. Recommended recipe for treatment which can be submitted to the control app 3. Consistent Graphical GUIs for user creating chained commands for filtering, stimulation chained stimulation, and ML analysis.

Embodiments may provide Modes such as: a. Full 32 channel EEG with SD recording 150-20000 HZ—Clinic b. Partial 8 channel EEG read for closed loop control—Clinic and consumer c. 2-4 channel electrical stimulation Clinic and consumer i. tDCS ii. tACS (CES) d. Optical stimulation thru nasal channel—Clinic i. 3-4 miniature LED from right materials 1. 370-480 wave length, red, blue, green, mixed

Embodiments may provide High Level Features/Product positioning such as: for a Clinical Version, i. High resolution EEG capability with ML analysis used for treatment of Insomnia (FDA approved),Depression (FDA approved), Pain (EU approved), and other neurologist in clinic treatments under trial ii. Electrical and Optical (Nasal) stimulation using either DC or AC pulsed or continuous currents iii. Broad assortment of software analysis, viewing and records tools using industry standards. iv. Wireless and portable with induction charging, Uses clinic's standard Windows PC or tablet for setup and monitoring v. Records to SD (128 GB) for high resolution recordings for deep analysis and records. For a Consumer Version, i. Used with Clinic setup and approve (FDA requirement) ii. Low resolution EEG capability with ML analysis used for treatment of Insomnia (FDA approved),Depression (FDA approved), Pain (EU approved) iii. Electrical Stimulation per neurologist setup (4-6) recipes stored in device based on treatment, time, current iv. Mobil Phone app to control with ML monitoring/alerts and provides these to the clinic thru internet connection

Embodiments may provide a Sensor section that provides features such as: a. EEG 32 channel soft cap b. Read sensors for 8 channel, c. Electrical Stimulation d. Optical stimulation thru nasal.

Embodiments may provide a Power/Battery ah budget such as a. Clinic 2-3×maximum treatment time plus do not discharge battery below 20% to get longer life from Lithium ION batteries. (Ideally one day of clinical treatments) b. Induction charging—Clinic c. Consumer 2×maximum treatment plus 20 d. USB C charging—Consumer e. Sensor voltage, current ranges, HZ supported.

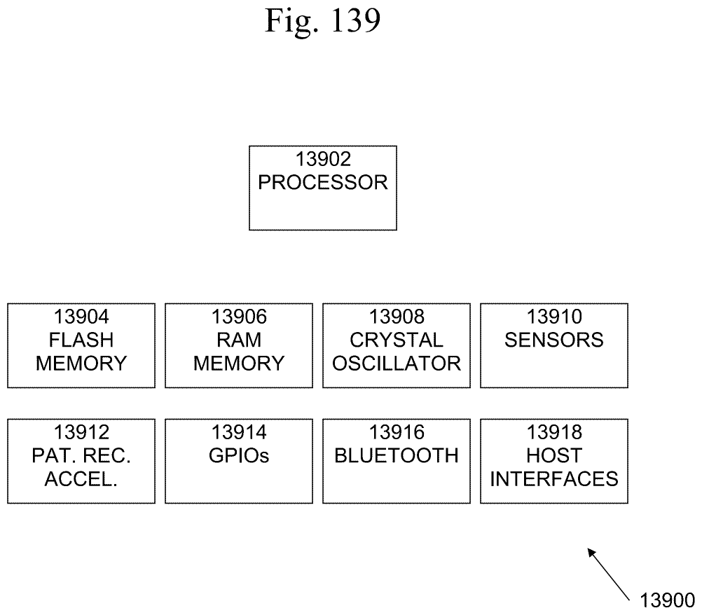

In embodiments, such as that shown in FIG. 139, the processing module 13900 may include a miniature hardware board (for example, 11.05×8.10 mm) and may include IoT technologies. In embodiments, the processing module may include a processor 13902, flash memory 13904, RAM memory 13906, a crystal oscillator 13908, and a combination of sensors 13910, pattern recognition accelerators 13912, GPIOs 13914, Bluetooth low energy 13916, and various host interfaces 13918. Even though the miniature size of the board and its broad spectrum of capabilities make it suitable for “wearing” electronics applications, there are limitations it enforces on the system performance.

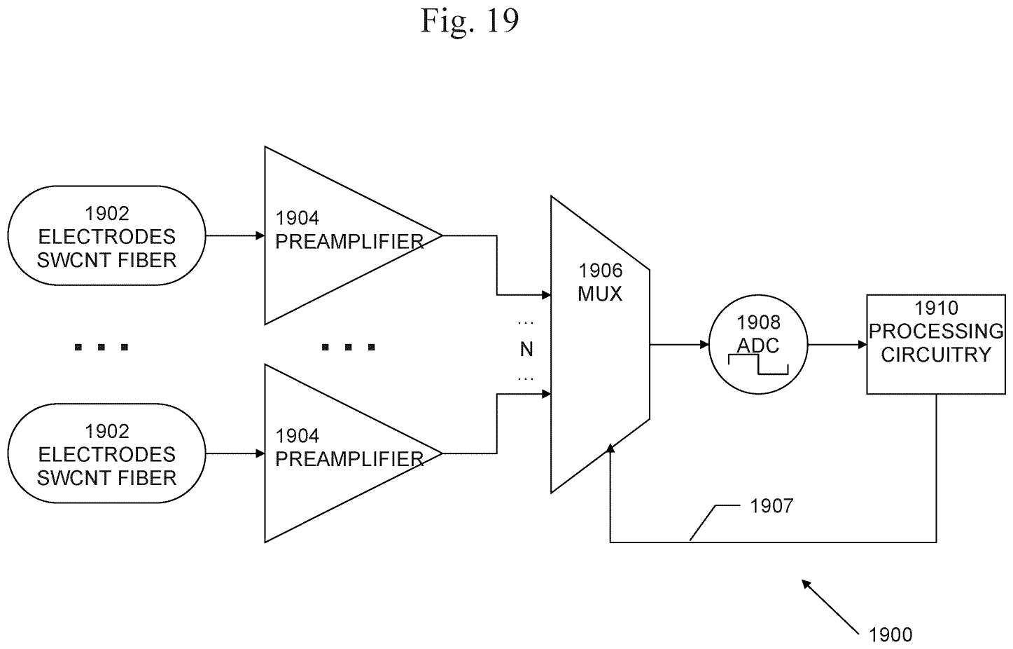

The Sampling speed limitation. Turning to FIG. 19, analog multiplexers (Mux) 1906 with N inputs may be used to latch the CMOS processor data onto an ADC 1908 in the device. The data rate of the Analogue-to-Digital ADC 1908 and dynamic parameters of analog multiplexers (Mux) 1906 determine the frame rate of the CMOS processor. Since Signal-to-Noise ratio of sampled data is proportional to the square of the number of data points, it may be desirable to maximize sampling speed.

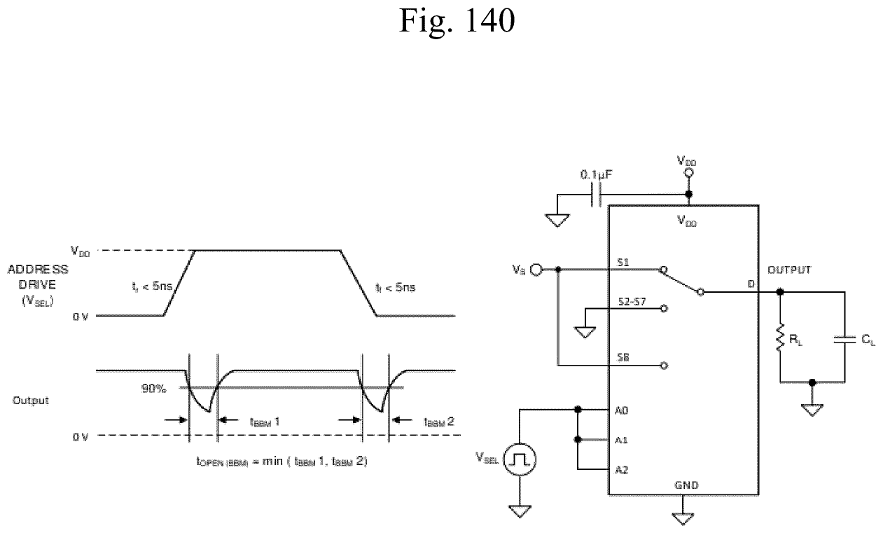

The speed limitation of a multiplexer may be determined by its dynamic parameters, for example, break-before-make delay, as shown, for example, in FIG. 140. Break-before-make delay is a safety feature that prevents two inputs from connecting when the multiplexer is commanded to switch between channels. The influence of this parameter may be demonstrated by selecting an exemplary multiplexer. Three low-voltage 8:1 input/output MUXs with bandwidth more than 20 MHz were taken as a reference. Comparing these three, one having a break before make time of 8 ns at 25° C., one having a break before make time of 16 ns at 25° C., and one having a break before make time of 1 ns at 25° C.

Ultimately, break-before-make time delay has a direct impact on the system speed. Taking the 1 ns MUX as a reference and assuming the hold-time of an average high-speed ADC to be 20 ns, it is possible to sample one of the analog inputs in 23 ns. Then, a processor that can generate a 32 MHz max output clock from its clock-out pin, in the best-case scenario (the high-speed ADC has a parallel interface), the microcontroller would need 31 ns to sample only one of the analog inputs. The clocking speed is the crucial factor in obtaining faster sampling. Longer break before make times will increase the time needed to sample each analog input.

Power consumption. In embodiments, a processing module may have a sleep mode power behavior. Typical processing modules may be designed to perform under battery-operating systems using 3.3 V or 1.8 V interfaces. An integrated power supervisor holds the system in reset state when the input voltage drops down below 2.9 Volts (for the 3.3 V interface), which emphasizes the importance of minimizing power surges in such devices. Smart application of transition between active and sleep states allows to maintain low-energy operation.

In embodiments, during the sleep mode, the core processor clock may be turned off while the Real-Time-Clock (RTC) may remain running, the sampling of data may be halted and the readout from the FIFO buffer may be disabled. In order to have a transition to the active state mode, the processor requires a wake event, such as enabled comparator, timers or RTC events. For example, the device may operate in Deep Sleep mode and have a Watch Dog Timer wake-up event every 5 seconds. Such an embodiment may, in the context of neural implants, operate with power consumption with Bluetooth low energy advertising at 100 ms and without motion sensor. The current drain may be approximately 153.67 uA, out of which 18.7 uA are drawn for maintaining the sleep state.

Due to many unknown parameters, the author strongly relies on assumptions to emphasize the necessity of possessing a considerably faster and more effective clocking routing structure. The timing mechanism of the Intel Quark SE Microcontroller is incapable of generating any output frequency due to the lack of an efficient Phase-locked loop (PLL) to satisfy potential sampling timing requirements. The Prescence of PLL can expand the possibilities of a CPU by reaching greater capabilities of sampling analogue electronics. The level of clocking routing structure is corresponding to the level of power consumption in a general System-on-Chip (SoC).

In embodiments of neural implant applications, the only time period where it will be possible to enter sleep-mode to maintain a lower power regime is the time period between making the FIFO full and transmitting the data to earbuds, implant, etc. The processor faces the obstacle of having big ratio of pixels/ADC available. Assuming that transmission speed of Bluetooth is 2-2.4 GHz and sampling of incoming data is 32 MHz, it is evident that the majority of time would be spent on sampling the data. Thus, the importance of such a sleep mode of the module for the neural implant application is not clear. An alternate embodiment may include such a processor, that has low value of draw current per clock cycles (uA/MHz) and internal generated clock without a need for a crystal oscillator (additional losses).

In embodiments, the operational principle may be based on role distributions. For example, the processor may be responsible for controlling electrical recording and stimulation, while an optical FPGA may be responsible for controlling optical recording and stimulation. In embodiments An optical FPGA may transfer the sampled optical data to the processor for a subsequent wireless transfer to earbuds. Embodiments with both the microcontroller and the optical FPGA may have increased computational flexibility, but also may have additional power consumption and increase in the physical size of the chip.

In order to minimize both power requirements and physical size, embodiments may possess multifunctional capabilities. Embodiments may utilize a CMOS matrix that will act as an executor for all recording and stimulating operations. Such a matrix would eliminate the need to use both optical FPGA and microcontroller.

Embodiments may utilize CMOS technology for electrical recording. A major advantage that CMOS image sensors enjoy over their CCD counterparts is the ability to integrate a number of processing and control functions, which lie beyond the primary task of photon collection, directly onto the sensor integrated circuit. These features generally include timing logic, exposure control, analog-to-digital conversion, shuttering, white balance, gain adjustment, and initial image processing algorithms. In order to perform all of these functions, the CMOS integrated circuit architecture more closely resembles that of a random-access memory cell rather than a simple photodiode array. The most popular CMOS designs are built around active pixel sensor (APS) technology in which both the photodiode and readout amplifier are incorporated into each pixel. This enables the charge accumulated by the photodiode to be converted into an amplified voltage inside the pixel and then transferred in sequential rows and columns to the analog signal-processing portion of the chip.

Thus, each pixel (or imaging element) may contain, in addition to a photodiode, a triad of transistors that converts accumulated electron charge to a measurable voltage, resets the photodiode, and transfers the voltage to a vertical column bus. The resulting array is an organized checkerboard of metallic readout busses that contain a photodiode and associated signal preparation circuitry at each intersection. The busses apply timing signals to the photodiodes and return readout information back to the analog decoding and processing circuitry housed away from the photodiode array. This design enables signals from each pixel in the array to be read with simple x,y addressing techniques, which is not possible with current CCD technology.

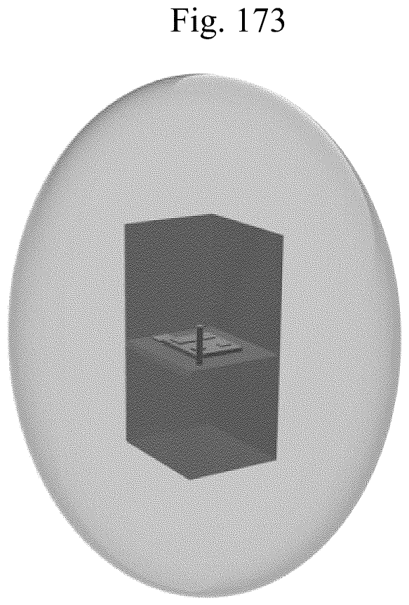

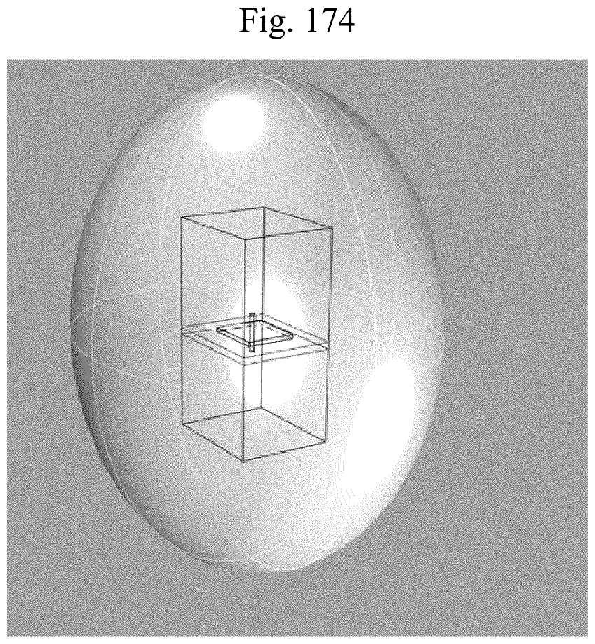

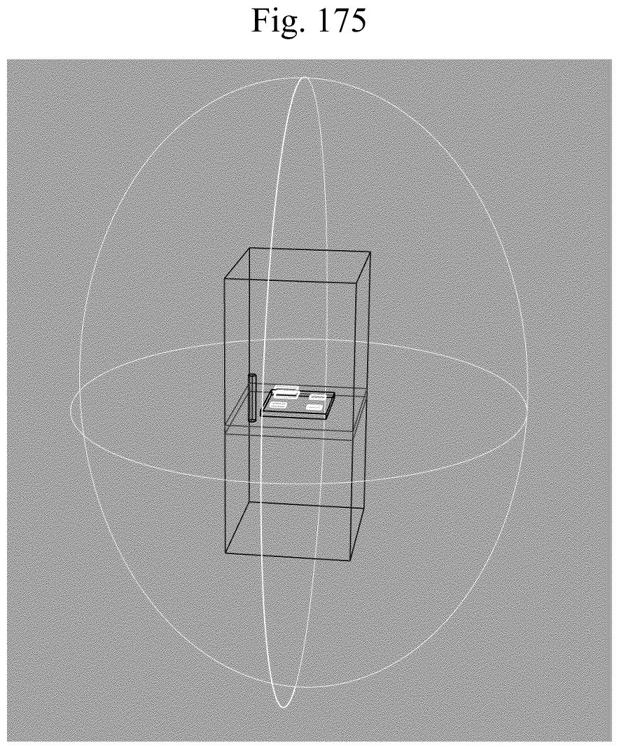

In order to minimize both power requirement and physical size, embodiments of modules may provide multifunctional capabilities. For example, embodiments may provide a cubic sensor construction locked within a spheroid with length of, for example, 2.2 mm and diameter of 1.2 mm, as shown in FIGS. 173, 174, and 175. The master electronics as well as the transmission mechanism is situated inside the sensor cube.

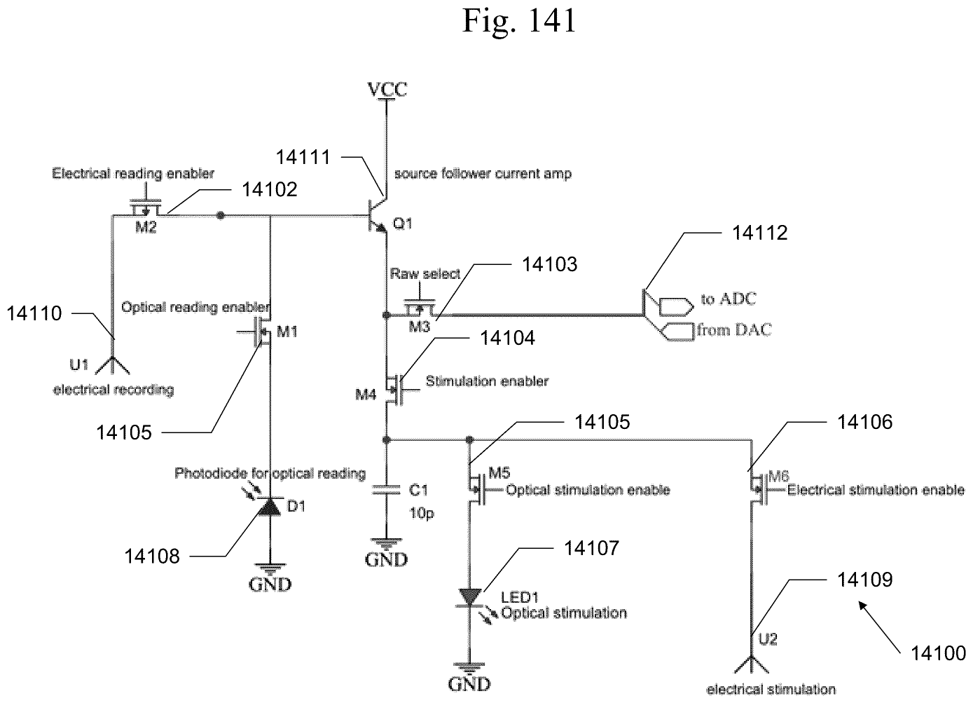

An exemplary embodiment of a multifunctional pixel or cell 14100 of a matrix or array of such multifunctional CMOS pixels or cells is shown in FIG. 141. Pixel 14100 may include a plurality of transistors 14101, 14102, 14103, 14104, 14105, 14106, one or more LEDs 14107, one or more photodiodes 14108, signal output 14109, and signal input 14110. By applying voltages to the gates of M1 14101, M2 14102, M5 14105, and M6 14106—the operation mode of pixel 14100 is selected. For example, applying a voltage to M1 14101 enables optical reading from photodiode 14108. Applying a voltage to M2 14102 enables electrical reading from signal input 14110. Applying a voltage to M3 14103 enables selection of terminal 14112 to send data to an ADC or receive data from a DAC and prepares the selected raw of pixels for the following write (from DAC) or read (to ADC) operation. Applying a voltage to M4 14104 enables a stimulation signal. Applying a voltage to M5 14105 further enables stimulation signal applied to LED 14107, which provides an optical stimulation signal. Applying a voltage to M6 14106 enables an electrical stimulation signal that is output using signal output 14109. Transistor Q1 14111 performs a follower current amplification role in the pixel. Signal output 14109 and/or signal input 14110 may be connected to electrodes, such as CNT electrodes, or other types of electrodes, while LED 14107 and/or may be optically connected to optical fibers and/or optrodes, such as CNT optrodes. Likewise, embodiments may include any suitable combination of connections of electrical and optical inputs and output to electrodes, optical fibers, optrodes, etc., as described elsewhere in this document.

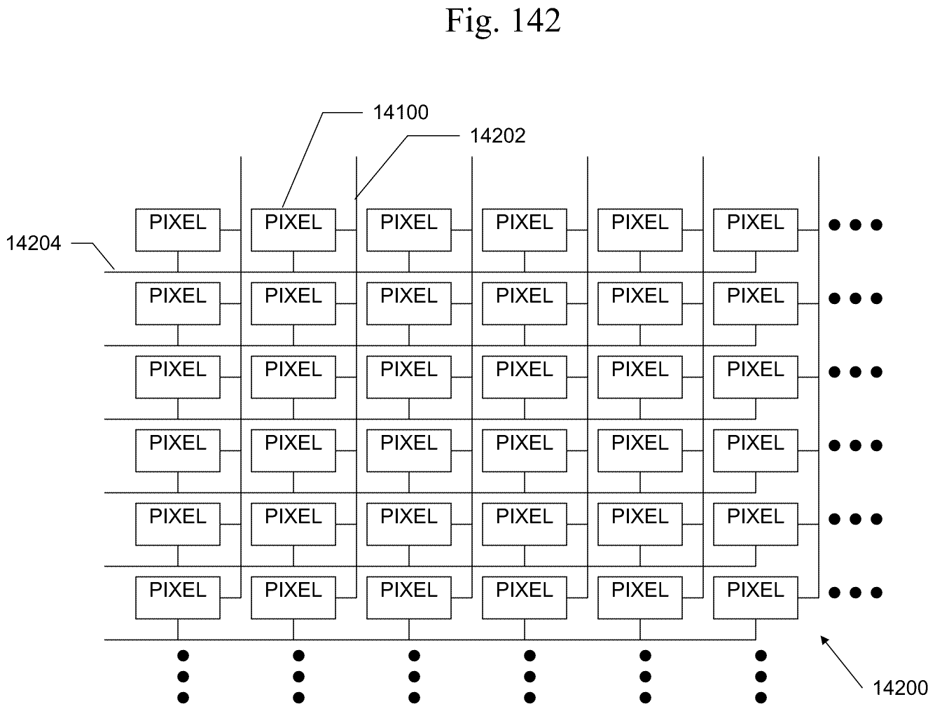

An exemplary multifunctional CMOS matrix or array 14200 is shown in FIG. 142. Array 14200 may include a plurality of multifunctional pixels 14100, such as shown in FIG. 141. Array 14200 may include lines 14202, 14204 that may include control lines to apply voltages to the gates of transistors M1 14101, M2 14102, M5 14105, and M6 14106. Array 14200 may include addressing and control circuitry to provide the capability to address and control individual pixels, selected groups of pixels, and/or all pixels in array 14200. For clarity, connections of electrical and optical inputs and output to electrodes, optical fibers, optrodes, etc. is not shown in FIG. 142.

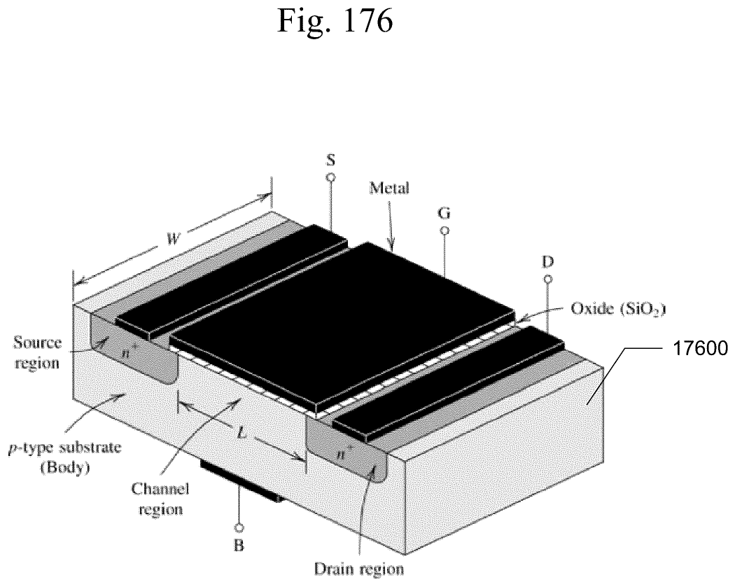

An example of a MOSFET transistor device 17600 is shown in FIG. 176. To derive power relationship of electrical and optical reading processes, the current flowing from source to drain must be derived and the structure of MOSFET must be considered. An electrical current is defined as a rate of electrical charge flow

$${I}_{d} = \frac{d q}{d t} .$$

The electrical charge, that condensates in the MOSFET's gate and in the substrate channel region, can be described as the product of capacitance of the gate-substrate channel region and the surface of the plate: q=CV. Which can be further expanded into:

$${dq}_{( x )} = \frac{{ε}_{0 x}}{{t}_{0 x}} WL ( {V}_{g s} - {V}_{( x )} - {V}_{( t )} ) .$$

Where ε₀ₓ is electrical permittivity of the SiO₂ insulator, t₀ₓ is the height of the SiO₂ insulator, W and L are width and length of the insulator respectively, and V_{gs}, V₍ₜ₎, V₍ₓ₎ are gate-source voltage difference, minimum gate threshold voltage, and voltage potential in the channel region. The latter is the function of distance x on the horizontal axis of the channel region: depending on the voltage applied at the gate, source, and drain of MOSFET, the distribution of electrical charge is varying, thus affecting the conductivity of the channel region.

After the expression of dq₍ₓ₎ is obtained,

$$\frac{1}{dt}$$

is to be defined. To find out

$$\frac{1}{dt}$$

and complete the equation of the current flowing, the process of electron flow in the channel region is observed. The speed of electron is the rate of change of distance travelled by an electron. Hence,

$$\frac{dx}{dt} = {μ}_{n} {E}_{( x )} = {μ}_{n} \frac{{dV}_{( x )}}{dx} ,$$

where μₙ is the mobility of electrons, E₍ₓ₎ is electric field strength at various point of distance, and V₍ₓ₎ voltage potential of an electron at various point of distance. Thus,

$$\frac{1}{dt} = {μ}_{n} {E}_{( x )} = {μ}_{n} \frac{{dV}_{( x )}}{{dx}^{2}} .$$

Hence, an electrical current flowing from source to drain can be derived:

$${I}_{d} = \frac{dq}{dl} = \frac{{μ}_{n}}{{dx}^{2}} \frac{{ϵ}_{ox}}{{t}_{ox}} WL ( {V}_{g s} - {V}_{( x )} - {V}_{t} ) {dV}_{( x )}$$

$${{\int }^{L}}_{0} {I}_{d} d {x}^{2} = {μ}_{n} \frac{{ϵ}_{ox}}{{t}_{ox}} * {{\int }_{0}}^{Vds} ( {V}_{g s} - {V}_{( x )} - {V}_{t} ) {dV}_{( x )} ;$$

$${I}_{d} {L}^{2} = {μ}_{n} \frac{{ϵ}_{o x}}{{t}_{ox}} W L ( ( {V}_{g s} - {V}_{( x )} ) {V}_{d s} - \frac{1}{2} {V}_{d s}^{2} )$$

Where V_{ds} is the voltage difference between drain and source gate potentials. When MOSFET is driven to the saturation region, the V_{ds} can be presented as the difference between gate-source voltage potential V_{gS} and the minimum threshold gate voltage Vₜ.

V_{ds}=V_{gs}−Vₜ

Thus,

$${I}_{d} {L}^{2} = {μ}_{n} \frac{{ϵ}_{o x}}{{t}_{ox}} WL ( ( {V}_{g s} - {V}_{t} ) ( {V}_{g s} - {V}_{t} ) — \frac{1}{2} {( {V}_{g s} - {V}_{t} )}^{2} )$$

Simplification of the above expression provides the final equation for the current flowing in the MOSFET:

$${I}_{d} = {μ}_{n} \frac{{ϵ}_{ox}}{{t}_{ox}} WL ( {( {V}_{gs} - {V}_{t} )}^{2} - \frac{1}{2} {( {V}_{gs} - {V}_{t} )}^{2} ) \\ {I}_{d} = \frac{{μ}_{n}}{2} \frac{{ϵ}_{ox}}{{t}_{ox}} \frac{W}{L} {( {V}_{gs} - {V}_{t} )}^{2}$$

For a MOSFET, this equation has the only variable—V_{gs}.

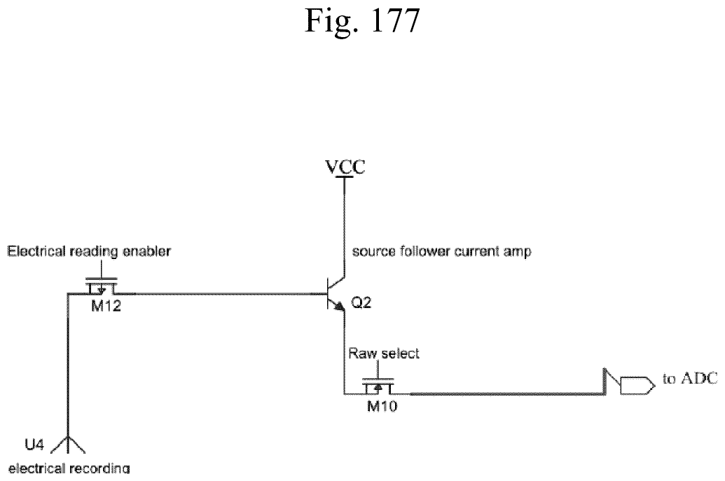

Operation of electrical and optical reading is based on the induced voltage by either an electrode (for electrical reading) or a photodiode (for an optical reading). Since the multifunctional pixel has a capability to perform one specific function at a time, the principle of superposition can be applied to derive the power required to perform the electrical reading procedure, as shown in FIG. 177, which shows an example of the superposition principle: isolated electrical recording procedure. And since the current path for both electrical recording and optical reading is same, the power consumption for each of the procedure can be derived by one equation.

As shown in FIG. 177, it is evident that when electrical reading enabler and raw selector are energized, the induced current flows through two MOSFETS and one Source follower current amplifier. Assuming that gain factor of an average transistor is from 50 to 100, most of the power consumed will be due to collector and emitter currents of the transistor source follower current amplifier.

$$\begin{matrix}{P}_{total} = {I}_{b} ( {V}_{gs 1} + {V}_{gs 2} ) + β {I}_{b} ( {V}_{BE} + {V}_{gs 2} ) = \\ = \frac{{μ}_{n}}{2} \frac{{ϵ}_{ox}}{{t}_{ox}} \frac{W}{L} {( {V}_{gs 1} - {V}_{t} )}^{2} {V}_{gs 1} + \frac{{μ}_{n}}{2} \frac{{ϵ}_{ox}}{{t}_{ox}} \frac{W}{L} {( {V}_{gs 2} - {V}_{t} )}^{2} {V}_{gs 2} ++ \\ β \frac{{μ}_{n}}{2} \frac{{ϵ}_{ox}}{{t}_{ox}} \frac{W}{L} {( {V}_{gs 2} - {V}_{t} )}^{2} ( {V}_{BE} + {V}_{gs 2} )\end{matrix}$$

Thus,

$${P}_{total} = \frac{{μ}_{n}}{2} \frac{{ϵ}_{ox}}{{t}_{ox}} \frac{W}{L} ( ( {( {V}_{gs 1} - {V}_{t} )}^{2} {V}_{gs 1} ) {V}_{gs 1} ) + {( {V}_{gs 2} - {V}_{t} )}^{2} ( 2 {V}_{gs 2} + {V}_{BE} )$$

It is evident that V_{gs1} impacts on how much power is to be consumed by the multifunctional pixel during recording an electrical activity from the brain. In case of Kiwi, such an equation indicates, that the power required during read-out depends on the voltage difference between gate and source, which is proportional to the voltage potential of the neural sampled activity. The bigger the neural activity—the more power is to be consumed during the reading-out process. Carrying reading-out power consumption is one of the drawbacks of CMOS technology: the power capabilities of a system should be carefully considered.

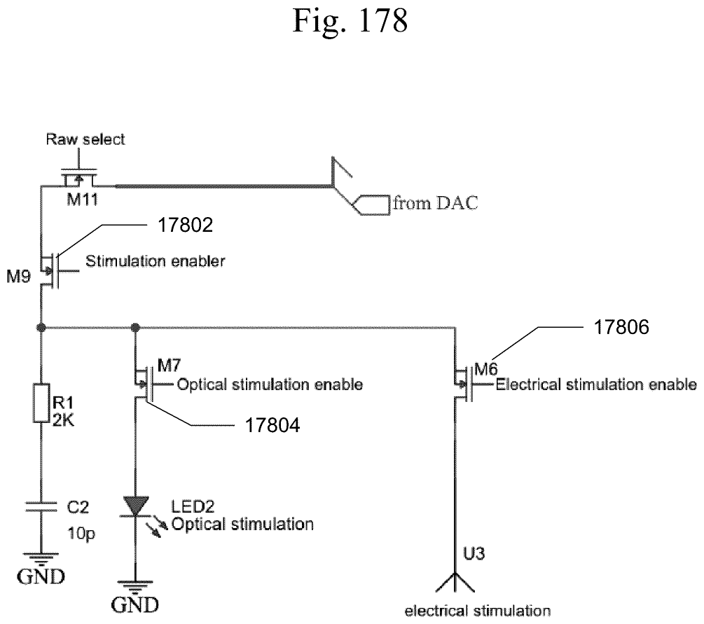

Following the superposition principle of the multifunctional pixel regarding its mutual exclusive modes of operation, the equivalent schematics for optical and electrical stimulation is shown in FIG. 178, which shows an example of the superposition principle: isolated stimulation. In order to perform the stimulation simultaneously across each and every pixel, the storage capacitor is used to temporary hold the voltage before until energizing on of the stimulation enablers. When M9 MOSFET 17802 is energized, Digital-to-Analogue converters (DACs) are dictating the voltage levels of capacitors accordingly. By energizing M7 17804 or M6 17806 MOSFETS, the operation mode (optical or electrical stimulation) is selected and the capacitor discharges through a selected line respectively. Since the schematics possesses “charge—release/stimulate” character, the power required can be calculated as the rate of change of energy of the capacitor.

$$E = \frac{1}{2} {V}_{cap}^{2}$$

Hence, the power charging the capacitor to the voltage level of DAC's output is:

$${P}_{total} = \frac{dE}{dt} = \frac{d ( \frac{1}{2} {V}_{cap}^{2} )}{dt}$$

Since the voltage across capacitor is the function of the voltage defined by the DAC VDAC, Capacitance C, Resistance R and time t, the power equation is expanded:

$$\begin{matrix}{P}_{total} = \frac{d ( \frac{1}{2} {V}_{cap}^{2} )}{dt} = \frac{d \frac{1}{2} {C ( {V}_{DAC} ( 1 - {e}^{\frac{- t}{RC}} ) )}^{2}}{dt} \\ = \frac{d \frac{1}{2} {CV}_{DAC}^{2} ( 1 - 2 {e}^{\frac{- t}{RC}} + {e}^{\frac{- t}{RC}} )}{dt}\end{matrix}$$

Solving the differential equation provides:

$${P}_{total} = \frac{1}{2} {CV}_{DAC}^{2} ( \frac{2}{RC} {e}^{\frac{- t}{RC}} - \frac{2}{RC} {e}^{\frac{- t}{RC}} ) = \frac{{V}_{DAC}^{2}}{R} ( {e}^{\frac{- t}{RC}} - {e}^{\frac{- 2 t}{RC}} )$$

Hence, the total rate of energizing the capacitor and its fluctuations depend on two variables: the voltage output updated on DAC dateline and amount of time for the charging operation. Other parameters in the equation remain constant. The power consumed is proportional to the square of the DAC voltage output, which indicates that the mismatch of power is proportional to the square of the voltage outputted by the DAC. Hence, the equation emphasizes the necessity of providing the circuitry with the DAC, which concentrates on low Integral Non-Linearity and Differential Non-Linearity errors minimizing the stimulation power errors.

Also it can be noted that

$$\frac{{V}_{DAC}^{2}}{R}$$

is the current output from the DAC, which is same at every point of the circuit when M7 17804 or M6 17806 are not energized. The current flowing from the DAC is the current flowing through MOSFET. Hence, the power equation cab be expressed via the MOSFET current M9 17802.

$${P}_{total} = \frac{{V}_{DAC}^{2}}{R} ( {e}^{\frac{- t}{RC}} - {e}^{\frac{- 2 t}{RC}} ) = {I}_{DAC}^{2} ( {e}^{\frac{- t}{RC}} - {e}^{\frac{- 2 t}{RC}} )$$

Since:

$${I}_{DAC}^{2} = {I}_{D} = \frac{{μ}_{n}}{2} \frac{{ϵ}_{ox}}{{t}_{ox}} \frac{W}{L} {( {V}_{gs} + {V}_{t} )}^{2}$$

Then,

$$\begin{matrix}{P}_{total} = {( \frac{{μ}_{n}}{2} \frac{{ϵ}_{ox}}{{t}_{ox}} \frac{W}{L} {( {V}_{gs} + {V}_{t} )}^{2} )}^{2} R ( {e}^{\frac{- t}{RC}} - {e}^{\frac{- 2 t}{RC}} ) \\ = \frac{{μ}_{n}^{2}}{4} \frac{{ϵ}_{ox}^{2}}{{t}_{ox}^{2}} \frac{{W}^{2}}{{L}^{2}} {( {V}_{gs} + {V}_{t} )}^{4} R ( {e}^{\frac{- t}{RC}} - {e}^{\frac{- 2 t}{RC}} )\end{matrix}$$

It is evident that power consumed is proportional to the 4th power degree of voltage difference between gate and source. Hence, small deviations in DAC output voltage can lead to deviation of voltage distribution across the circuitry. The smallest deviation of voltage across gate-source electrodes results into the 4th degree power consumption error. The necessity of high-performance DAC possessing high dynamic and static factors is confirmed.

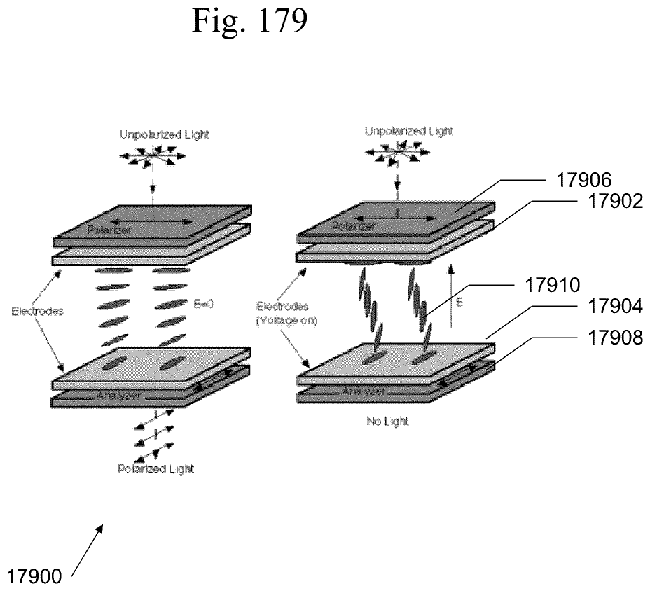

An exemplary twisted nematic device architecture 17900 is shown in FIG. 179. Twisted Nematic cell 17900 may include two bounding plates, each possessing a transparent and electrically conductive coating that performs role of an electrode 17902, 17904, a polarizer 17906, an analyzer 17908, and Nematic liquid crystal material 17910. Conventionally indium tin oxide is used for the electrically conductive coating that performs role of an electrode, yet there are various alternatives due to rapid advancement in SWCNTs.

In the last decade the improvement of technology of manufacturing semiconductors coupled with progress in neurosciences have led to the development of novel neural recording technologies. The exponential growth of simultaneously recorded neurons emphasizes the biggest challenge to implantable technologies: long-term accurate reading of big quantities of data simultaneously without damaging the neural tissue. The current recording electrode technologies are divided into 2 categories: the ones that are used for recording and stimulating, and the other ones are used for recording purposes only. The latter group's technology is rapidly increasing the numbers of available recording electrodes. It took only 2 years from developing 256-channels NeuroGrid to developing 960-channels Neuropixels and 1024-channels Polymers array. The current multifunctional electrode technologies, such as flexible polymer fibers, Silicon probe with integrated micro-LEDs, or Mesh electronics, do not possess more than 128 electrodes. However, multi-functional electrode technologies have a tendency to achieve much bigger stability.

An alternative to CMOS technology that is capable of operating with thousands of channels is the CCD-type matrix. Compared to CMOS, the CCD is lacking in terms of rate of frames due to reading the data pixel by pixel. However, CCD possesses better analytical properties: bigger dynamic range allows it to store more electrons in the p-type semiconductor thus providing a much bigger sensitivity and less overall noise. Hence, this results in more accurate frames compared to the one obtained via CMOS technology. The noiseless operation of CCD can be favored over the CMOS data readout speed when the accuracy of the technology is the priority. Sony ICX 285 is taken as general reference of CCD technology to demonstrate the reading capabilities: 1344×1024 electrode matrix is read at 20 frames per second speed. The time taken to read one frame is almost linearly proportional to the amount of recording electrodes. Hence, the CCD array would allow data to be read from thousands of channels at a frequency of tens of kilohertz. At some point in time, the state of the art will reach the point when hundreds of thousands of electrodes can be implanted. However, even in this case the choice between data quality and speed will not shift to the latter one due to an option of duplicating CCD arrays, thus creating a hybrid technology with a parallel interface while preserving lower levels of noise and bigger sensibility.

The major drawback of the CCD technology compared to the CMOS is increased power demand, which is the severe point for the Kiwi neural implant. Yet, the research is pushing for the trend of increasing the number of recording and stimulating electrodes. Despite being more power-hungry technology, CCD eliminates the need to cram multiplexers, to provide an big number of ADCs, which vastly reduce the computational load of the selected CPU and required physical size. Coupled with application of LC, the power needed for reading-out the data is reduced even further.

Despite various advantages, the majority existing neural implants' rigid electrodes are exposed to data quality worsening. The need for increasing the sampling volume leads to the inevitable challenge of scaling the electrode sizes. Reduction of an electrode's diameter leads to an increase of impedance and thermal noise, ultimately distorting the recording of signals. Coupled with the parallel interface of mostly-used CMOS technology, the additional interchannel crosstalk noise is induced, thus further corrupting the recorded data. The total data corruption therefore rises with increases in the amount of recording electrodes, thus compromising the CMOS-type neural implants' potential for a successful signal recording operation with tens and hundreds of thousands of channels. Such a challenge requires the alternative device concept to minimize noise restrictions while preserving the capability for infinite scaling of electrodes.

In comparison with rigid electrodes, the optrodes do not possess the scaling problem. Reducing the size of optrode does not influence the quality of a signal and does not degrade Signal-to-Noise ratio. The conventional way of using photodiodes is one of the potential ways of converting light to an electrical signal, however it limits the amount of possible applications down to the electronic circuit level. Novel applications of Liquid Crystal-based transducers, on the other hand, open new window of possibilities for converting the electrical neural signal into the optical at the place of contact. Liquid crystals come in different phases based on the molecules orientation as well as other factors such as temperature. The twisted nematic is the type of liquid crystals, where state of liquid crystals is formed by molecules that don't possess a specific order in their positioning yet tend to align in the same direction. When the electric field is applied to such LC, twisted nematic untwists itself altering its light-conductive properties.

Embodiments may provide a closed loop neural implant for recording and stimulating neurons. The KIWI implant is a nanoscale/miniaturized, wireless brain computer interface with novel geometric and material properties for reading and writing in the mammalian brain from layers I-VI. For example, a KIWI device may provide large scale recording and manipulating capabilities to record and stimulate neurons in a closed loop fashion. Embodiments may be able to record and modulate 10,000 neurons, at <25 m resolution, at multiple spatial and temporal scales, with 60 dB channel isolation and stimulate 10,000 neurons with optical and electrical stimulation down to layer VI of the brain.

The human brain is made up of billions of neurons connected by quadrillions of synapses using electrical, and chemical signals to communicate. Neurological conditions and psychiatric disorders are a result of disrupted networks or misfiring neurons. Being able to record large amounts of neurons and simultaneously stimulate specific neurons would open new frontiers in neuroscience research and clinical applications. However, scientists have had limited access to recording neurons due to deficiencies in electrodes and brain interface technologies.

Embodiments may provide a closed-loop brain computer interface (BCI) that is capable of recording large amounts of data from neurons and modulating specific neurons with electrical and optical stimulation. Embodiments may provide a closed loop, minimally invasive KIWI for humans capable of recording 1,000,000 neurons and stimulating 1,000,000 neurons in real time.

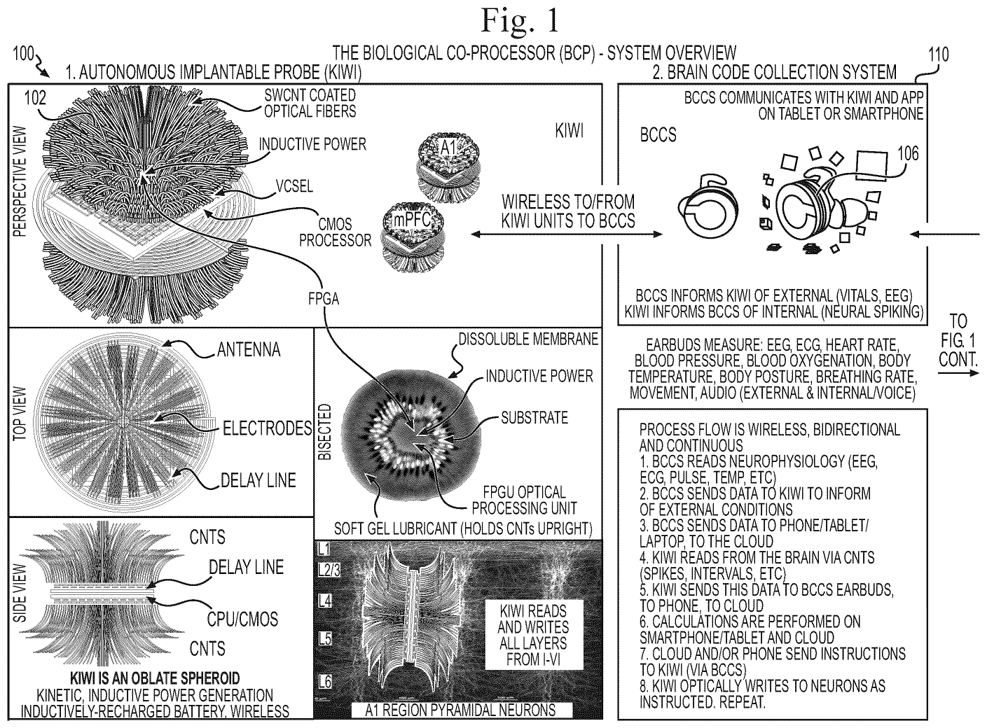

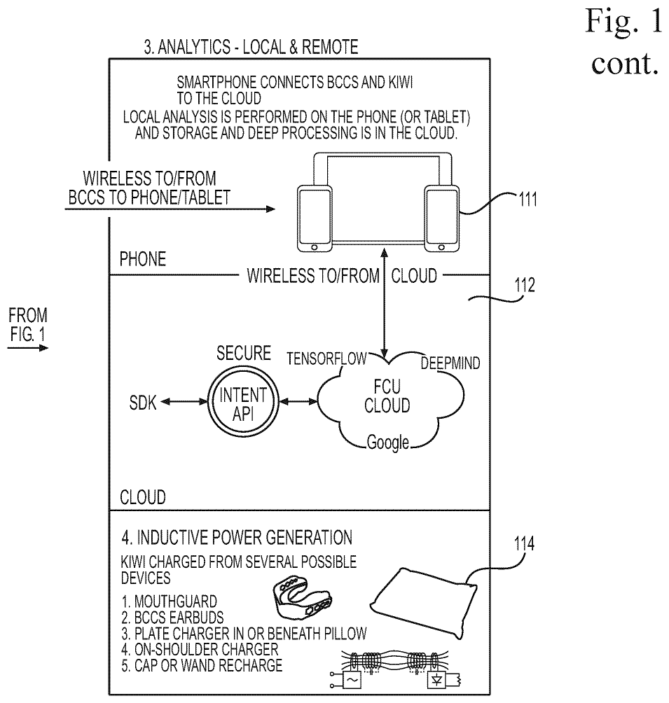

Embodiments may provide an embedded AI processor, optical neurostimulation capabilities and electrical recording capabilities. Embodiments may provide two types of microfabricated carbon nanotube (CNT) neural interfaces, a processor unit for radio transmission and I/O, a light modulation and detection silicon photonic chip, an inductive coil for remote power transfer and an independent receiver system, where the signal processing will preside. Each implant may be an oblate spheroid, a design inspired by the radial characteristics of a KIWI fruit. In the center of the implant is a nucleus surrounded by a fleshy membrane (see bisected view, FIG. 1). The nucleus may house the primary processing unit, including a low power CPU for local preprocessing, read and write instructions, the modulation scheme, and an optical FPGA dedicated for real time optical modulation. It may also contain a CMOS dedicated integrated front-end circuit developed for a pre-amplification and multiplexing of the neural signals recorded, 4G-MM for offline storage, wireless transceiver, inductive power receiver, and an optical modulation unit. Covering the nucleus may be 10,000 to 1 million fibers made of, for example, single walled carbon nanotubes (SWCNT) and 1100 geometrically distributed optical fibers coated with SWCNT, connected in the same manner as the SWCNT fibers, wrapping around a central primary processing nucleus. Fibers may be built on a flexible interface substrate and surrounded by a gel/flesh membrane. When implanted, the membrane casing may slowly dissolve, naturally exposing the probes to a cellular environment with limited risk of rejection. The lubrication of the CNT probes will soften the attraction of neurons to the implant. The implant may be able to record from pyramidal layers II-III down to layer VI of any cortical region.

Embodiments may provide the capability to perform, for example, the following four procedures simultaneously: electrical registration of neural activity, electrical stimulation of the neural circuits to be used for the deep brain stimulation, novel optical recording and registration approach based on light-emitting proteins (fluorescence). Embodiments may provide any desired signal as opposed to the current state-of-art multi-electrode arrays. Such stretching of capabilities spectrum will provide new means to study the brain functions.

CNT fibers may be used as recording electrodes at an unprecedented scale within a highly dense geometry. CNT have a unique combination of electrical, mechanical and nanoscale properties that address the key challenges that currently limit long-term use of neural electrodes. Carbon nanotubes are ideal for integration into a neural interfaces and the technical feasibility of doing so is well documented. Integrating SWCNT technology with optical fibers enables both optogenetic writing and electrical neurostimulation modulation and transduction. CNTs are biologically compatible, enabling the KIWI to be stably implanted for long periods of time and attract neurons to self-attach and grow on the CNT probes reducing risk of neuron damage. A dissolvable membrane, such as Dextrane, Gelatine, or Collicoat, will further reduce the risk of damaging sensitive surface tissue during surgery and minimize adverse tissue reactions following implant insertion. This will protect both the patient and the CNTs.

Compared to traditional metal or glass electrodes, polymers such as CNT are flexible, strong, extremely thin, highly biocompatible, highly conductive, and have low contact impedance, which permits bidirectional interfacing with the brain. CNT fibers will allow for bidirectional input and output. These properties are especially valuable for the construction of high-density electrode arrays designed for chronic and/or long-term use in the brain. CNTs will also enable more biocompatible, longer-lasting designs whereas current neural implants work well for short periods of time, chronic or long-term use of neural electrodes has been difficult to achieve. The main reasons for this are: 1) degradation of the electrode, 2) using oversized electrodes to attain sufficient signal-to-noise ratio during recording, 3) the body's natural immune response to implantation.

CNT possess outstanding electrical and mechanical characteristics; their application has demonstrated electrical characteristics similar to the conventional rigid electrodes, but providing more stability in time (reference). In terms of biocompatibility, the application of CNT allows direct interconnection between the tissue and a devices' electrodes. In comparison to conventional electrode technologies, it has been demonstrated that CNTs promote cell attachment and enable neurite growth from attached neurons. With regards to the nanoscale size, the applications of CNT fiber lead to developing MEA devices with much higher electrode density and more simultaneous recording and stimulating channels. The CNT fiber MEA has not provided evidence of recorded signal deteriorating in time, emphasizing the stability of recorded data validity up to 5 months. The application of SWCNT allows designing a biocompatible electrode coating, which is used to improve recording and stimulating performance in the tissue by further decreasing electrode impedance and prevent toxic dissolving of CNTs. For registration of electrical activity SWCNT fiber will be implemented. The application of an optrode covered by SWCNT coating will allow KIWI to perform optical stimulation, optical recording, and electrical stimulation from the same channel site simultaneously.

The choice of materials reflects the positive results of recent studies demonstrating the impact of flexibility and density of implanted probes on CNT neural interface tissue responses. CNTs are not only biocompatible in robust coatings, but they are supportive to neuron growth and adhesion. It has been found that CNTs actually promote neurite growth, neuronal adhesion and viability of cultured neurons under traditional conditions. The nanoscale dimensions of the CNT allow for molecular interactions with neurons and the nanoscale surface topography is ideal for attracting neurons. In fact, they have been shown to improve network formation between neighboring neurons by the presence of increased spontaneous postsynaptic currents, which is a widely accepted way to judge health of network structure. Additionally, functionalization of CNT can be used to alter neuron behavior significantly. In terms of the brain's immune response, CNT have been shown to decrease the negative impact of the implanted electrodes. Upon any injury to neuronal tissue, microglia (the macrophage-like cells of the nervous system) respond to protect the neurons from the foreign body and heal the injury, and astrocytes change morphology and begin to secrete glial fibrillary acidic protein to form the glial scar. This scar will encapsulate an electrode and separate it from the neurons. However, carbon nanomaterials have been shown to decrease the number and function of astrocytes in the brain, which in turn decreases the glial scar formation.

The application of SWCNT allows for the development of a 3-D biocompatible neural implant capable of performing neural recording and neural modulation throughout I-VI brain layers. We propose to fabricate a miniaturized neural implant in a spherical obloid shape made of biocompatible material that is surgically implanted in brain parenchyma, rather than tethering the implant to the skull, which can be a major contributor to adverse tissues reactions. Whereas most probe designs use a flat and square “bed of nails” design, the KIWI uses a 3D design. The dissoluble membrane shell protects both the patient and the implant during surgery and the lubricant and contraction encourages neural encroachment and adherence to CNTs upon dissolution. This design maximizes neural connectivity and adhesion, while minimizing implant size. The KIWI's size is further reduced through inductive charging.

The geometrical and biocompatible properties of KIWI allows placing CNT-coated electrodes and optrodes throughout I-VI brain layers, which provides an innovative recording and stimulation volume. Applying the specific lubricant on the CNT-coating will allow the conductive parts of the electrode to be exposed to neurons a few weeks post-surgery. Such a method leads to maximizing the biocompatible properties of electrodes and optrodes for the neurons to grow on it.

Embodiments may provide a combination of electrical and optical sensors for registration of neural activity. Combined electro and optogenetic approach enables precise (ON/OFF) control of specific target neurons and circuits. Unary controls in combination with rapid closed loop controls in the KIWI's microchip will enable the observation of neural synapse firings with both intensity and frequency modulation. Closed-loop, activity-guided control of neural circuit dynamics using optical and electro stimulation, while simultaneously factoring in observed dynamics in a principled way is a powerful strategy for causal investigation of neural circuitry. In particular, observing and feeding back the effects of circuit interventions on physiologically relevant timescales is valuable for directly testing whether inferred models of dynamics, connectivity, or causation is as accurate in vivo.

A cloud platform may house the parallel data flow and analytic engine powered by neurocomputational algorithms and deep machine learning. Data from the KIWI will be uploaded to the cloud wirelessly from the KIWI implant. A suite of algorithms may analyze the aggregate datastream and formulate instructions for optimal electrical and/or optical neuromodulations in a closed loop feedback system. Integrated stimulation/control, recording/readout and modulated stimulation parameters will allow simultaneous optical and/or electrical recording and stimulation. The cloud platform may also provide the massive processing power required to analyze huge data sets. The patented machine learning algorithms provide common temporal and spatial coordinates for the KIWI data for consistent mapping across different data types. The algorithms provide extremely high rates of data compression (>90%), association and throughput, enabling the KIWI to transcribe neural signals in high volume.

The application of AI with a corresponding server for the data storage, hones the effectiveness of the closed-loop operation and allows us to autonomously monitor and stimulate required neural circuits. The computational power provided by the neurocomputational framework allows us to develop a multielectrode array with big conversion capabilities, which takes away some limitations from transmission speed, number of channels and quality of electronic sensors. Closed-loop, autonomous architecture could enable dynamic informed response, based on real-time analysis. Closed-loop control could guide perturbations of neural systems (neurons and circuits). Sophisticated, real-time control over neural dynamics will serve to generate, refine and confirm circuit-based models of the underlying system. In closed-loop optogenetics, for example, the control input is a structured, time-varying light stimulus automatically modulated based on the difference between desired and measured outputs, which may include behavioral, electrophysiological, or optical readouts of activity generated by the biological system. Such a system allows adaptation, plasticity, and neural state changes, as well as online tuning of optogenetic inputs, to be explored in vivo.

Embodiments may provide a suite of neurocomputational algorithms for transducing neuron output into digital information. These algorithms are theoretically grounded, computational models corresponding to the theory of similarity computation in Bottom-Up and Top-Down signal interaction. These neurally-derived algorithms use mathematical abstractions of the representations, transformations, and learning rules employed by the brain, which will correspond to the models derived from the data and correspond to general dynamic logic, accounting for uncertainty, as well as providing predictive analytical capabilities for events yet to take place. Embodiments may provide improved similarity estimation, generalization from a single exemplar, and recognition of more than one class of stimuli within a complex composition (“scene”) given single exemplars from each class. Combined, these provide both global (brain-wide) and fine detail (i.e. communication between and within cytoarchitectonic areas) modalities for reading and writing across different timescales.

Embodiments may provide CMOS technology for electrical recording. A major advantage that CMOS image sensors enjoy over their CCD counterparts is the ability to integrate a number of processing and control functions, which lie beyond the primary task of photon collection, directly onto the sensor integrated circuit. These features generally include timing logic, exposure control, analog-to-digital conversion, shuttering, white balance, gain adjustment, and initial image processing algorithms. In order to perform all of these functions, the CMOS integrated circuit architecture more closely resembles that of a random-access memory cell rather than a simple photodiode array. The most popular CMOS designs are built around active pixel sensor (APS) technology in which both the photodiode and readout amplifier are incorporated into each pixel. This enables the charge accumulated by the photodiode to be converted into an amplified voltage inside the pixel and then transferred in sequential rows and columns to the analog signal-processing portion of the chip. For example, the operational principle may be based on role distributions: The Intel Curie module is responsible for controlling electrical recording and stimulation meanwhile an optical FPGA is responsible for controlling optical recording and stimulation. An optical FPGA transfers the sampled optical data to the Intel Currie for a subsequent wireless transfer. Despite carrying a bigger computational flexibility, having both the microcontroller and optical FPGA results in additional power consumption and increase in the physical size of the chip.

Embodiments may provide a wireless multielectrode array to make it a one-time-only implantable solution. Embodiments may provide a miniature, wireless multielectrode array. Embodiments may be capable of performing both optical and electrical recording and stimulation. Hence, the amount of available channels for listed operations, and power consumption of the whole system will strongly depend on the controlling circuit's characteristics. The size of transferred data, determined by the parameters of a CPU and design of KIWI, strongly influence the wireless transmission protocol used to transfer the data. For example, the ESP32 is a single 2.4 GHz Wi-Fi-and-Bluetooth combo chip designed with the TSMC ultra-low-power 40 nm technology. It is designed to achieve improved power and RF performance, showing robustness, versatility and reliability in a wide variety of applications and power scenarios. ESP32 is designed for mobile, wearable electronics, and Internet-of-Things (IoT) applications. It features all the state-of-the-art characteristics of low-power chips, including fine-grained clock gating, multiple power modes, and dynamic power scaling. For instance, in a low-power IoT sensor hub application scenario, ESP32 is woken up periodically only when a specified condition is detected. Low-duty cycle is used to minimize the amount of energy that the chip expends. The output of the power amplifier is also adjustable, thus contributing to an optimal trade-off between communication range, data rate and power consumption. ESP32 is a highly-integrated solution for Wi-Fi-and-Bluetooth IoT applications, with around 20 external components. ESP32 integrates an antenna switch, RF balun, power amplifier, low-noise receive amplifier, filters, and power management modules. As such, the entire solution occupies minimal Printed Circuit Board (PCB) area.

ESP32 uses CMOS for single-chip fully-integrated radio and baseband, while also integrating advanced calibration circuitries that allow the solution to remove external circuit imperfections or adjust to changes in external conditions. As such, the mass production of ESP32 solutions does not require expensive and specialized Wi-Fi testing equipment.

ESP32 has a lot more features than ESP8266
Single or Dual-Core 32-bit LX6 Microprocessor with clock frequency up to 240 MHz.
520 KB of SRAM, 448 KB of ROM and 16 KB of RTC SRAM.
Supports 802.11 b/g/n Wi-Fi connectivity with speeds up to 150 Mbps.
Support for both Classic Bluetooth v4.2 and BLE specifications.
34 Programmable GPIOs.
Up to 18 channels of 12-bit SAR ADC and 2 channels of 8-bit DAC
Serial Connectivity include 4×SPI, 2×I2C, 2×I2S, 3×UART.
Ethernet MAC for physical LAN Communication (requires external PHY).
1 Host controller for SD/SDIO/MMC and 1 Slave controller for SDIO/SPI.
Motor PWM and up to 16-channels of LED PWM.
Secure Boot and Flash Encryption.
Cryptographic Hardware Acceleration for AES, Hash (SHA-2), RSA, ECC and RNG.

The ESP32 die, which can be connected to external circuitry via semiconductor bridges or bonding, and can be ordered directly from the manufacturer without remaining parts of the SoC. The scales of the die are 2.96 mm×2.85 mm×0.21 mm. Hence, assuming that the die will have a cover, that is encapsulated in KIWI elipsoid shape, the height of KIWI will be 4 mm and the total volume occupied will be 3.61 mm\hat{ }3.

To derive the dependency of the overall recording frequency from the number of multifunctional pixels, the timing process for one sample of information has to be considered. Each cycle of the recording-transmission consists of 5 stages:
1. Capturing the analogue samples via charging the capacitors within pixels
2. Digitizing the sample using the embedded 12-bit ADC with 2 Msps sampling frequency
3. Switching the Multiplexer to sample next out of 18 ADC channel
4. Compress the digitized data via LZO protocol
5. Transmit the data to external receiver via 802.11 b/g/n Wi-Fi at 150 Mbps

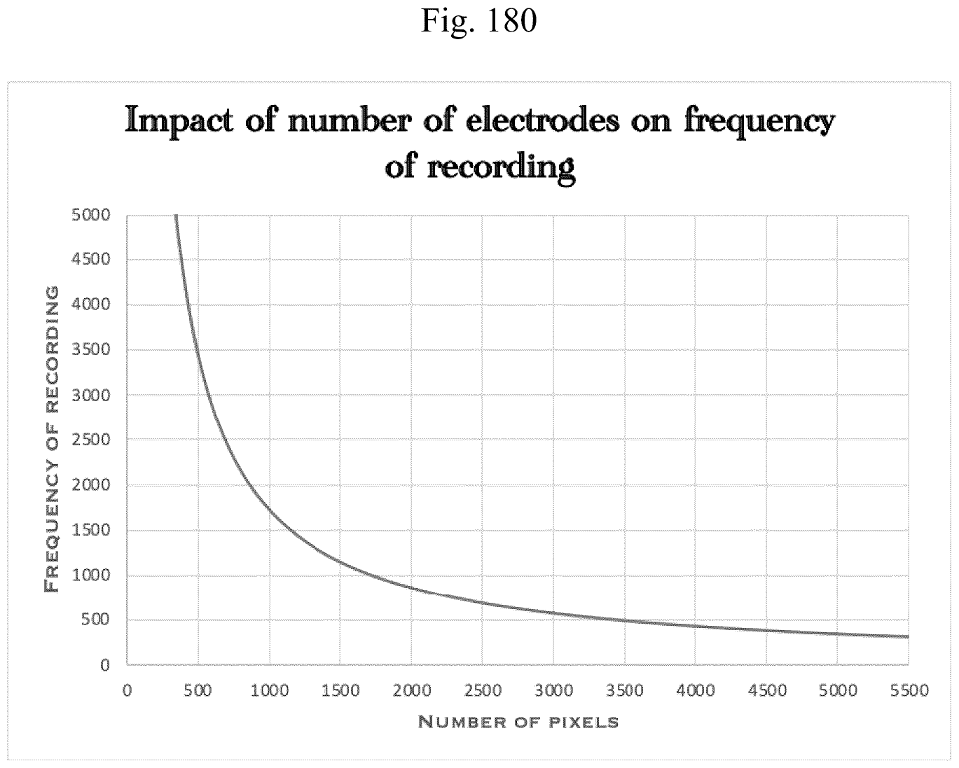

Dual-core characteristics allows ESP32 to apply threading, thus performing multiple stages of the timing process simultaneously. Hence, the overall timing for the 5 stages is reduced to only 2 stages per one cycle of transmission. ESP32 possess 2×Xtensa® 32-bit LX6 Microprocessors cores. Accordingly, the timing for the procedure that determines the overall frequency of signal recording is dictated only by ADC conversion time and data transmission via Wi-Fi 802.11 n time. Each data samples is converted to 12 bit value at 2 Msps speed and compressed with some experimental ratio. Then the time for each cycle of signal recording may be derived, with results shown in FIG. 180. Each channel consists of recording electrodes made of SWCNT fiber and an optrode with SWCNT coating. The configuration of the latter allows to combine optical procedures (recording and stimulation) with electrical stimulation procedure due to conductive properties of SWCNT coating. The KIWI chip performs recording an electrical and optical neural activity as well as stimulating the desired activity by injecting electrical and optical signals, as shown in FIG. 180. The procedures are performed via the CMOS pixel matrix, controlled by ESP32. Each pixel performs both stimulating and recording procedures simultaneously.

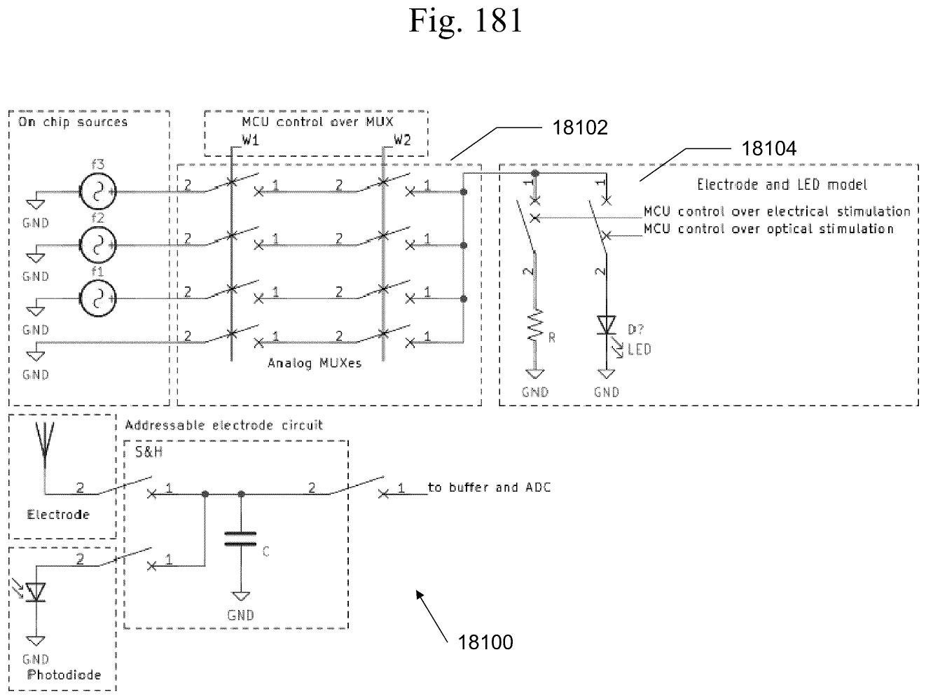

An exemplary individual pixel circuit 18100 is shown in FIG. 181. In embodiments, pixel stimulation circuitry may use an external multi-phase H-bridge inverter connected to one DAC, each pixel is supplied with a choice of selecting a stimulating voltage pattern from the global voltage lines that are common and provided to every pixel. Selection of the stimulating voltage signal for each pixel as well as the nature of stimulation—optical (LED) or (SWCT electrode)—is performed by ESP32 via analogue multiplexer and switching elements 18102, respectively. Each switching element may be executed as a bilateral CMOS couple 18104 to eliminate signal clipping whenever the voltage changes the polarity. Analog circuit design may be performed with SPICE and MATLAB simulations using available CMOS models.

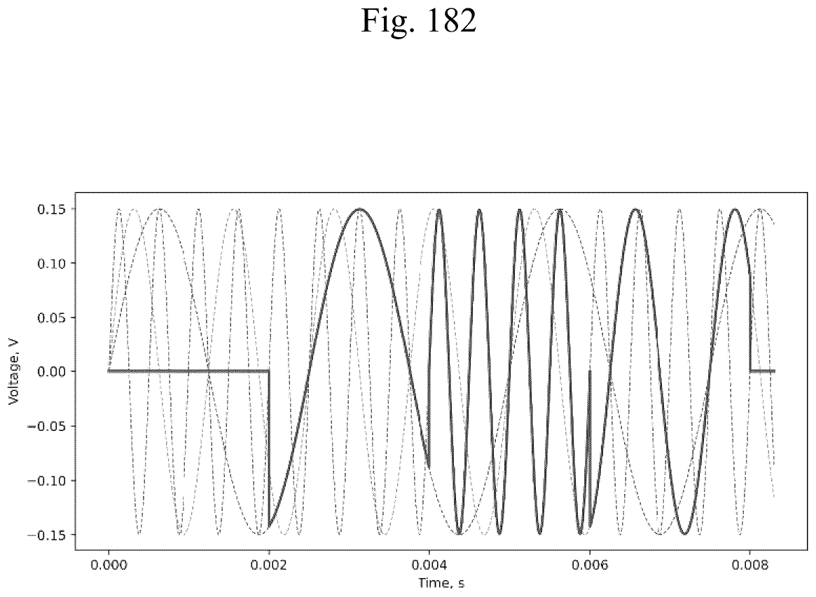

An exemplary stimulation simulation in SPICE using 65 nm transistors is shown in FIG. 182. The output voltage across the electrode (solid) is constantly changing to one of the provided common voltage patterns (dotted) The simulation demonstrates that ESP32 switches various reference stimulation patterns, leading to changes in the stimulation voltage across the electrode. No signal degradation or phase shift is observed during the switching, emphasizing the stimulation method's flexibility.

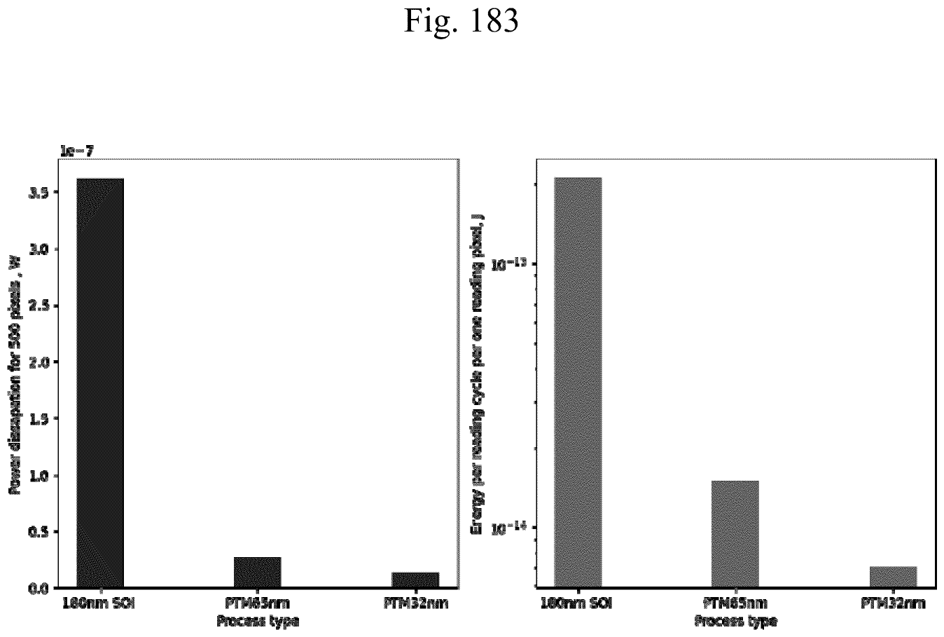

The total power consumption of the stimulation circuitry of all individual pixels depends on the amount and nature of reference patterns provided to the pixel. An example of the recording power consumption estimation per cycle and continuous operation on different process nodes using SPICE models is shown in FIG. 183. The power consumption estimation for the example of a 500-pixel matrix performing simultaneous signal recording was estimated using SPICE simulation. Three different exemplary process nodes—180 nm SOI, 65 and 32 nm PTM models were used for the evaluation. The results demonstrate that power dissipation is heavily dependent on the node used. However, the power consumption varies from 10 to 350 nanowatts indicating almost zero readout power and emphasizing the low-power performance of the matrix.

The use of CNT allows for one unit to function as recording electrodes and stimulating optical fibers. CNTs allow for the use of smaller electrodes by reducing impedance, improving signal-to-noise ratios while improving the biological response to neural electrodes. Measurements show that the output photocurrent varies linearly with the input light intensity and can be modulated by bias-voltage. The quantum efficiency of CNT are about 0.063% in 760 Torr ambient, and becomes 1.93% in 3 mTorr ambient. A SWCNT fiber bundle can be stably implanted in the brain for long periods of time and, in several studies, have been found to attract neurons to grow or self-attach to the probes. The optical fibers will be coated with SWCNT to make electrical connections with the underlying delay line. The delay line will be transparent to allow light from the VCSELs to reach the optical fibers. The delay lines may potentially make the electrical signal position-dependent by comparing the time between pulses measured at the outputs. Provided the pulses are of sufficient intensity and individual pulses are sufficiently separated in time (>1 μs or so), the difference between pulse arrival times could be related to the position on the array. Combining this with spatially controlled optical excitation (i.e., by turning on specific VCSELs) would further help to quantify position, as VCSEL pulses excite a small region at the end of the adjacent fiber. These pulses are measured at a position on the delay line close to this fiber, so if neighboring neurons fire, they are sensed by nearby fibers (i.e., the SWCNTs on the fibers) and would generate additional pulses that could then be tracked over time with the delay line, enabling us to map out the path. The SWCNT coated fiber array will be randomly connected to the underlying VCSEL array, as we will not have control over the fiber locations in the bundle. The substrate connectors will be graphitic nano-joints and SWNT. We will also utilize the IBM CNT connect technique for other connectors. The optical transceivers will be integrated as a separate die on a silicon substrate, tightly-coupled to logic dice (a.k.a. “2.5D integration”).

Embodiments may use optogenetic tools to enable precise silencing of specific target neurons. Optical neuromodulation has many benefits over traditional electrode-based neurostimulation. Using unary controls in combination with rapid closed-loop controls will enable neural synapse firings with highly precise timing, intensity, and frequency. We will use optogenetics to facilitate selective, high-speed neuronal activation. Optogenetics is a technology in which light-sensitive ion channels are expressed in target neurons allowing their activity to be controlled by light. By coating optical fibers (˜8 m) with dense, thin (˜1 m) CNT conformal

Embodiments may provide optical modulation units within the nucleus of the KIWI implant that can deliver light to precise locations deep within the brain while recording electrical activity at the same target locations. We have shown that the light-activated proteins channelrhodopsin-2 and halorhodopsin can be used to activate and inhibit neurons in response to light of different wavelengths and we are now developing precisely targetable fiber arrays and in vivo-optimized expression systems to enable the use of this tool in awake, behaving primates.

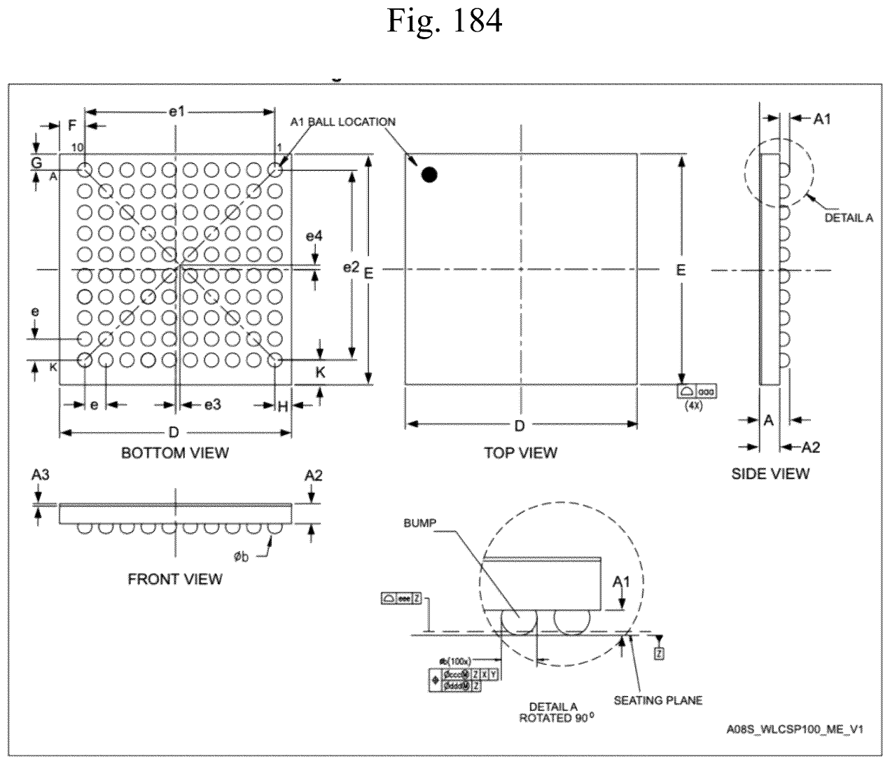

The amount of the data, determined by the parameters of a CPU and design of Kiwi, strongly influence the transmission protocol used to transfer the data. To surpass characteristics of Intel Curie module in terms of speed, computational powers and size, Embodiments may utilize an Ultra-low power STM32WB55VY microcontroller, shown in FIG. 184. STM32WB55VY possesses two cores: Cortex m-4 core operating at 64 MHz and Cortex m-0+core operating at 32 MHz with capability of threading [20]. The m-4 core features a Floating point unit (FPU) single precision that supports all Arm single-precision data-processing instructions and data types. It also implements a full set of DSP instructions and a memory protection unit (MPU) that enhances application security. The combination of cores possessing such characteristics becomes essential feature in minimizing the recording and transmission time. The device embed a powerful and ultra-low radio compliant with the Bluetooth LE SIG specification 5.2 and with IEEE 802.15-4-2011, providing the data rate of 2 Mbps. The devices embed high-speed memories (up to 1 Mbyte of Flash memory for STM32WB55VY, 256 KB SRAM including 64 KB with hardware parity check, and Quad-SPI Flash memory interface. Also, the device provides an extensive range of enhanced I/Os and peripherals, such as 12-bit ADC with sampling frequency 4.26 Msps and 200 uA/Msps power consumption. The device operates from 1.71 to 3.6 Volts and requires 13 nA in shutdown mode and 600 nA Standby mode. Lastly, the package WLCSP100 is a 100-ball, 4.390×4.371 mm, 0.4 mm pitch, wafer level chip scale package with 72 fast I/Os, 70 of which are 5V tolerant.

As it was said earlier, the limits of the data quantity is determined by the design demands. Taking into considerations scales of the chip, the volume of the STM-32 is calculated:

V_{STM}=4.390 mm*4.371 mm*0.38 mm=7.29 mm3

In embodiments, the chip may be located inside the cubic structure made up from multifunctional pixels. Assuming that the chip occupies 80% of the volume of cubic structure, the volume of the cube is calculated accordingly:

$${V}_{CUBE} = \frac{7.29 {mm}^{2}}{0.8} = 9.11 {mm}^{2}$$

The cubic structure made of multifunctional pixels is located inside the spheroid—the external shell of the Kiwi. Assuming the cube occupies 80% of the spheroid, the volume of shell of Kiwi is found:

$${V}_{SHELL} = \frac{9.11 {mm}^{2}}{0.8} = 11.38 {mm}^{2}$$

To complete the mechanical design the parameters of the spheroid are to be found. Assuming that the height b and length c of the elipsoid is 6 mm, the diameter of the spheroid a is found::

$${V}_{SHELL} = \frac{π}{6} abc \\ a = \frac{6 {V}_{SHELL}}{π bc} = \frac{6 * 11.38 {mm}^{2}}{π * {( 6 mm )}^{2}} = 0.6 mm$$

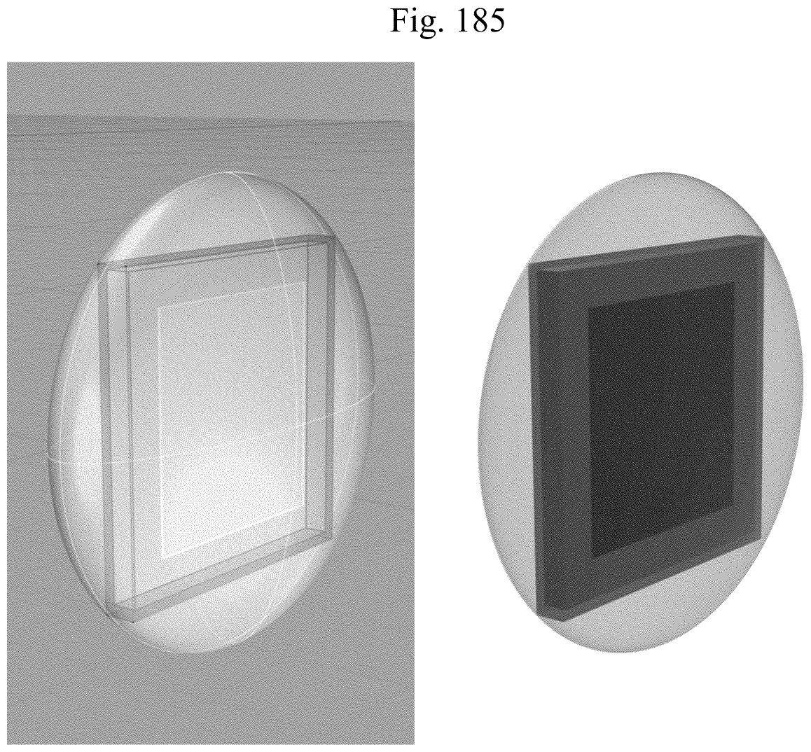

The materials that are making up the shell as well as the details of contact with SWCNTs fiber are described below. The amount of pixels of the cube determines how much information is available, which raises the question about the data transfer limits. Hence, to assume the number of pixels to be used in the design, it is essential to calculate the maximum number of pixels the cube area can contain. First, with volume of the cube known, the sides of the cube a, b, and c to be calculated. The a and b are assumed to be 4.5 mm, which is almost 0.1 mm bigger than the biggest parameter of the stm-32 chip. Hence, the depth is calculated to be 0.45 mm. The Chip 3-D model based on the calculated geometrical parameters is shown in FIG. 185, which shows the STM32WB55VY: WLCSP100 integrated in Kiwi.

Hence, the area of the surface cube is found:

S_{CUBE}=4.5 mm*4.5 mm*2+4.5 mm*0.45 mm*4=48.6 mm²

Apart from pixels, the cube area contains peripherals, multiplexors, databus connections, row & column selectors, etc. Thus, the effective area of the cube containing pixels only is smaller than the surface area. It is assumed, that effective area of the cube's surface is 90% of the overall area.

S_{eff}=S_{CUBE}*0.9=43.74 mm²

The pitch size is assumed to be 9 um, since it is not the prospect CMOS pixel scaling and it is easy to fabricate. Then, the area of one pixel can be approximated to 81 squared micrometers. Hence, the maximum possible number of pixels located on the surface of the cube structure can be calculated:

$${n}_{max} = \frac{43.74 {mm}^{2}}{81 {µm}^{2} \frac{{µm}^{2}}{pixel}} = 540000 pixels$$

Obtaining the physical threshold value demonstrates the potential reach of the proposed technology given the future advances in wireless transmission protocols. To derive the dependency of the overall recording frequency from the number of multifunctional pixels, the timing process for one sample of information has to be considered. Each cycle of the recording-transmission consists of 5 stages:
1. Capturing the analogue samples via charging the capacitors within pixels.
2. Digitizing the sample using the embedded 12-bit ADC with 4.26 Msps sampling frequency.
3. Switching the Multiplexer to sample next column/row
4. Compress the digitized data via LZO protocol.
5. Transmit the data to external receiver via Bluetooth 5.2 Low Energy transmitting at 2 Msps.

Dual-core characteristics allows STM32WB55VY to apply threading, thus performing multiple stages of the timing process simultaneously. Hence, the overall timing for the 5 stages is reduced to only 2 stages per one cycle of transmission.
1. Cortex m-0+: capturing the analogue samples via charging the capacitors within pixels. Cortex m-4: transmitting the previous data packet to an external receiver
2. Cortex m-0+: digitizing the sample using the embedded 12-bit ADC with 4.26 Msps sampling frequency.
   Cortex m-4: compressing each incoming individual sample data using LZO protocol and switching the multiplexers to sample next column/row. This becomes possible due to the Cortex m-4 being twice faster than cortex m-0+. Also, possessing 73 I/O pins allows STM32WB55VY to handle several dozens of multiplexer.

Repeat the Procedure

Accordingly, the timing for the procedure that determines the overall frequency of signal recording is dictated only by ADC conversion time and data transmission via Bluetooth 5.2 time. Each data samples is converted to 12 bit value at 4.26 Msps speed and compressed with some experimental ratio. Then the time for each cycle of signal recording is derived:

$${t}_{record} = {n}_{pixels} ( \frac{\frac{12}{a}}{2 * 1 {0}^{6} bps} + \frac{1}{4.26 * 1 {0}^{6} bps} )$$

Where a is the combined compression ratio of LZO and Bluetooth 5.2. The ratio is assumed to be equal to 4, since the coefficient is fairly achievable for dual-core microcontrollers.

$${t}_{record} = {n}_{pixels} ( \frac{3}{2 * 1 {0}^{6} bps} + \frac{1}{4.26 * 1 {0}^{6} bps} )$$

The frequency of the signal recording is found accordingly:

$$f = \frac{1}{{n}_{pixels} ( \frac{3}{2 * 1 {0}^{6} bps} + \frac{1}{4.26 * 1 {0}^{6} bps} )}$$

The derived expression demonstrates that the dependency of the frequency of signal recording from amount of pixels obeys

$$y = \frac{k}{x} .$$

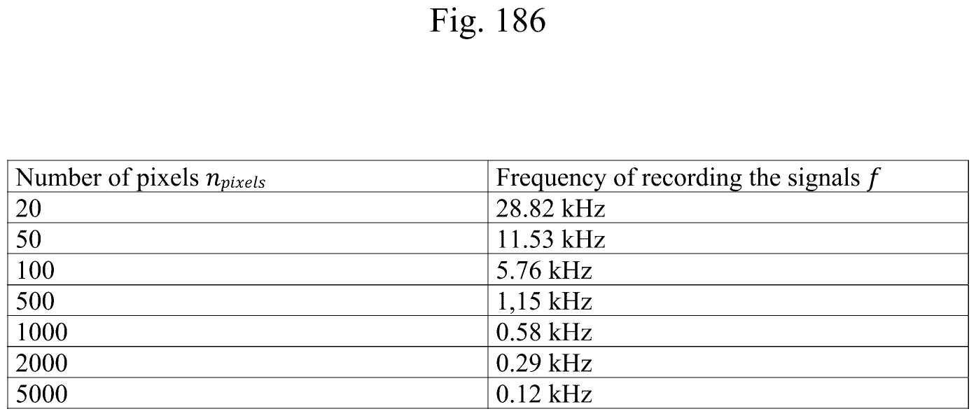

Function. Seven values were taken and placed inside the equation to construct the table of dependency of frequency on the number of pixels. Thus, the Numbers of pixels vs Frequency of recording is shown in FIG. 186. Covering the 1-5500 pixels, the graph shown in FIG. 180 is plotted to visually demonstrate the behavior of the function.

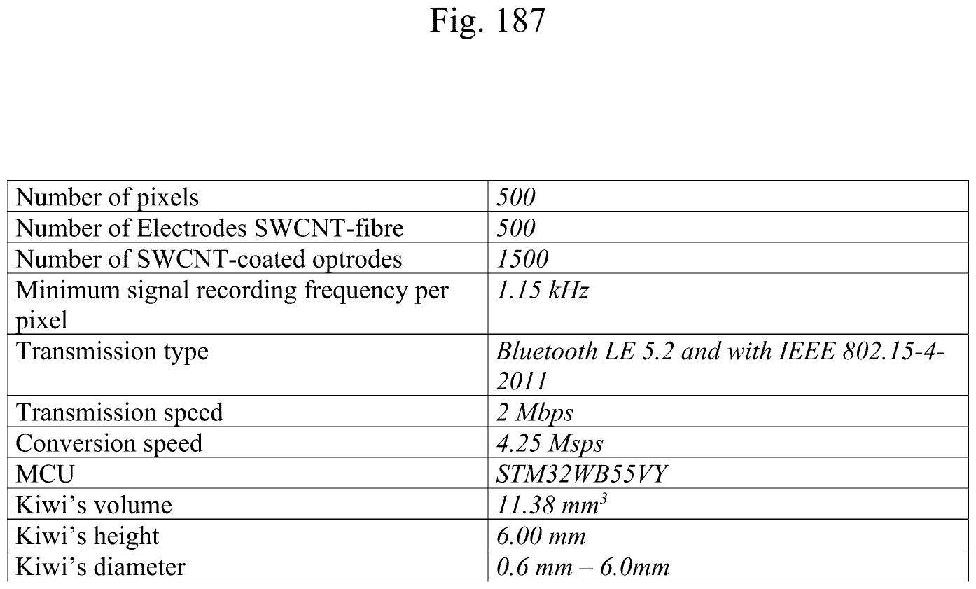

Embodiments may provide, for example, 500 multifunctional pixels. According to the FIG. 186, 500 pixels point is located at the maximum slope change. Thus, selecting the 500 pixels as the reference, the data quantity, transmission characteristics as well as geometrical parameters of an embodiment are summarized in as shown in FIG. 187, which shows conversion, transmission and geometrical characteristics for an STM-32 based embodiment.

ESP32 is a single 2.4 GHz Wi-Fi-and-Bluetooth combo chip designed with the TSMC ultra-low-power 40 nm technology. It is designed to achieve the best power and RF performance, showing robustness, versatility and reliability in a wide variety of applications and power scenarios. ESP32 is designed for mobile, wearable electronics, and Internet-of-Things (IoT) applications. It features all the state-of-the-art characteristics of low-power chips, including fine-grained clock gating, multiple power modes, and dynamic power scaling. For instance, in a low-power IoT sensor hub application scenario, ESP32 is woken up periodically only when a specified condition is detected. Low-duty cycle is used to minimize the amount of energy that the chip expends. The output of the power amplifier is also adjustable, thus contributing to an optimal trade-off between communication range, data rate and power consumption. ESP32 is a highly-integrated solution for Wi-Fi-and-Bluetooth IoT applications, with around 20 external components. ESP32 integrates an antenna switch, RF balun, power amplifier, low noise receive amplifier, filters, and power management modules. As such, the entire solution occupies minimal Printed Circuit Board (PCB) area.

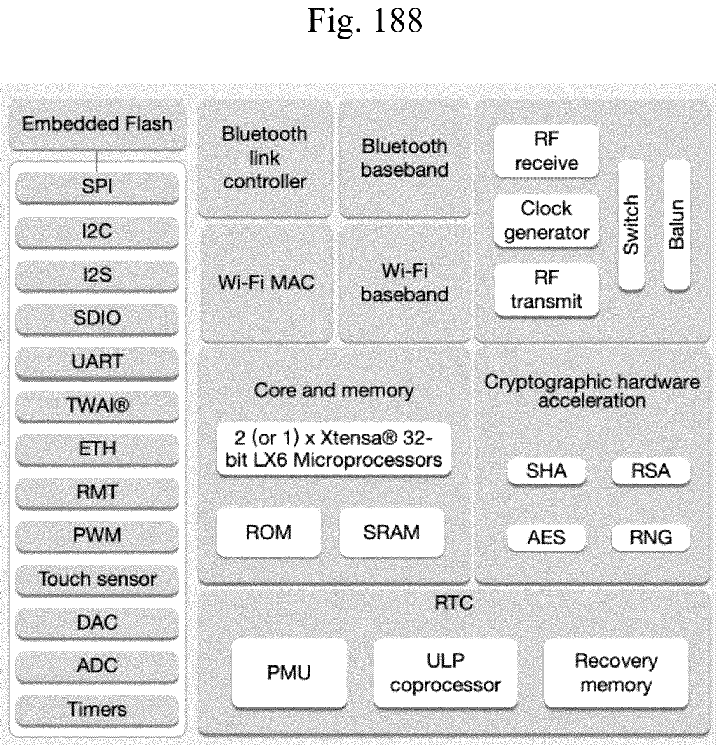

ESP32 uses CMOS for single-chip fully-integrated radio and baseband, while also integrating advanced calibration circuitries that allow the solution to remove external circuit imperfections or adjust to changes in external conditions. As such, the mass production of ESP32 solutions does not require expensive and specialized Wi-Fi testing equipment. An overview of features of the ESP32 is shown in FIG. 188. Sample specifications may include:
Single or Dual-Core 32-bit LX6 Microprocessor with clock frequency up to 240 MHz.
520 KB of SRAM, 448 KB of ROM and 16 KB of RTC SRAM.
Supports 802.11 b/g/n Wi-Fi connectivity with speeds up to 150 Mbps.
Support for both Classic Bluetooth v4.2 and BLE specifications.
34 Programmable GPIOs.
Up to 18 channels of 12-bit SAR ADC and 2 channels of 8-bit DAC
Serial Connectivity include 4×SPI, 2×I2C, 2×I2S, 3×UART.
Ethernet MAC for physical LAN Communication (requires external PHY).
1 Host controller for SD/SDIO/MMC and 1 Slave controller for SDIO/SPI.
Motor PWM and up to 16-channels of LED PWM.
Secure Boot and Flash Encryption.
Cryptographic Hardware Acceleration for AES, Hash (SHA-2), RSA, ECC and RNG.

Even though ESP32 is a SoC with physical attributes similar to STM32 described above, the ESP32 die, which can be connected to external circuitry via semiconductor bridges or bonding, and can be ordered directly from the manufacturer without remaining parts of the SoC.

The scales of the die are 2.96 mm×2.85 mm×0.21 mm. Thus, repeating the calculating procedure above, the volume of the ESP32 die is calculated.

V_{STM}=2.96 mm*2.85 mm*0.21 mm=1.77 mm³

Embodiments may provide the chip is located inside the cubic structure made up from multifunctional pixels. Assuming that the chip occupies 70% of the volume of cubic structure, the volume of the cube is calculated accordingly:

$${V}_{CUBE} = \frac{1.77 {mm}^{2}}{0.7} = 2.35 {mm}^{2}$$

The cubic structure made of multifunctional pixels is located inside the spheroid—the external shell of the Kiwi. Assuming the cube occupies 70% of the spheroid, the volume of shell of Kiwi is found:

$${V}_{SHELL} = \frac{2.21 {mm}^{2}}{0.7} = 3.61 {mm}^{2}$$

To complete the mechanical design the parameters of the ellipsoid, where the cube is incapsulated in, are to be found. Assuming that the height b of the and the maximum length c are 4 mm, the diameter of the ellipsoid a is found:

$${V}_{SHELL} = \frac{π}{6} abc$$

$$a = \frac{6 {V}_{SHELL}}{π b c} = \frac{6 * 3.61 {mm}^{2}}{π * {( 4 mm )}^{2}} = 0.43 mm$$

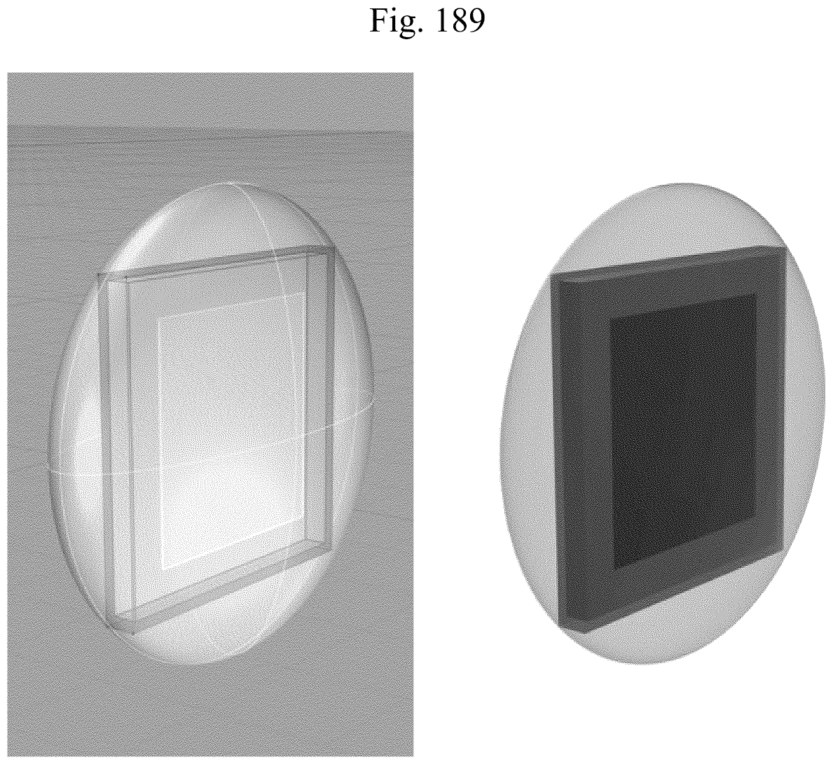

The materials that are making up the shell as well as the details of contact with SWCNTs fiber are described below. The amount of pixels of the cube determines how much information is available, which raises the question about the data transfer limits. Hence, to assume the number of pixels to be used in the design, it is essential to calculate the maximum number of pixels the cube area can contain. First, with volume of the cube known, the sides of the cube a, b, and c to be calculated. The a and b are assumed to be 3.1 mm.. Hence, the depth is calculated to be 0.27 mm. The Chip 3-D model based on the calculated geometrical parameters is shown in FIG. 189, which shows the ESP32 integrated in Kiwi.

Hence, the area of the surface cube is found:

S_{CUBE}=3.1 mm*3.1 mm*2+3.1 mm*0.27 mm*4=22.57 mm²

Apart from pixels, the cube area contains peripherals, multiplexors, databus connections, row & column selectors, etc. Thus, the effective area of the cube containing pixels only is smaller than the surface area. It is assumed, that effective area of the cube's surface is 90% of the overall area.

S_{eff}=S_{CUBE}*0.9=20.31 mm²

The pitch size is assumed to be 9 um, since it is not the prospect CMOS pixel scaling and it is easy to fabricate. Then, the area of one pixel can be approximated to 81 squared micrometers. Hence, the maximum possible number of pixels located on the surface of the cube structure can be calculated:

$${n}_{max} = \frac{2 0.31 {mm}^{2}}{81 {μm}^{2}} = 250740 pixels$$

Obtaining the physical threshold number of pixels demonstrates the potential reach of the proposed technology given the future advances in wireless transmission protocols. To derive the dependency of the overall recording frequency from the number of multifunctional pixels, the timing process for one sample of information has to be considered. Each cycle of the recording-transmission consists of 5 stages:
1. Capturing the analogue samples via charging the capacitors within pixels
2. Digitizing the sample using the embedded 12-bit ADC with 2 Msps sampling frequency
3. Switching the Multiplexer to sample next out of 18 ADC channel
4. Compress the digitized data via LZO protocol
5. Transmit the data to external receiver via 802.11 b/g/n Wi-Fi at 150 Mbps

Dual-core characteristics allows ESP32 to apply threading, thus performing multiple stages of the timing process simultaneously. Hence, the overall timing for the 5 stages is reduced to only 2 stages per one cycle of transmission. ESP32 possess 2×Xtensa® 32-bit LX6 Microprocessors cores.
1. Xtensa 1st: capturing the analogue samples via charging the capacitors within pixels. Xtensa 2nd: transmitting the previous data packet to an external receiver.
2. Xtensa 1st digitizing the sample using the embedded 12-bit ADC with 2 Msps sampling frequency.
   Xtensa 2nd: compressing each incoming individual sample data using LZO protocol and switching the multiplexers to sample next column/row. The capability of providing hundreds of recording channels is supported by 18-channel ADC. Switching the channels and controlling multiplexers with few dozens of GPIO, sampling thousands of electrode recordings becomes possible.

Repeat the Procedure

Accordingly, the timing for the procedure that determines the overall frequency of signal recording is dictated only by ADC conversion time and data transmission via Wi-Fi 802.11 n time. Each data samples is converted to 12 bit value at 2 Msps speed and compressed with some experimental ratio. Then the time for each cycle of signal recording is derived:

$${t}_{record} = {n}_{pixels} ( \frac{12}{150 * 1 {0}^{6} bps} + \frac{1}{2 * 1 {0}^{6} bps} )$$

The frequency of the signal recording is found accordingly:

$$f = \frac{1}{{n}_{pixels} ( \frac{12}{150 * 1 {0}^{6} bps} + \frac{1}{2 * 1 {0}^{6} bps} )}$$

The derived expression demonstrates that the dependency of the frequency of signal recording from amount of pixels obeys

$$y = \frac{k}{x} .$$

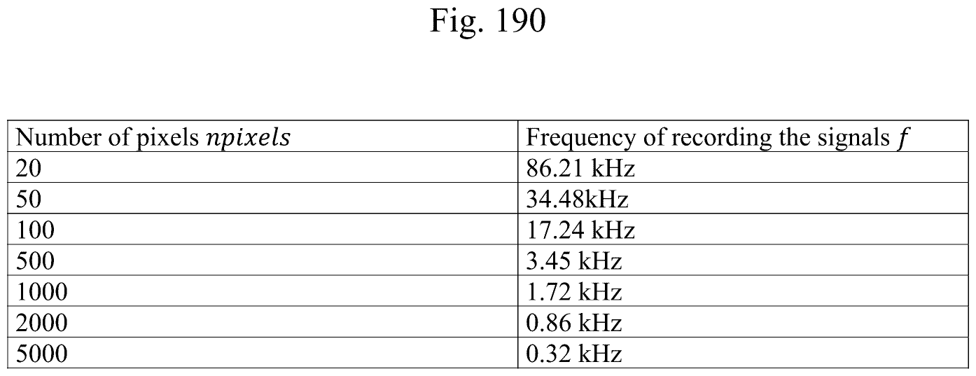

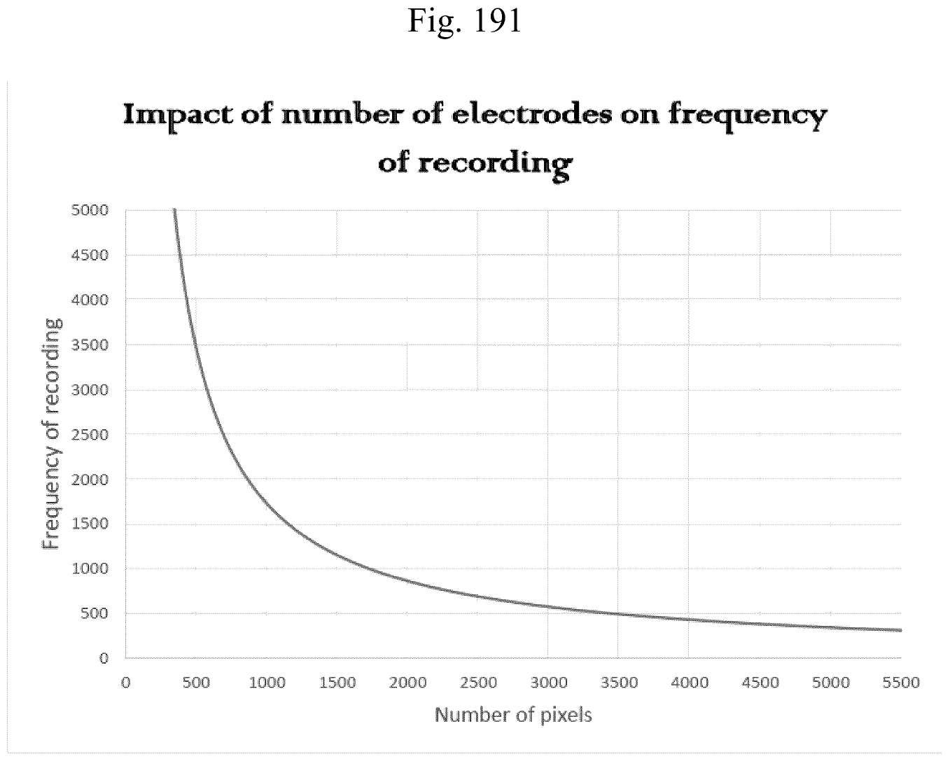

Function. Seven values were taken and placed inside the equation to construct the table of dependency of frequency on the number of pixels shown in FIG. 190. Covering the 1-5500 pixels, the graph of numbers of pixels vs Frequency of recording for the ESP32-based KIWI shown in FIG. 191 is plotted to visually demonstrate the behavior of the function.

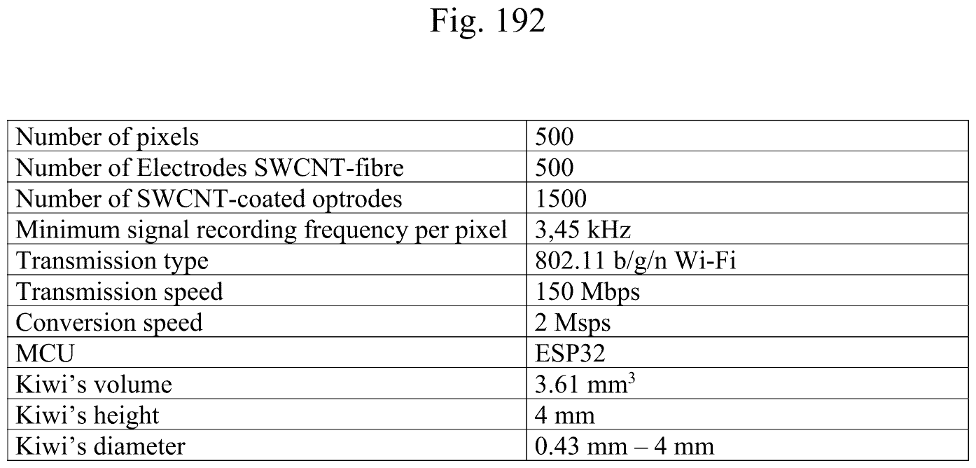

The summarized physical parameters along with transmission, conversion, and design characteristics are presented in the table shown in FIG. 192, which shows conversion, transmission and geometrical characteristics for the ESP32-based KIWI.

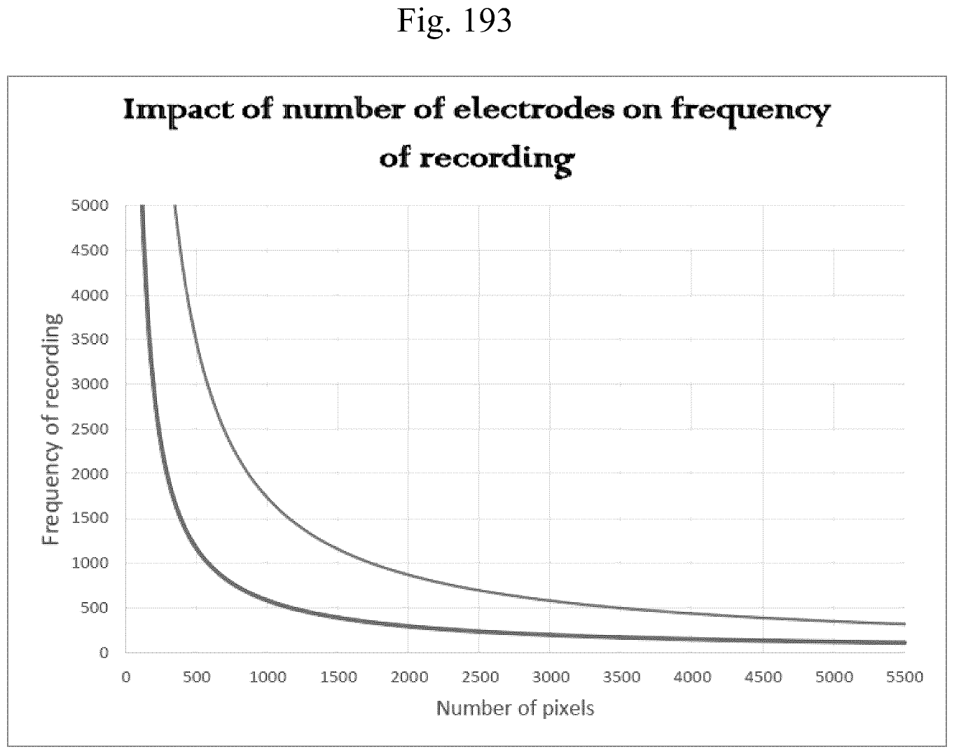

The comparison between performance of the STM-32 based embodiments and ESP-32-based embodiments in terms of number of pixels against recording speed shown in FIG. 193. It is visually evident, that increasing number of multifunctional pixels from 2500-3000 onwards carries little change of the recording frequency for both embodiments. In this matter, ESP-32 demonstrates better speed of recording. However, the final frequency will be varying in 200-400 Hz ratio, which is not satisfying for a neural recording technology. The proposed technologies demonstrate satisfying results for a number of pixels less than 1500. Hence, three following statements are made:

1. The proposed technologies fits all the requirements for comfortable neural recording
2. The proposed technologies possess a good potential for multi-thousand array, since the frequency of recording has a minimal changes while numbers of pixels scaled higher significantly (from a specific number of pixels onwards)
3. The proposed method can be used to create the novel technology, which will possess threshold frequency 3-4 times faster than ones of the proposed technology (this will be discussed below)

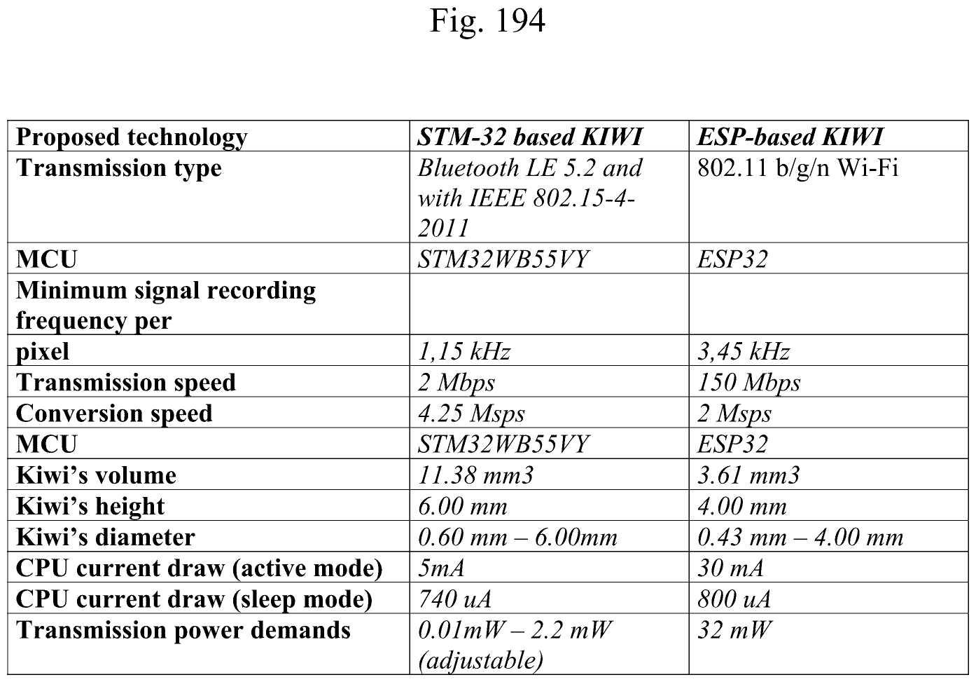

To inspect the difference between the proposed technologies, 500 multifunctional pixels are taken as a reference. The speed, size, and power parameters of the proposed technologies are summarized in the table shown in FIG. 194, which shows conversion, transmission and geometrical characteristics for the STM-32- and ESP32-based embodiments.

ESP-32 offers 75 times faster recording frequency than STM-32. However, the ESP-32-based KIWI technology provides only 3 time faster frequency speed. The nature behind this calculation is the recording speed limitation, which was placed upon the designs by sampling speed of ADCs. Taking ESP-32-based design as the reference, the limitation of sampling speed can be observed by assuming that transmission time is zero.

$${t}_{limit} = {n}_{pixels} ( 0 + \frac{1}{2 * 1 {0}^{6} sps} ) = \frac{500}{2 * 1 {0}^{6} sps} = 250 μs$$

$${f}_{limit} = \frac{1}{\frac{500}{2 * 1 {0}^{6} sps}} = 4 kHz$$

Meaning that no matter the advance of wireless transmission systems' speed, the threshold recording frequency for 500 multifunctional pixels will be 4 kHz for the ESP32-based technology.

The volume of the ESP-32-based KIWI is approximately 3 times smaller than the volume of the STM-32 based KIWI technology. Despite the a disadvantage of STM-32-based KIWI concept in recording speed and occupied space, it possesses better power consumption characteristics. Total current demands of running cortex m-0+ and m-4 are 6 times smaller than current demands of two Xtensa CPUs. During sleep mode, both families of CPU require almost the same amount of power. Yet, the maximum transmission power demands of STM-32 are 14 times smaller than typical transmission power demands of ESP-32. The key advantage of ESP-32 is that it's target TX power is configurable based on device or certification requirements. By decreasing the transmission power of Wi-Fi protocol, the range of transmission gets reduced. This could become a key advantage for the potential Kiwi applications due to Kiwi transmitting the data to the earbuds, which are located only 20-30 centimeters apart. Since the Wi-Fi protocol is considered to have range of dozens of meters, vast percentage of the power demands can be reduced to transfer the data across 20-30 centimeters distance only. The scale of how much it can be reduced and it's comparison to Bluetooth 5.2 LE can be found only empirically. Nevertheless, the demands for the CPUs of ESP-32 are still significantly higher, which allows to make the conclusion: the STM-32-based KIWI carries an advantage in power efficiency to ESP-32-based KIWI.

Hence, the core of the choice between the 2 proposed technologies lies in the compromise between speed/volume and power demands. If the recording speed or decrease in size is being prioritized, ESP-32 satisfies the needs better. On the other hand if the power required is the most crucial factor, then STM-32 is an optimal solution.

Embodiments may combine best features of the two proposed technologies by developing and constructing the individual ASIC with implemented Wi-Fi transmission protocol and the befitting ADC and DACs. The main two reasons behind this approach are:
1. SoCs and microcontrollers are developed and fabricated to be versatile and flexible for various amount of applications. The flexibility comes at a cost of increased power demands for all peripherals, the clocking structures, modules, etc.
2. The majority of microcontroller possess DACs and ADCs, which have the average performance characteristics (2-5 Msps, serial, 5 INL, 10 DNL, etc.). As it was demonstrated before, the sampling speed of ADC is the limiting factor and one of the biggest challenges towards multi-thousands neural recording system.

The developed and constructed ASIC should meet the following requirements:
1. The size of the ASIC should be smaller in geometrical parameters than ESP-32 die to reduce the Kiwi shell even further
2. The power demands for developed CPU, sampling, generating, and transmission modules for the ASIC should not exceed 10 mW
3. The ASIC has to be able of recording thousand channels and be capable of having the amount of pixels scaled to dozens of thousand electrodes

The third condition presupposes having a fast wireless protocol and an ADC capable of sampling at 15-20 Msps. Developing and constructing an individual ADC, as an embedded module of the ASIC, can bring a noticeable impact on the system. Taking as a reference example of ADC1173—low power 8-bit 15 Msps ADC, that consumes from 15-30 mW and has 7.15 mm×4.15 mm physical parameters.

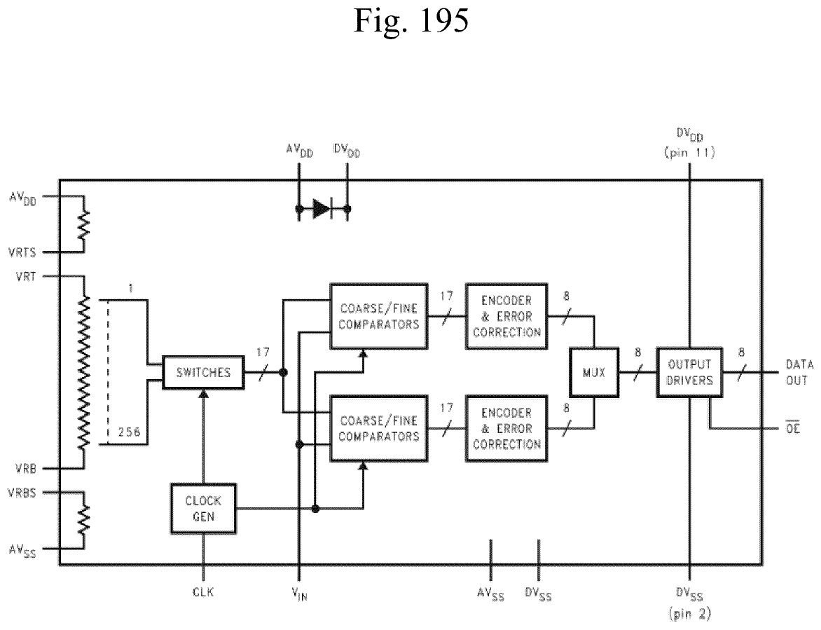

Consumption of 15 mW to 30 mW does not satisfy the 2nd requirement listed above. Yet, the special feature of the flash type ADC is that power demands rise and fall with increase and decrease of the number of switches, as shown in FIG. 195, which is a block diagram on an exemplary ADC, the ADC1173. Developing and fabricating the ADC with an almost identical structure and bits resolution of 6 bits would approximately reduce power demands to 4-7 mW. Assuming the proposed ASIC possess an ADC of such parameters and minimal LZO compression

$$f = \frac{1}{{n}_{pixels} ( \frac{4}{150 * 1 {0}^{6} sps} + \frac{1}{15 * 1 {0}^{6} sps} )} = \frac{1}{500 ( \frac{4}{150 * 1 {0}^{6} sps} + \frac{1}{15 * 1 {0}^{6} sps} )}$$

$$f = 21.42 kHz$$

The resulting recording frequency is and 6.2 times greater than recording frequency of ESP-32-based Kiwi and 18.6 times faster than recording frequency of STM-32 based Kiwi correspondingly. The threshold recording frequency, beyond which it is both physically and theoretically impossible to record is also found:

$${f}_{limit} = \frac{1}{500 ( 0 + \frac{1}{15 * 1 {0}^{6} sps} )} = 30 kHz$$

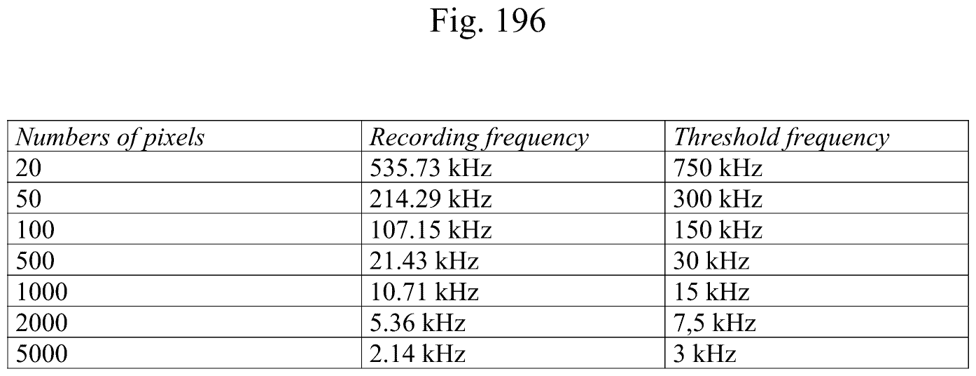

Which is 7.5 times higher than the threshold recording frequency of the proposed ESP-32-based Kiwi. Varying the numbers of electrodes, the range of possible recording and threshold frequencies is summarized in the table shown in FIG. 196, which shows conversion, transmission and geometrical characteristics for the ESP32-based KIWI.

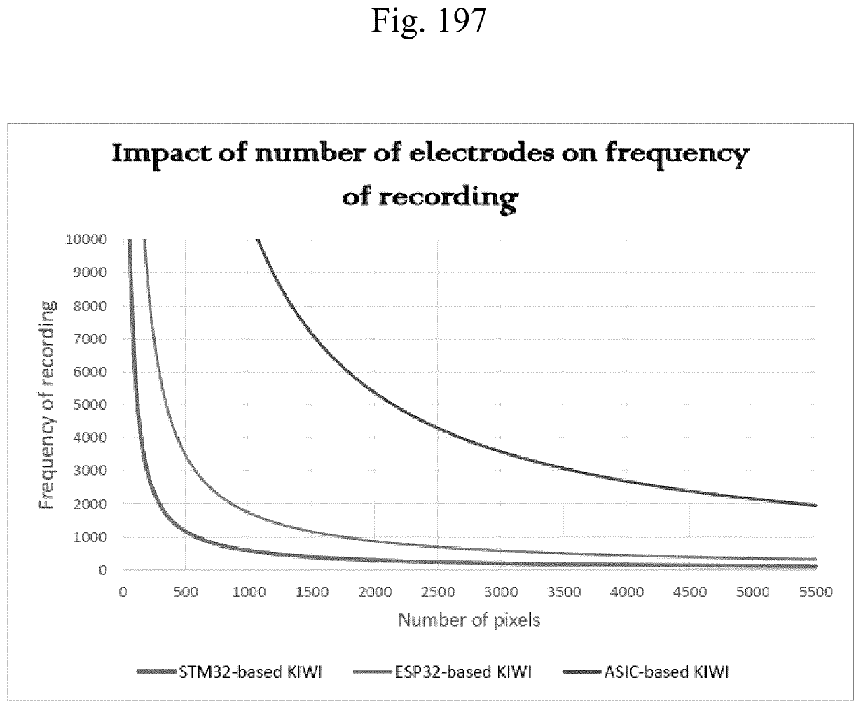

FIG. 197, which shows the Numbers of pixels vs Frequency of recording for the STM-32, ESP-32, and individual ASIC, illustrates the beneficial impact of developing an individual ASIC with the described features.

Embodiments may provide stimulation techniques, such as electrical stimulation techniques, chemical stimulation techniques, and optical stimulation techniques. Such techniques may be invasive or non-invasive. For example, non-invasive electrical stimulation techniques may include Transcranial Direct Current Stimulation (tDCS) and Transcranial Direct Current Stimulation (tACS), while invasive electrical techniques may include electrodes implanted in the body, such as in brain tissue. For example, chemical stimulation techniques may include structures to stimulate neurotransmitters in vivo. For example, non-invasive optical stimulation techniques may include Transcranial Photobiomodulation (tPBM), while invasive optical stimulation techniques may include optical fibers to emit photons to affect neural tissue. Further, optrodes may provide some or all of electrical, chemical, and optical stimulation.

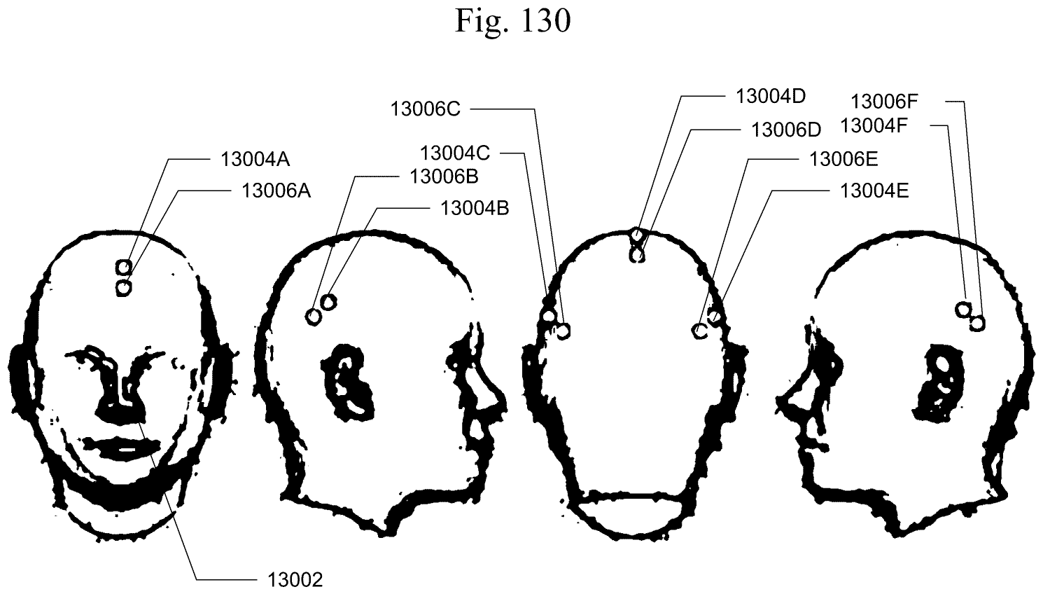

Embodiments may provide what may be termed a Next Generation Transcranial Kiwi (tKiwi), which may include a combination of Transcranial Photobiomodulation (tPBM), Transcranial Direct Current Stimulation (tDCS), Transcranial Direct Current Stimulation (tACS) with dynamic closed loop feedback such as may be provided by an Electro Encephalogram (EEG). For example, as shown in FIG. 130, embodiments may provide tPBM using 4-8 Red LEDs at 810 nm wave length with, for example, one Nasal LED 13002 configured to be mounted in a nostril of a person, plus 3-7 head mounted LEDs 13004A-F, 13006A-F. Nasal LED 13002 may have parabolic focused reflectors to increase penetration and direction light from of Nasal LED13002. Likewise, head mounted LEDs 13004A-F, 13006A-F may include parabolic focused reflectors.

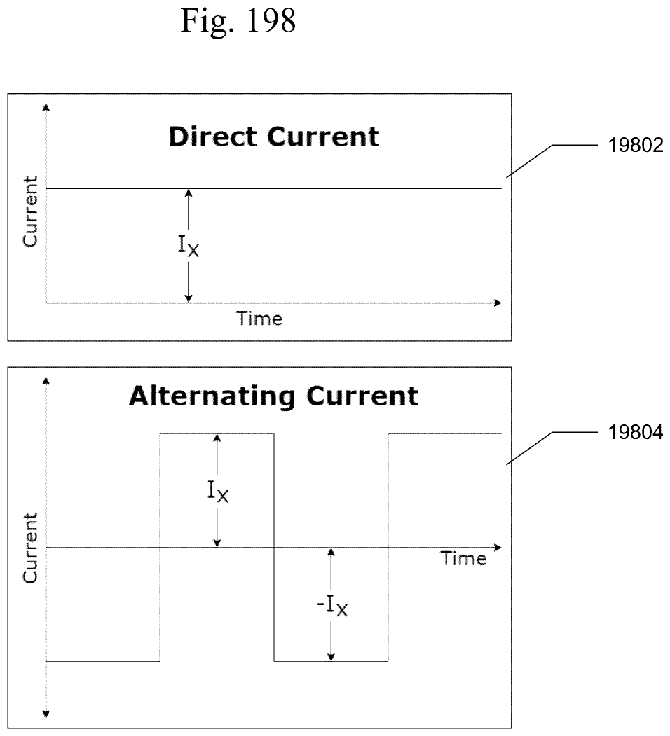

Turning briefly to FIG. 198, exemplary waveforms of Transcranial Current Stimulation (tCS) including Transcranial Direct Current Stimulation (tDCS) 19802 and Transcranial Alternating Current Stimulation (tACS) 19804 are shown. tDCS 19802 has no frequency content (0 Hz) and involves delivering a set value of current, Iₓ, delivered continuously from one electrode to the other. tACS 19804 may, for example, provide signals having frequencies up to 40 Hz, or in embodiments, up to 500 Hz. The frequencies may be fixed, variable, or varying during delivery. The delivered signals may have a fixed duty cycle, for example, 25%, 50%, 75%, etc., or may have variable duty cycles, or duty cycle that vary during delivery. Set values of current, −Iₓ to Iₓ, to alternate directions between electrodes may be delivered. In embodiments, variable, or varying values of current may be delivered.

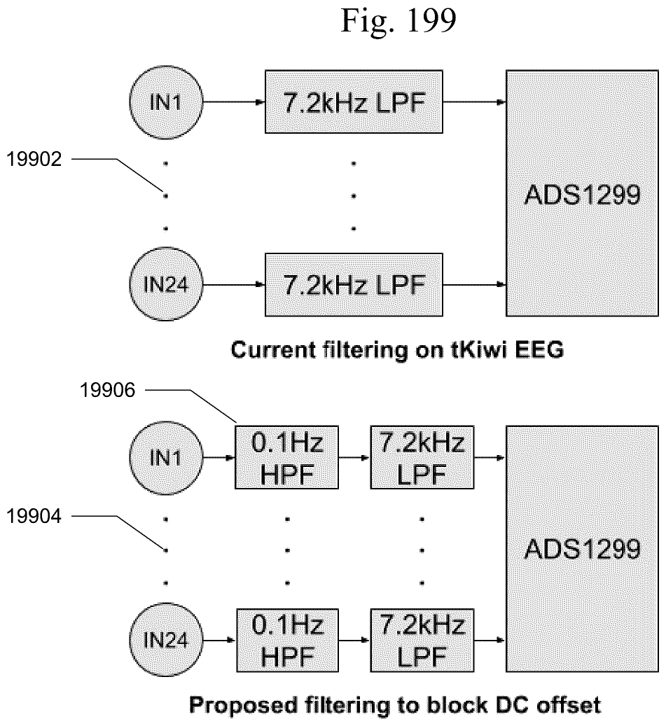

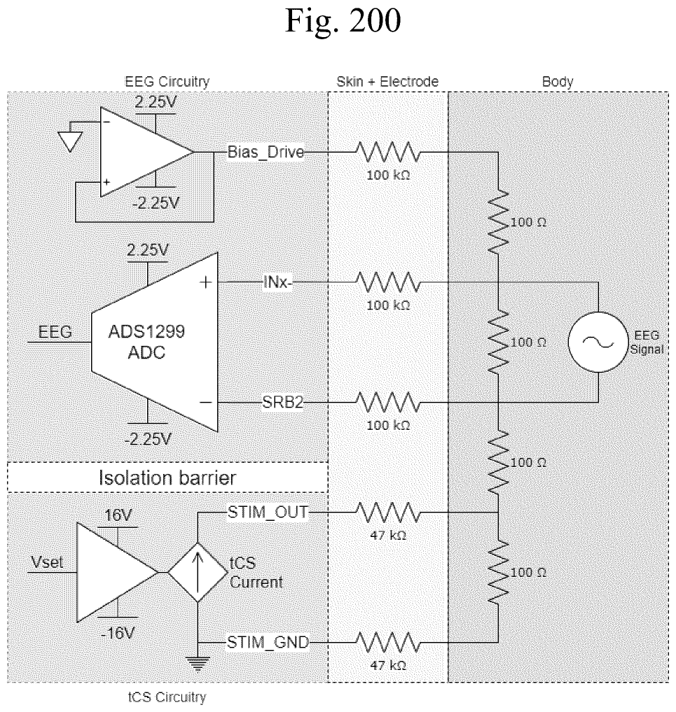

As described elsewhere herein, embodiments may provide simultaneous tDCS or tACS stimulation and EEG monitoring. Exemplary embodiments of circuitry implementing such a feature are shown in FIGS. 199 and 200. Typically, delivery of stimulation signals during EEG monitoring may interfere with the monitoring of the EEG. As shown in FIG. 199, embodiments may include a high-pass filter on the input for EEG signals to reduce such interference. For example, EEG input circuitry 19902 does not include such high-pass filtering, while EEG input circuitry 19904 does include such high-pass filtering 19906, which blocks the DC offset caused by tDCS stimulation. However, while such embodiments may reduce interference from tDCS stimulation, they may not effectively reduce interference from tACS stimulation.

FIG. 200 illustrates an exemplary embodiment in which the grounds between the EEG monitoring circuitry and the tDCS and tACS stimulation circuitry are isolated. Isolation would separate EEG monitoring circuitry from rest of circuitry. The ensures that the EEG circuitry will be able to measure accurately during stimulation. The embodiment provides for both tDCS/tACS and simultaneous EEG monitoring. Further, such isolation will reduce parasitic PCB noise generated by tPBM and tDCS/tACS circuitry.

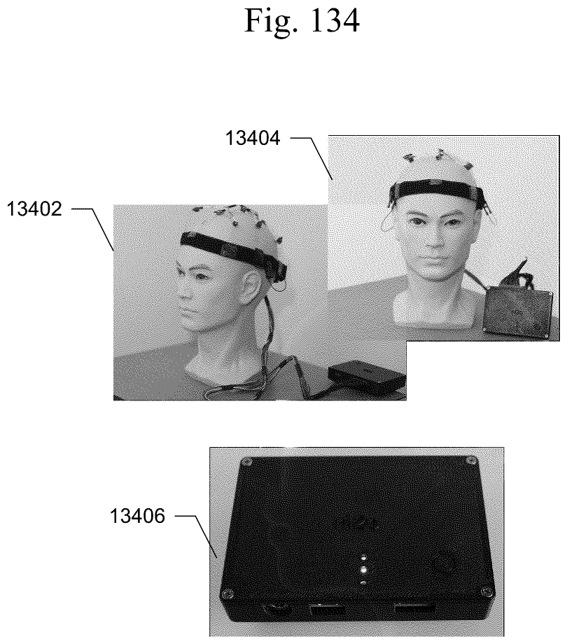

Turning briefly to FIG. 134, shown are examples 13402, 13404 of a tKiwi headset, and of a tKiwi controller 13406. In embodiments, a tKIWI system may comprise a wearable neuromodulation/EEG headset 13402, 13404 to manage and treat symptoms with, for example, 97% accuracy. For example, conditions that may be treated may include depression, Parkinson's disease, dyslexia, and other mental health disorders and neurodegenerative diseases

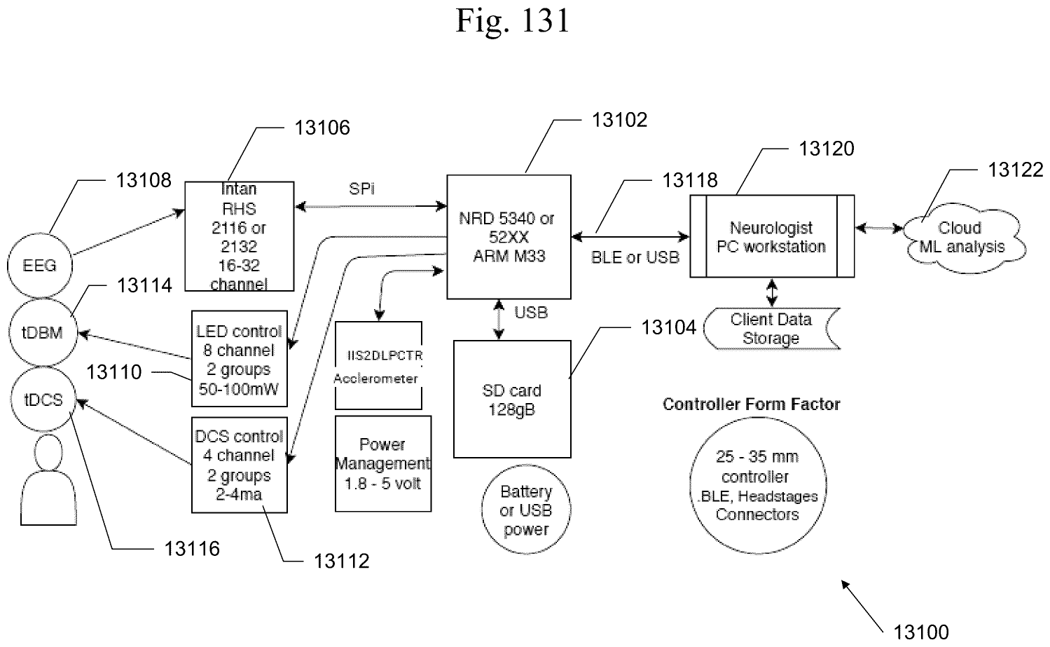

An exemplary block diagram of tKiwi system 13100 is shown in FIG. 131. In this example, system 13100 includes processor 13102, memory 13104, interfaces 13106 to Electro-encephalogram (EEG) sensors 13108, interfaces 13110, 13112 to tPBM LEDs 13114 and tDCS electrodes 13116, communications interface 13118, workstation 13120, and the cloud 13122. Processor 13102 may include a processor, such as a microprocessor or microcontroller, memory, and interface circuitry to communicate with other devices in system 13100. Memory 13104 may include mass storage, such as flash memory, etc. Interface 13106 may include multi-channel input circuitry to receive and process signals from EEG sensors 13108. Interface 13110 may include multi-channel output circuitry to send control signals to tPBM LEDs 13114. Interface 13112 may include multi-channel output circuitry to send control signals to tDCS electrodes 13116. Communications interface 13118 may include input output circuitry, such as wired interfaces, such as Ethernet or USB, and/or wireless interfaces such as Bluetooth, WiFi, etc. Communications interface 13118 may provide communications with workstation 13120, which may provide control, analysis, and display functions, and may communicate with the cloud 13122, which may provide further analysis, such as Machine Learning (ML) analysis.

Turning to FIG. 130, embodiments may provide features such as Nasal LED 13002 may be independently controlled from head mounted LEDs 13004A-F, 13006A-F and can be removed or not used. Each LED 13002, 13004A-F, 13006A-F on/off and intensity may be individually controlled or group selectable settings may be used. LEDs 13002, 13004A-F, 13006A-F may be strobe pulsed (frequency and intensity). For example, 10 Hz Alpha wave modulation may be used for Sleep, 20 Hz Beta Wave modulation may be used for Motion Learning, 40 Hz for Gamma Wave modulation may be used for Memory focus and learning activities, etc. In embodiments, LEDs 13002, 13004A-F, 13006A-F may all output the same wavelength light, while in other embodiments, at least some LEDs may output different wavelength light. For example, LEDs 13004A-F may output a first wavelength of light, such as Red 810 nm, while LEDs 13006A-F may output a different wavelength of light.

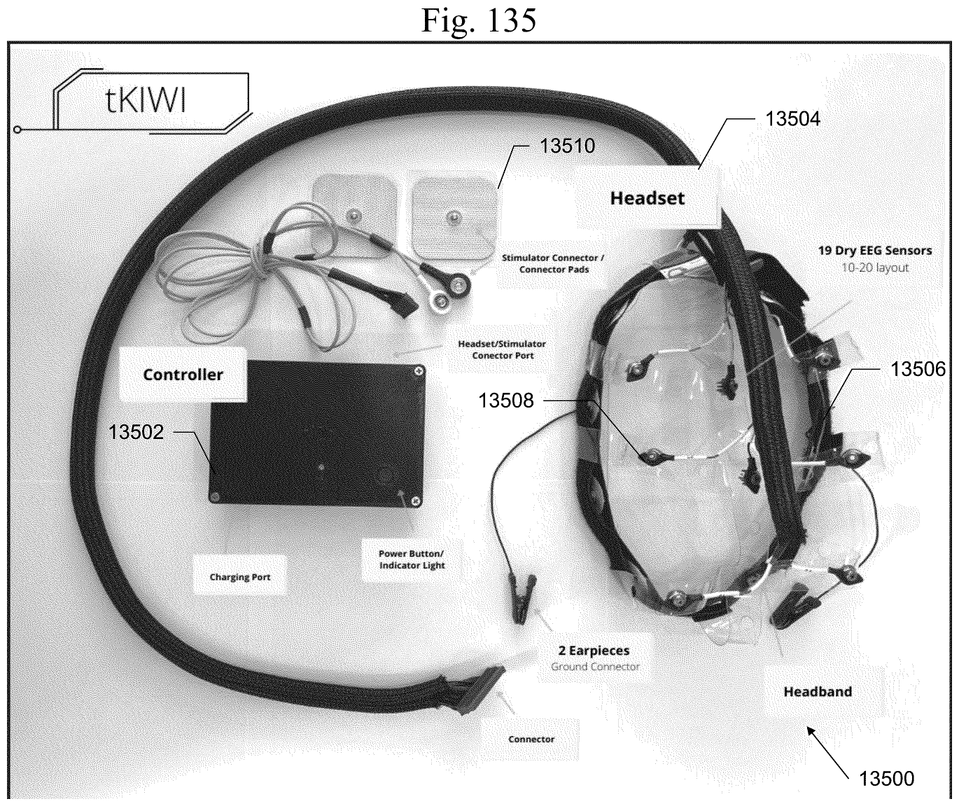

Turning to FIG. 135, an exemplary embodiment of a tKiwi system 13500 is shown. In this example, components such as a controller 13502, a headset 13504, a headband 13506, EEG sensors 13508, stimulator pads 13510, etc., are shown.

Embodiments may provide tDCS using, for example, 4-8 electric dry probes, 4 placeable (semi-damp) ground connectors, one with Nasal LED to control flow of electrons from the positive sources selected thru initial placement and dynamically selected thru closed loop feedback controller. For example, at the locations shown for LEDs 13006A-F in FIG. 130, electrodes may be placed, rather than LEDs. Embodiments may provide electrode currents of, for example, 1-4 ma, time intervals of, for example, 0.5 ms to shut off (30 min), and selection of sources and grounds as controlled by the feedback controller, which may be implemented in processor 13102.

Embodiments may provide tDCS using, for example, a triangular location algorithm to influence ion flow by dynamically switching the negative and positive terminals that the current is applied to. Flow of DC current in cells is from positive to negative. This provides somewhat of a beam forming electron flow due to variable pulsed current dynamically changing ground and sources selected by the AI controlled controller to target a given area of brain.

Embodiments may provide EEG functionality, using for example, 8-24 EEG signals that may be used as feedback to the controller implemented in processor 13102 to help determine stimulation recipes and dynamic selection of placement. Embodiments may provide ML/AI: analysis of EEG from applying LED intensity and pulse HZ and the different DC Probe timings of volts, current, and positions to dynamically determine and provide closed loop recipes to controller. Embodiments may measure voltage and current between DC positive and negative terminals to determine amount of flow of electrons (+ions) thru the brain and use to adjust light intensity, LED strobe frequency, and which LEDs to dynamically use.

Embodiments may include Controller and Probes. For example, a clinical/research embodiment may include a Headset with Probes and EEG sensors. This embodiment may include a Nasal probe with 810 nm wave length LED in tube with semi solid parabolic reflector with silver coating. The headset may include 2 or 3 adjustable bands going from ear to ear with limited adjustable electrical and LED stimulators as above. A Neurologist embodiment may include an EEG socking cap. The controller, interface, and communication circuitry may be mounted in the headset. Embodiments may include a battery. Based on the size needed, the battery may be external to headset or custom to fit in headset bands. The battery life may be at least 60 minutes. USB C charging or industry standard phone induction charging may be provided. Bluetooth connection to PC App or Phone App may be provided.

A consumer embodiment may include a detachable nasal probe with 4-8 LEDs and/or Electrical Probes, 2-4 EEG Probes permanently attached at the best locations. The probes may be mounted on a two band headset with controller inset in the bands. A Phone App may control On/OFF, Battery status, 6-8 selectable stimulation recipes set by a neurologist, sleep monitoring without nasal probe, possible deep learning mode for therapy training or sports.

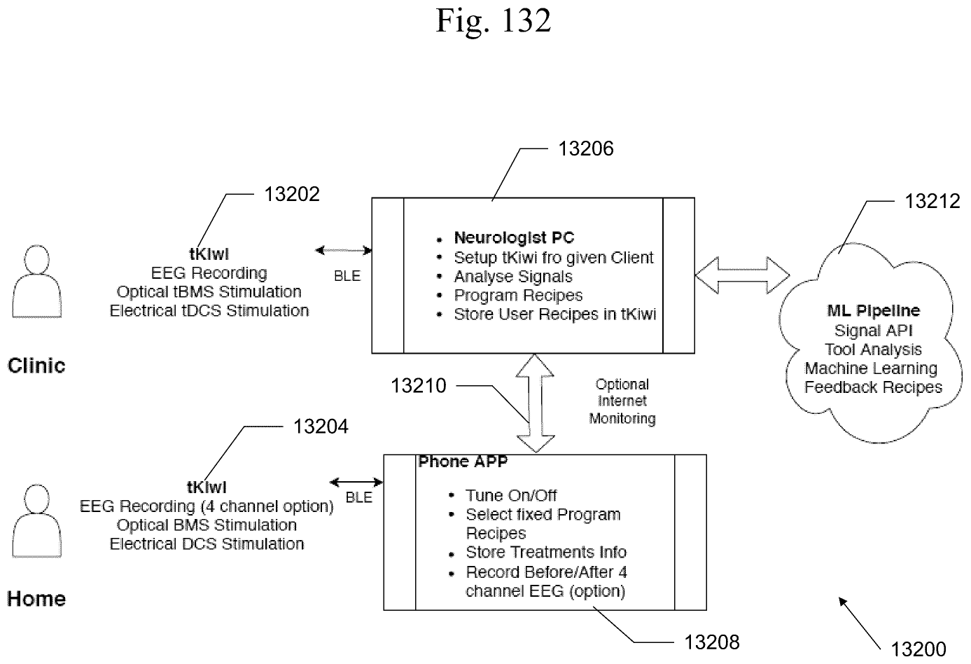

An exemplary block diagram 13200 of overall operation of the tKiwi system is shown in FIG. 132. As shown in FIG. 132, this example, may include clinical tKiwi 13202, home tKiwi 13204, Neurologist PC 13206, Phone App 13208, Internet monitoring 13210, and machine learning (ML) pipeline 13212. Clinical tKiwi 13202 and home tKiwi 13204 are described above. Neurologist PC 13206 may include software to provide the capability for a Neurologist or Researcher to setup, control and analyses of results obtained from clinical tKiwi 13202, and with Internet monitoring 13210, results obtained from home tKiwi 13204. Neurologist PC 13206 software may provide the capability to setup clinical tKiwi 13202 and/or home tKiwi 13204 for each client. For example, the software may provide the capability to define a plurality of stimulation recipes for each client and to store at least some of them on each tKiwi device. Neurologist PC 13206 may further include additional software tools such as EEGLab, InTan tools, BrainLab, etc., with converters if needed for compatible formats. Neurologist PC 13206 may further include a Research App to build and analyze ML pipelines 13202.

Phone App 13208 is for client use with a consumer version of tKiwi, such as home tKiwi 13204. Embodiments may be limited to stored recipes set by a neurologist, for example, using Neurologist PC 13206. Home tKiwi 13204 may communicate with Phone App 13208 using, for example, Bluetooth. Internet monitoring 13210 may be performed via an Internet connection to the clinic for monitoring if desired. Treatment information and treatment sessions may be recorded for the neurologist by Phone App 13208 and/or in memory 13104. Phone App 13208 may provide a sleep monitoring mode, deep learning thru stimulation for therapy or sports, etc.

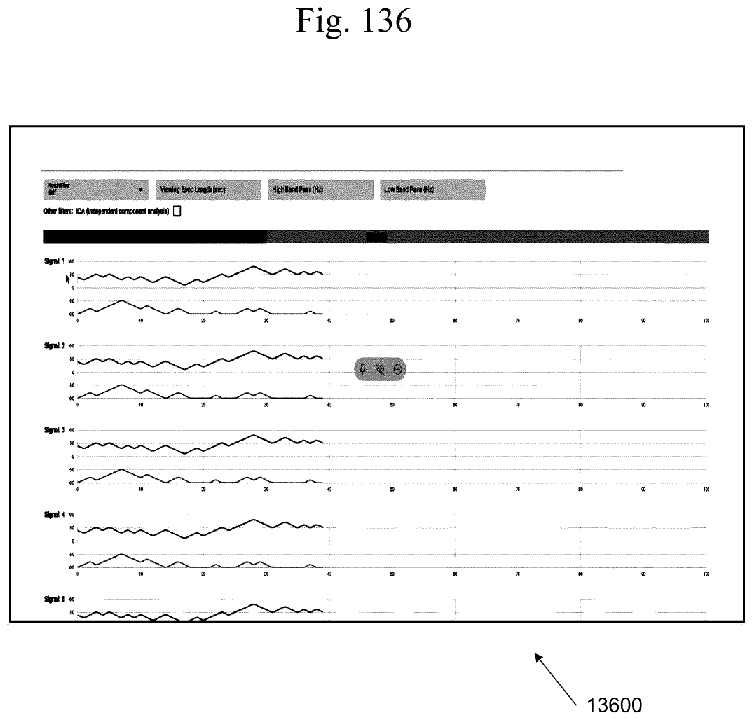

Turning to FIG. 136, an example of personalized treatment is shown. Personalized Treatment settings, or “Recipes”, may utilize features such as AI/Deep Learning (DL), an EEG sensor headset, such as with 8 or 16 channels and 2-4 adjustable dry electrodes, tDCS and tACS stimulation, software to detect brain states and display via the PC app 13600, and a number of brain disorders coded to track treatment improvement over time.

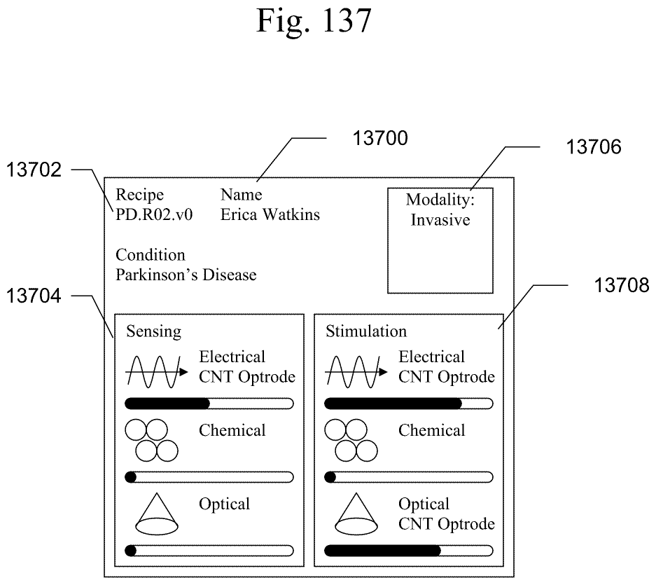

An example of a recipe 13700 is shown in FIG. 137. Recipes are sets of code and AI models deployed to one or more treatment devices, whether implanted or used as a wearable. For example, recipes may utilize sensing techniques, such as electrical sensing techniques, chemical sensing techniques, and optical sensing techniques. Such techniques may be invasive or non-invasive. For example, recipes may utilize stimulation techniques, such as electrical stimulation techniques, chemical stimulation techniques, and optical stimulation techniques. Such techniques may be invasive or non-invasive

These recipes determine how different sensing and stimulation techniques are combined to maximize efficacy for each individual patient's therapy. Recipe 13700 may include version identification 13702, sensing setup 13704, modality 13706, and stimulation setup 13708. VERSIONING 13702—The initial version of a recipe may provide a baseline set of instructions for the therapy. SENSING 13704—Sensing may be based on sensing techniques, such as electrical sensing techniques, chemical sensing techniques, and optical sensing techniques. Such techniques may be invasive or non-invasive. In this example, sensing based individual neuron activations (action potentials) for implantable chip, EEG and other technologies for wearables is shown. MODALITY 13706—Recipes may be delivered through implantables and wearables. STIMULATION 13708—Stimulation techniques may include electrical stimulation techniques, chemical stimulation techniques, and optical stimulation techniques. Such techniques may be invasive or non-invasive. In this example, a carbon nanotube optrode delivers electrical and optical signals.

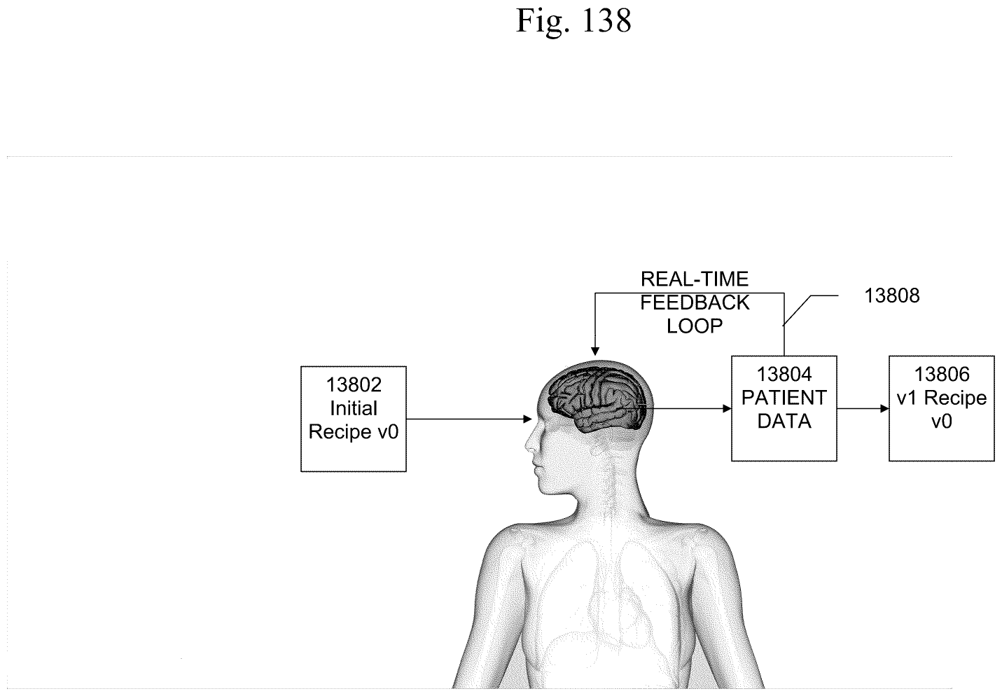

Recipes may be modified, as shown in FIG. 138. For example, an initial recipe 13802, version 0 may be applied to a patient and data 13804 relating to the effect of that recipe on the patient may be sensed and used to modify the recipe to form a new version of the recipe 13806. For example, we may begin with basic recipes that may be informed by past treatment protocols. These starting recipes may be adjusted based on the needs of an individual patient to form an initial recipe 13802 and may be revised as more data 13804 is collected during therapy. During therapy, a real-time feedback loop may be used to adjust the treatment in real-time. This adjusted treatment data 13804 may be used to modify the recipe to form a new version of the recipe 13806.

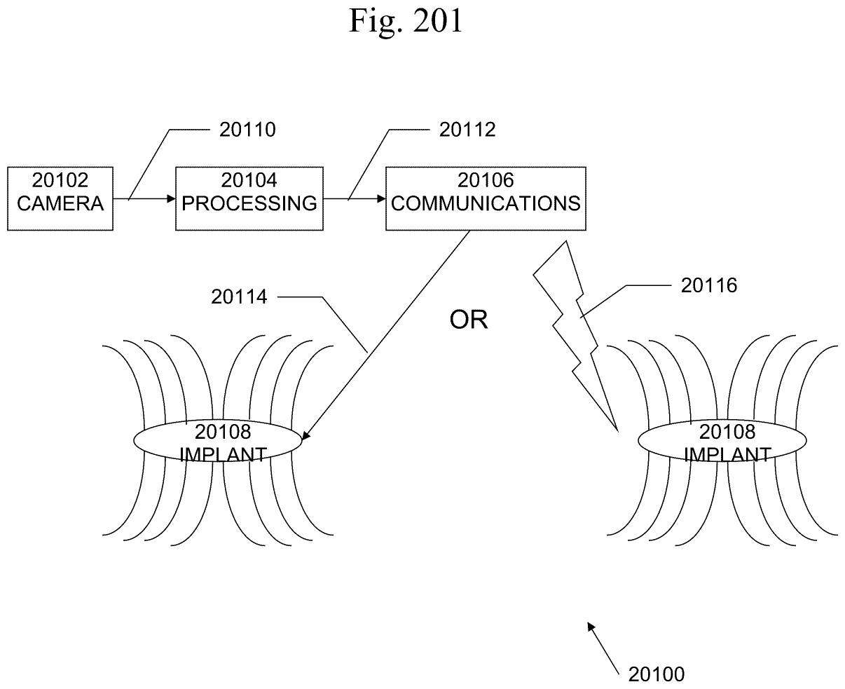

An exemplary block diagram of an embodiment of an artificial vision system 20100 is shown in FIG. 201. System 201 may include a camera 20102, processing circuitry 20104, communications circuitry 20106, and one or more implant devices 20108. Camera 20102 may be used to gather visual information 20110, which may correspond to or approximate visual information in a field of view of a person. Visual information 20110 may be input to processing circuitry 20104. Processing circuitry may transform the visual information into control signals 20112 to generate stimulation signals. Control signals 20112 may be input to communications circuitry 20106 for transmission 20114, 20116 to implant device 20108. Such transmission may be over a wired connection 20114 or a wireless connection 20116. Implant device may receive the control signals, generate stimulation signals based on the control signals, and apply the stimulation signals to neural tissue, such as the visual cortex of the brain to provide neural stimulation representative of the original visual information.

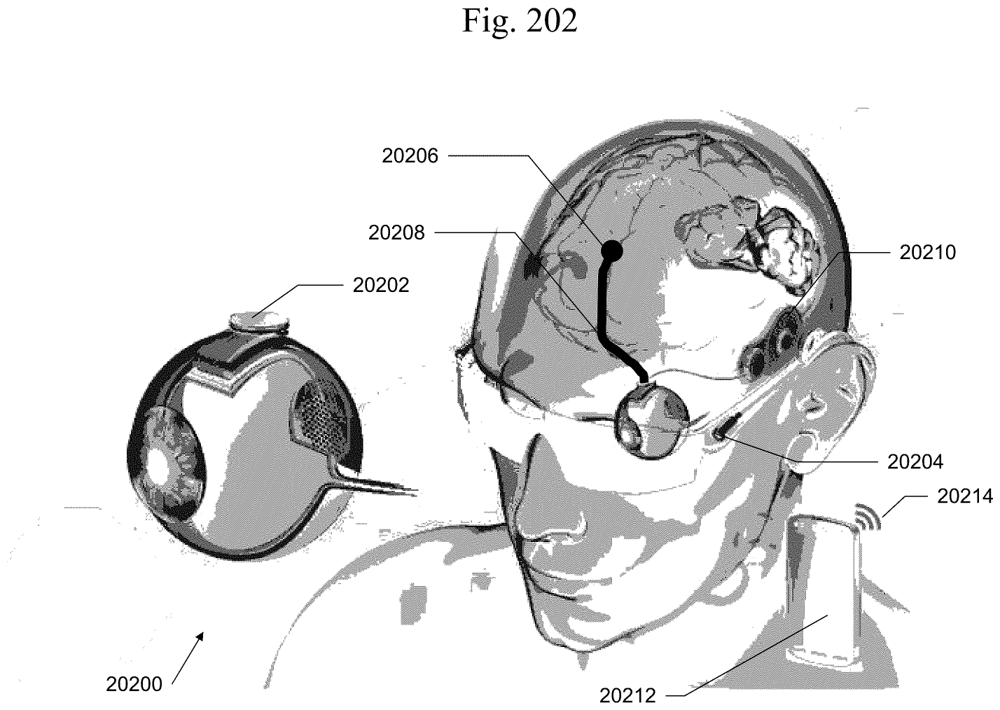

An example of an embodiment of an artificial vision system 20200 is shown in FIG. 202. Examples of a camera, as shown in FIG. 201, may include a prosthetic eye 20202, or an external camera 20204. Prosthetic eye 20202 or external camera 20204 may gather visual information and input the gathered visual information to processing circuitry, such as processing circuitry 20104, shown in FIG. 201, which may transform the visual information into control signals. Processing circuitry 20104 may be included in prosthetic eye 20202, external camera 20204, or in an external unit 20212. In embodiments, the gathered visual information may be transmitted to the processing circuitry over a wired connection or a wireless connection. Likewise, in embodiments, the control signals may be transmitted to the implant device 20206 over a wired 20208, wireless 20214 connection, or wired/wireless 20110, 20114 connection. The precise configuration of which connections are wired and which connections are wireless are engineering considerations based on, for example, the size and/or power requirements of the various components.

In embodiments, the implant device may include embodiments of the implant devices described herein. Such implant devices may provide features described herein, including miniature size, minimally invasive, wireless charging, wireless communication, device lifespan=patient lifespan, embedded receiver/transmitter, 1 Gb/s transmission due to compression technology, 100,000 or more stimulation sites, etc. In embodiments, the stimulation signal may be electrical, optical, or a combination of electrical and optical.

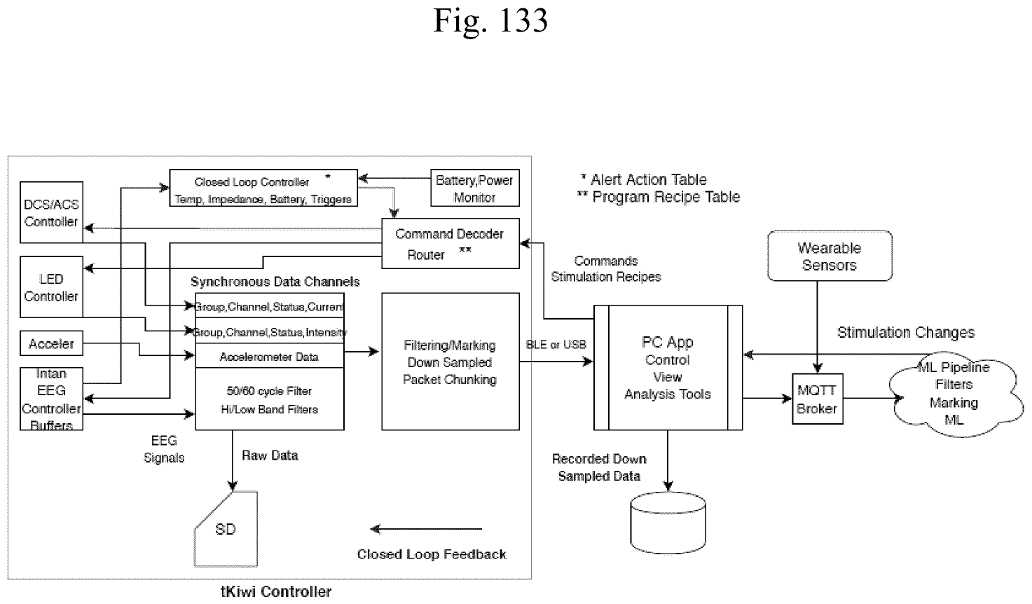

An example of an embodiment of a tKiwi system data flow is shown in FIG. 133.

Embodiments may provide EEG acquisition. The EEG signals may be recorded, for example, at 256 Hz sampling rate and a bandwidth of 0.43-80 Hz. The amplifier may be low-noise DC-sensitive feeding a 24-bit analog-to-digital converter. For the EEG capture, for example, we used a 19-Channel free-cap set may be used, which allows EEG to be recorded during tPBM delivery. The EEG channels may be located at a 10-20 montage on the elastic net. The data may be collected, for example, during 10-minute sessions with the subjects at rest and with the eyes closed, before and after Neuro Gamma stimulation.

Embodiments may provide transcranial direct current stimulation (tDCS) and, in addition, transcranial alternating current stimulation (tACS). Embodiment may provide features such as up to 32 channels of electroencephalography (EEG) data recording, on board accelerometer for motion sensing, USB Type C connector for 5V power and data connection, transcranial photobiomodulation (tPBM) control for 2 head groups each with one 810 nm wavelength LED and 1 nasal application group with an LED with a wavelength in the range 600 nm to 699 nm.

As shown in FIG. 130, there may be a plurality of groups of LED, such as a first group of LEDs 13004A-F and a second group of LEDs 13006A-F. In embodiments, the two groups for the head may operate independently from one another and will be controlled by software. In embodiments, LEDs within a group may operate independently or non-independently. The LEDs may be controlled by software, but controlling individual LEDs within a group may or may not be implemented depending on the embodiment. The software may control a group to strobe between, for example, 10-40 Hz at 50% duty cycle. The LED Irradiance may be, for example, between 75 mW/cm² and 150 mW/cm² inclusive.

In embodiments, there may be only one LED 13002 for the nasal cavity. It may operate independently from the head groups and may be controlled by software. The software may control the nasal LED to strobe between, for example, 10-40 Hz at 50% duty cycle. The LED Irradiance may be, for example, 25 mW/cm² target LED irradiance. In embodiments, nasal LED 13002 may emit, for example, a 624 nm or 633 nm wavelength red with an 8° viewing angle.

Embodiments may have tDCS and tACS capabilities such as, for example, 5 probe connections total: 1 group with 4 single ended channels and 1 ground/reference channel for return. In embodiments, the channels may operate independently or non-independently. Examples of channel settings include ±0.5 to ±2 mA controlled current output with 0.1 mA increment resolution, 2-100 Hz settable frequency for tACS with 1 Hz increment resolution. The tACS signal may be a square wave that alternates between +“x” and −“x”, where “x” is the user setting between 0.5 mA and 2 mA. The tDCS signal may be direct current output that will operate at ±“x”, where “x” is the user setting between 0.5 mA and 2 mA

Embodiments of the present techniques may provide a small design that allows it to be implanted via far less invasive means, decreasing the risk of surgical complications and adverse effects. For example, in embodiments, a chip may be implanted through blood vessels without opening the skull. This approach may involve insertion via a blood vessel in the neck and guidance to the brain using real-time imaging. Once the chip reaches the target location it expands and attaches to the walls of the blood vessel to read the activity of the nearby neurons.

Embodiments may provide a Kinetic Intelligent Wireless Implant KIWI) comprising an ultra-low power computer device with interconnects that can attach to nerve or brain tissue and read signals/voltages and/or stimulate those tissues with electrical or optical pulses. Thus, KIWI provides the capability to implement a Brain Code Collection (and Stimulus) System (BCCS). The interaction between the KIWI and the tissue may be performed through arrays of optic fibers coated with single wall carbon nanotubes (CNTs). CNTs have been chosen as they have been shown to readily attach to tissue and possess remarkable electric properties. Effectively, CNTs serve as electrochemical and optical sensors as well as measurement/stimulation electrodes.

An exemplary block diagram of an embodiment of a KIWI system 12000 in which the present techniques may be implemented is shown in FIG. 120. KIWI system 12000 may include a charging and communication unit 12002, a propagator 12004, and an implant device 12006, including at least one array 12008 of optic fibers coated with single wall carbon nanotubes (CNTs). Charging and communication unit 12002 may transmit data to a Gateway device such as a cell phone or other nearby computer which can in turn analyze data, give input to KIWI system 12000, or send the data to the Cloud for deep analysis. Therefore, KIWI system 12000 provides a revolutionary brain computer interface for research in Neuroscience and medicine, as it is the first closed-loop neural modulator informed by both internal and external conditions.

NEURAL SENSING—Embodiments may support channel types such as Optrodes (optical electrode) and Electrodes. Optrodes are capable of optical and electrical recording and stimulation. Optrodes may be composed of optical fibers coated with single walled carbon nanotubes. Optical fibers are used for transporting light signals bidirectionally. Electrodes can perform electrical recording and stimulation. Carbon nanotubes may be used to transport electric signals and can detect neurotransmitters in neural tissue through fast-scan cyclic voltammetry (FSCV).

ELECTRODES—The configuration and implant position of the KIWI system 12000 offers superior conditions for multi-point electric stimulation. For example, KIWI system 12000 may be able to connect to layers I to VI due to the length and geometrical configuration of the CNTs. Due to its 2000+CNT fibers, KIWI system 12000 has a far greater number of stimulation points, offering a far superior spatial resolution.

VOLTAGE SENSING—In the simplest use case, CNTs can be used for deep brain recordings of voltages from neural tissues in their vicinity. CNT based electrode arrays enable high-density neural connections in a manner that is non-destructive to the neuronal tissue. This method is feasible and efficient because of CNT's superior mechanical, thermal and electrical properties.

CHEMICAL SENSING—CNTs can be used in yarn macrostructures (which are several parallel CNTs) to detect neurotransmitters in vivo. Disk-shaped CNT yarns have been developed to detect electro-active neurotransmitters. The technique to detect these neurotransmitters, Fast-scan cyclic voltammetry (FSCV), monitors changes in the extracellular concentration of electroactive molecules when the electrode is ramped up versus time, up to a certain threshold, and then it is ramped in the opposite direction to return to the initial potential.

OPTICAL MULTIPLEXING—The different geometries of the carbon atoms in CNTs determine different electronic properties. The different electronic properties are correlated with different optical properties because their electronic band-gap between valence and conduction band make the single walled CNTs fluorescent in the near infrared (NIR, 900-1600 nm). This property enables CNTs to be used for optical multiplexing because every chiral configuration may be used as a single color.

OPTRODES—In addition to KIWI system 12000 having the ability to read and write electric and electrochemical signals to and from the neurons through the CNTs, KIWI system 12000 may also have the capability of reading optical signals and optically stimulating neurons through an array of optical fibers using a process called optogenetics. Optogenetics and fiber photometry are neuro-modulation technologies in neuroscience that utilizes a combination of light and genetics to control and monitor neurons in vivo. Optogenetics is a method for brain stimulation/modulation by inducing well-defined neuronal events at a millisecond-time resolution, enabling optical control of the neural activity.

OPTICAL SENSING OF NEUROTRANSMITTERS—optical fiber sensing of neurotransmitters may provide advantages over the electrochemical sensing techniques. For example, the lower limit of detection can reach a nanomolar range or less (compared, for example, to a 300 nm LOD for dopamine detection using electrochemical sensing by CNT yarn microelectrodes. The broad optical spectrum allows for the interference from other chemical species to be minimized. Optical fiber sensing may provide high spatial resolution. The release and uptake of neurotransmitters occurs in a highly localized fashion. The sensors are small enough to identify which neurons are involved in specific chemical interactions. Optical fiber sensing may provide high temporal resolution. Neurotransmitter release and uptake processes occur within a millisecond time range. The sensors possess a sampling rate that is high enough to detect the concentration changes.

NEURAL STIMULATION & NEUROMODULATION—KIWI system 12000 may stimulate neural tissue through the same methods by which it can detect incoming signals, through optical, electrophysiological stimulation and electrochemical means.

OPTICAL STIMULATION—Optogenetics is a technology in which light-sensitive ion channels are virally expressed in target neurons, allowing their activity to be controlled by light. Optogenetics can be used to silence certain neural pathways in live mammals and can precisely control the function of single-cells. Optogenetics can be used as a side-effect-free method for alleviating symptoms of neurological diseases which occur through either neuronal overexcitability (i.e. epilepsy) or underactivity (i.e. schizophrenia). One of its practical advantages is that it has minimal instrumental interference with simultaneous electrophysiological techniques.

By coating optical fibers with dense, thin CNT conformal coatings, KIWI system 12000 may include optical modulation units within the nucleus of the KIWI implant that can deliver light to precise locations deep within the brain while recording electrical activity at the same target locations. We are currently developing precisely targetable fiber arrays and in vivo-optimized expression systems to enable the use of these tools in awake, behaving primates.

The light-activated proteins Channelrhodopsin-2 (ChR2), a light-sensitive ion channel, Halorhodopsin(NpHR), an optically activated chloride pump, and Archaerhodopsin (Arch), a proton pump, can be used to activate and inhibit neurons in response to light of different wavelengths. It has been shown that ChR2 and NpHR can be genetically expressed in neurons using a viral approach. Conventionally these viruses are injected in the neural tissue, but embodiments may provide the virus vector carried on the tips of the CNTs. Due to their small dimensions, these viruses do not interfere with the reading and stimulation processes.

There are several types of Channelrhodopsins, each one responding to a particular wavelength. Some Channelrhodopsins stimulate neuronal activity (ChR2), while others inhibit it (NpHR). Therefore, the optical sensitivity of these proteins enables both the increasing/activation and decreasing/silencing of the voltage inside neurons, by targeted laser beams of blue and yellow light, respectively. The technique is deemed as safe, precise, and reversible.

KIWI system 12000 may include software that can be synchronized with optogenetic actuators and sensors and fiber photometry devices to allow for the acquisition of behavioral data during experiments by using TTL (transistor-transistor logic) as well as a specially developed software interface. This will allow the possibility of simultaneous control of biochemical events of living, freely behaving animals and the high-throughput and real-time collection of this data, creating a new realm of research.

In order to both monitor and modulate biochemical events in animals, the animals must be able to move freely without being restricted by wires or tethers. This is supported by the KIWI as all data exchanges and power delivery are wireless. This is ideal for studies such as mapping function of the amygdala in instances such as fear conditioning, targeting pharmacotherapies and addiction via the nucleus accumbens, expression of pyramidal neurons in Prefrontal cortex (PFC) and genetic components of social behavior and drug efficacy in neuropsychiatric disorders.

ELECTROPHYSIOLOGICAL STIMULATION—Electrophysiology is a tool for deep brain stimulation in which electrical current is applied via electrodes implanted on/in the brain parenchyma. While optical stimulation is able to target specific neurons very precisely, electrical stimulation implies current dissipation in the surrounding area. CNTs connected to nanoelectrodes implanted directly in the brain parenchyma can achieve electrophysiological stimulation.

NEURONAL RECORDING—In embodiments, KIWI system 12000 may record, for example, 2000 channels simultaneously. Since the KIWI features both optic fibers and CNTs that can have multiple roles, the KIWI will be able to record neuronal data via the same electrophysiological, optical and electrochemical methods mentioned previously.

Therefore, Neurons on Augmented Human (NOAH) can operate on specialized KIWIs that feature only one type of neural interaction, or on hybrid KIWIs that feature all types of interaction. In the latter case, complex AI algorithms will need to be used for CNTs management according to their properties.

ELECTROPHYSIOLOGICAL RECORDING—In electrophysiology, the oldest strategy for neural recording, an electrode is used to measure the local voltage at a recording site, which conveys information about the spiking activity of one or more nearby neurons. Electrophysiological recording functionality in the KIWI relies on the special current carrying capacity of the CNTs. The number of recording sites may be smaller than the number of neurons recorded since each recording site may detect signals from multiple neurons in the area.

OPTICAL RECORDING—For optical recording, neurons will have to be genetically modified to have fluorescent capabilities and will need to be illuminated to trigger fluorescence. The fluorescence will vary based on the voltage that is going through the membrane of the neuron, so the recorded light intensities will correspond to the voltage strength of the neurons.

Given that the optic fibers in the optrode are used for both optical stimulation and recording, a Beam Splitter is positioned close to the optrode to convert the two-way light circuit into two one-way light circuits.

Optical recording may be performed in two ways:

Firstly, KIWI system 12000 may use an on-board light-source to activate the fluorescent cells and use the dedicated optical fibers to record and transmit the data to an Application Specific Integrated Circuit (ASIC). Secondly, the fluorescent CNTs (polymer functionalized CNTs) may be used to optically identify the release of certain neurotransmitters.

ELECTROCHEMICAL RECORDING—The electrochemical recording functionality of the KIWI allows for the detection of released neurotransmitters based on analyzing the shape of the curve obtained by plotting current intensity over electric potential in fast-scan cyclic voltammetry.

Although called electrochemical “recording”, this functionality will rely on the ability of KIWI system 12000 to electrically stimulate the neural tissue (stimulation) and compute the current intensity (processing) by determining the electrical resistivity.

The method involves subjecting the neural tissue to an electric potential linearly increasing over time up to a certain threshold. After reaching the threshold, the electric potential is linearly ramped down to the initial value.

HYBRID RECORDING—Given that the electrophysical recording pipeline is built separately from the optical recording pipeline, depending on the number of CNTs assigned to each one of the two methods, KIWI system 12000 may be able to simultaneously record both electro-physically and optically. By combining both methods, we can record more complex and novel insights about the functionality of the brain.

TRAINING THE ALGORITHMS—Based on data recorded from the KIWI, neuroscientists may build AI/ML models that can be used by practitioners to treat various brain related maladies such as Parkinson's and Alzheimer's disease. Researchers will need to train NOAH algorithms using data from many patients. These algorithms can take an extended time to train, but they will be able to process gigabytes of data every second.

As input, researchers may be able to select patient data according to various criteria (such as age or disease). The output of this pipeline may be the resulting trained models, along with statistics detailing how well they performed (accuracy, loss).

Machine learning models targeted for specific diseases may also be available. Exemplary targets may include Alzheimer's and Parkinson's disease.

In the case of Parkinson's, for example, these machine learning models may be trained to recognize when the patient is having motor difficulties, such as bradykinesia or excessive tremors. During detection, a signal would be sent to activate neurons in the appropriate region, preventing symptoms before they begin.

In the case of Alzheimer's, the machine learning models may be used to recognize when a patient has difficulty recalling previously learned concepts, and stimulation would be applied to improve memory. Logistic regression has already been demonstrated to predict when a word will not be remembered.

The cloud system will also implement the Fundamental Code Unit framework to analyze and correlate all patient data, starting from low-level neurotransmitter levels and neural spiking data, to high level behavioral data such as language and gait analysis. (Howard and Hussain 2018).

THE GATEWAY—The KIWI may be connected to a specific KIWI Gateway component 12010, which may execute processes such as transmitting neural recording streams, receiving control commands, receiving configuration commands, etc. The responsibilities of the Gateway may include receiving high speed data streams from KIWI components 12002, 12006, buffering KIWI recorded data, compressing the data, and streaming the data securely to Cloud 12012 for processing and analysis, receiving complex control commands from Cloud 12012 and delivering them to KIWI components 12002, 12006 as neuron stimulation commands, sending configuration commands to KIWI components 12002, 12006, and requesting KIWI status information.

Thus, Gateway 12010 may be understood as a “black box” that has sufficient processing power to handle communication with multiple KIWIs and simultaneously stream data to the receive commands from the Cloud.

The software may be designed so that it will run on types of gateways such as wearable gateways (smartphone/tablet), home gateways, and deep clinic (hospital) gateways. Each of the gateway types may use the same data transfer and security protocols but may allow for much different data rates, buffering and analysis tools, and even have different associated KIWI operation modes.

THE CLOUD—There will be a large number of KIWIs which will send their data to the Cloud 12012. Thus, on Cloud 12012, there is a need for high scalability in recording this data and a demand to store a large amount of data. For example, technologies that can accommodate the constant increase of KIWIs and high parallelism of incoming data are those based on the Publish/Subscribe Paradigm. In this specific case of data processing, the KIWIs will act as data publishers while Cloud 12012 processes the data and acts as a subscriber.

Cloud 12012 may include clusters of nodes on which different microservices are deployed. The Data Processing Service is responsible for collecting the KIWIs data, decompressing it (if need be), and storing it for later usage. The Command Service will be able to execute commands received by the cloud, such as software updates, stimulation patterns, activation/inactivation, and recording control.

CLOUD USER INTERFACE (UI)—PATIENTS—A UI for the patients may be focused on data visualization. Patients will be able to see real time activity as it comes in from the KIWI. Patients will also be able to select from a list of stimulation commands that were prescribed by the doctor. These commands can be either based on their current activity (sleep, walk, etc.) or based on their physiological state (tremors, inability to focus). Patients will also be able to annotate certain time segments with activities they were involved in during that time span (to indicate when they were doing physical activities, mental tasks, etc.).

DOCTORS—Doctors will be able to access individual patient data. For each patient, they will have the option to apply different predefined machine learning models—presented as software based prescriptions—in order to determine the best treatment going forward.

Doctors will be able to configure the KIWI, based on the output of the previous models. They will be able to set different modes of operation for the KIWI, and change its recording/stimulation parameters.

They will also be able to visualize the data of the patient and flag certain patients for detailed analysis from neuroscientists.

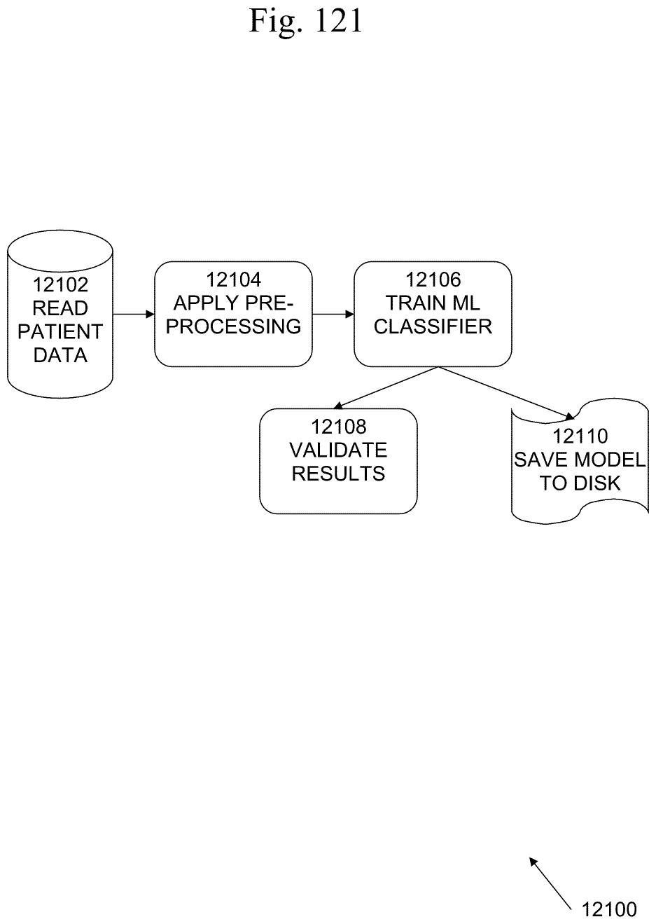

RESEARCHERS—Researchers can compose pipelines to process the data from many patients. An example of such a pipeline 12100 is shown in FIG. 121. In this example, patient data may be read 12102, pre-processed 12104, and used to train a machine learning classifier 12106. At 12108, the results may be validated and at 12110, the trained model may be saved. The direction of information flow is shown by the arrows.

KIWI CYBER SECURITY—Given the medical nature of the data handled by the system, great care must be taken to avoid unauthorized access to the data or any commands sent by unauthorized agents.

Analog signals such as sound or electromagnetic waves can be used as part of “transduction attacks” to spoof data by exploiting the physics of sensors. Transduction attacks target the physics of the hardware that underlies that software, including the circuit boards that discrete components are deployed on, or the materials that make up the components themselves. Although the attacks target vulnerabilities in the hardware, the consequences often arise in the software system, such as the improper functioning or denial of service to a sensor or actuator.

Hardware and software have what might be considered a “social contract” that analog information captured by sensors will be rendered faithfully as it is transformed into binary data that software can interpret and act on it. But materials used to create sensors can be influenced by other phenomena—such as sound waves. Through the targeted use of such signals, the behavior of the sensor can be interfered with and even manipulated.

KIWI system 12000 will be tested and measures will be taken against vulnerability to accidental or malicious wave interferences. All the RPCs (Remote Procedure Calls) issued between the various microservices that make up the system will be encrypted using the latest SSL encryption standards. Data that is streamed from the Gateway will also be encrypted to prevent tampering and snooping. To prevent unauthorized physical access in data centers, the data should always be stored with encryption.

There will be an Identity and Access Management layer which will be used for giving permissions to each actor to access and execute only user specific data and commands. For example:

Patients will be able to view only their own data and send to the KIWI commands that have been authorized by a doctor.

Doctors will only be able to view the full data of their patients, use pretrained models to prescribe new software based treatments for their patients and send commands to their patients' KIWIs.

Researchers will have access only to anonymized patient data that they can use to derive new scientific insights using the AI Research Interface provided in the Cloud environment.

Embodiments may support neural interfacing systems, such as vision and auditory systems, head/eye systems, auditory processors, and autonomous devices (including specialized medical devices) whose physical architecture and design principles are based upon those of the biological nervous system. Further, the properties of CNTs allows for the KIWI to be implanted in other parts of the body to attach to the nervous system. The possible therapeutic applications of the KIWI are unlimited. For example, the KIWI could be used for treatment of chronic pain, spinal cord injury, stroke, sensory deficits, and neurological disorders such as epilepsy, Parkinson's, Alzheimer's, and PTSD, all of which have evidence supporting the efficacy of neurostimulation therapy.

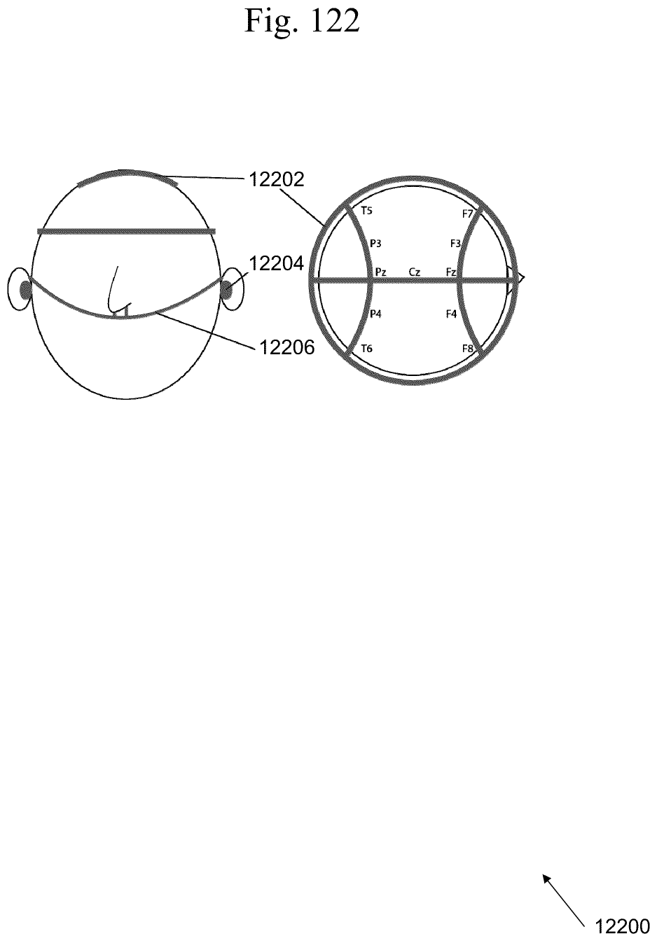

A Brain Code Collection System (BCCS) may include a KIWI system, as well as other monitoring and stimulation technologies. For example, given the interest in combining EEG monitoring with either direct transcranial magnetic or current stimulation, as well as the preliminary use in those with aphasia, most commonly seen as part of the clinical sequelae introduced by stroke and neurodegenerative disease where global cognitive function is likely to be impacted (such as Alzheimer's disease), a minimalist design to the apparatus is the primary feature that would be desirable. A helmet would provide infrastructure to house the full spectrum of EEG electrodes and stimulation apparatus, cover the ears for photic stimulation and could house a stem leading to an intranasal diode. However, as it is likely to be composed of heavy hardware, it is possible that a complete helmet may only be worn for 2-3 hours maximum at any one time and may be less conducive to those prone to agitation and delirium. Hence, as shown in FIG. 122, a frame 12200 able to support a small EEG array 12202, leaving space for stimulation apparatus (particularly over central and frontal regions), which itself could be detachable to allow stimulation of different cranial regions depending on the desired outcome, may be preferable. This could be combined with molded hearing aids featuring wireless diodes 12204 and an illuminated nasal cannula, which may be hollow to also allow oxygen entry 12206.

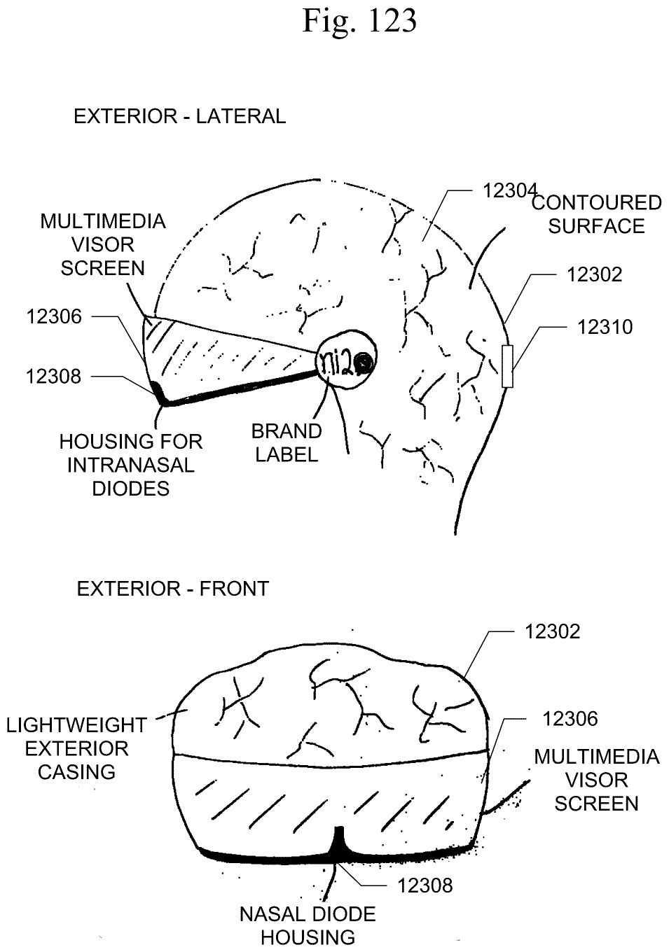

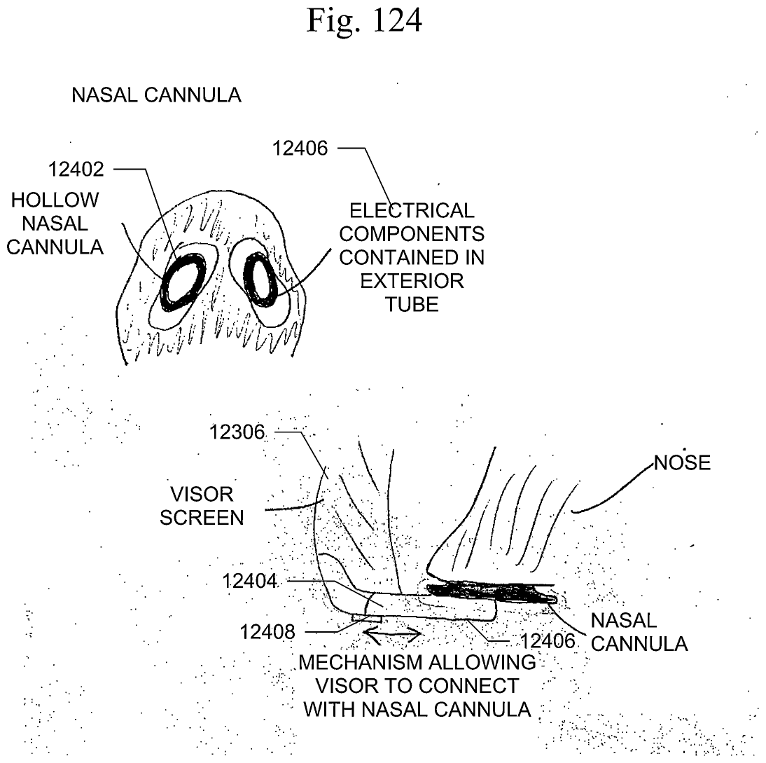

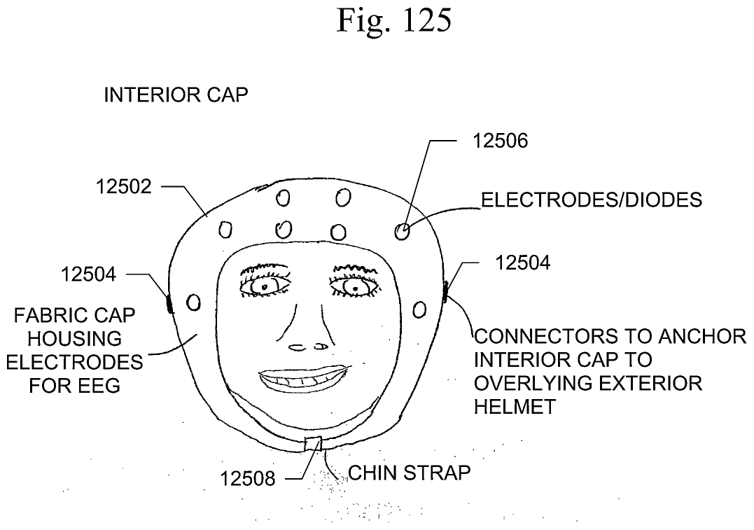

As shown in FIG. 123, embodiments of KIWI/BCCS may include features such as a lightweight exterior helmet 12302 with contoured surface 12304, which may be aesthetically pleasing with vivid colors and futuristic neuron pattern, multimedia visor 12306 that houses connectors 12308 to nasal cannula, an adjustable dial 12310 at the rear of the helmet to ensure comfortable fit. As shown in FIG. 124, embodiments may include features such as a hollow nasal cannula 12402 attached to an exterior tube 12404 containing electrical components 12406 and the exterior visor screen 12306 houses electronic components which connect to visor via a sliding mechanism 12408. As shown in FIG. 124, embodiments may include features such as molded earpieces 12204 (shown in FIG. 122) (wireless or connected to overlying helmet), an interior soft fabric EEG cap 12502, connectors 12504 to anchor interior cap to overlying exterior helmet, electrodes/diodes 12506, wherein the electrodes collect brain signals and the diodes provide stimulation, and may be secured to the head with a chin strap 12508 and connect to the exterior helmet via magnetic clips 12504 and chin strap 12508. Embodiments may allow greater comfort and improved fit as fabric cap can mold to the shape of the wearers head.

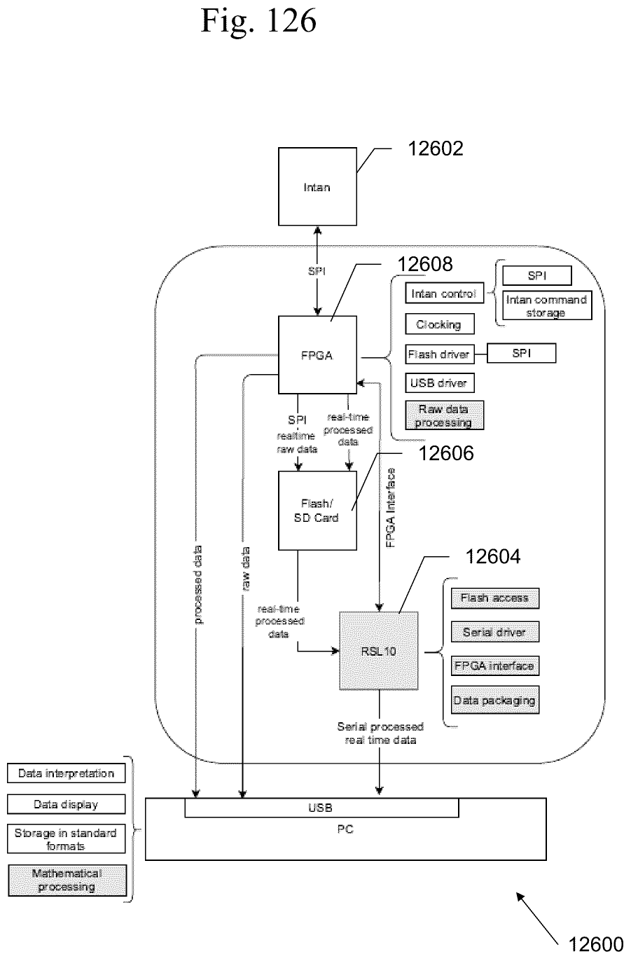

An exemplary block diagram of a KIWI/BCCS system 12600 is shown in FIG. 126. This example includes Neural recording and stimulating headstage circuitry 12602, such as provided by INTAN TECHNOLOGIES®, a processor 12604 and associated circuitry, such as an RSL10 System on a Chip provided by ON SEMICONDUCTOR®, memory 12606, such as a flash memory/SD card, and interface and other circuitry 12608, for example, provided by a Field Programmable Gate Array or other circuitry.

For example, embodiments of system 12600 may include a Thresholding engine module and a Stimulation engine module. Thresholding engine module may perform real time thresholding on raw data. The module detects the peak of signal corresponding to a channel by comparing three samples with threshold value. The sample to shall be higher than threshold value, higher than sample tₙ₋₁ and sample tₙ₊₁. A local memory will store 32 channel frames of three samples. A channel frame is a vector of 32 channels. Each time the engine detects a spike, a value of 1 will be written in encoded frame register. The frame counter stores the channel frame number. Each time the encoded register has at least one spike detected, it will be loaded into a local stack. When the stack fills nearly to its top the FPGA will signal the RSL10 that it can read the stack. The stack contains frame counter and channel occurrence. This piece of information and the raw data stored on SD card are enough to extract the spikes from the waveforms.

Stimulation engine module may generate square bipolar square waves with adjustable duty cycle, frequency and offset. Stimulation engine will be dependent on a local timebase that set the waveform resolution and will have parameters as a piecewise linear current source made out of points (Iₙ, tₙ). Embodiments may stimulate repeatedly the same piecewise linear waveform. Further, embodiments may load more complex waveforms in high speed cache memory and then send that waveform to stimulate the tissue.

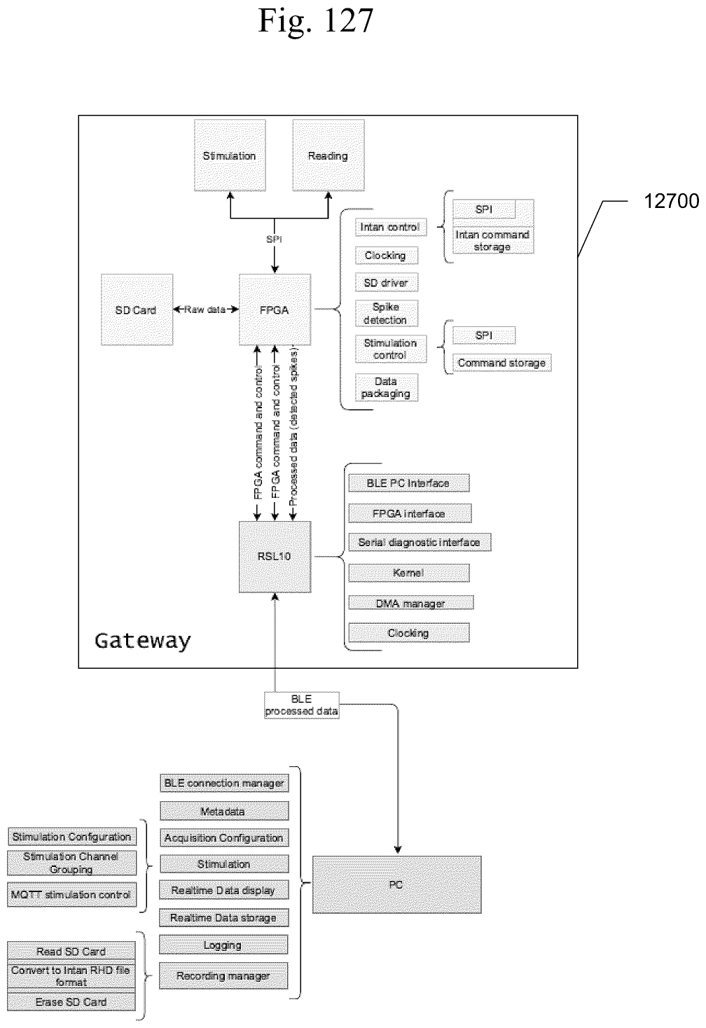

An exemplary block diagram of a Gateway 12700 is shown in FIG. 127. In this example, Gateway 12700 is a proprietary design including processor, memory, and other circuitry. In embodiments, Gateway 12700 may be implemented in standard hardware, for example, as an app on a smartphone or other device.

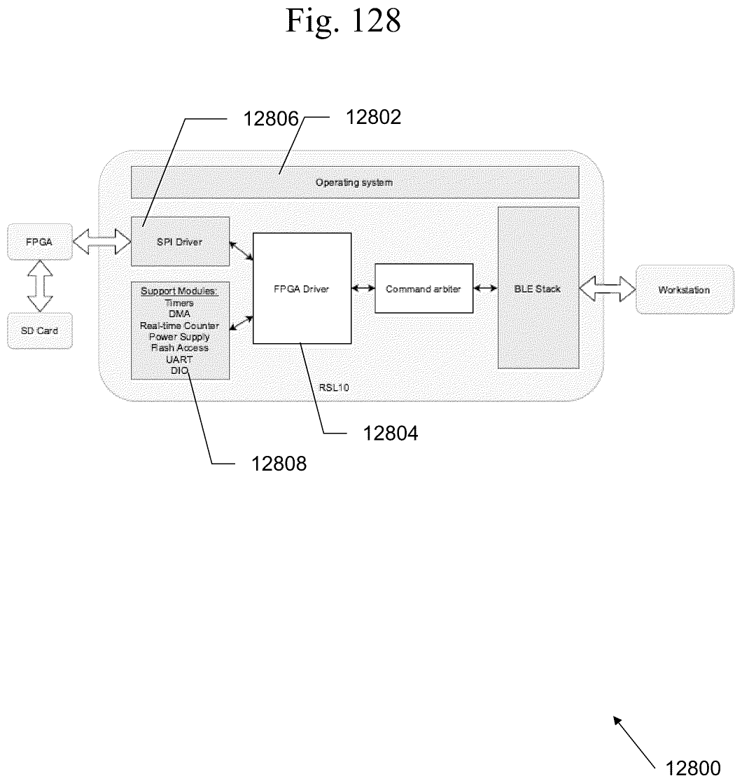

An exemplary block diagram of KIWI/BCCS firmware 12800 is shown in FIG. 128. Firmware 12800 may include modules such as operating system 12802, drivers 12804, 12806, support modules 12808, etc.

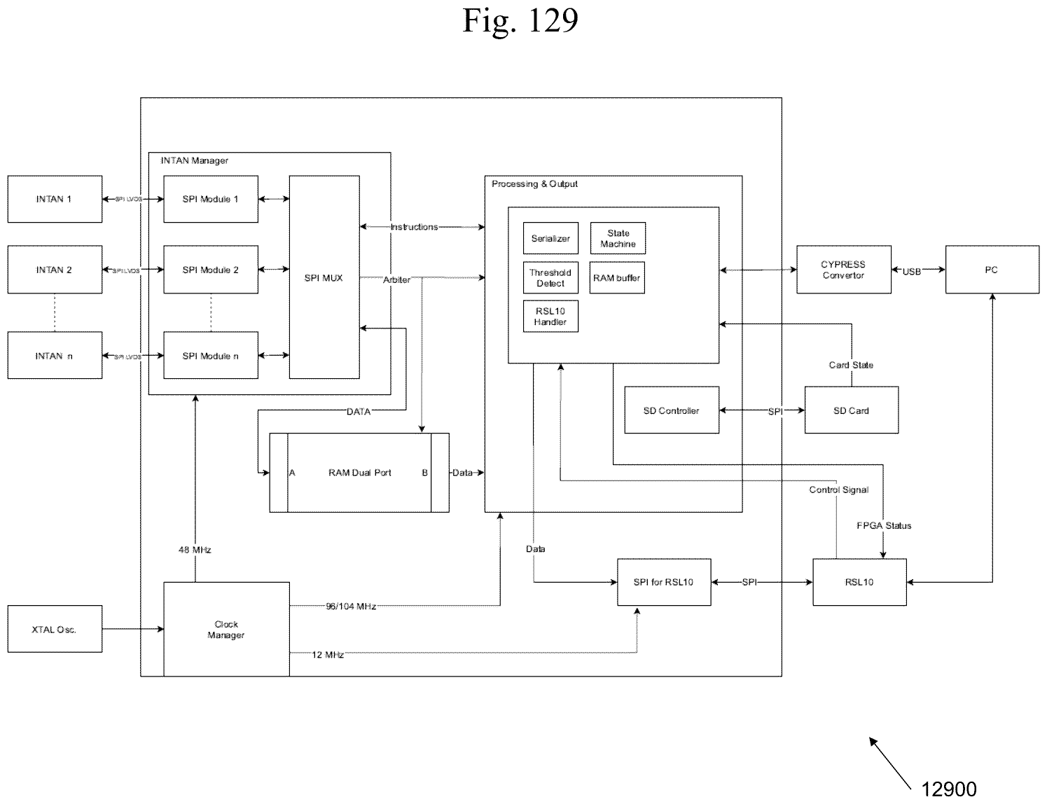

An exemplary block diagram of a KIWI/BCCS system 12900 is shown in FIG. 129. This example includes Neural recording and stimulating headstage circuitry 12902, such as provided by INTAN TECHNOLOGIES®, a processor 12904 and associated circuitry, such as an RSL10 System on a Chip provided by ON SEMICONDUCTOR®, memory 12906, such as a flash memory/SD card, and interface and other circuitry 12908, for example, provided by a Field Programmable Gate Array or other circuitry.

Embodiments may provide an implantable, miniaturized, for example, smaller than a pea, low-power, wireless probe with induction charging that may be used, for example, for treating neurodegenerative diseases like Parkinson's and Alzheimer through continual neuromodulation, as a Brain Machine Interface to monitor and prevent other medical condition, such as migraines, and to perform cognitive functions augmentation. Embodiments may provide a wireless and implantable probe with induction charge that may include carbon nanotube (CNT) probes that may facilitate a scaffolding for neuron growth and regeneration and may provide artificial intelligence (AI) Analytics

In embodiments, a carbon nanotube (CNT) based electrode array may serve as a building block enabling high-density neural connections in a manner that is non-destructive to tissue. These electrodes may be integrated with solid-state imager readout circuitry (ROIC). For example, modem imager ROIC devices may have pixel densities on a micron pitch scale, which may be configured for single neuron voltage readout. Likewise, CNT electrodes and LED diodes (for optical stimulation) may be heterogeneously integrated on single a ROIC that could both optically stimulate and read the electrical potential from individual neurons.

In embodiments, a large number of electrically active brain-probing sites may be provided, along with long-term use. In embodiments, an implantable neural connecting probing system may be enabled by compliant, biocompatible, carbon nanotube (CNT) electrical wires. In embodiments, these contacts may directly stimulate and readout a high density of individual neural signals using read-out integrated circuit technology (ROIC) similar to that employed in focal plane arrays used in imaging applications.

In embodiments, an ROIC may include a large array of “pixels”, each consisting of a photodiode, and small signal amplifier. In embodiments, the photodiode may be processed as a light emitting diode, and the input to the amplifier may be provided by the CNT connection to the neuron. In this manner, neurons may be stimulated optically, and interrogated electrically. In embodiments, CNT electrical connection to neural tissue may be provided. In embodiments, a small pitch (2-20 micron) CNT array may be compatible with ROIC designs.

An exemplary embodiment of a Biological Co-Processor System (BCP) 100 is shown in FIG. 1. In embodiments, BCP 100 may include a neuromodulatory system comprising one, two, or more inductively-recharged neural implants 102 (the implant device), two earbuds 106, which may include wireless and various sensors, together known as the Brain Code Collection System (BCCS) 110. These devices may work independently, but together may form a closed-loop system that provides the BCP 100 with bidirectional guidance of both internal (neural) and external (behavioral and physiological) conditions. The BCCS earbuds 106 may read the brain for oscillatory rhythms from internal onboard EEG and analyze their co-modulation across frequency bands, spike-phase correlations, spike population dynamics, and other patterns derived from data received from the implant devices 102, correlating internal and external behaviors. The BCP may further comprise Gateway 111, which may include computing devices, such as a smartphone, personal computer, tablet computer, etc., and cloud computing services, such as the Fundamental Code Unit (FCU) 112 cloud computing services, which is a mathematical framework that enables the various BCCS 110 sensor feeds and implant device 102 neural impulses to be rapidly and meaningfully combined.

The FCU 112 may provide common temporal and spatial coordinates for the BCP 100 and resides in all components of the system (implants, earbuds, app, cloud) ensuring consistent mapping across different data types and devices. FCU 112 algorithms may provide extremely high rates of data compression, association, and throughput, enabling the implant device 102 to transcribe neural signals in high volume. Each implant device 102 may have an embedded AI processor, optical neurostimulation capabilities and electrical recording capabilities. The implant device 102 may consist of two types of microfabricated carbon nanotube (CNT) neural interfaces, a processor unit for radio transmission and I/O, a light modulation and detection silicon photonic chip, an inductive coil for remote power transfer and an independent receiver system, where the signal processing may reside. The BCP 100 system may comprise four components: (1) the implant device 102 implant(s), (2) the BCCS 110 and (3) the cloud services (with API and SDK) and (4) an inductive power supply.

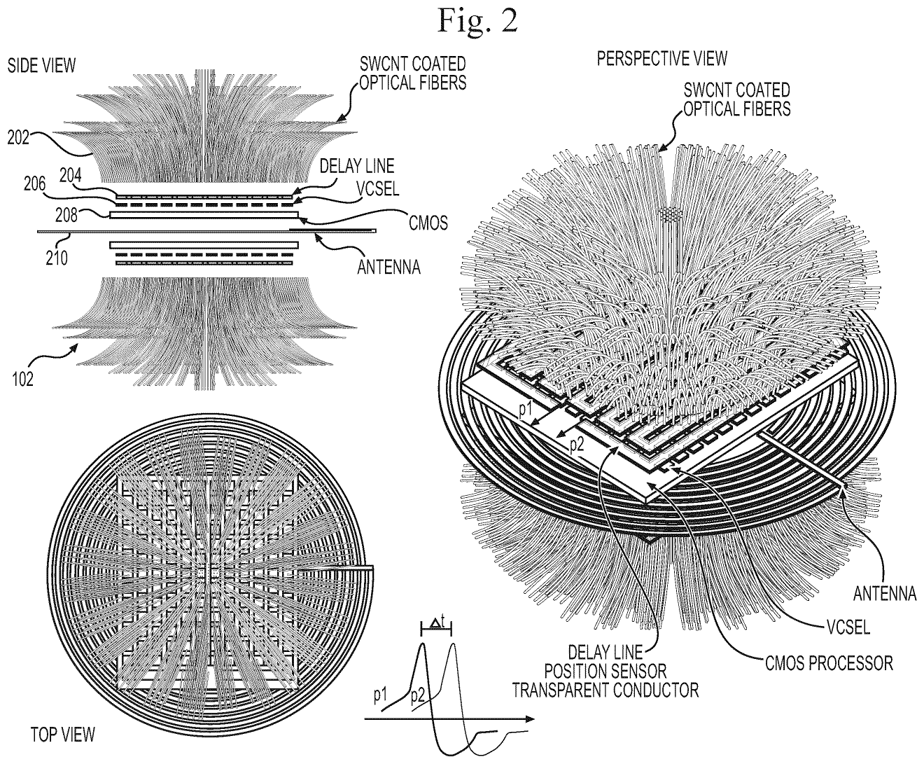

The implant device, an example of which is shown in FIG. 2, may be an ultra-low power computing device with interconnects that can attach to nerve and/or brain tissue and read signals/voltages and/or stimulate those tissues with electrical or optical pulses. This multi-physics interaction between the implant device and the tissue may be performed through two back-to-back arrays of optic fibers coated with single wall carbon nanotubes (CNTs). The CNTs may be chosen due to their structure, which has been shown to readily attach to tissue and also due to their remarkable electrical properties. Effectively, the CNTs may serve as electrochemical and optical sensors and measurement/stimulation electrodes. The device may be implanted in the brain or other parts of the body to attach to the nervous system, although this document focuses on attaching to the brain to treat neurological disorders. The implant device may include a communication module to transmit data to a Gateway device such as cell phone or other nearby computer which can in turn analyze data, give input to the implant device, and/or send the data to the Cloud for deep analysis.

The implant device may provide a revolutionary brain-computer interface for research in Neuroscience and medicine, being a closed-loop neural modulator informed by internal and external conditions. The possible therapeutic applications are numerous. For example, the implant device could be used for treatment of chronic pain, spinal cord injury, stroke, sensory deficits, and neurological disorders such as epilepsy, Parkinson's, Alzheimer's, and PTSD, all of which have evidence supporting the efficacy of neurostimulation therapy.

Turning briefly to FIG. 2, each implant device 102 implant may be, for example, an oblate spheroid (for example, 0.98×0.97×1.0 cm), a design inspired by the radial characteristics of an implant device 102 fruit. In the center of the implant is a nucleus surrounded by a fleshy membrane. The nucleus may house the processing, transmitting, and receiving circuitry 208, including an embedded processor for local preprocessing, read and write instructions, the modulation scheme, and an optical FPGA dedicated for real time optical modulation. It may also contain a CMOS dedicated integrated front-end circuit developed for a pre-amplification and multiplexing of the neural signals recorded, 4G-MM for offline storage, wireless transceiver, inductive power receiver, and an optical modulation unit. Covering the nucleus are, for example, 1 million fibers 202 made of single walled carbon nanotubes (SWCNT) and, for example, 1100 geometrically distributed optical fibers coated with SWCNT, connected in the same manner as the SWCNT fibers, wrapping around a central primary processing nucleus. Fibers may be built on a flexible interface substrate and surrounded by a gel/flesh membrane. When implanted, the membrane casing will slowly dissolve, naturally exposing the probes to a cellular environment with limited risk of rejection. For example, the gel may be relatively solid at about 25° C. and liquid at about 37° C. The lubrication of the CNT probes will attract neurons to the implant. The implant device 102 implant will be able to record from pyramidal layers II-III down to layer VI of any brain cortex region. Also shown in FIG. 2 are delay line devices 204, light sources, such as vertical-cavity surface-emitting lasers 206 (VCSELs), and antenna 960.

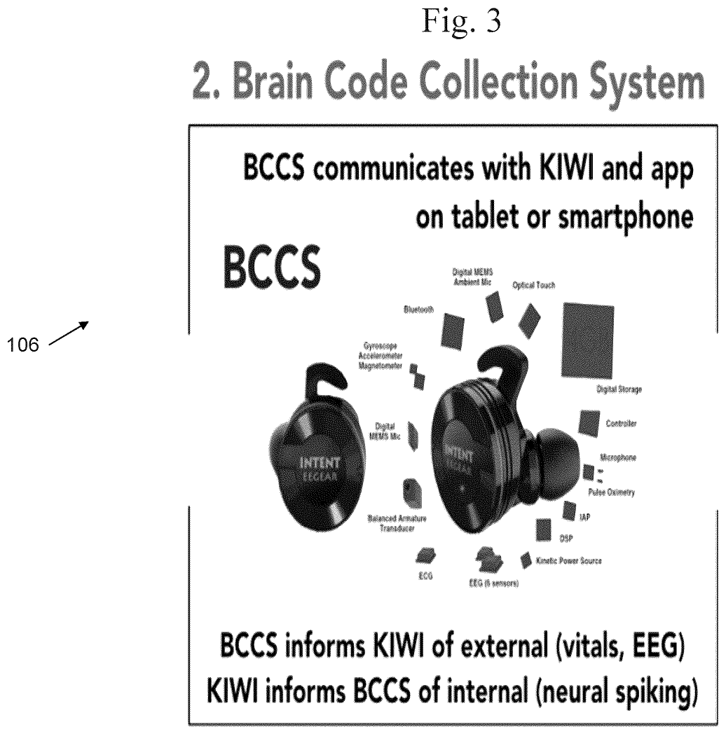

Returning to FIG. 1, the BCCS earbud 106, also shown in FIG. 3, wirelessly communicates with the implant device 102. The earbud contains a signal amplifier and a relay for modulation schemes, algorithms, and instructions to and from the implant. The BCCS earbud 106 also has additional functions, such as EEG and vestibular sensors, which will serve as crosscheck metrics to measure efficacy and provide global behavioral, physiological, and cognitive data along with neural data on the same timescale.

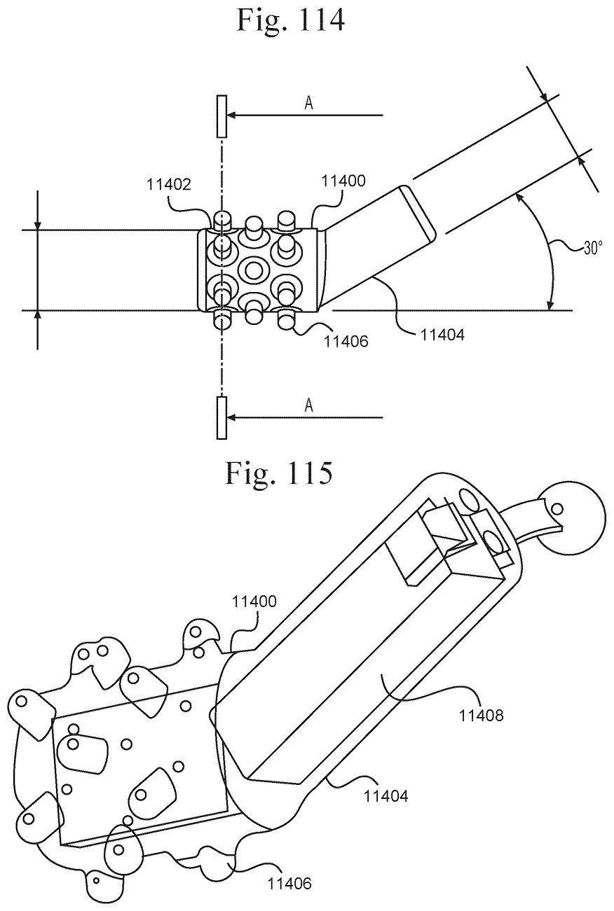

An exemplary embodiment of a BCCS earbud 11400 is shown in FIG. 114. In this example, device 11400 may include an inserted portion 11402 and a protruding portion 11404. Inserted portion 11402 may be inserted in an ear canal during use, while protruding portion 11404 may protrude from the ear during use, as shown in FIG. 121. Inserted portion 11402 may include a plurality of protrusions 11406, which may provide retention of device 11400 within the ear canal. Protrusions 11406 may be implemented as millimeter scale conductive rubber electrodes. In this example, a battery or power cell 11408 may be disposed within protruding portion 11404.

BCCS earbud 11400 wirelessly communicates with the KIWI. The earbud contains a signal amplifier and a relay for modulation schemes, algorithms, and instructions to and from the implant. The BCCS earbud also has additional functions, such as EEG and vestibular sensors, which will serve as crosscheck metrics to measure efficacy and provide global behavioral, physiological, and cognitive data along with neural data on the same timescale.

In embodiments, BCCS earbud 11400 may be a non-permanent integrated solution for unobtrusive monitoring of activities of daily living. The device may be a platform for biometrics and may have two main embodiments—1) a hearing aid headset for hearing—impaired patients and 2) a wireless audio streaming device and hands-free headset. In conjunction with a smartphone, BCCS earbud 11400 may be capable of Neural activity monitoring (EEG), Performing electrocardiography (EKG), Measuring core body temperature, Breathing Monitoring, Activity Tracking, Blood oxygen saturation measurement (SpO2), Blood pressure monitoring, etc.

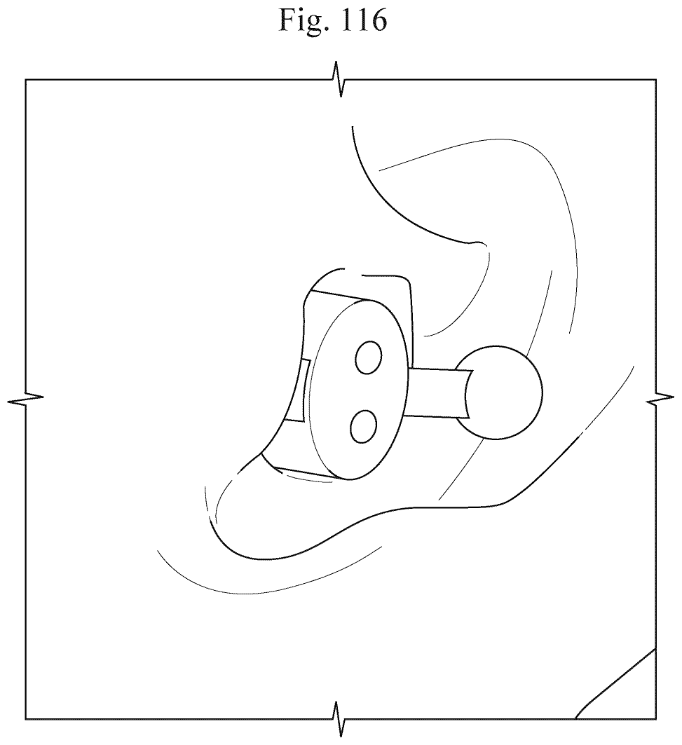

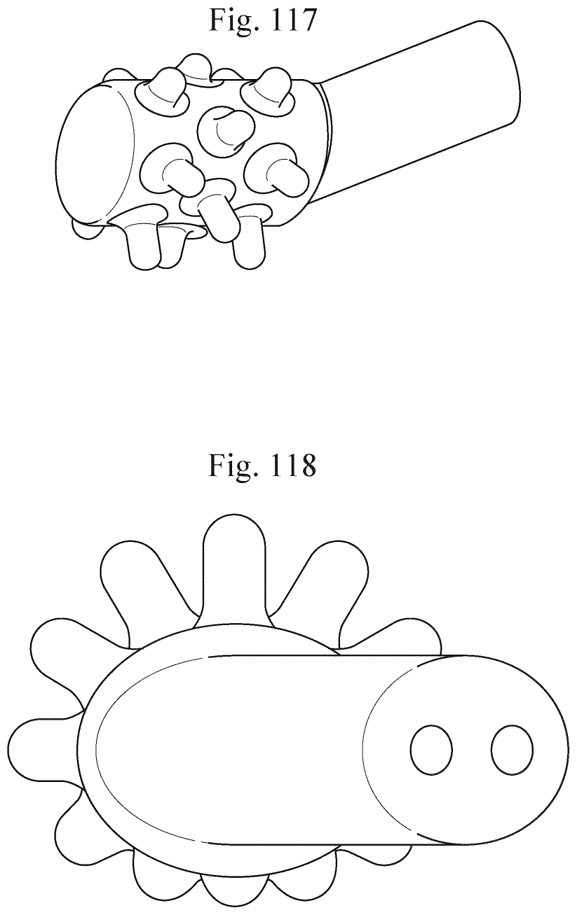

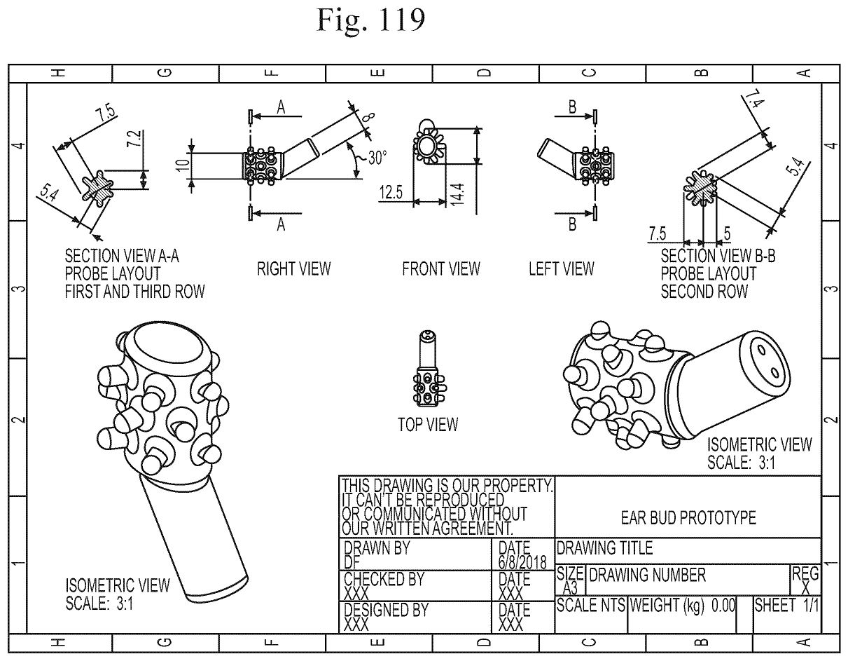

An exemplary embodiment of BCCS earbud 11400 in use is shown in FIG. 122. An exemplary embodiment of BCCS earbud 11400 including rubber probes arranged in a staggered fashion is shown in rear view in FIG. 117 and in side view in FIG. 118. An exemplary mechanical drawing of an embodiment of BCCS earbud 11400 is shown in FIG. 119.

Embodiments may include features such as Rechargeable and replaceable Li—Po battery, Dry electrodes made of conductive rubber for adherence and comfort to perform ECG and EEG, Blood pressure measurement using PTT (Pulse Transit Time) and PWV (Pulse Wave Velocity), Tympanic membrane infrared temperature measurement, Accelerometer measuring of heart rate (HR), breathing rate (BR) and activity tracking, PPG (Photoplethysmography) optical measurement of blood volume changes, and may provide hearing aid and/or music streaming capabilities with noise cancellation.

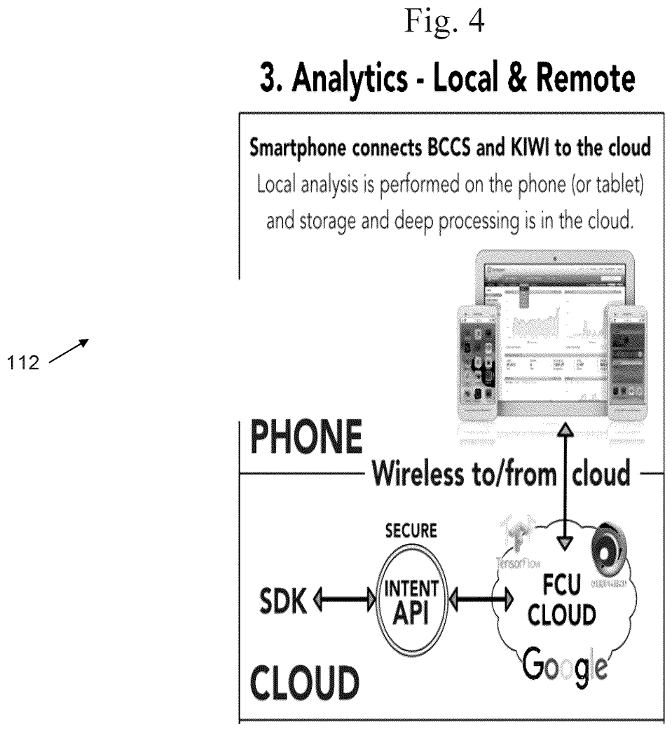

A cloud platform 112, also shown in FIG. 4, may include the parallel data flow and FCU 112 analytic engine powered by neuro-computational algorithms and extreme machine learning. EEG, ECG, and other physiological data (external and internal) will be uploaded to the cloud wirelessly from the BCCS 110 and implant device 102. A suite of algorithms will analyze the aggregate data stream and formulate instructions for optimal electrical and/or optical neuromodulations in a closed loop feedback system. Integrated stimulation/control, recording/readout and modulated stimulation parameters will allow simultaneous optical and/or electrical recording and stimulation.

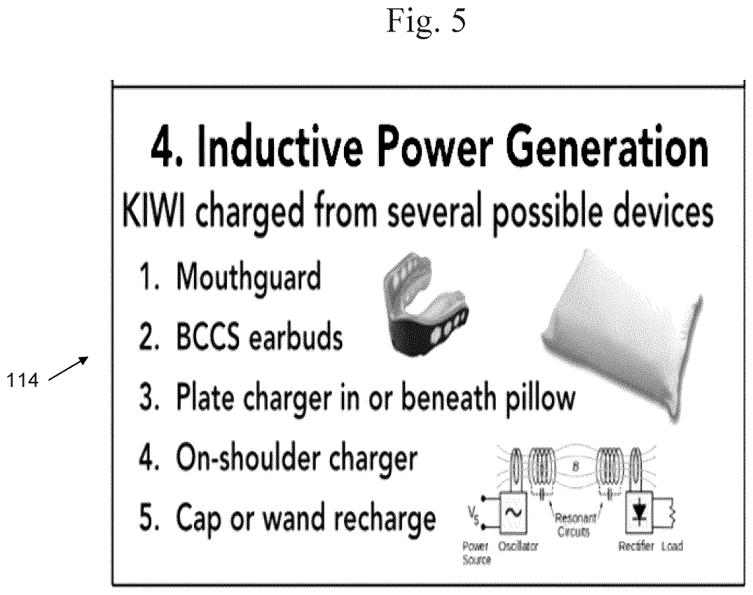

An inductive powering system 114, also shown in FIG. 5, may be used recharge the implant device 102 implant (see FIG. 1). Various wearable and/or kinetic inductive power technologies may be utilized during the design phase, including a retainer/mouthguard, a head-mounted cap to be worn at night, or an under the pillow charging mat.

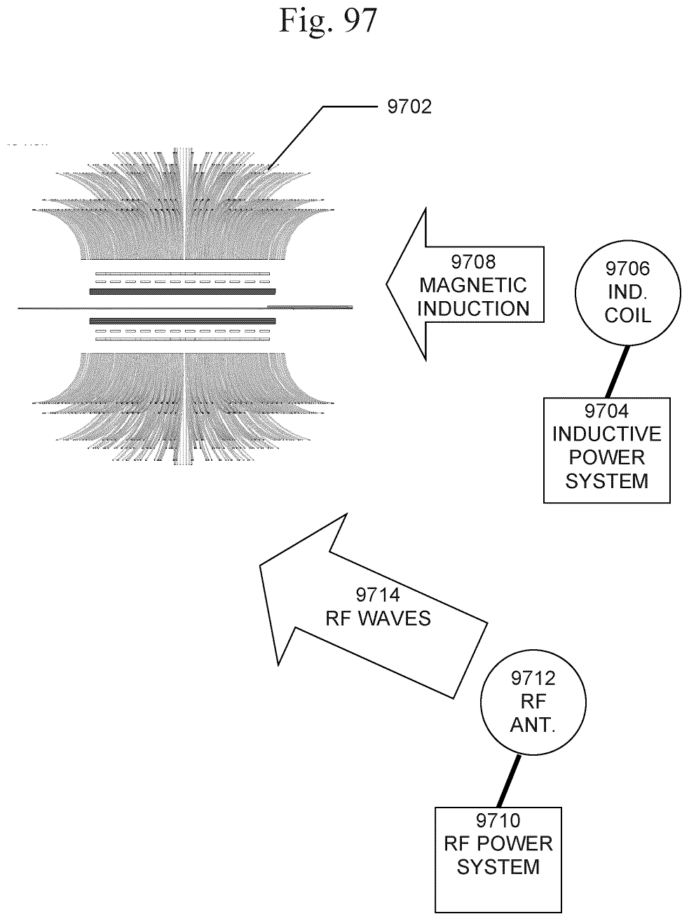

As shown in FIG. 97, in embodiments, implant device 9702 may be directly powered by inductive power system 9704, which connects to induction coil 9706 and powers implant device 9702 directly using magnetic induction 9708. In embodiments, implant device 9702 may be directly powered by RF power system 9710, which connects to RF antenna 9712 and powers implant device 9702 directly using RF waves 9714. RF waves 9714 transmit power electromagnetically in a manner similar to a Radio-frequency identification (RFID) system. RFID systems are typically only powered on intermittently. In embodiments, implant device 102 may be powered intermittently by electromagnetic power, In embodiments, implant device 102 may be powered substantially continuously by electromagnetic power. Such embodiments of implant device 102 may have no batteries, or may have batteries of greatly reduced size. Embodiments that do not have a battery may be of reduced size. They may even be tiny, such as about the size of a grain of rice or smaller. Medical devices that can be ingested or implanted in the body may provide new ways to diagnose, monitor, and treat many diseases. For example, such devices may deliver drugs, monitor vital signs, and detect movement of the GI tract. In embodiments, implant device 102 may deliver electrical or optical signals, even though they may include no batteries, or only very small batteries.

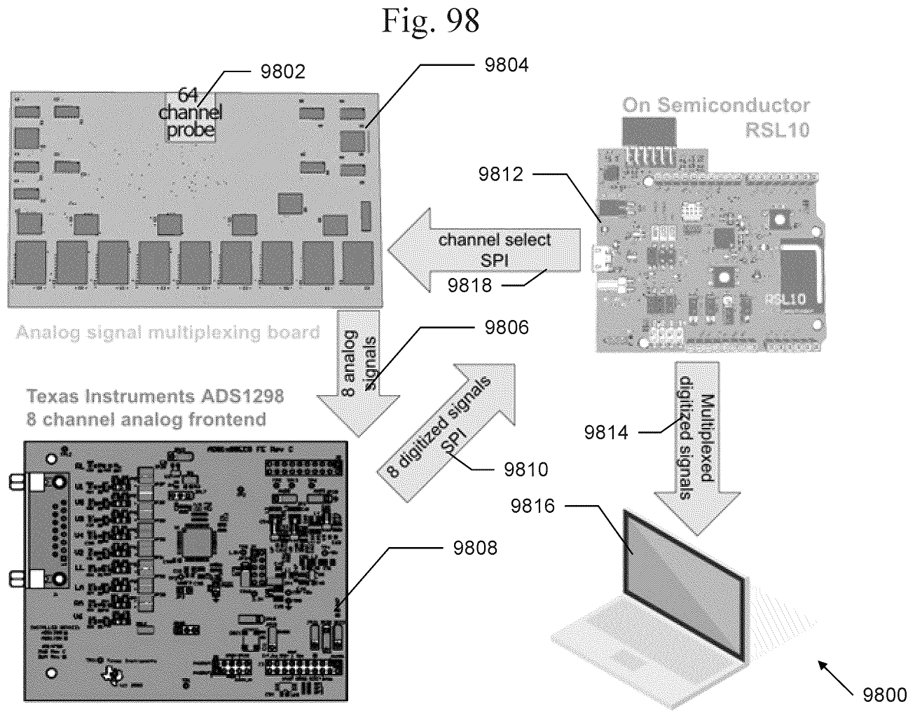

An example of high-level system data flow in an embodiment of a Brain-Machine Interface system 9800 is shown in FIG. 98. In this example, multiple channels of probes may connect 9802 to an analog signal multiplexer 9804, which may multiplex the multiple analog channels onto a lower number of multiplexed channels 9806. Multiplexed channels 9806 may be input to multiple channels of analog-to-digital converter circuitry (ADC) 9808, which may convert the analog signal to digital signals representative of the analog signals and may transmit 9810 the multiplexed digitized signals, for example, using a protocol such as Simple Peripheral Interface (SPI) to a gateway processor 9812. Gateway processor 9812 may process and compress the multiplexed digitized signals and may transmit 9814 them to a computer system 9816 for analysis using, for example, Fundamental Code Unit (FCU), Brain Code (BC) and artificial intelligence (AI) algorithms. Further, gateway processor 9812 may perform real-time spike detection on the multiplexed digitized signals, probe calibration, and control of probes, such as probe channel selection 9818.

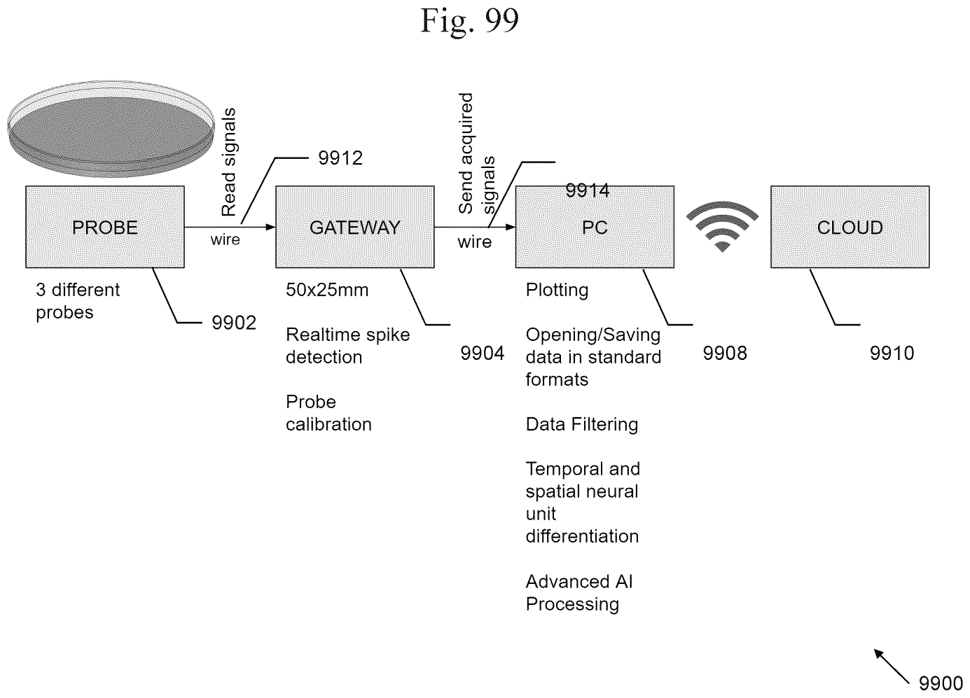

An exemplary high-level system block diagram of an embodiment of a Brain-Machine Interface system 9900 is shown in FIG. 99. In this example system 9900 may include a plurality of probes 9902, as described herein, gateway processor 9904, computer system 9908, and cloud computing resources 9910. Gateway processor 9904 may process and compress the multiplexed digitized signals 9912 and may transmit 9914 them to a computer system 9908. Further, gateway processor 9904 may perform real-time spike detection on the multiplexed digitized signals, probe calibration, and control of probes, such as probe channel selection. Computer system 9908 may perform analysis using, for example, FCU, BC, and AI algorithms, as well as reporting and plotting the analyzed signals, saving/retrieving data, for example in standard formats, filtering of data, temporal and spatial neural unit differentiation, and advanced AI processing.

Embodiments may include features such as: a mobile gateway of the size of a nickel (size limited by the battery), frontend analog interfaces, implemented as a system-on-chip (SoC) that includes, for example, ARM Cortex M3 processor, Bluetooth 5 Low Energy, for Wireless PC communication, DSP core for spike detection acceleration, support for multiple types of probes, such as those described herein, as well as commercially available probes, several days battery stand-by before recharging, and 8 hours in record mode, wired or wireless charging for the Gateway using a miniature power connector. Embodiments may include AI Systems, such as support for popular analytics platforms, AI based signal analysis, models of diseases diagnostics, etc.

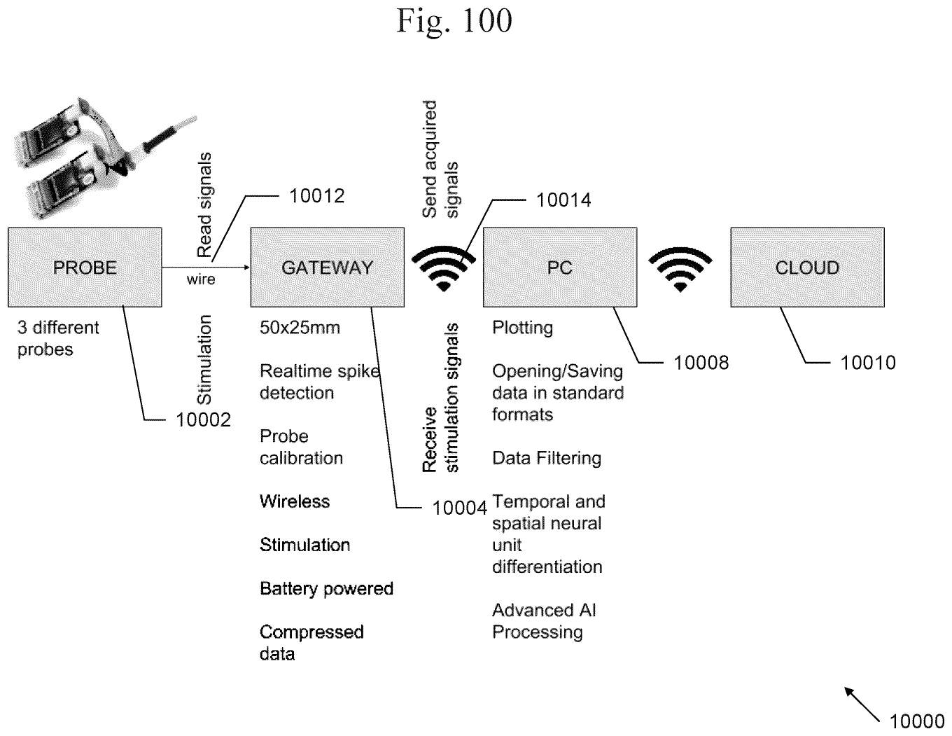

An exemplary high-level system block diagram of an embodiment of a Brain-Machine Interface system 10000 is shown in FIG. 100. In this example system 10000 may include a plurality of probes 10002, as described herein, gateway processor 10004, computer system 10008, and cloud computing resources 10010. Gateway processor 10004 may process and compress the multiplexed digitized signals 10012 and may transmit 10014 them to a computer system 10008. Further, gateway processor 10004 may perform real-time spike detection on the multiplexed digitized signals, probe calibration, and control of probes, such as probe channel selection, generation of stimulation signals, wireless communications, and charging, etc. Computer system 10008 may perform analysis using, for example, FCU, BC, and AI algorithms, as well as reporting and plotting the analyzed signals, saving/retrieving data, for example in standard formats, filtering of data, temporal and spatial neural unit differentiation, and advanced AI processing.

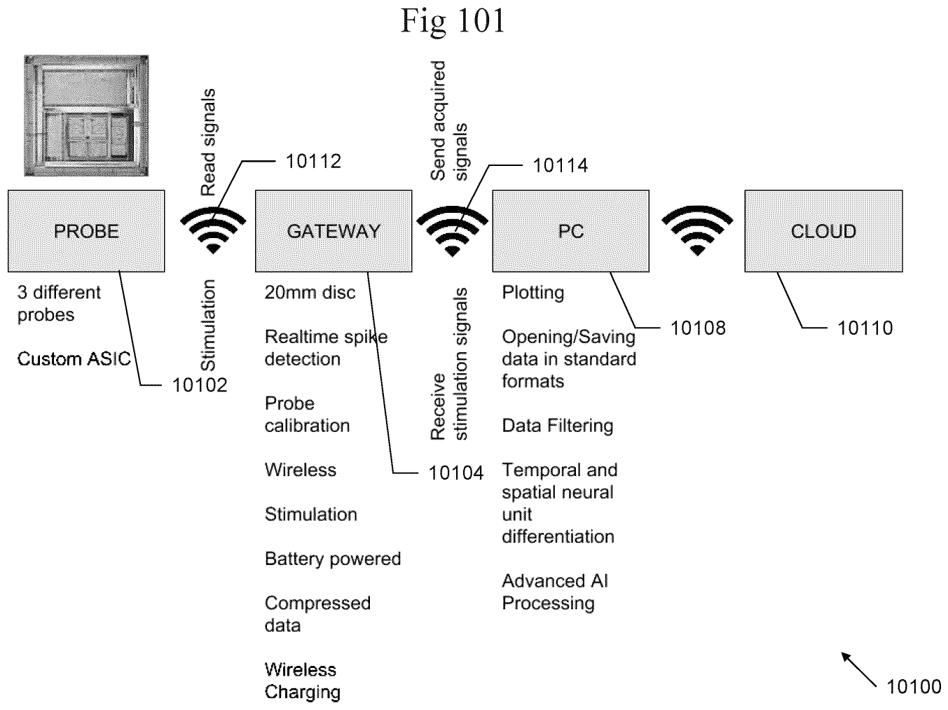

An exemplary high-level system block diagram of an embodiment of a Brain-Machine Interface system 10100 is shown in FIG. 101. In this example system 10100 may include a plurality of probes 10102, as described herein, gateway processor 10104, computer system 10108, and cloud computing resources 10110. Gateway processor 10104 may process and compress the multiplexed digitized signals 10112 and may transmit 10114 them to a computer system 10108. Further, gateway processor 10104 may perform real-time spike detection on the multiplexed digitized signals, probe calibration, and control of probes, such as probe channel selection, generation of stimulation signals, wireless communications, and charging, etc. Computer system 10108 may perform analysis using, for example, FCU, BC, and AI algorithms, as well as reporting and plotting the analyzed signals, saving/retrieving data, for example in standard formats, filtering of data, temporal and spatial neural unit differentiation, and advanced AI processing.

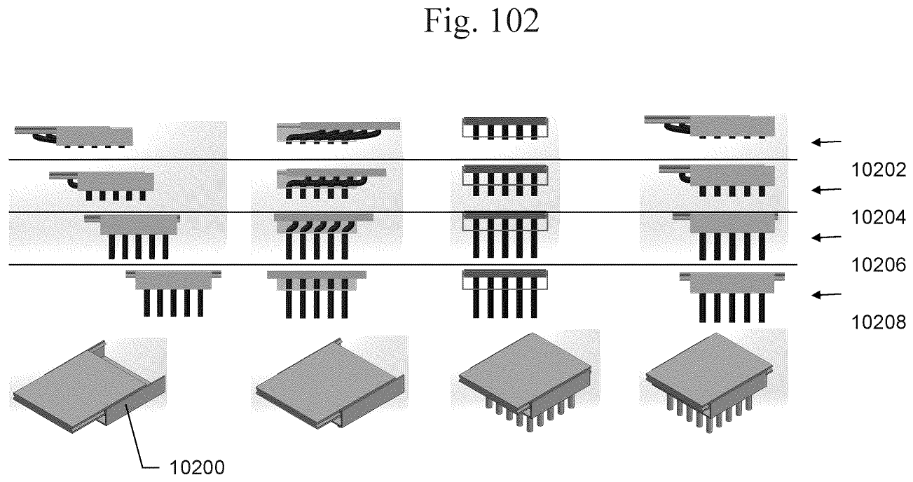

As the length of insertion of the probes increases there will be increasingly large issues with buckling of the individual CNT fibers when pressed down during initial insertion. This is because the cross-sectional area and area moment of inertia of the a single CNT compared to its overall effective length is quite small. The larger this slenderness ratio (l/k) is the lower the critical force is before buckling. Accordingly, as shown in the example of FIG. 102, embodiments may include an integrated sliding support brace 10200 to be used in conjunction with the chip and CNT arrays that will cut the effective length of the CNT's during insertion this preventing this buckling phenomena. As pressure is applied 10202 onto the assembly to aid in CNT penetration the support brace will act like a comb or and support the CNT's much closer the outer surface of the brain. As the chip slides toward the comb 10204 it will feed more and more 10206 of the rest of the CNT fibers through the orifices in the comb, which will bend them downwards and inserted deeper into the cerebral cortex. Once the chip has been slid into position the CVT arrays will be fully straight and at maximum depth of insertion 10208. In embodiments, the chip and comb may have a tight V-shaped tongue and groove connection to insure stability, accuracy and function of the probes and assembly both during insertion and after during the long term.

Embodiments may include microfluidic chamber drives. A CNT bundle may be pulled through a “pipe” like structure. To increase flow, the electrode may be pulled through a flow increasing jet. By frictional forces, the CNT bundle may be practically pulled towards the implantation area. In order to remove unwanted liquid, a liquid output port may follow the jet.

Combined electro and optogenetic approach enables precise (ON/OFF) control of specific target neurons and circuits. Unary controls in combination with rapid closed loop controls in the implant device's microchip will enable neural synapse firings with intensity, and frequency modulation.

Integrating SWCNT nanotechnology with optical fibers enables both optogenetic writing and electrical neurostimulation capabilities.

CNTs are biologically compatible, enabling the implant device to be stably implanted for long periods of time.

A dissolvable membrane, such as Dextrane, Gelatine, or Collicoat, will limit the risk of damaging sensitive surface tissue during surgery and minimize adverse tissue reactions following the implant insertion trauma. This will protect both the patient and the CNTs.

The implant device will be in the brain parenchyma, rather than tethering the implant to the skull, which can be a major contributor to adverse tissue reactions.

The implant device's open hardware architecture can record data from all pyramidal layers II-III down to layer VI offering several advantages in terms of data quality.

Closed loop architecture enables dynamic, informed response based on live internal and external conditions.

Big data approach utilizing smartphone apps, SDKs, and websites/APIs will provide visual, aggregate, and actionable real-time biofeedback and software modification capabilities.

Big data approach utilizing cloud API will provide storage to capture extremely large volumes of data. The cloud platform also provides the massive processing power required to analyze these huge data sets across subject profiles and a plurality of research databases (PPMI, PDRS, etc.).

Open software architecture SDK will allow the creation of new applications and different protocols for clinical and research use, by partners, researchers and third parties.

The BCCS will be able to synchronously capture EEG, ECG, PulseOx, QT intervals, BP, HR, RR, true body temperature, body posture, movement, skin conductance, vestibular data, and audio data to provide a rich set of multimodal data streams to dynamically correlate internal states read by the implant device and external states observed by the BCCS, a process which will help to effectively map neural pathways and function.

A passive inductive power unit and the BCCS earbud amplifier will be used external to the cranium, allowing the implant device to be small, low power and of low energy consumption. Any design for an extended-use implant without such an external component would need to be considerably larger (and of a finite lifespan).

The BCP data flow (internal and external) allows machine learning, prior experience, and real time biofeedback to autonomously guide implant device neuromodulation. Eventually the BCP will achieve an advanced level of sensitivity and will be able to autonomously sense neuron activity and guide light and/or electrical stimulation as needed.

Autonomous stimulation will be guided by intuitive algorithms and operational self-monitoring during awake state and sleep. Personal profiles and personalized signatures of neural activity will be learned and coded over time.

The BCP system takes two distinct but complementary approaches: a direct approach by means of recording brain activity and an indirect approach deduced from the multimodal aggregate analysis of peripheral effectors such as temperature, cardiac activity, body posture and motion, sensory testing etc. This simultaneous and coupled analysis of the interplay between the brain “activities and functions” (including physiological, chemical and behavioral activities) and its peripheral effectors and the influence of the effectors on the brain “activities and functions” has never been done before.

Simultaneous brain recording and stimulation of the same region allows us to take account of the initial state of the neurons and their environment, enabling comprehension of the neurons properties and network as well as brain functions (as the data are only valid for the specific conditions in which they were obtained). Methods which are forced to ignore this initial state have limited potential for understanding the full system.

implant device Development—in an embodiment, an approach to solving density challenges combines traditional photolithographic thin-film techniques with origami design elements to increase density and adaptability of neuronal interfaces. Compared to traditional metal or glass electrodes, polymers such as CNT are flexible, strong, extremely thin, highly biocompatible, highly conductive, and have low contact impedance, which permits bidirectional interfacing with the brain (Vitale et al., 2015). These properties are especially valuable for the construction of high-density electrode arrays designed for chronic and/or long-term use in the brain. Our approach to precision and accuracy supersedes the current state of the art (SOA), which is limited to only being able to fit certain regions of the brain. These limits are due both to the physical design of the interface inserted and also to the limits of tethered communication within deeper cortical areas. The implant device, on the other hand, is wireless and inductively powered, and so is implantable anywhere in the brain with a subdural transceiver, to allow reading of neurons both at the surface and in 3D. CNT fibers will allow for bidirectional input and output. CNTs will also enable more biocompatible, longer-lasting designs—current neural implants work well for short periods of time, but chronic or long-term use of neural electrodes has been difficult to achieve. The main reasons for this are: 1) degradation of the electrode, 2) using oversized electrodes to attain sufficient signal-to-noise ratio during recording, and 3) the body's natural immune response to implantation. Although there is a strong desire among neurologists to record chronic neural activity, electrodes used today can damage brain tissue and lose their electrical contacts over time (McConnell et al., 2009, Prasad et al., 2012). This is of particular concern in the case of deep cortical implants, so alternative materials, design principles, and insertion techniques are needed. CNT is a biocompatible material that has been studied for long-term use in the brain.

Optogenetics may be used to facilitate selective, high-speed neuronal activation; a technology in which light-sensitive ion channels are expressed in target neurons allowing their activity to be controlled by light. By coating optical fibers (˜8 μm) with dense, thin (˜1 μm) CNT conformal coatings, optical modulation units may be built within the nucleus of the implant device that can deliver light to precise locations deep within the brain while recording electrical activity at the same target locations. The light-activated proteins channelrhodopsin-2 and halorhodopsin may be used to activate and inhibit neurons in response to light of different wavelengths. Precisely-targetable fiber arrays and in vivo-optimized expression systems may enable the use of this tool in awake, behaving primates.

A suite of brain to digital and digital to brain (B2D:D2B) algorithms may be used for transducing neuron output into digital information. These algorithms may be theoretically-grounded computational models corresponding to the theory of similarity computation in Bottom-Up and Top-Down signal interaction. These neurally-derived algorithms may use mathematical abstractions of the representations, transformations, and learning rules employed by the brain, which will correspond to the models derived from the data and correspond to the general dynamic logic and mathematical framework, account for uncertainty in the data, as well as provide predictive analytical capabilities for events yet to take place. The BCP analytics may provide advantages over conventional systems in similarity estimation, generalization from a single exemplar, and recognition of more than one class of stimuli within a complex composition (“scene”) given single exemplars from each class. This enables the system to generalize and abstract non-sensory data (EEG, speech, movement). Combined, these provide both global (brain-wide) and fine detail (for example, communication between and within cytoarchitectonic areas) modalities for reading and writing across different timescales.

The implant device may be a microfabricated carbon nanotube neural implant that may provide, for example, reading from ≥1,000,000 neurons, writing to ≥100,000 neurons, and reading and writing simultaneously to ≥1,000 neurons. The BCCS may include multisensory wireless inductive earbuds and behavioral sensors and provide wireless communication with implant device, inductively recharge implant device, provide Bluetooth communication with a secure app on smartphones, tablets, etc., and may provide interfacing with cloud—API, SDK and secure website for clinicians, patients (users)

The implant device and BCCS devices may be used in combination with FCU, BC and IA algorithms to translate audial cortex output, matching internal and external stimulus (for example, output) to transcribe thought into human readable text.

The BCP may provide advantages over conventional systems by providing a closed loop neural interface system that uses big data analytics and extreme machine learning on a secure cloud platform, to read from and intelligently respond to the brain using both electrical and optical modulation. The FCU unary framework enables extremely high-speed compression, encryption, and abstract data representation, allowing the system to process multimodal and multi-device data in real-time. This capability is of great interest and benefit to both cognitive neurosciences and basic comprehension of brain function and dysfunction because: (1) it combines high dynamic spatiotemporal and functional resolution with the ability to show how the brain responds to demands made by change in the environment and adapts over time through its multiple relationships of brain-behavior and brain-effectors; (2) it assesses causality because the data streams are exhibited temporally relative to the initial state and each state thereafter by integrating physiological and behavioral factors such as global synchrony, attention level, fatigues etc. and (3) data collection does not affect, interfere, or disrupt any function during the process.

The BCP may provide advantages over conventional systems by recording from all six layers of the primary AI cortex and simultaneously from the mPFC, with very high spatial resolution along the axis of the penetrating probe by combining CNT with fiber optic probes that wrap around a central nucleus. By including the principal input layer IV and the intra columnar projection layers, as well as the major output layers V and VI, brain activity can be monitored with unprecedented resolution. The recording array will be combined with optogenetic stimulation fibers, which are considerably larger and stiffer than electrode arrays. CNT fibers will be used as recording electrodes at an unprecedented scale and within a highly dense geometry.

Carbon nanotubes address the most important challenges that currently limit the long-term use of neural electrodes and their unique combination of electrical, mechanical and nanoscale properties make them particularly attractive for use in neural implants. CNTs allow for the use of smaller electrodes by reducing impedance, improving signal-to-noise ratios while improving the biological response to neural electrodes. Measurements show that the output photocurrent varies linearly with the input light intensity and can be modulated by bias-voltage. The quantum efficiency of CNTs are about 0.063% in 760 Torr ambient, and becomes 1.93% in 3 mTorr ambient. A SWCNT fiber bundle can be stably implanted in the brain for long periods of time and attract neurons to grow or self-attaching to the probes. CNT and optical fibers will be an excellent shank to wrap a polymer array around.

Returning to FIG. 2, the optical fibers 202 will be coated with SWCNTs and make electrical connections with the underlying delay line. The delay line 204 will be transparent to allow light from the vertical-cavity surface-emitting lasers 206 (VCSELs) to reach the optical fibers. The delay lines 204 potentially make the electrical signal position-dependent by comparing the time between pulses measured at the outputs. Provided the pulses are of sufficient intensity and individual pulses are sufficiently separated in time (>1 μs or so), the difference between pulse arrival times could be related to the position on the array. Combining this with spatially controlled optical excitation (i.e., by turning on specific VCSELs 206) would further help to quantify position, as VCSEL pulses excite a small region at the end of the adjacent fiber. These pulses are measured at a position on the delay line close to this fiber, so if neighboring neurons fire, they are sensed by nearby fibers (i.e., the SWCNTs on the fibers) and would generate additional pulses that could then be tracked over time with the delay line, mapping out the path. The SWCNT coated fiber array 202 would be randomly connected to the underlying VCSEL array as we will not have control over the fiber locations in the bundle. The substrate connectors will be graphitic nano joints to a single-walled carbon nanotube, we will also utilize the IBM CNT connect technique for other connectors.

Carbon nanotubes are ideal for integration into a neural interface and the technical feasibility of doing so is well documented. The use of CNT allows for one unit to function as recording electrodes and stimulating optical fibers. The optical transceivers will be integrated as a separate die on a silicon substrate, tightly-coupled to logic dice (a.k.a. “2.5D integration”). The choice of materials reflects the positive results of recent studies demonstrating the impact of flexibility and density of implanted probes on CNNI tissue responses. CNTs are not only biocompatible in robust coatings, but they are supportive to neuron growth and adhesion. It has been found that CNTs actually promote neurite growth, neuronal adhesion, and viability of cultured neurons under traditional conditions. The nanoscale dimensions of the CNT allow for molecular interactions with neurons and the nanoscale surface topography is ideal for attracting neurons. In fact, they have been shown to improve network formation between neighboring neurons by the presence of increased spontaneous postsynaptic currents, which is a widely accepted way to judge health of network structure. Additionally, functionalization of CNT can be used to alter neuron behavior significantly. In terms of the brain's immune response, CNT have been shown to decrease the negative impact of the implanted electrodes. Upon injury to neuronal tissue, microglia (the macrophage-like cells of the nervous system) respond to protect the neurons from the foreign body and heal the injury, and astrocytes change morphology and begin to secrete glial fibrillary acidic protein to form the glial scar. This scar encapsulates the electrode and separates it from the neurons. However, carbon nanomaterials have been shown to decrease the number and function of astrocytes in the brain, which in turn decreases the glial scar formation.

Optogenetic tools may be used to enable precise silencing of specific target neurons. Using unary controls in combinations and in rapid closed loop controls within the implant device will enable neural synapse firings with highly precise timing, intensity, and frequency modulation. Optical neuromodulation has many benefits over traditional electrode-based neurostimulation. This strategy will allow precision stimulation in near real time.

The implant device uses a 3D design (and dissoluble membrane), both of which may provide advantages over conventional systems. The dissoluble membrane protects both the patient and the implant during surgery and the lubricant and contraction encourages neural encroachment and adherence to CNTs upon dissolution. This design maximizes neural connectivity and adhesion, while minimizing implant size. implant device size is further reduced through inductive charging.

The BCP system aims at producing a significant leap in neuroscience research not only in scale but also in precision. The method of optical reading and writing at the same time, using SWCNT optrodes, can be combined with current cell marking techniques to guide electrodes and optic fibers to specific regions of the brain. One of the biggest challenges facing neuroscientists is to know for certain if they are hitting the right spot when performing in vivo experiments, whether it is an electrophysiological recording or an optogenetic stimulation. Cell marking techniques, on the other hand, have made a lot of progress during the past 20 years with the use of new viral approaches as well as Cre-Lox recombination techniques to express cell markers in specific sites of the brain. This has allowed, for example, the expression of fluorescent Calcium indicators in target locations without affecting surrounding regions, which is commonly used in in vivo Calcium imaging. Our technique of simultaneous optical reading and writing makes it possible to insert optrodes and guide them through brain tissue until they “sense” optical changes corresponding to the activity of target cells that express a Calcium indicator. This will reduce, to a great extent, the probability of off-target recordings and stimulations.

The synchronous connection between the implant device and BCCS will likely lead to rapid advances in understanding the key circuits and language of the brain. The BCP provides researchers with a more thorough (and contextual) understanding of neural signaling patterns than ever before, enabling far more responsive brain-machine interfaces (for example, enabling a paralyzed patient to control a computer, quadcopter, or mechanical prosthetic). A wireless implanted device might allow a PD patient to not only quell tremors but actually regain motor capacity, even just minutes after receiving an implant. By combining these technologies with behavioral and physiological metrics, we hope to open up new horizons for the analysis of cognition. Our multimodal diagnostic and analysis allows for an approach of analyzing brain machinery at higher data resolution. The data method could be considered a first step in progressing medicine from snapshots of macro anatomo-physiology to continuous, in-vivo monitoring of micro anatomo-physiology. The in-vivo study of a brain's parcel may give us a real-time relationship of the different components and their functionality, from which the complex functional mechanism of the brain machinery could be highlighted. Giving rise to new medical approaches of diagnosis, treatment, and research. If the animal experiences of two implants prove efficacy and lack of any harm to animal or humans, the BCP may allow us to define a powerful new technique for brain-functional mapping which could be used to systematically analyze and understand the interconnectivity of each brain region, along with the functionality of each region.

Therapeutic aims may include use of the device as a brain stimulator, and indirect by data from recordings highlighting the mechanism(s) by which several diseases occur, owing to implant device's ability to record a basic global neuronal state of a brain region and the dynamic neuronal interplay. The modifications which occur during its normal activity enable us to understand the neuronal properties and the function of a given brain region. Our device is able to give us the dynamic continuum of the whole activity of the considered region and thus provide important insights into the fundamental mechanisms underlying both normal brain function and abnormal brain functions (for example, brain disease). The potential for these findings to be translated into therapies are endless because this device may be used in any region of the brain and represents the first synthesis of a closed-loop neural modulator informed by internal and external conditions. The BCP provides a large amount of information and could be used to explore any brain disease within a real dynamic, in vivo condition. If successful, the potential of this device for the diagnosis of organic brain diseases is enormous and it could be an important complement to MRI for the diagnosis of non-organic disease. The possible therapeutic use of this device may also include chronic pain, tinnitus, and epilepsy. The device could be used in focal epileptic zone owing to its optogenetic capacity to control excitability of a specific populations of neurons. Even if the device does not cure epilepsy, it may help to control otherwise refractory seizures and help to avoid surgery. Nonetheless optimizing the place of this device in therapy for epilepsy will require further study and clinical experience.

Recent demonstrations of direct, real-time interfaces between living brain tissue and artificial devices, such as with computer cursors, robots, and mechanical prostheses, have opened new avenues for experimental and clinical investigation of Brain Machine Interfaces (BMIs). BMIs have rapidly become incorporated into the development of ‘neuroprosthetics,’ which are devices that use neurophysiological signals from undamaged components of the central or peripheral nervous system to allow patients to regain motor capabilities. Indeed, several findings already point to a bright future for neuroprosthetics in many domains of rehabilitation medicine. For example, scalp electroencephalography (EEG) signals linked to a computer have provided ‘locked-in’ patients with a channel of communication. BMI technology, based on multi-electrode single-unit recordings, a technique originally introduced in rodents and later demonstrated in non-human primates, has yet to be transferred to clinical neuroprosthetics. Human trials in which paralyzed patients were chronically implanted with cone electrodes or intracortical multi-electrode arrays allowed the direct control of computer cursors. However, these trials also raised a number of issues that need to be addressed before the true clinical worth of invasive BMIs can be realized. These include the reliability, safety and biocompatibility of chronic brain implants and the longevity of chronic recordings, areas that require greater attention if BMIs are to be safely moved into the clinical arena. In addition to offering hope for a potential future therapy for the rehabilitation of severely paralyzed patients, BMIs can be extremely useful platforms to test various ideas for how populations of neurons encode information in behaving animals. Together with other methods, research on BMIs has contributed to the growing consensus that distributed neural ensembles, rather than the single neuron, constitute the true functional unit of the CNS responsible for the production of a wide behavioral repertoire (reference).

When designing an interface between a living tissue and an electronic device, there are important factors to consider. Particularly, the structural and chemical differences between these two systems; the electrode ability to transfer charge; and the temporal-spatial resolution of recording and stimulation. Traditional multi-electrode array (MEAs) for neuronal applications present several limitations: low signal to noise ratio (SNR), low spatial resolution (leading to poor site specificity) and limited biocompatibility (easily encapsulated with non-conductive undesirable glial scar tissue) which increases tissue injury and immune response. Neural electrodes should also accommodate for differences in mechanical properties, bioactivity, and mechanisms of charge transport, to ensure both the viability of the cells and the effectiveness of the electrical interface. An ideal material to meet these requirements is carbon nanotubes (CNTs). CNTs are well suited for neural electrical interfacing applications owing to their large surface area, superior electrical and mechanical properties, and the ability to support excellent neuronal cell adhesion. Over the past several years it has been demonstrated as a promising material for neural interfacing applications. It was shown that the CNTs coating enhanced both recording and electrical stimulation of neurons in culture, rats, and monkeys by decreasing the electrode impedance and increasing charge transfer. Related work demonstrated the single-walled CNTs composite can serve as material foundation of neural electrodes with chemical structure better adapted with long-term integration with the neural tissue, which was tested on rabbit retinas, crayfish in vitro and rat cortex in vivo.

Using long CNTs implanted into the brain has many advantages, for instance an optical fiber with CNTs protruding from it, but this technology has not been trialed in vivo or expanded to very large numbers of recording channels. Characterization in vitro showed that the tissue contact impedance of CNT fibers was lower than that of state-of-the-art metal electrodes, chronic studies in vivo in parkinsonian rodents also showed that CNT fiber microelectrodes stimulated neurons as effectively as metal electrodes. Stimulation of hippocampal neurons in vitro with vertically multiwalled CNTs electrodes suggested CNTs were capable of providing far safer and efficacious solutions for neural prostheses than metal electrode approaches. CNT-MEA chips proved useful for in vitro studies of stem cell differentiation, drug screening and toxicity, synaptic plasticity, and pathogenic processes involved in epilepsy, stroke, and neurodegenerative diseases. Nanotubes are a great feature for reducing adverse tissue reactions and maximizing the chances of high-quality recordings, but squeezing a lot of hardware into a small volume of tissue will likely produce severe astroglial reactions and neuronal death. At the same time, CNTs could extend the recording capabilities of the implant beyond the astroglial scar, without increasing the foreign body response and the magnitude of tissue reactions. Implantation of traditional, rigid silicon electrode arrays has been shown to produce a progressive breakdown of the blood-brain barrier and recruitment of an astroglial scar with an associated microglia response.

Neural implant geometry and design is highly dependent on animal model used, where larger animals will see a somewhat less dramatic deterioration in recording quality and quantity, so early trials in rats probably shouldn't be too focused on obtaining very long-term recordings on a very large number of channels. While loss of yield due to abiotic failures is a manufacturing process and handling problem, biotic failures driven hostile tissue reactions can only be addressed by implementing design concepts shown to reduce reactive astrogliosis, microglial recruitment and neuronal death (Prasad, A. et al., 2012; McGonnell, G C. et al., 2009).

Conventional thin film probes can fit hundreds of leads into one penetrating shank. Rolling up a planar design would come with several benefits: first, it would decrease the amount of tissue damage a wide 2D-structure would produce. This is essential for the very high densities we are aiming for. Second, it would stiffen the probe, making it easier to penetrate tissue. Thirdly, a round cross section is preferable for reducing the foreign body response in the brain parenchyma. Finally, this design allows for potentially extremely dense architectures, as by combining several of these probes into a 10×10 array of 1 cm², an implant using this technology could potentially deploy several tens of thousands of leads in a multielectrode array, and could be conceivably combined with optical fibers for stimulation within an electronic-photonic microarray implant. A design of an implantable electrode system may be a 3D electrode array attached to a platform on the cortical surface. Said platform would be used for signal processing and wireless communication.

Why coatings or composites with CNT? The unique combination of electrical, mechanical and nanoscale properties of carbon nanotubes (CNT) make them very attractive for use in NE. Recent CNT studies have tried different CNT coatings or composites on metal electrodes and growing full electrodes purely from CNT. Edward W. Keefer et al., (2008) was the first to do a recording study using different coatings made with CNT on electrodes. They found that CNT can help improve the electrode performance during recording by decreasing impedance, increasing charge transfer, and increasing signal-to-noise ratio. CNT may improve the biological response to neural electrodes by minimizing risk of brain tissue rejection.

Why ICA for analysis? ICA signal separation is performed on a sample by sample basis where no information about spike shape is used. For this reason, it is possible to achieve good performance of sorting accuracy in terms of misses and false positives, especially in cases where the background noise is not stationary but fluctuate throughout trials, which is the fact based on biophysical and anatomical considerations but is ignored by most current spike sorting algorithms One assumption underlying this technique is that the unknown sources are independent, which is the case under the assumption that the extracellular space is electrically homogeneous, pairs of cells are less likely to be equidistant from both electrodes. The other assumption of this approach is that the number of channels must equal or greater than the number of sources, which can yield advantages for large-scaled recordings.

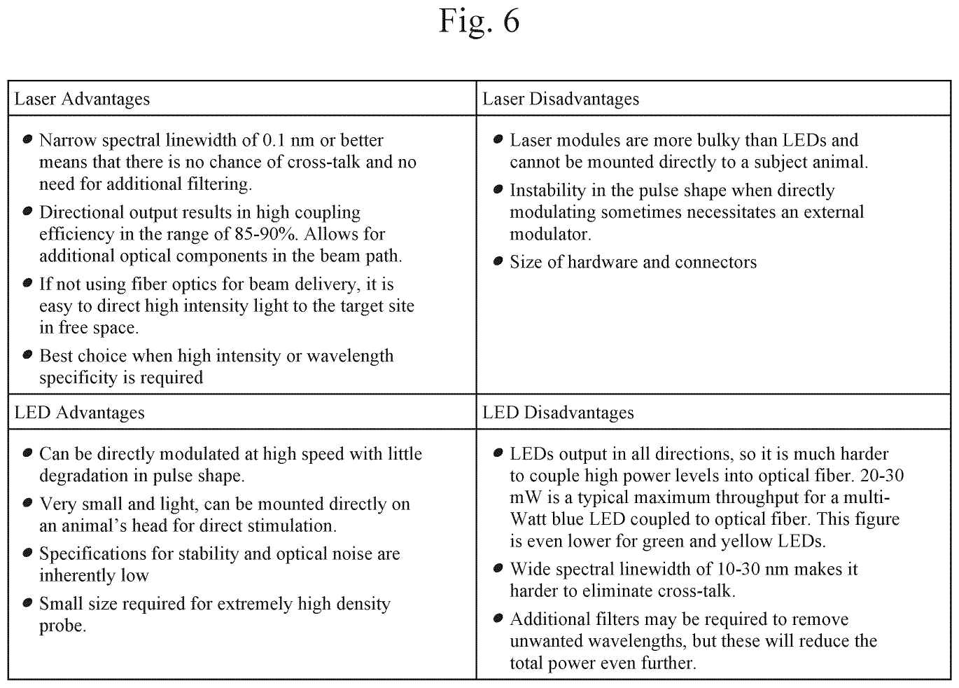

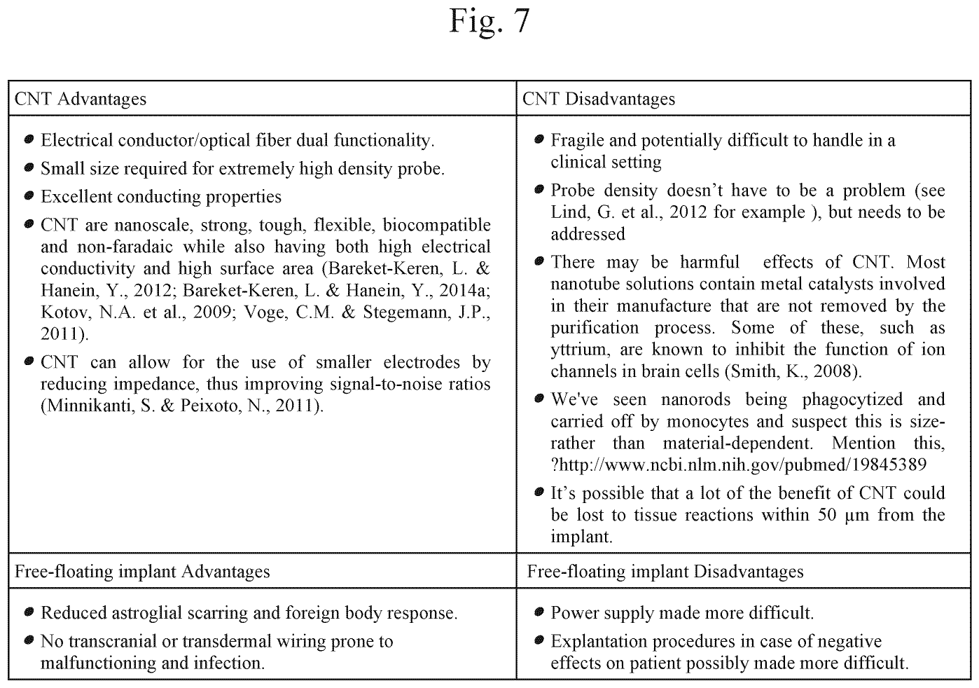

Exemplary tables of advantages of aspects of technologies that may be utilized by embodiments are shown in FIGS. 6 and 7.

The two-implant device's may be implanted within the mPFC in addition to the AI primary auditory cortex because this cortical area may be implicated in the pathogenesis of PTSD. Dopaminergic modulation of high-level cognition in Parkinson's disease and the role of the prefrontal cortex may be revealed by PET, as may widely distributed corticostriatal projections. The mPFC may also be implicated in psychiatric aspects of other disorders, for example deficits in executive functions, anxiety, and depression. By recording from the selected sensory areas and implanting two kiwis at same time, the chance of needing further surgical corrections may be reduced, and data recording may be increased. Knowledge may be extracted that may lead to corrections of associated cognitive deficit in conditions like PTSD but in general to cognitive decline as it occurs for many unknown indicators.

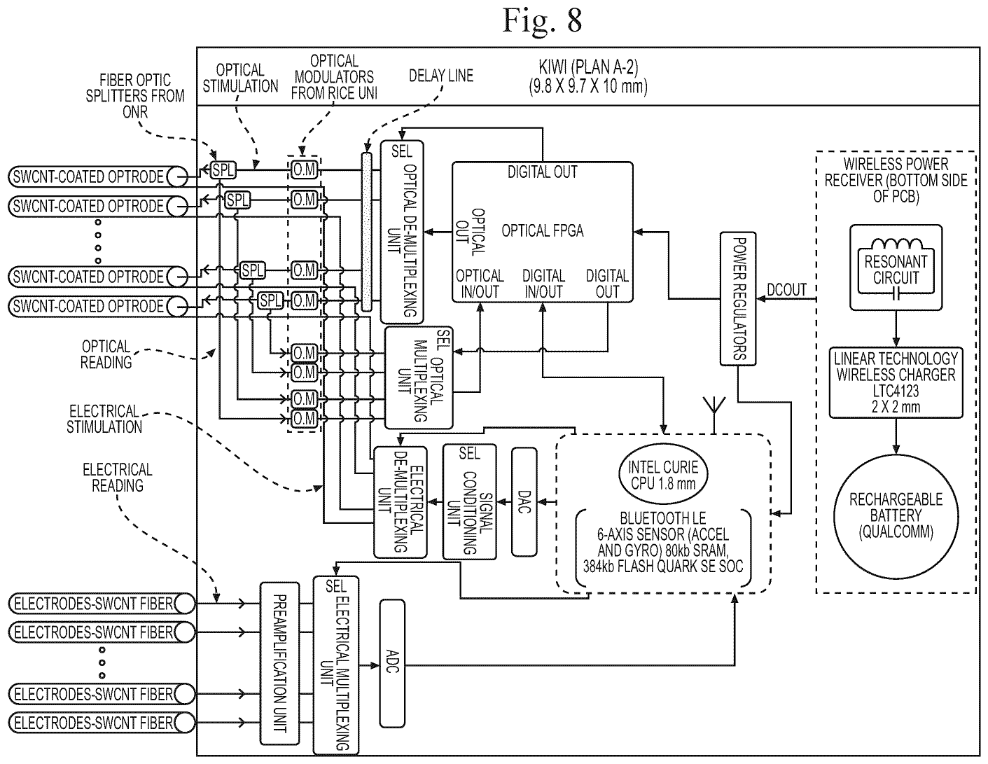

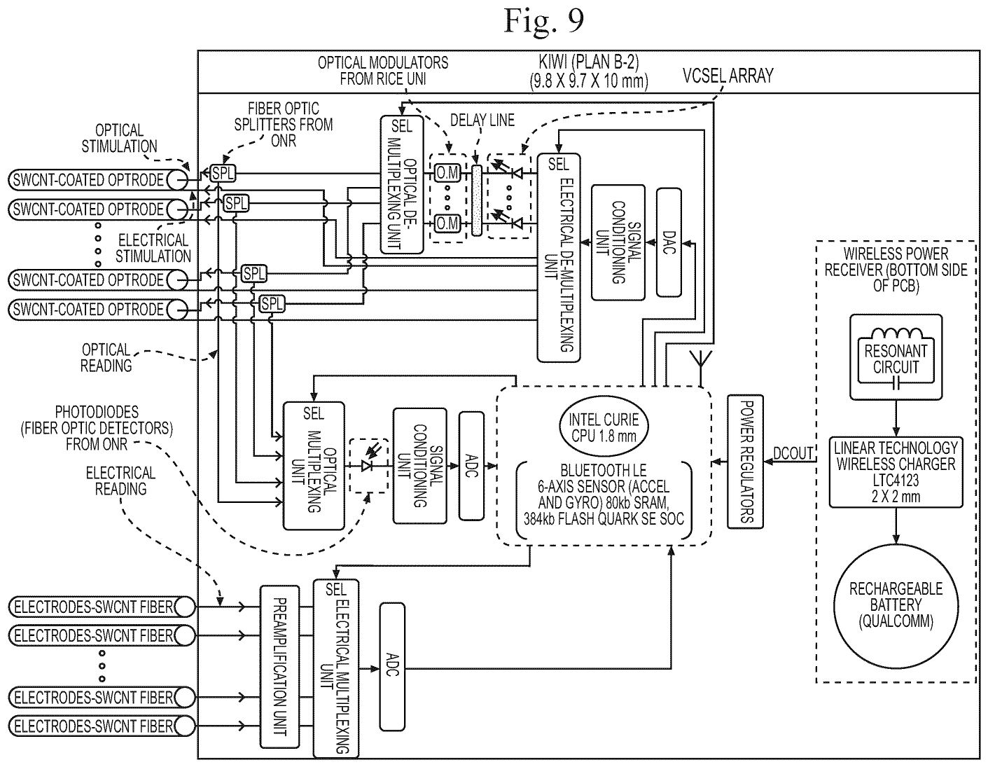

In an embodiment, the BCP hardware may be fabricated using electronic components available on the market today. In an embodiment, the implant device may be made with a microfabricated carbon nanotube (CNT) neural interface, a light modulation and detection silicon photonic chip, and an independent Central Processing Unit (CPU) where all the processing will preside. RF communication between the implant device and BCCS may carried out either by making use of the processor's Bluetooth capability or by implementing an independent RF transceiver in each of the two devices. The BCCS device may be calibrated to and securely integrated with the implant device. Exemplary block diagrams of embodiments of an implant device 800 is shown in FIGS. 8 and 9, and are described further below.

In embodiments, the implant device may be constantly ON without rebooting, patient-wise, and it does not need to be re-programmed or re-uploaded with data from erasable non-volatile memory (eNVM). Once implanted, it always works: records and, stimulates neural activity. In embodiments, a custom-made ASIC may be preferable to the exploitation of MCU. Such an ASIC allows for higher speed and power efficiency, which are the key factors in this application.

Voltage/Power—The solution is intended to be ON all the time. In our case, the operating voltage may be a factor.

3. eNVM—In embodiments, the size of eNVM may be sufficient to store one or more entire neural “frames”. In embodiments, the eNVM may be minimized as much as necessary to meet the performance in speed and power to interface with ADCs, latch and sample the incoming data, and transmit it to the external world. Sampled data may be stored in SRAM and erased once transferred to eNVM.

Embodiments may provide application of ultra-wide band (UWB) communications in invasive implants. In embodiments, the receiver should be as close as possible. Embodiments may have a wearable electronic module on a wrist or as a shoulder band, keeping the receiver within a 0.5-1m range. Embodiments may reduce the range even further using a headset.

In embodiments, there may be only one implant device to be implanted per individual. But this strongly depends on how many electrodes are used, which is determined by the design restrictions and characteristics. In embodiments, one implant device may suffice. However, multiple implant devices may be implanted due to their small size, and they can work in a mesh accordingly.

In embodiments, a cognitive quantum computer chip (CQCC) may have eNVM requirement and relatively small memory sizes are a MCU as opposed to MPU SoC. For example, a 40-800 Mhz-1 GHz selector is fast for an MCU as it cannot execute a single cycle instruction from on-chip eNVM (e.g. eFLASH, MRAM ) at those speeds. Accordingly, at such speeds single cycle instruction may execute form SRAM. NXP has i.MX RT “crossover MCU/MPU” family of products at with this performance.

RF Connectivity—WiFi may not be an optimum solution for power usage depending on requirements for data rate, distance, QoS. UWB maybe be a better option.

Embodiments may include Intel 16 as a target node depending on Duty cycle, RF and eNVM selections.

Voltage/Performance/Power—In embodiments, an implant device may be on all the time, not off for long periods For Active power operating voltage dominates; for sleepy products, leakage dominates.

eNVM—eFLASH does not scale economically below 40 nm. Below 40 nm other technologies such as MRAM, ReRAM, PCM, FeFET, etc., may be better for Code and data storage. IFS does have MRAM on Intel16. Embodiments may include external Octal/QSPI eXecute In Place (XIP) NOR flash die in the package (this is architecture for NXP i.MX RT)

Data exchange—Embodiments may include truly embedded and not tethered RF or inductive communications. UWB should be able to support 150 Mb.; WiGig supports high data rate. Embodiments may compress communications stream. In embodiments, multiple implant devices may be implanted and may form a mesh or multiple node network.

DSPs/NPUs—Several (AIoT) AI/ML IPs available, such from Cadence (Tensilica), Synopsys (ARC), ARM, and start-ups like Roviero.

Packaging—Embodiments may be implemented on a very small die.

In embodiments, examples of voltages used may include Digital circuit Voltage <=1.0 V, I/O analog <=1.8V, clocking speed—800 MHz-1 GHz. Embodiments may include a battery source such as a Rechargeable Battery

Power consumption—This is an important parameter, since the implant device is a remote device operating autonomously. It is a primary element of the system, hence power demands determine the design of the implant device. Ideally, power management would allow the implant device to be a self-sustainable implant upon a once-in-a-lifetime installation.

Logic gate count—may be approximately, for the processor 12-25 k, SRAM 1.3 M, Flash 6.15 M. This is a rough approximation. Both SRAM and Flash memory size can be reduced, ultimately decreasing the number of gates.

Embodiments may provide resources such as on chip memory—SRAM—80 kB, Flash −384 kB, analog precision—Bit-resolution 10 bits, sampling 200 Msps, Signal-to-noise and distortion ratio (SNDR) 50+dB, Integral nonlinearity (INL) ±2 LSB, Differential nonlinearity (DNL) ±2LSB, and Effective number of bits (ENOB) 9+.

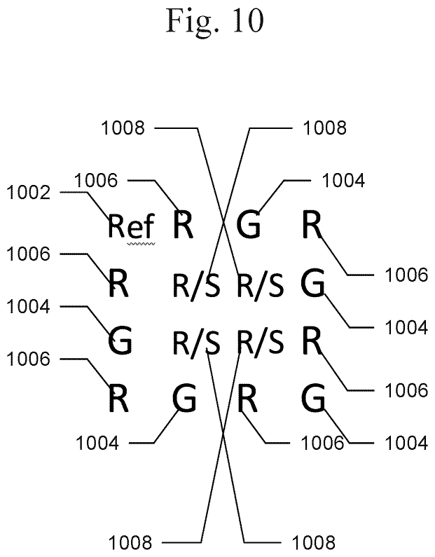

As may be seen from FIG. 2, the implant device may be composed of two such hardware components in a back to back configuration, each one functioning independently. In embodiments, each of the two boards may be split into, for example, 100 tiles with 16 I/O pins. An exemplary embodiment of such a tile design is shown in FIG. 10. Each tile may include, for example, one Reference Pin 1002, five Ground Pins 1004, six Recording Pins 1006, and four pins for either Recording or Stimulation 1008. The specific function of each pin is described below. On one side the tile cells may be attached to CNTs, while on the other side, the tiles may interface with the hardware components needed to process the analog signals.

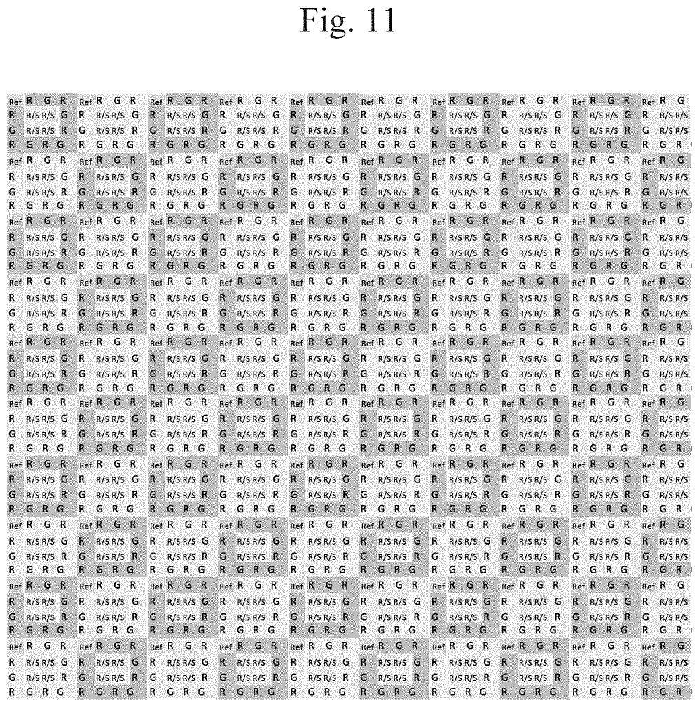

An exemplary embodiment of an arrangement of tiles is shown in FIG. 11. In this embodiment, the tiles may be physically arranged in a 10×10 matrix as shown. Each integrated circuit (application-specific integrated circuit (ASIC), field-programmable gate array (FPGA), etc.) may be connected to a tile block that is composed of, for example, 10×10 tiles. Thus, the integrated circuit may simultaneously read 10×10×10=1000 channels and simultaneously stimulate up to 10×10×4=400 channels. In an embodiment, the implant device may include two integrated circuits and be able to read up to 2000 channels and write up to 800 channels simultaneously.

Channel types that may be supported may include Optrodes and Electrodes. Optrodes (optical electrodes), may perform optical and electrical recording and stimulation. Optrodes may be composed of optical fiber coated with single walled carbon nanotubes. The optical fiber may be used for transporting light signals bidirectionally. Electrodes may perform only electrical recording and stimulation. The carbon nanotubes may be used to transport electric signals. In embodiments, the configuration may depend on the goals of the device implant for each individual patient. Thus, in embodiments, the implant device may support different configurations in terms of channels (number and type (electrical, optical, or chemical) of stimulation and/or recording channels) and Computing Power.

In embodiments, the power budget of the implant device may be in the range of about 100 μW to 1 mW. Embodiments of battery options, assuming an implant device autonomy of 72 hours may include:

Rechargeable Li-ion Battery: In embodiments, the battery may be as small as a grain of rice. The energy of such a battery would be only 3 mWh, or maybe less in normal operating conditions. If a more likely nominal capacity of 2 mWh is considered, this equates to a power budget of 30 μW over a period of 72 hours, in the case that a custom integrated circuit is not needed.

Rechargeable Silver Oxide Battery: In embodiments, a cylindrical Silver Oxide battery with a volume of about 30 cmm (cubic millimeters) may have a nominal capacity of 11 mWh. Over a period of 72 hours this equates to a power budget of about 160 μW. However, due to the chemistry of the Silver Oxide battery, it can only allow a limited number of recharge cycles.

Rechargeable Li—Po Battery: While expensive compared to the other two options, the Li—Po batteries promise about 1200 Wh/L, which would equate to 36 mWh for the same volume of 30 cmm. Over a period of 72 hours, this equates to a power budget of about 500 μW. Due to its high power density, the Li—Po battery has since long been used for pacemakers and may be used in embodiments of this application as well.

For safety reasons, the battery should not heat up more than 1° C. during charging.

Typical implant methods and medical implications. In the field of neural modulation, DBS surgery has been used for the symptomatic treatment of Parkinson's disease for a long time. The intervention implies the drilling of the skull and the insertion of the stimulation electrodes deep within the brain. After this step, another intervention inserts the pulse generator under the skin of the patient's chest, close to the collar bone. Severe intraoperative adverse events included vasovagal response, hypotension, and seizure. Postoperative imaging confirmed asymptomatic intracerebral hemorrhage (ICH), asymptomatic intraventricular hemorrhage, symptomatic ICH, and ischemic infarction, and was associated with hemiparesis and/or decreased consciousness. Long-term complications of DBS device implantation not requiring additional surgery included hardware discomfort and loss of desired effect in 10. Hardware-related complications requiring surgical revision included wound infections, lead malposition and/or migration. component fracture, component malfunction, and loss of effect.

Under DARPA's Reliable Neural-Interface Technology (RE-NET) program, scientists have developed the stentrode, a chip that is far less invasive due to the fact that it is implanted to the brain through blood vessels without opening the skull. This approach was tested on sheep and the chip was inserted via a blood vessel in the neck and guided to the brain using real-time imaging. Once the chip reaches the target location it expands and attaches to the walls of the blood vessel to read the activity of the nearby neurons.

In embodiments, different implantation procedure may be used, and each has advantages and disadvantages.

Implant device Cyber Security. Billions of sensors that are already deployed lack protection against attacks that manipulate the physical properties of devices to cause sensors and embedded devices to malfunction. Analog signals such as sound or electromagnetic waves can be used as part of “transduction attacks” to spoof data by exploiting the physics of sensors.

A “return to classic engineering approaches” may be needed to cope with physics-based attacks on sensors and other embedded devices, including a focus on system-wide (versus component-specific) testing and the use of new manufacturing techniques to thwart certain types of transduction attacks.

Transduction attacks may target the physics of the hardware that underlies that software, including the circuit boards that discrete components are deployed on, or the materials that make up the components themselves. Although the attacks target vulnerabilities in the hardware, the consequences often arise in the software system, such as improper functioning or denial of service to a sensor or actuator. Hardware and software have what might be considered a “social contract” that analog information captured by sensors will be rendered faithfully as it is transformed into binary data that software can interpret and act on. But materials used to create sensors can be influenced by other phenomena—such as sound waves. Through the targeted use of such signals, the behavior of the sensor may be interfered with and even manipulated.

In embodiments, the implant device may take measures against vulnerability to accidental or malicious wave interferences.

Neuron Connection Interface. Due to their extraordinary properties, CNTs may be used in different roles, such as electrophysiological reading, electrophysiological stimulation, electrochemical detection, optical reading, and optical stimulation. Embodiments may include specialized implant devices that feature only one type of CNTs or hybrid implant devices with multiple types of CNTs, which may use artificial intelligence (AI) to manage them according to the nature of the application.

Carbon Nanotubes (CNTs) are a material with broad application, such as additives, polymers, and catalysts; in autoelectron emission, flat displays, gas discharge tubes, absorption and screening of electromagnetic waves, energy conversion, lithium battery anodes, hydrogen storage, composite materials, nanoprobes, sensors and supercapacitors. CNTs may be used as super-miniaturized chemical and biological sensors based on the fact that their voltage-current (V-I) curves change as a result of adsorption of specific molecules on their surface. Furthermore, the boundary (tip) of the CNT may be modified by functional groups, metal nanoparticles, polymers, and metal oxides to increase the selectivity of the detectors built based on them, adding filtering capabilities to it.

CNTs have remarkable mechanical, thermal, and electrical properties. For example, the Young's modulus of CNTs, which is a measure of axial tensile stiffness, may be over 1TPa (Aluminum has 70 GPa). CNTs may have a strength-to-weight ratio 500 times greater than Aluminum. The thermal conductivity of CNTs may be very high (approximately 3000 W/mK) in the axial direction and very small in the radial direction. CNTs may have a very high current carrying capacity and may have an electrical conductivity six orders of magnitude higher than copper. Due to their high mechanical and thermal stability and resistance to electromigration, CNTs may sustain current densities of up to 109 A/cm2. Depending on their chirality—the geometric orientation of the carbon atoms network—the electrical properties of the CNTs may change—they may behave either as conductors or semiconductors. In an electronic device this may allow both the active devices and interconnects to be made of CNTs.

In embodiments, CNTs may be used as Sensors, for functions such as Electrophysiological Recording, measuring the electrical potential in neural tissue by using CNTs as conductors, Electrochemical Recording, detecting neurotransmitters in neural tissue through fast-scan cyclic voltammetry (FSCV), Optical Recording, making CNTs sensitive to fluorescent substances by changing their chiral configuration, Neural Stimulators, Electrophysiological Stimulation, stimulating the brain neurons by using CNTs as conductors, Optical Stimulation, using Optogenetics techniques, and Electrochemical Stimulation.

Connection Method. When implant device is inserted in the brain, the CNTs may establish strong adhesive contact with the neuronal tissue, becoming able to measure the electrical field in their vicinity. The following approximate calculations provide an intuition on how the implant device CNTs will fit over the neural network. The brain cells may be in the range of 10-50 micrometers in diameter. The width of a CNT may be in the range of 0.7-50 nanometers. In embodiments, the optrodes (the CNT coated optic fibers) or electrodes (with CNT fiber) may be organized in 100 tiles arranged in a square configuration. Each tile may be made of a 4 by 4 array of optrodes. Therefore, the CNTs may be arranged in a 400×400 matrix. Given that one side of the KWI optrode array may be about 1 cm, the interaxial distance between the CNTs is about 25 micrometers.

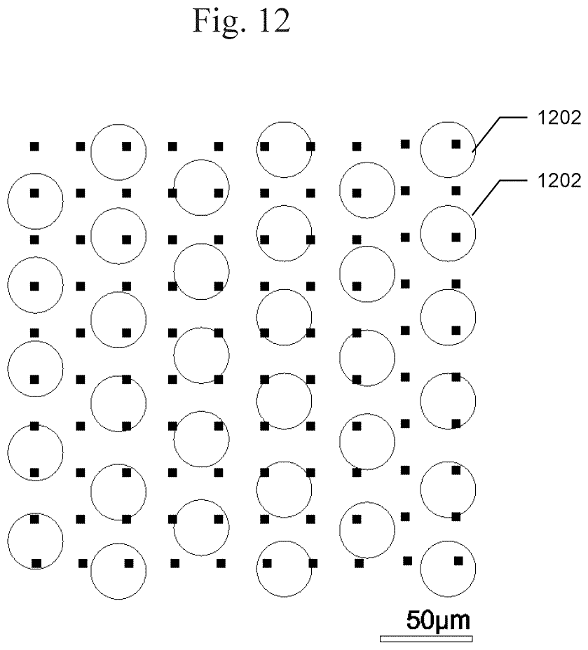

An exemplary illustration of an approximate representation of how the optrode array could fit over a dense neural network is shown in FIG. 12. In this example, the following assumptions have been made. The brain cells 1202 have been represented as circles 30 microns in diameter and 50 microns apart (distance between centers). The centers of the optrodes have been represented as squares 25 microns apart. The diameter of the CNT may be about 1000 times smaller than the diameter of the brain cell, so the CNTs would hardly be visible if they were drawn to scale. For better readability, an array of only 10 by 10 optrodes has been represented.

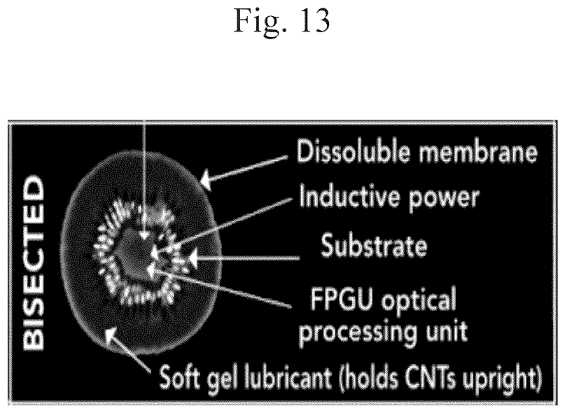

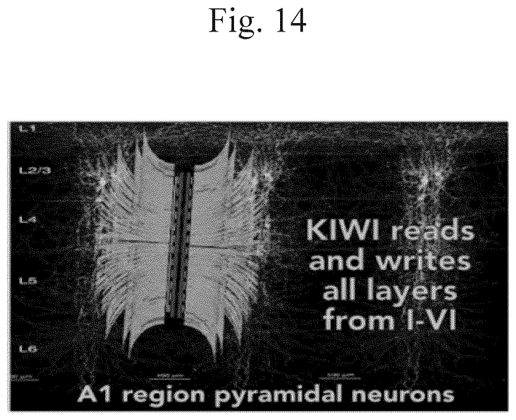

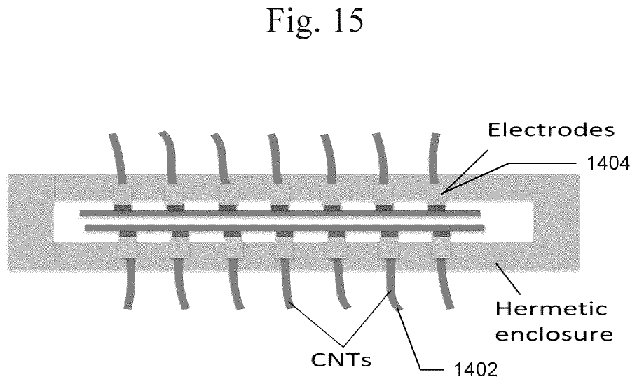

In order to obtain a clear reading from one single point of contact with the brain tissue and avoid electrical short circuit, it is important for the CNTs to remain upright and not stick to each other, which would naturally happen due to the force of molecular adhesion (van der Waals interactions). Soft lubricant gel may be used to ensure their upright position, as shown in FIG. 13. After the implant, due to its size, position and optrodes configuration, the implant device may be able to connect to all neuron layers from I to VI, as shown in FIG. 14. At the other end, the CNTs 1502 may connect to the electrodes 1504 through which the neuron stimulation and reading will be performed, as shown in FIG. 15.

Electrophysiologic Detection of Voltage. In embodiments, CNTs may be used for deep brain recordings of voltages from neural tissues in their vicinities. For this task, CNT, based electrode arrays may be used that enable high-density neural connections in a manner that is non-destructive to the neuronal tissue. This method is feasible and efficient because of all the above-mentioned properties of CNTs—mechanical, thermal, and electrical.

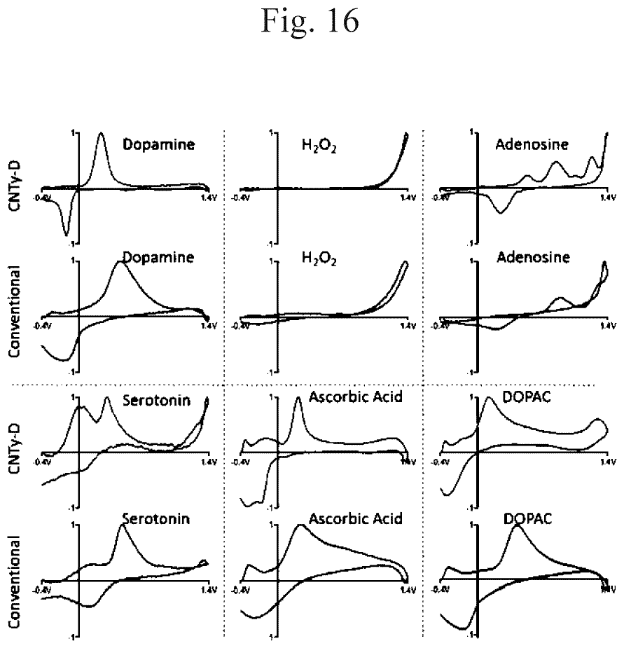

Electrochemical Detection of Neurotransmitters. In embodiments, CNTs may be used in yarn macrostructures (which are several parallel CNTs) to detect neurotransmitters in vivo. Disk-shaped CNT yarns may detect electro-active transmitters, as shown in FIG. 16, which is a fast-scan cyclic voltammetry diagram of CNT yarn disk shaped (CNTy-D) microelectrodes and conventional microelectrodes detecting different neurotransmitter species. The method employed, fast-scan cyclic voltammetry (FSCV), is a technique by which changes in the extracellular concentration of electroactive molecules may be monitored when the electrode is ramped up to a certain threshold over time, and then it is ramped down to return to the initial potential.

Different surface structures (chirality) of the CNTs may result in different CV (Cyclic Voltage) responses towards each neurotransmitter species. The sensitivity of the CNT yarn microelectrodes may also be enhanced by different modification approaches: laser treatment may increase sensitivity towards dopamine, O₂ plasma etching may increase sensitivity towards dopamine, and anti-static gun treatment may increase surface area by increasing the roughness.

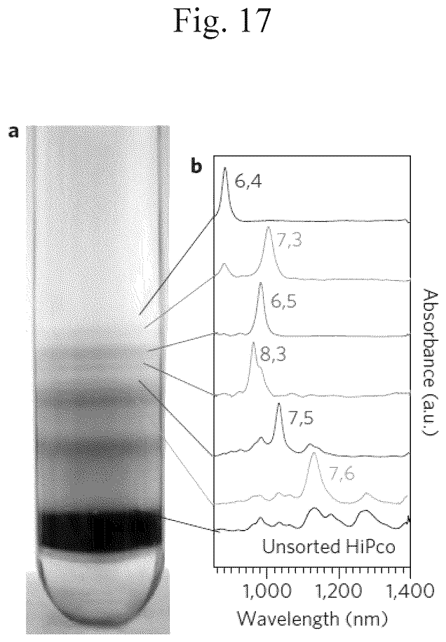

Fluorescent Carbon Nanotubes. The different geometries of the carbon atom network making up a CNT may determine different electronic properties. The different electronic properties may be correlated with different optical properties because their electronic band-gap between valence and conduction band may make the single walled CNTs fluorescent in the near infrared (NIR, 900-1600 nm). This property may enable the CNTs to be used for optical multiplexing because every chiral configuration could be used as a single color. An example of how carbon nanotube color changes with chiral index is shown in FIG. 17. The colors of the CNTs arise due to the absorption of light in the visible range. In this example, a sample with separated SWCNT of different chiralities and corresponding absorption and fluorescence spectra are shown, labelled with the main (n,m) chiral index component. Further, single walled CNTs used as optical sensors may exhibit a near Infrared emission range that coincides with the tissue transparency window.

The unique composition of the polymeric functionals used with single walled CNTs may enable them for the selective detection of neurotransmitters with high spatial resolution. For example, a fluorescent nanosensor array based on single-walled CNTs may be used for sensing dopamine from PC12 neuroprogenitor cells at high temporal (100 ms) and spatial (20.000 sensors per cell) resolution.

CNT arrays as a solution for spatially distributed current release. Techniques have been developed to map electrical microcircuits in the brain at far more detail than existing techniques, which are limited to tiny sections of the brain (or remain confined to simpler model organisms, like zebrafnsh).

In the brain, groups of neurons that connect up in microcircuits help us process information about things we see, smell and taste. Knowing how many neurons and other types of cells make up these microcircuits would give scientists a deeper understanding of how the brain computes complex information.

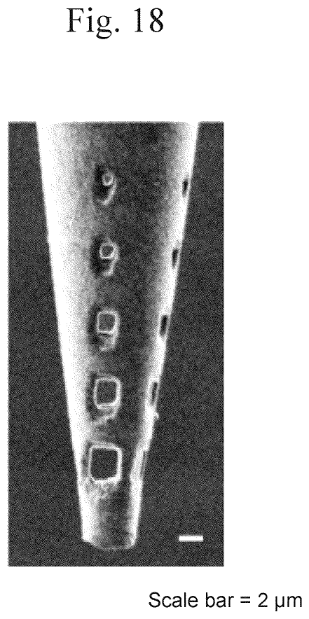

Nanoengineered microelectrodes. Embodiments may use “nanoengineered electroporation microelectrodes” (NEMs). Electroporation is a microbiology technique that applies an electrical field to cells to increase the permeability (ease of penetration) of the cell membrane, allowing (in this case) fluorophores (fluorescent, or glowing dyes) to penetrate into the cells to label (identify parts of) the neural microcircuits (including the “inputs” and “outputs”) under a microscope. Such electrodes may be used to map out cells that make up a specific microcircuit in a part of a brain for a particular function. The electrodes may include a series of tiny pores (holes) near the end of a micropipette, produced using nano-engineering tools. The new design distributes the electrical current uniformly over a wider area (up to a radius of about 50 micrometers the size of a typical neural microcircuit), with minimal cell damage. An example of an embodiment of a NEM can be seen in FIG. 18. By releasing the current through multiple openings, multiple neuron layers may be stimulated using the NEM. Multiple release points mean the current will be distributed in a wider area so that neurons will not suffer from a local current concentration (which one would create to stimulate a larger volume of tissue )

In embodiments, the configuration and implant position of the implant device may provide conditions for multi-point electric stimulation. With regards to reaching multiple layers of neurons, the implant device may connect to layers I to VI, due also to the length and geometrical configuration of the CNTs. With regards to the electrical potential distribution in the tissue, due to the 2000+CNT fibers populating it, the implant device may have a greater number of stimulation points, offering a superior spatial resolution.

Optical Fibers. In addition to embodiments of the implant device being able to read/write electric and electrochemical signals from/to the neurons through the CNTs, embodiments of the implant device may also have the capability of optically stimulating the neurons and reading optical signals from them. The optical interaction between the brain and the implant device may take place through an array of optical fibers in a process called optogenetics.

Optogenetics and fiber photometry are neuro-modulation technologies in neuroscience that utilizes a combination of light and genetics to control and monitor neurons in vivo. In embodiments, optogenetics and fiber photometry may provide the capability to map the amygdala, such as for fear conditioning, to perform studies for targeting pharmacotherapies and addiction via nucleus accumbens, for expression of pyramidal neurons in PFC, and for genetic components of social behavior and drug efficacy in neuropsychiatric disorders etc.

Optical Stimulation. Optogenetics is a technology in which light-sensitive ion channels may be virally expressed in target neurons allowing their activity to be controlled by light. By coating optical fibers with dense, thin CNT conformal coatings, embodiments may include optical modulation units within the nucleus of the implant device that may deliver light to precise locations deep within the brain, while recording electrical activity at the same target locations. As described below, the light-activated proteins Channelrhodopsin-2 and Halorhodopsin may be used to activate and inhibit neurons in response to light of different wavelengths and we are currently developing precisely targetable fiber arrays and in vivo-optimized expression systems to enable the use of this tools in awake, behaving primates.

The implant device software may be synchronized with optogenetic actuators and sensors and fiber photometry devices allowing for acquisition of behavioral data during experiments by using TTL (transistor-transistor logic) and a specially developed software interface. This brings research into a new realm with the possibility of simultaneous control of biochemical events of living freely behaving animals and the collection of this data in both high-throughput and real-time.

In order to be able to monitor and modulate the biochemical events in behaving animals, the animals must be able to move freely without being restricted by wires and tethers. Embodiments of the implant device may provide this capability due to the fact that all data exchanges and power delivery are wireless.

Embodiments of the implant device may be used for experiments mapping function of the amygdala such as fear conditioning, studies for targeting pharmacotherapies and addiction via nucleus accumbens, expression of pyramidal neurons in PFC and genetic components of social behavior and drug efficacy in neuropsychiatric disorders, etc. In embodiments, examples of optogenetic/fiber photometry systems that may be used may include SEIZURESCAN®, HOMECAGESCAN®, GROUPHOUSESCAN®, FREEZESCAN®, CHAMBERSCAN®, GAITSCAN®, TREADSCAN®, RUNWAYSCAN®, TOPSCAN®, AND SOCIALSCAN®.

Optical Sensing of Neurotransmitters. The optical sensing of neurotransmitters may have advantages over the electrochemical sensing techniques. For example, improved Lower limit of detection (the smallest substance concentration/quantity that can be detected), often reaching a nanomolar range or less (compared, for example, to 300 nm. for dopamine detection using electrochemical sensing by CNT yarn microelectrodes. The broad range of optical spectrum may allow for the interference from other chemical species to be minimized. Optical sensing may provide high spatial resolution. The release and uptake of neurotransmitters may occur in a highly localized fashion, therefore the high spatial resolution refers to that fact that the sensors are small enough to identify which neurons are involved in these chemical interactions. Optical sensing may provide improved temporal resolution. The neurotransmitter release and uptake processes occur in a millisecond time range. Optical sensors may have a sampling rate that is high enough to detect the concentration changes.

Neuronal Data Recording. In embodiments, the implant device may include both optical fibers and CNTs that can have multiple roles. In such embodiments, the implant device may record neuronal activity data using, for example, any of the following three methods: Electrophysiological Recording, Optical Recording and Electrochemical Recording. In embodiments, specialized implant devices may be used that feature only one type of neural interaction, hybrid implant devices may be used that feature all types of interaction. In the latter case, complex AI algorithms may be used for CNT management according to their properties.

The Electrophysiological Recording functionality relies on the special current carrying capacity of the CNTs. The Optical Recording may, for example, be performed in two ways. First, the implant device may use an on-board light-source to activate fluorescent cells and may use the dedicated optical fibers to record and transmit the data to the circuitry. Second, the fluorescent CNTs (polymer functionalized CNTs) may be used to optically identify the release of certain neurotransmitters.

The Electrochemical Recording functionality of the implant device may provide for the detection of released neurotransmitters based on analyzing the shape of the curve obtained by plotting current intensity over electric potential in fast-scan cyclic voltammetry.

Recording Capacities. In embodiments, the implant device may record up to 2,000 channels simultaneously. For example, such an embodiment may use the tile architecture described above (implant device Design), which includes 2 electrode/optrode boards, 10×10 tiles per board, and up to 10 recording channels per tile.

In embodiments, the reading and stimulation circuitry may be in the form of a readout-integrated circuit (ROIC), which may be similar to or a modification of, for example, a solid-state imaging array. The ROIC may include a large array of “pixels”, each consisting of a photodiode, and small signal amplifier. In embodiments, the photodiode may be processed as a light emitting diode, and the input to the amplifier may be provided by the CNT connection to the neuron. In this manner, neurons may be stimulated optically, and interrogated electrically. The ROIC may include CCD or CMOS photodiodes or other imaging cells, to receive optical signals, electrical receiving circuitry, to receive electrical signals, light outputting circuitry, such as LED or lasers, to output optical signals, and electrical transmitting circuitry, to transmit electrical signals.

Electrophysiological Recording. In electrophysiology—the oldest strategy for neural recording, an electrode is used to measure the local voltage at a recording site, which conveys information about the spiking activity of one or more nearby neurons. The number of recording sites may be smaller than the number of neurons recorded since each recording site may detect signals from multiple neurons in the area.

An example of an electrophysiological recording pipeline 1900 is shown in FIG. 19. Pipeline 1900 may include a plurality N of electrodes 1902, such as SWCNT fibers. The SWCNT fibers may each be connected to a preamplifier 1904, which may convert the weak electrical signal coming from the neurons into an output signal that is strong enough to be noise-tolerant and processing ready. The output signal from each preamplifier 1904 of a plurality N of preamplifiers 1904 may be input into an electrical Multiplexing Unit (MUX) 1906 having N inputs. Between the processing circuitry 1910 and MUX 1906 is a Select Line 1907, through which processing circuitry 1910 may communicate to MUX 1906 the channel to read through at that time. In order to be able to select from N inputs, the Select Line may specify log₂(N) bits, which means that it may contain that many connections. In an embodiment, there may be 1000 or more recording channels. In such an embodiment, it may be difficult to have a single Multiplexer that can switch among all of the inputs. Accordingly, in embodiments, the circuitry may include, for example, with two layers of multiplexers with 16 input channels each, as follows: 64 multiplexers connected to the CNTs, which feed into 4 multiplexers. In embodiments, there may be another layer of multiplexing as well. Embodiments may include any convenient arrangement of multiplexers to handle the number of recording channels.

From MUX 1906, the selected signal goes into Analog to Digital Converter (ADC) 1908, which converts the received analog value into a digital value, for example, 8, 10 or 12 bits, which is then passed along to processing circuitry 1910. Processing circuitry 1910 may include digital processing circuitry, such as one or more microprocessors, microcontrollers, digital signal processors (DSPs), custom or semi-custom circuitry, such as application specific integrated circuits (ASICs), field programmable circuitry, such as field programmable gate arrays (FPGAs), etc., or any other digital processing circuitry.

In order to minimize the interference between the recording and stimulation signals, in embodiments, the CNTs that are used for electrical recording may be used only for recording. Even so, given the proximity of all the CNTs, in embodiments, the recorded signal may be cleaned of the electric stimulation signal, which is may be much stronger than the signal input from the neurons.

Recording Formula. For calculating the recorded electrical voltage, embodiments may use the Ground that is closest to the Recording channel, and the Reference for negative values. Without the Reference, the negative values would be clipped to 0, and by this valuable information may be lost.

Optical Recording. In embodiments, the implant device may also record optically using optical properties of CNTs and/or optical fibers coated with CNTs. For Optical Recording, the neurons that have been modified, for example, genetically, to have fluorescent capabilities may be illuminated to trigger the fluorescence. The fluorescence may vary based on the voltage that is going through the membrane of the neuron. So, the recorded light intensities may correspond to the voltage strength of the neurons. In embodiments, the optic fiber in the optrode may be used for both optical stimulation and recording by way of a Beam Splitter, which may be positioned close to the optrode, to convert the two-way light circuit into two one-way light circuits.

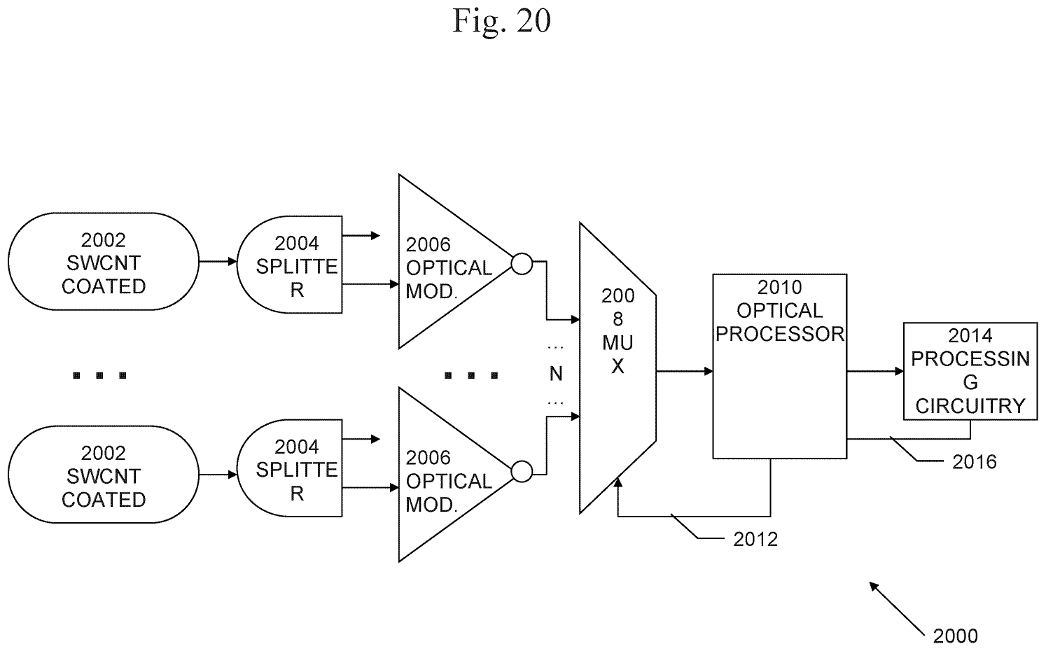

An example of an embodiment of an optical recording pipeline 2000 is shown in FIG. 20. In this example, pipeline 2000 may include a plurality N of optrodes 2002, such as SWCNT coated optical fibers. The signal that comes from each optrode 2002 goes through a beam splitter 2004 into an Optical Modulator 2006, which may transform it from a baseband signal to a bandpass signal, that can be processed by the Optical processor 2010.

From the Optical Modulator 2006, the optical signal may be input to Optical Multiplexing Unit 2008, where based on the selection signal on select line 2012 from the Optical processor 2010, one channel may be selected to be read. The Select Line between Optical Multiplexing Unit 2008 and the Optical processor 2010 may, for example, be a digital electrical signal. The Optical processor 2010 may receive the selection instructions (which channel to read) from the processing circuitry 2014 over select line 2016.

The selected light signal from Optical Multiplexing Unit 2008 may be input to Optical processor 2010 through an optical connection. Optical processor 2010 may convert the light signal into a digital electrical signal, for example, 8, 10 or 12 bits, and outputs the digital signal to processing circuitry 2014. Processing circuitry 2014 may include digital processing circuitry, such as one or more microprocessors, microcontrollers, digital signal processors (DSPs), custom or semi-custom circuitry, such as application specific integrated circuits (ASICs), field programmable circuitry, such as field programmable gate arrays (FPGAs), etc., or any other digital processing circuitry.

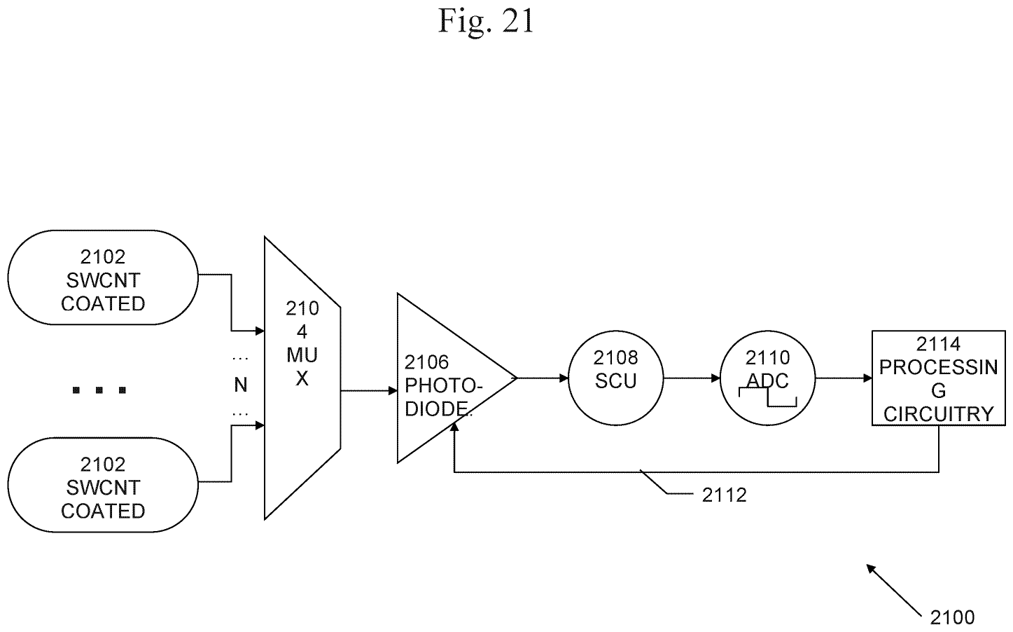

An example of an embodiment of an optical recording pipeline 2100 is shown in FIG. 21. In this example, pipeline 2100 may include a plurality N of optrodes 2102, such as SWCNT coated optical fibers. The signal that comes from each optrode 2102 goes into Optical Multiplexing Unit 2104, where based on the selection signal on select line 2112 from processing circuitry 2114, one channel may be selected to be read. The Select Line between Optical Multiplexing Unit 2108 and the Optical processor 2110 may, for example, be a digital electrical signal.

The selected light signal from Optical Multiplexing Unit 2108 may be input to Photodiode 2106, which converts it into an analog electrical signal. This analog electrical signal may be passed than through a Signal Conditioning Unit 2108, which may perform filtering and amplification on the analog electrical signal. The processed analog electrical signal may then be input into Analog to Digital Converter (ADC) 2110, which may convert it into a digital electrical signal, for example, 8, 10 or 12 bits, and output the digital signal to processing circuitry 2014. Processing circuitry 2114 may include digital processing circuitry, such as one or more microprocessors, microcontrollers, digital signal processors (DSPs), custom or semi-custom circuitry, such as application specific integrated circuits (ASICs), field programmable circuitry, such as field programmable gate arrays (FPGAs), etc., or any other digital processing circuitry.

Electrochemical Recording. Although called Electrochemical Recording, in embodiments, this functionality may rely on the ability of embodiments to electrically stimulate the neural tissue (stimulation) and compute the current intensity (processing) by knowing the electrical resistivity. Electrochemical recording may be performed through the CNTs and may be based on the fast-scan cyclic-voltammetry (FSCV) technique to detect the neurotransmitters' release and uptake. The method involves subjecting neural tissue to an electric potential linearly increasing over time up to a certain threshold. After reaching the threshold, the electric potential is linearly ramped down to the initial value.

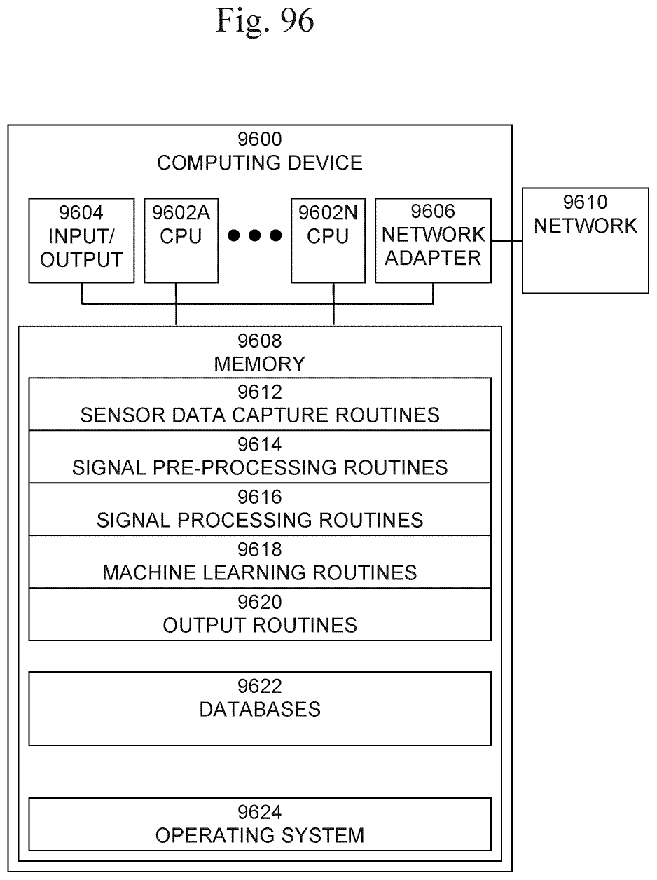

An example of a conceptual diagram of the cyclically applied potential is shown in FIG. 96.

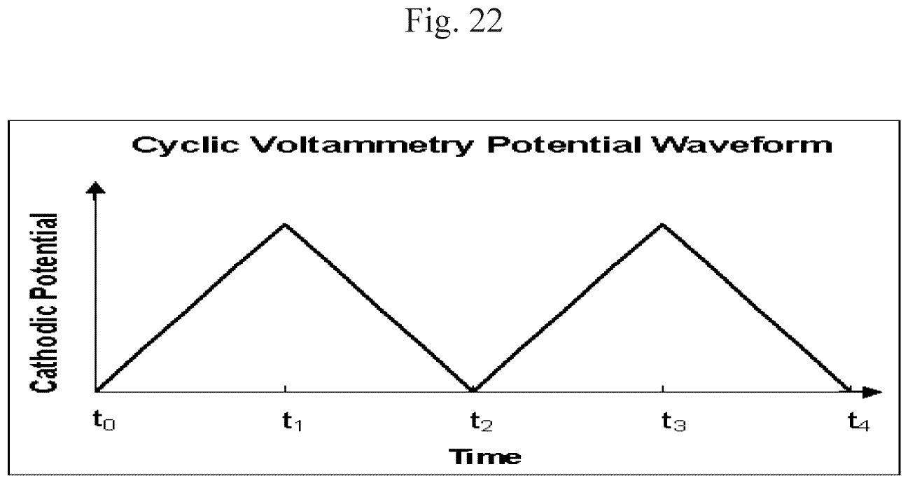

The FSCV stimulation potential may be applied through a specific command given by the processing circuitry through the stimulation pipeline described below. The current at the working electrode is plotted versus the applied voltage to give the cyclic voltammogram trace. A few examples of how these cyclic voltammogram traces look are shown in FIG. 22. Therefore, the released neurotransmitters may be identified based on knowing the shape of their specific cyclic voltammogram trace.

Hybrid Recording: Justification and Specifics. Given that the Electrophysical Recording Pipeline may be built separately from the Optical Recording Pipeline, depending on the number of CNTs assigned to each one of the two methods, embodiments may be able to simultaneously record both electro-physically and optically. By combining both methods, embodiments may record more complex and novel insights about the functionality of the brain.

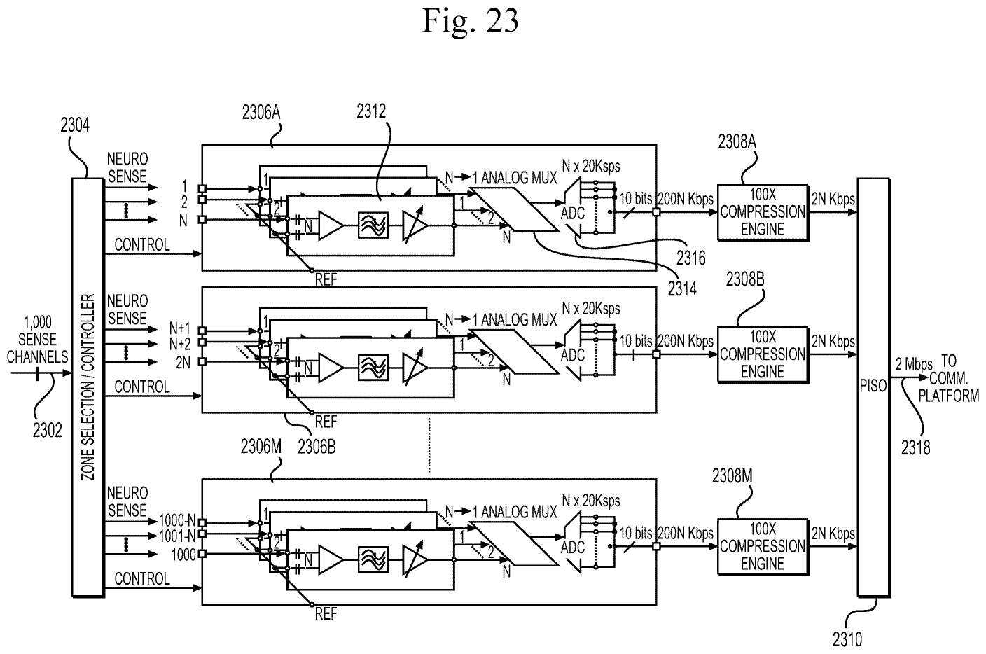

Pipeline Summary. An example of a high-level architecture 2300 of the pipelines presented above, as well as compression and data transmission to Gateway (Communication Platform) is shown in FIG. 23. The sense channels pipeline architecture highlights the components used for propagating the neurons recorded voltages to the Gateway component. As shown in this example, the architecture may include a plurality of sense channels 2302, zone selection/controller circuitry 2304, a plurality of recording pipelines 2306A-M, a plurality of data compression engines 2308A-M, and Parallel-In-Serial-Out Converter (PISO) 2310. Sense channels 2302, for example, electrical and/or optical sense channels including CNTs, SWCNTs, optical fibers, etc., may be input to zone selection/controller circuitry 2304. Zone selection/controller circuitry 2304 may select groups or zones of sense channels 2302 for input to recording pipelines 2306A-M. Recording pipelines 2306A-M may convert analog electrical and/or optical signals to digital electrical signals. Each recording pipeline 2306A-M may handle a plurality of sense channels 2302 and may include a plurality of instances of recording pipeline circuitry. For example, each instance of recording pipeline circuitry may include signal conditioning circuitry 2312, such amplifiers, filters, variable gain stages, etc., N to 1 analog MUX 2314, and ADC 2316. Each instance of recording pipeline circuitry may convert analog electrical and/or optical signals to digital electrical signals at a rate of 20 Kilo-samples per second (Ksps) per input sense channel 2302. Assuming, for this example, 10 bits per sample, each instance of recording pipeline circuitry may generate 200 Kilobits per second (Kbps) of data. As each analog MUX may multiplex N signals, ADC 2316 may generate 200N Kbps of data. The data from each recording pipeline 2306A-M may be input to a data compression engine 2308A-M, which may, for example, provide 100 times compression. Thus, in this example, each 200N Kbps data channel may be compressed to a 2N Kbps data channel. The outputs from each data compression engine 2308A-M may be input to PISO 2310, in which the M parallel 2N Kbps data channels may be serialized to form a single serial output data channel 2318, which may be input to processing circuitry (not shown). In this example, with 1000 sense channels 2302, serial output data channel 2318 may handle 2 Mega-bits per second (Mbps). The maximum sample rate and data rate may depend on the particular engineering design, such as the specifications of the processing circuitry, such as processor and memory.

Although in this example, ADC 2316 may provide 10-bit samples, any resolution ADC may be used. For example, ADCs with resolutions of 24 bits per sample are readily available. However, ADCs having less resolution may consume less power and may take up less space. Accordingly, ADCs having resolutions from 8 bits per sample to 12 bits per sample may provide a good tradeoff between resolution and power and space consumption. Likewise, ADCs having a variable number of bits per sample may be used. For example, such an ADC may provide a variable number of bits per sample of from 8 bits per sample to 12 bits per sample.

The measured data for each sense channel 2302 may represent the voltage from a small region of neural tissue. In embodiments, range of sample rates may be from about 1000 samples/second to about 20,000 samples/second. In embodiments, depending upon the number of sense channels 2302, the maximum compressed data generated throughput may be about 4 Mbps. In embodiments, data representing simultaneously recorded voltages may be grouped into data frames, where the number of recorded values encapsulated in one data frame may depend on the number of simultaneously active reading channels 2302, and on the transfer rate capabilities to the Gateway at that time. The recording process may adapt to the specific use case and the available transfer bandwidth to the Gateway using a recording rate and channel selection module. In embodiments, the same data sequential order within a frame may be maintained and the order of recordings in the frame may follow the physical distribution of the Recording Channels on the tile matrix. In embodiments, processing circuitry, such as input/output (I/O) Control circuitry and/or software may control and configure PISO 2318 and MUX 2314 capabilities.

Neural Activity Modulation. In embodiments, neural tissue may be stimulated using one or more of several techniques, such as Optical Stimulation (Optogenetics), Electrophysiological Stimulation, and Electrochemical Stimulation.

Optical Stimulation. Optogenetics is a method for brain stimulation/modulation by inducing well-defined neuronal events at a millisecond-time resolution, enabling optical control of the neural activity. The method may utilize physiological processes such as Channelrhodopsin-2 (ChR2): a light-sensitive ion channel, Halorhodopsin (NpHR): an optically activated chloride pump, and Archaerhodopsin (Arch): a proton pump. ChR2 and NpHR may be genetically expressed in neurons using a viral approach. Conventionally these viruses are injected in the neural tissue, but in embodiments, the virus vector may be carried on the tips of the CNTs. Due to their small dimensions, these viruses do not interfere with the reading and stimulation processes.

There are several types of Channelrhodopsins, each one responding to a particular wavelength. Some Channelrhodopsins stimulate neuronal activity (ChR2), while others inhibit it (NpHR). Therefore, the optical sensitivity of these proteins enables both the increasing/activation and decreasing/silencing of the voltage inside neurons, by targeted laser beams of blue and yellow light, respectively. The technique is deemed as safe, precise, and reversible.

Optogenetics may be used as a side-effect-free method for alleviating symptoms of neurological diseases which occur through either neuronal overexcitability, such as epilepsy, or underactivity, such as schizophrenia. One practical advantage is that optogenetics may have minimal instrumental interference with simultaneous electrophysiological techniques.

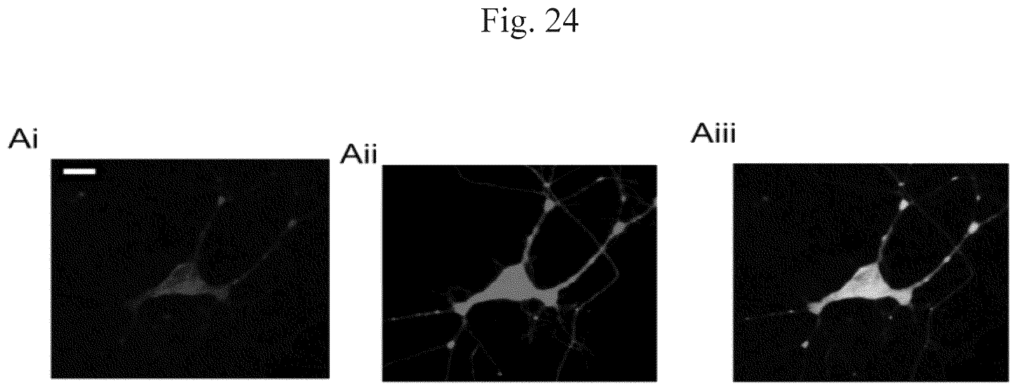

Examples of spike trains of ChR2 and NpHR expressing neurons when subjected to light beams of different wavelengths are shown in FIG. 24. FIG. 24, Ai shows an example of neuron expressing channelrhodopsin-2 fused to mCherry. FIG. 24, Aii shows an example of neuron expressing halorhodopsin fused to GFP. FIG. 24, Aiii shows an example of an overlay of Ai and Aii.

Optogenetics enable the optical control of individual neurons, but even neurons with no genetic modification have light sensitivity, such as in a circuit mediated by neuropsin (OPN5), a bistable photopigment, and driven by mitochondrial free radical production. This bistable circuit is a self-regulating cycle of photon-mediated events in the neocortex involving sequential interactions among 3 mitochondrial sources of endogenously-generated photons during periods of increased neural spiking activity: (a) near-UV photons (˜380 nm), a free radical reaction byproduct; (b) blue photons (˜470 nm) emitted by NAD(P)H upon absorption of near-UV photons; and (c) green photons (˜530 nm) generated by NAD(P)H oxidases, upon NAD(P)H-generated blue photon absorption. The bistable nature of this nanoscale quantum process provides evidence for an on/off (UNARY+/−) coding system existing at the most fundamental level of brain operation and provides a solid neurophysiological basis for the FCU. This phenomenon also provides an explanation for how the brain is able to process so much information with slower circuits and so little energy —quantum tunneling. Computers built from such material would be orders of magnitude faster than anything developed to date. The atomic scale of CNTs could potentially enable interfacing with this naturally optosensitive layer of the brain in the future, a system many orders of magnitude smaller than the neuron.

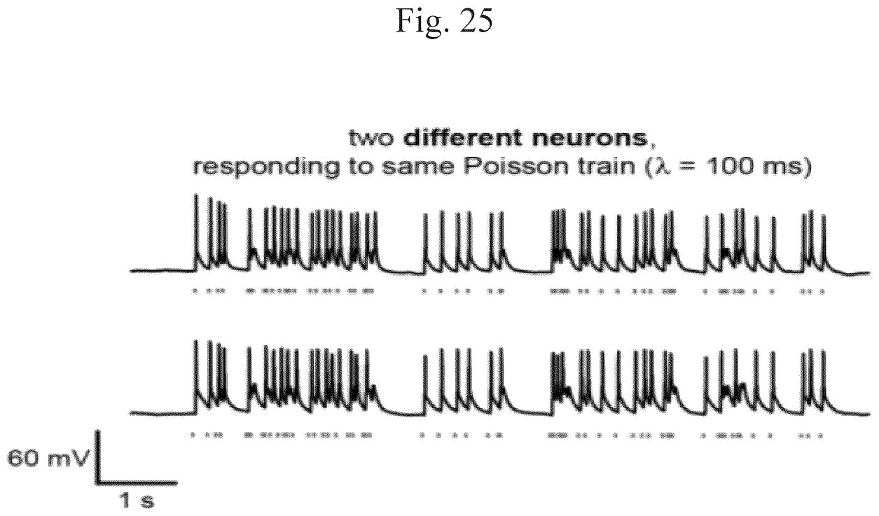

FIG. 25 illustrates an example of Poisson trains of spikes elicited by pulses of blue light (dashes), in two different neurons.

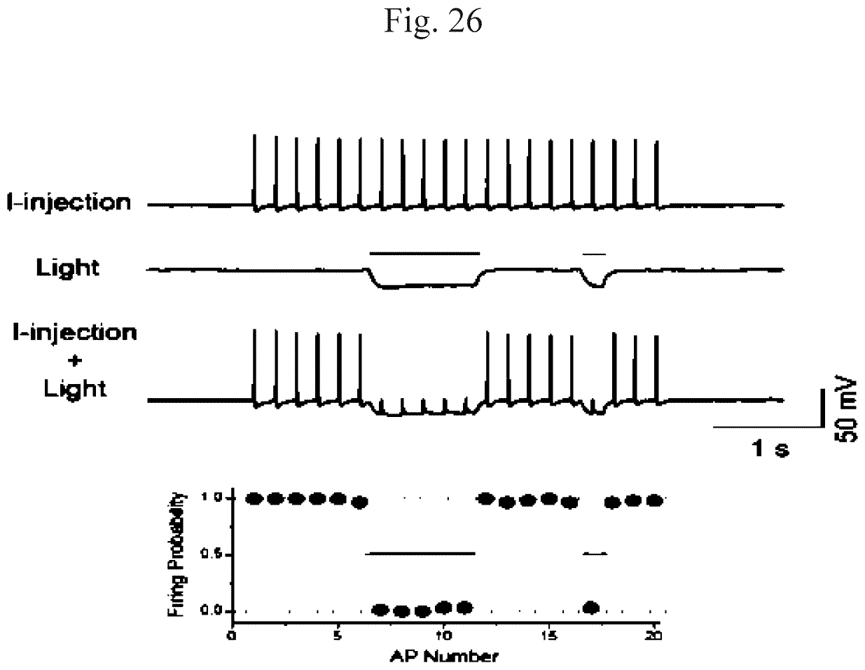

FIG. 26 illustrates an example of a light-driven spike blockade, demonstrated for (TOP) a representative hippocampal neuron, (BOTTOM) a population of 7 neurons. This example illustrates I-injection, neuronal firing induced by pulsed somatic current injection(300 pA, 4 ms). This example illustrates light, hyperpolarization induced by periods of yellow light (bars). This example illustrates I-injection+Light, yellow light drives Halo to block neuron spiking, leaving spikes elicited during periods of darkness intact.

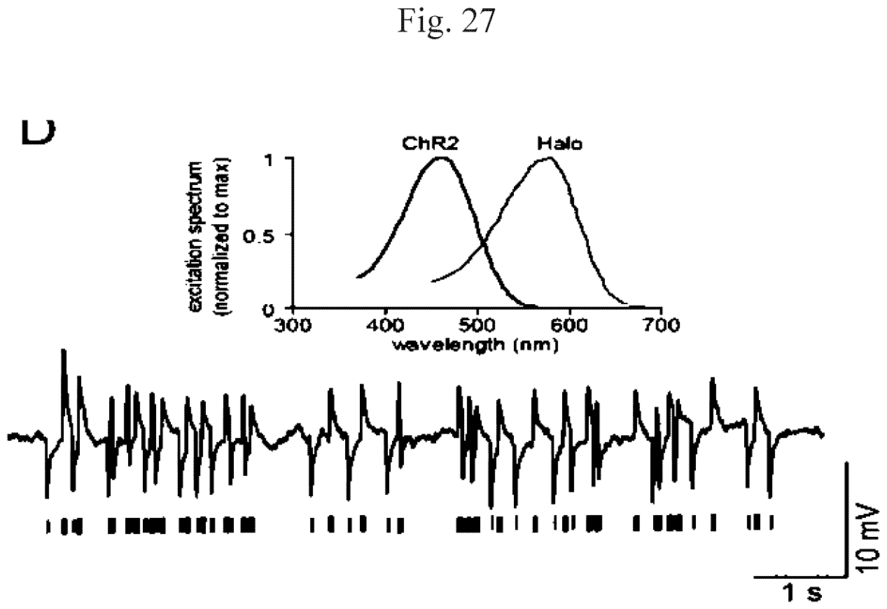

FIG. 27 illustrates an example of (TOP) an action spectrum for ChR2 overlaid with absorption spectrum for N. pharaonis halorhodopsin and (BOTTOM) Hyperpolarization and depolarization events induced in a representative neuron by a Poisson train of alternating pulses (10 ms) of yellow and blue light.

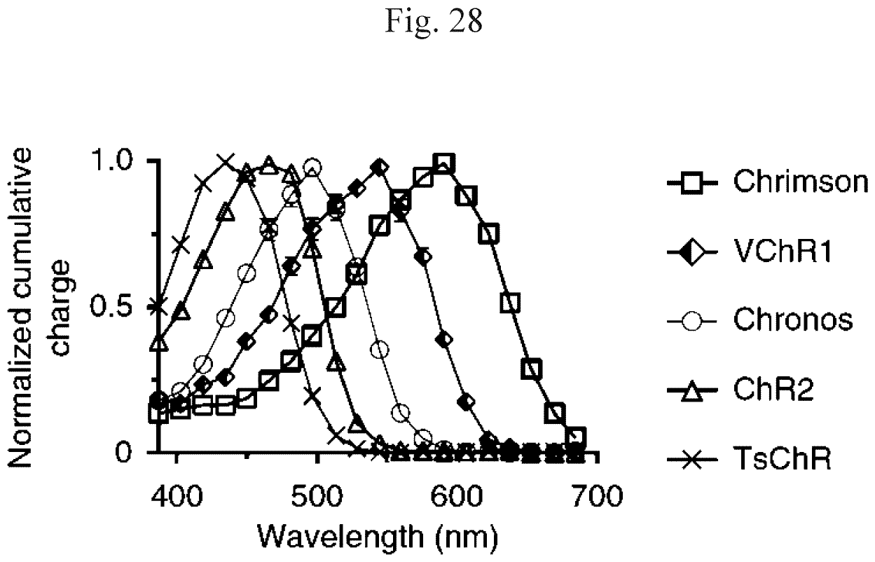

FIG. 28 illustrates examples of the correlation between wavelengths (nm) and normalized cumulative charge for a number of different Channelrhodopsins expressing neurons. From all the Channelrhodopsins discovered types, Chrimson red light stimulation is the most suited because in its case, the light intensity is proportional to how deep it travels in the brain.

In embodiments, the circuitry may be in the form of a readout-integrated circuit (ROIC), which may be similar to or a modification of, for example, a solid-state imaging array. The ROIC may include a large array of “pixels”, each consisting of a photodiode, and small signal amplifier. In embodiments, the photodiode may be processed as a light emitting diode, and the input to the amplifier may be provided by the CNT connection to the neuron. In this manner, neurons may be stimulated optically, and interrogated electrically. The ROIC may include CCD or CMOS photodiodes or other imaging cells, to receive optical signals, electrical receiving circuitry, to receive electrical signals, light outputting circuitry, such as LED or lasers, to output optical signals, and electrical transmitting circuitry, to transmit electrical signals.

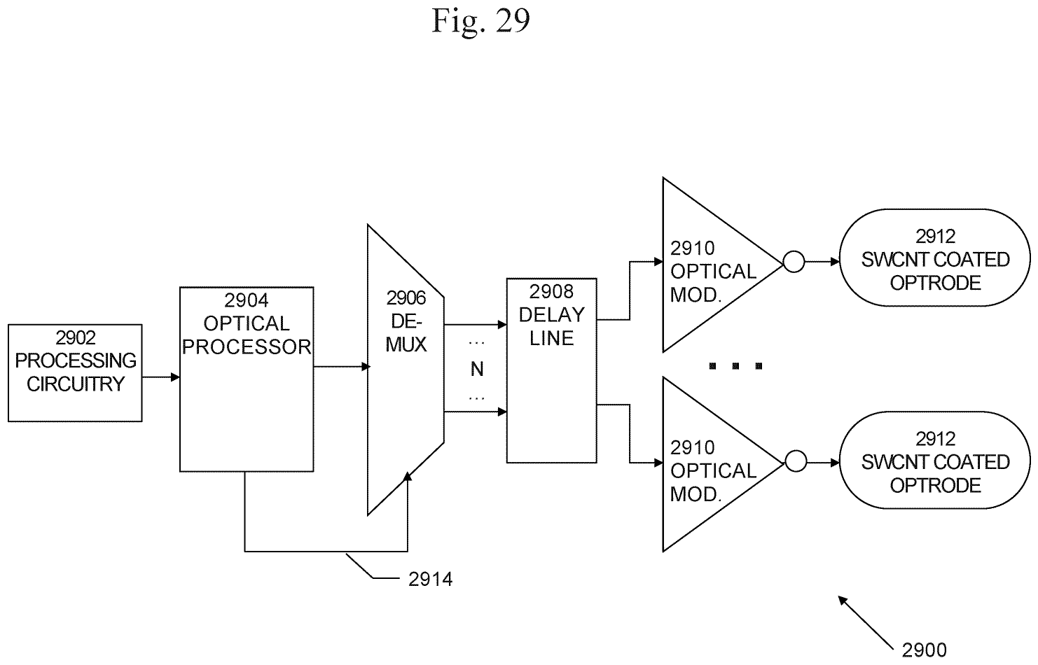

An example of an embodiment of an optical stimulation pipeline 2900 is shown in FIG. 29. In this example, pipeline 2900 may include processing circuitry 2902. Processing circuitry 2902 may include digital processing circuitry, such as one or more microprocessors, microcontrollers, digital signal processors (DSPs), custom or semi-custom circuitry, such as application specific integrated circuits (ASICs), field programmable circuitry, such as field programmable gate arrays (FPGAs), etc., or any other digital processing circuitry.

Processing circuitry 2902 may encode stimulation commands for modulation of optical signal. For example, such commands may be 5 bits, for up to 32 different modulation commands. Processing circuitry 2902 may send one of the 32 possible commands and the data identifying the channel to be stimulated. Each command may be mapped into a wavelength and a light intensity, which may be encoded digitally and sent to optical processor 2904 on its digital in/out port, together with the channel on which the light may be transmitted.

Optical processor 2904 may transform the input digital electrical signal into an optical signal of the appropriate wavelength and intensity. Optical processor 2904 may then transmit the light signal to Optical Demultiplexing Unit (DEMUX) 2906, along with the desired channel on the Select Line 2914.

Optical Demultiplexing Unit 2906 may forward the light signal on the appropriate channel. Each light signal may pass through a Delay Line 2908 and then through an Optical Modulator 2910, which may adjust and amplify the signal to its appropriate values. The light signal then be transmitted through optrodes 2912, through the fibers, to the neurons.

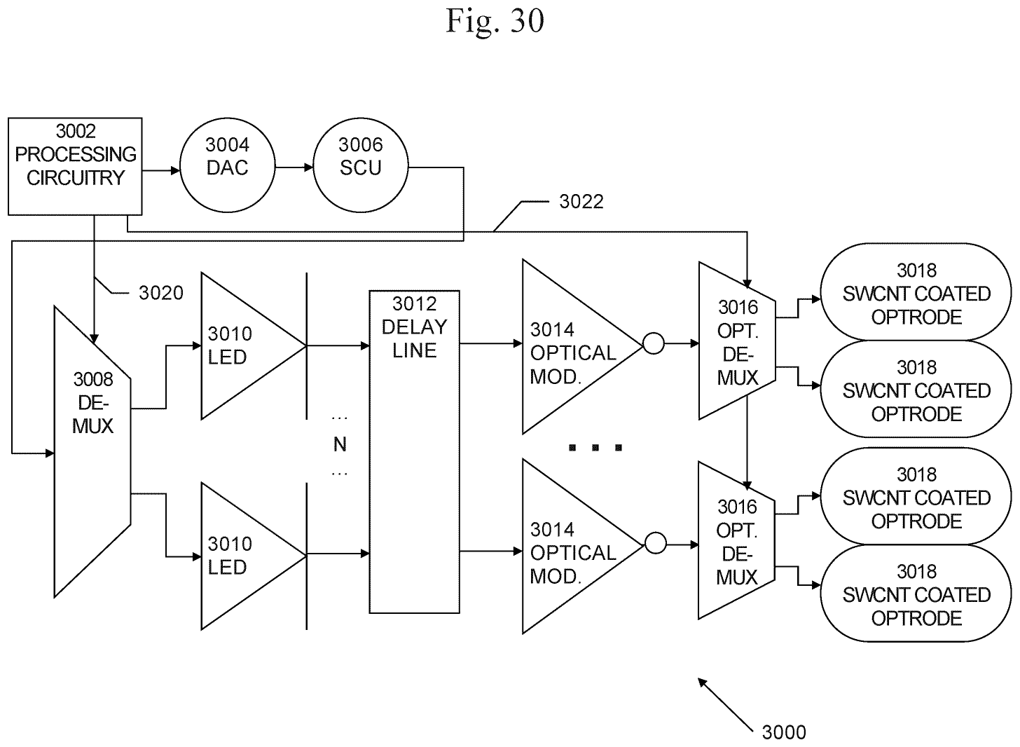

An example of an embodiment of an optical stimulation pipeline 3000 is shown in FIG. 30. In this example, pipeline 3000 may include processing circuitry 3002. Processing circuitry 3002 may include digital processing circuitry, such as one or more microprocessors, microcontrollers, digital signal processors (DSPs), custom or semi-custom circuitry, such as application specific integrated circuits (ASICs), field programmable circuitry, such as field programmable gate arrays (FPGAs), etc., or any other digital processing circuitry.

Processing circuitry 3002 may encode stimulation commands for modulation of optical signal. For example, such commands may be 5 bits, for up to 32 different modulation commands. Processing circuitry 3002 may send one of the 32 possible commands and the data identifying the channel to be stimulated. Each command may be mapped into a wavelength and a light intensity, which may be encoded digitally and sent to DAC 3004, in which the digital electrical signal may be converted to an analog electrical signal.

The analog electrical signal may be amplified by a Signal Conditioning Unit 3006, to increase its amplitude to useful levels. From Signal Conditioning Unit 3006, the analog electrical signal may be input to an electrical Demultiplexing Unit (DEMUX) 3008. Based on the signal that comes from the processing circuitry 3002 on Select Line 3020, DEMUX 3008 may transmit the analog electrical signal on an appropriate channel to the LED 3010 that generates an optical signal of the required wavelength. LED 3010 may generate an optical signal, which may be transmitted through a Delay Line 3012, to an Optical Modulator 3014. From the Optical Modulator 3014, the optical signal may travel through an Optical Demultiplexing Unit, which, based on the received signal on select line 3022 from processing circuitry 3002, may forward the light beam to the correct optrode 3018.

In this exemplary embodiment, there are two demultiplexing units: an electric one 3008, which leads to the LED of the right wavelength, and an optical one 3016 which sends the light down the correct channel. Accordingly, embodiments may have as many light sources as wavelengths to be generated.

Electrophysiological Stimulation. Alzheimer's disease produces irreversible degradation to the brain to the point where there are not many treatment options. There are only a few medications available, which unfortunately cannot stop the symptoms from getting progressively worse or even fatal.

However, one potential treatment for diseases such as Alzheimer's may be deep brain stimulation. Deep brain stimulation works by continuously tickling neurons in the frontal lobe of the brain with electrodes. Patients who have these electrodes implanted may maintain more of their mental faculties than a group of control patients, who started out at similar stages of the disease.

Electrophysiology is a tool for deep brain stimulation in which electrical current is applied via electrodes implanted on/in the brain parenchyma. While optical stimulation is able to target specific neurons very precisely, electrical stimulation implies current dissipation in the surrounding area.

Electrophysiological Stimulation may be used for neuron stimulation by applying electrical current via CNTs that are connected to nanoelectrodes and are implanted directly in the brain parenchyma.

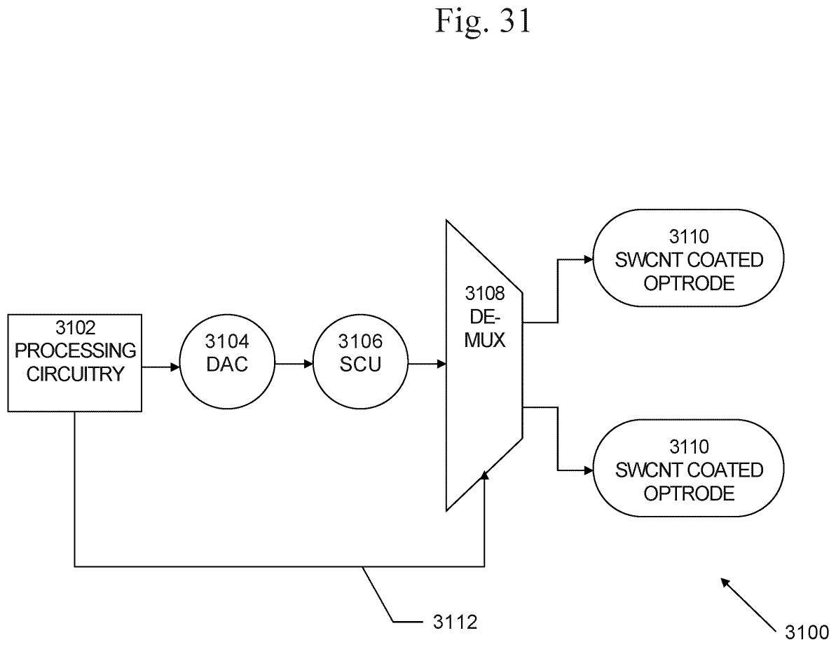

An example of an embodiment of an optical stimulation pipeline 3100 is shown in FIG. 31. In this example, pipeline 3100 may include processing circuitry 3102. Processing circuitry 3102 may include digital processing circuitry, such as one or more microprocessors, microcontrollers, digital signal processors (DSPs), custom or semi-custom circuitry, such as application specific integrated circuits (ASICs), field programmable circuitry, such as field programmable gate arrays (FPGAs), etc., or any other digital processing circuitry.

Processing circuitry 3102 may encode stimulation commands for the output signal. For example, such commands may be 5 bits, for up to 32 different modulation commands. Processing circuitry 3102 may send one of the 32 possible commands and the data identifying the channel to be stimulated. Each command may be mapped into a stimulation voltage, which may then be sent out from processing circuitry 3102 to Digital to Analog Converter (DAC) 3104, which converts the digital electrical signal to an analog electrical signal. The analog electrical signal may be amplified by Signal Conditioning Unit 3106, to provide the proper amplitude signal. From Signal Conditioning Unit 3106, the signal may be input into an electrical Demultiplexing Unit (DEMUX) 3108. Based on the signal that comes from processing circuitry 3102 on Select Line 3112, the DEMUX 3108 may transmit the stimulation signal to the corresponding CNTs 3110, which will stimulate the neurons in their vicinity.

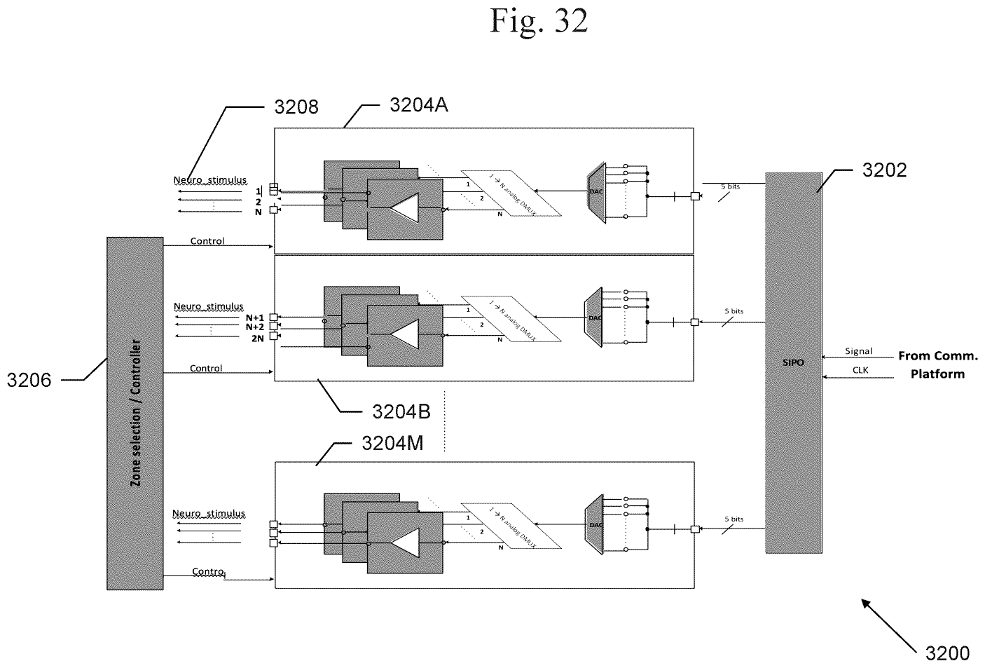

Pipeline Summary. An example of a high-level architecture 3200 of the stimulation pipelines described above is shown in FIG. 32. In embodiments, electrical stimulation CNTs may be mixed with optical stimulation and recording CNTs, as there may be little interference between them. As shown in this example, the architecture may include a Serial-In-Parallel-Out converter (SIPO) 3202, a plurality of stimulation pipelines 3204A-M, and zone selection/controller circuitry 3206.

Processing circuitry (not shown) may transmit a serial stream of digital electrical stimulation signals to SIPO 3202. The processing circuitry may translate stimulation commands into a stimulation operation having a particular stimulation signal. SIPO 3202 converts the serial stream to a plurality of parallel digital electrical signals, which may be transmitted to one or more stimulation pipelines 2306A-M. Each stimulation pipeline 2306A-M may convert its input digital electrical signals to electrical or optical neuro stimulation signals 3208, as described above. Neuro stimulation signals 3208 may then be transmitted to zone selection/controller circuitry 3206, which may route each neuro stimulation signal 3208 to an appropriate electrical stimulation electrode or optical stimulation optrode.

Embodiments may contain two units with 100 tiles each. Each tile may contain four selectable stimulation channels which may be controlled independently. In embodiments, up to 400 channels may be used for stimulation at any time. In embodiments, command values may be arranged in a matrix format that corresponds to the physical representation of the stimulation channels. In embodiments, each stimulation command may include the channel reference which represents the address of the optrode that will be used for stimulation. In embodiments, each stimulation command may include the commands array which represents the stimulation values. In embodiments, the commands array may contain the type of stimulation and the stimulation pattern (potential/intensity, timing). In embodiments, the intensity of the light beam may depend upon how far the neuron is in the tissue (and therefore how strong the light source should be in order to reach it). In embodiments, each stimulation command may depend on its specific goal, which will dictate whether the task is to increase or decrease voltage inside the targeted neuron(s). In embodiments, the optical stimulation commands shall specify the features of the stimulation pattern (light wavelength, light intensity, frequency, and duration). In embodiments, the electrical stimulation commands may specify the discrete voltage values to be applied through the stimulation channels at each time step. In embodiments, the command values may be arranged in a matrix format (10×10 commands for tile) that corresponds to the physical representation of the stimulation channels. In embodiments, a DAC may convert the digital signal into an analog signal. In embodiments, a stimulation light may have wavelengths between 400-650 nm. In embodiments, each stimulation command may be encoded as 5 bits, resulting in a total of 32 different possible stimulation commands.

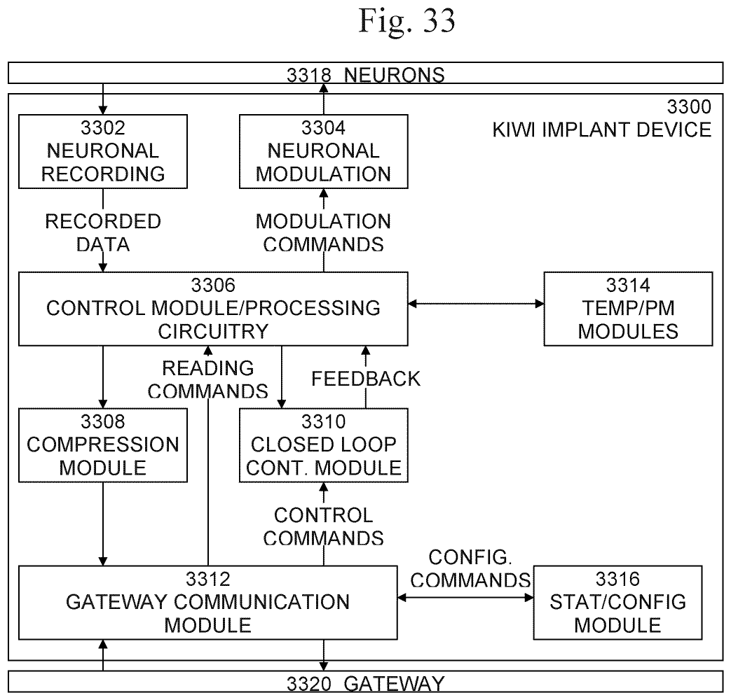

Architecture Overview. An exemplary block diagram of an embodiment of an implant device 3300 is shown in FIG. 33. In this example, implant device 3300 may include neuronal recording circuitry 3302, neuronal modulation or stimulation circuitry 3304, control module/processing circuitry 3306, compression module 3308, closed loop control module 3310, gateway communication module 3312, temperature and power management module 3314, and status and configuration module 3316. In this example, implant device 3300 may further be electrically, optically, and/or communicatively connected to neural tissue neurons 3318 and gateway 3320. It is to be noted that the circuitry shown in FIG. 33 may also include, or be associated with, software to cause the circuitry to perform the desired functions.

Neuronal recording circuitry 3302 may include circuitry, such as that described above, for recording electrical and/or optical signals from neurons 3318. Neuronal modulation or stimulation circuitry 3304 may include circuitry, such as that described above, for generating and transmitting electrical and/or optical stimulation signals to neurons 3318. Control module/processing circuitry 3306 may include circuitry, such as that described above, for receiving data from neuronal recording circuitry 3302 representing recorded electrical and/or optical signals from neurons 3318 and for generating and transmitting command data neuronal modulation or stimulation circuitry 3304 to generate and transmit electrical and/or optical stimulation signals to neurons 3318. Compression module 3308 may include circuitry for receiving recorded data from control module/processing circuitry 3306 and compressing the recorded data. Closed loop control module 3310 may include circuitry for receiving neural recording data and updating stimulation command data based on the received neural recording data to achieve closed-loop control of the stimulation process. Gateway communication module 3312 may include circuitry for communicating data to and from gateway 3320. Temperature and power management module 3314 may include circuitry for monitoring and controlling implant device temperature, power consumption, battery charging and discharging, etc. Status and configuration module 3316 may include circuitry for monitoring implant device status and for managing the configuration of the implant device.

Software Architecture.

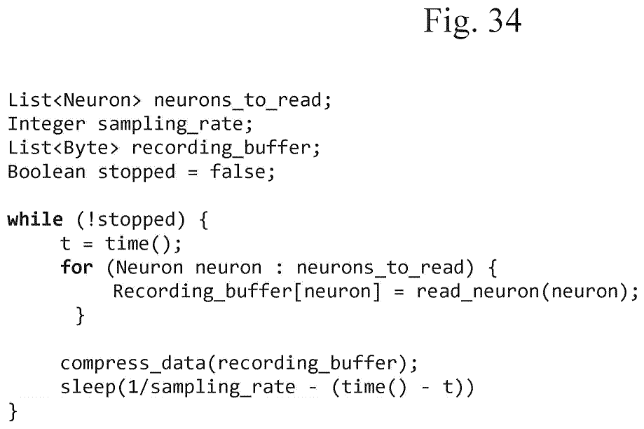

Neuronal Recording Interface. Control module/processing circuitry 3306 may make reading requests to the neuronal recording circuitry 3302 specifying the desired sampling rate and the target CNTs. An example of pseudocode for data recording is shown in FIG. 34.

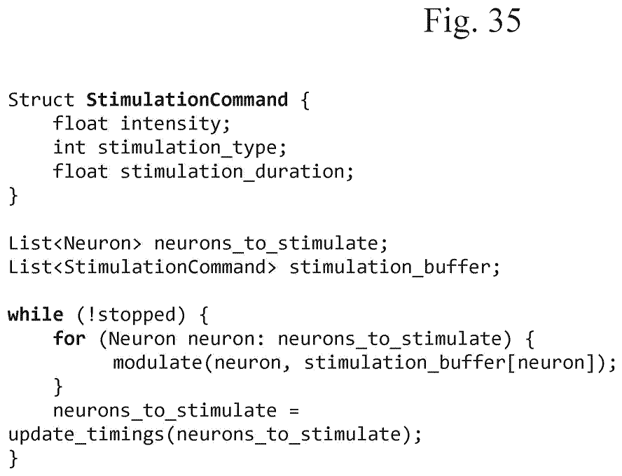

Neuronal Modulation Interface. Control module/processing circuitry 3306 may make neuron modulation requests to the Neuronal modulation or stimulation circuitry 3304. An example of pseudocode for stimulation requests is shown in FIG. 35.

Control module/processing circuitry Input/Output (I/O) Interactions.

Stimulation Scheduler. In embodiments, there are options regarding what circuitry will be responsible for keeping track of the stimulation command duration. In an embodiment, closed loop control module 3310 may be responsible for keeping track of time. In this case, closed loop control module 3310 may send a stimulation command to control module/processing circuitry 3306, which may apply that stimulation recipe until otherwise instructed. An advantage of this approach is that control module/processing circuitry 3306 does not have to feature a function for stimulation time management. However, control module/processing circuitry 3306 still may have to deal with timing issues for recording (the sampling rate).

In an embodiment, the time management function may be implemented in control module/processing circuitry 3306. In this case, closed loop control module 3310 may send a stimulation command to control module/processing circuitry 3306, along with a time period value. Control module/processing circuitry 3306 may apply that stimulation recipe for the specified duration. When the specified stimulation time ends, the stimulation on that channel may stop and the control module/processing circuitry 3306 may waits for further instructions. If a new command is received while the previous one is active, the previous one may be overwritten. The advantage of this approach is that closed loop control module 3310 is entirely free from managing time and can focus on I/O management.

In embodiments, modules may modify the list of active channels for recording, such as closed loop control module 3310 and gateway communication module 3312. Gateway communication module 3312 may modify the list of active channels for recording in order to read a different set of channels than the ones that are in use by closed loop control module 3310.

Throttling Side-channel. Control module/processing circuitry 3306 may also communicate with temperature and power management module (TPMM) 3314. In embodiments, when TPMM 3314 detects that the temperature of the implant device is rising, approaching the thermal safety limits, it may send a SLOW signal to control module/processing circuitry 3306 to start throttling the I/O activity. When receiving the SLOW signal, control module/processing circuitry 3306 may decrease the recording sampling rate and communicate to closed loop control module 3310 to reduce the rate of stimulation commands. If the temperature exceeds the thermal safety threshold, TPMM 3314 may send a STOP signal (by flipping another bit) to control module/processing circuitry 3306, which may then cease all recording and stimulation activities.

TPMM 3314 may also monitor the battery level of the implant device. If the battery level falls below a threshold B1, TPMM 3314 may send a SLOW signal to control module/processing circuitry 3306 to start throttling the I/O activity. If the battery level falls below a lower threshold B2, TPMM 3314 may send a STOP signal to control module/processing circuitry 3306 in order to preserve battery life.

In embodiments, this side channel may be focused only on activity and process control, therefore no neural data may be sent or received on it.

Data Flow. In embodiments, an efficient data flow between the modules may be implemented, which will take into account the constraints in terms of memory and processing resources.

For example, in embodiments, control module/processing circuitry 3306 may place the recorded data in a memory buffer (an array) from which data will be shared with the other modules, according to the protocol described above. Closed loop control module 3310 may store the stimulation commands in a memory buffer (an array) from which the commands may be used by the control module/processing circuitry 3306 for stimulation.

TPMM 3314 may send signals to control module/processing circuitry 3306 by flipping a corresponding bit in memory. This bit may also be shared with closed loop control module 3310 and may trigger the slowing down of the stimulation activities.

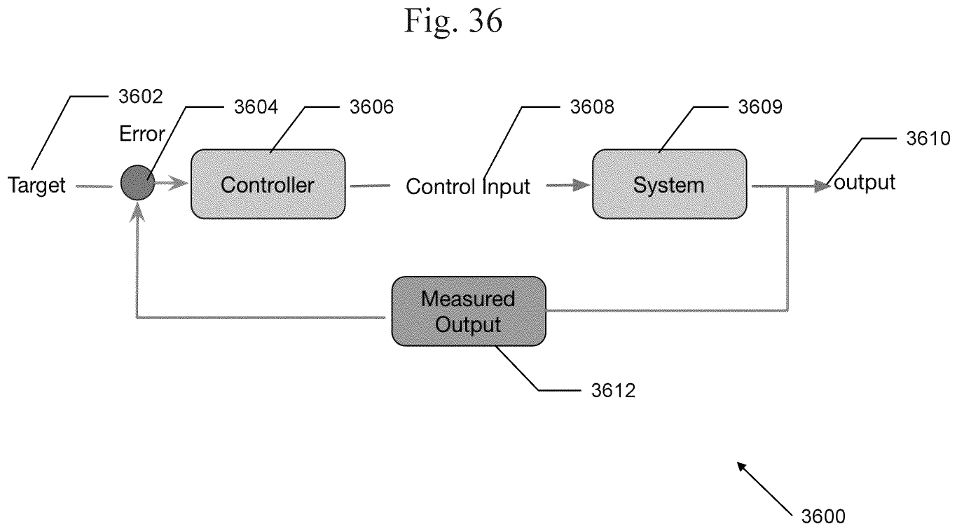

Closed-Loop Control (Command & Recording). In embodiments, brain stimulation may be more effective when it is applied in response to specific brain states, via Closed Loop Monitoring, as opposed to continuous, open loop stimulation. An example of a conceptual sketch of a closed loop control system 3600 is shown in FIG. 36. In this example, a target signal 3602, which may indicate a desired output 3610 from system 3600, may be input to system 3600. An error circuit 3604 may determine a difference (error signal) between target signal 3602 and a measurement 3612 of output 3610. The error signal may be input to a controller 3606, which may generate a control input signal 3608 to control system 3609 to generated the desired output 3610 indicated by target signal 3602. Output 3610 may be measured 3612 and feedback to error circuit 3604. In overall operation, closed loop control system 3600 may continuously adjust its operation so that the actual desired output 3610 corresponds to the desired output indicated by target signal 3602.

Closed-loop, activity-guided control of neural circuit dynamics using optical and electrical stimulation, while simultaneously factoring in observed dynamics in a principled way may be a powerful strategy for causal investigation of neural circuitry. In particular, observing and feeding back the effects of circuit interventions on physiologically relevant timescales may be valuable for directly testing whether inferred models of dynamics, connectivity, or causation are as accurate in vivo testing.

In embodiments, Neuronal Response Latency (NRL) may measure a time-lag between the extracellular stimulation and the intracellularly recorded evoked spike. The NRL of the same neuron may vary among extracellular stimulating electrodes depending on their position; however, for a given stimulating electrode it may be reproducible qualitatively (for low stimulation frequencies). For example, the NRL may range between about 1-15 ms.

In embodiments, spike-detecting, closed-loop Single Input Multiple Output (SIMO) control may use template matching to do online spike detection on 32-channel tetrode recordings (system outputs) and may use detected spikes to control optogenetic stimulation through a single fiber optic (system input) at ˜8 ms closed-loop latency in awake rats. Further, simulated closed-loop control in an all-electrical Multiple Input Multiple Output (MIMO) systems for Electrical Deep Brain Stimulation (EDBS) may raise key points directly relevant to closed-loop optogenetics for MIMO systems, showing that a properly designed MIMO feedback controller may control a subset of simulated neurons to follow a prescribed spatiotemporal firing pattern despite the presence of unobserved disturbances. Such disturbances may be typical in neural systems of interest, as most of the brain will remain unobserved. Further, a simplified linear-nonlinear model may be quite effective in controlling firing rates, despite strong simplifying assumptions (this is important for systems where speed dictates hard computational constraints). In addition to the practical goal of safer, more effective deep-brain stimulation, the resulting spatiotemporal patterns identified may themselves be of intrinsic value in providing new insights into how neural circuits process information.

Additional theoretical work may involve optimal control theory to design control inputs that evoke desired spike patterns with minimum-power stimuli in single neurons and ensembles of neurons using electrical current injection. Robust computational models may use similar methods for optimal control of simple models of spiking neural networks and for individually controlling coupled oscillators using multilinear feedback. Given that converging evidence suggests that abnormalities in synchronized oscillatory activity of neurons may have a role in the pathophysiology of some psychiatric disease and considering their established role in epilepsy, it may be fruitful to continue considering oscillations themselves as a direct target of closed-loop optogenetic control alongside control of spiking neurons.

As described above, in closed-loop optogenetics, the control input 3608 may be a structured, time-varying light stimulus that is automatically modulated based on the difference between desired and measured outputs. Measured outputs may include behavioral, electrophysiological, or optical readouts of activity generated by the subject.

In embodiments, optrodes-MEA are may be used as a hybrid approach for optical neuron stimulation and electrophysiological neuron recording. Embodiments may use optical fibers ‘coated’ with CNTs in order to support this hybrid approach, being able to record and stimulate both optically and electrically.

The advantage of optical over electrical interaction with the neurons is that, while electrical stimulation implies current dissipation in the surrounding area, optical stimulation is able to target specific neurons with greater precision, and it incurs minimal interference with simultaneous electrophysiological recording techniques.

Control Techniques. Depending on the specific neural modulation task associated to the disease that is being treated, embodiments may use different closed loop control packages, which may be uploaded to the implant device. These may be implemented in the control module/processing circuitry.

In embodiments, different types of control techniques may be used for closed loop control. For example, such techniques may include simple on/off control, Proportional Integral Derivative (PID) control, Model Predictive Control (MPC), robust control, adaptive control, and optimal control. Each of these techniques may have different tradeoffs, for example, between obtaining more accurate results and being more computationally costly. The control technique may be chosen based on both the available hardware resources and on the task at hand. In embodiments, the closed loop controller module may use a simple on/off technique, or any other closed-loop control technique.

The control technique may rely on machine learning models trained both offline and online. For example, offline, gathered data may be processed in the Cloud with the purpose of deriving new insights for treatment and encapsulated in new models. This task may be advantageously performed remotely from the implant device due to the greater processing power and memory resources that may be available remotely, such as in the Cloud.

Online, the models obtained in the Cloud may be used on the implant for neuron modulation. In this way, computationally costly but necessary processing may be run offline, yielding new models appropriate for fast online conditional stimulation of the neural activity. In addition to the implant device applying the models computed in the Cloud, it may also be able to run simpler machine learning techniques on a dedicated hardware component. However, in embodiments, the models computed offline may have priority over those computed online due to the Cloud's ability to process larger amounts of data and use more advanced machine learning techniques.

In embodiments, models used by the control algorithm may be personalized for each individual user employing transfer learning. A general model may be trained on a large amount of data gathered from a large number of patients and may then be refined by training on data recorded from each individual patient. In this way, each patient may have their own personalized model, with the same generic architecture, but unique weights. Hence, transfer learning may be used to enable use of large amounts of general collected data for the benefit of individual patients and model personalization may be an appropriate approach due to the fact that neural activity has features that are specific to each patient depending on several factors (e.g. age, health condition, etc.)

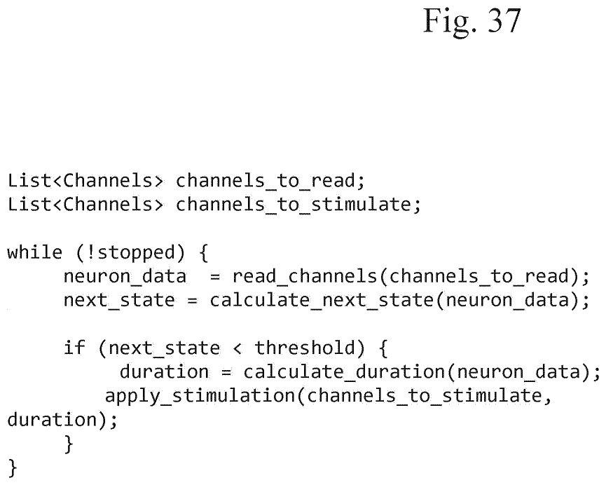

Closed Loop Module. In embodiments, the closed-loop controller module may have a well-defined interface, common to all the controller modules, which may be used to read data and to send commands. In embodiments, the closed-loop controller module may have a simple on/off algorithm, for example, sketched in pseudocode shown in FIG. 37. For example, in the memory improvement task, the calculate_next_state function may run a logistic regression model to predict whether the currently heard word will be remembered, while the calculate_duration function would return a constant duration of X ms.

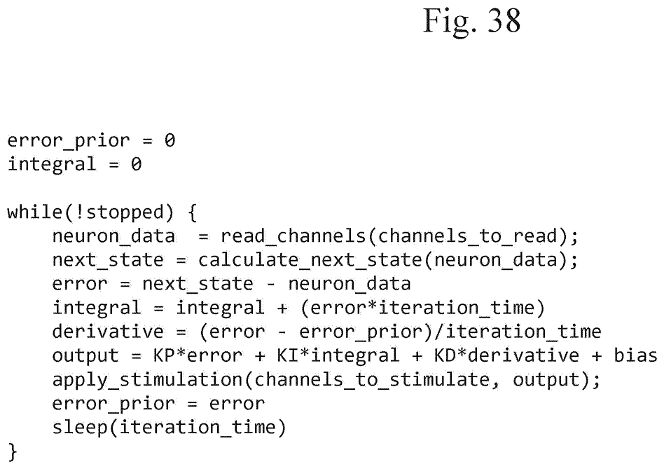

An example of a PID algorithm is shown in pseudocode FIG. 38. In this example, The KP, KI, KD and bias are constants that may be tuned for every implant.

Closed Loop Control Conditions. In embodiments, decisions to stimulate taken by the implant device may be sent to the Gateway/Cloud for further processing and fine-tuning of the online model. Due to time constraints (for example, <8 ms latency may be required), the decision to stimulate may be taken internally by the implant device. Using machine learning techniques, the implant device may also compute the optimal optic or electric response that minimizes the difference between current and ideal neural activity. The closed loop control module may monitor voltage levels inside neurons through electrical and optical recording.

In embodiments, the closed loop control module may output the appropriate stimulation pattern in less than 8 ms from when the neuronal measurement was taken. The implant device may allow the Gateway to replace or update the closed feedback loop technique (controller) according to what best fits the task at hand. The task-specific technique may be used to process the recorded data to determine the appropriate stimulation pattern. The closed loop control module may output (to the Stimulation Module) the appropriate stimulation pattern encoded in one of, for example, 32 control commands. All the controller modules may take into account the safety thresholds described below.

Control Module/Processing Circuitry. The raw data as it comes from the CNTs may not be interpreted directly. It may be preprocessed and filtered for noise removal. Before it can be sent to the Cloud, it also may be compressed. Also, for processing with the Closed Loop Control Module, first the state of the neurons(spiking or not) may be identified.

Data Types.

Neuronal Recording. In embodiments, the measured data may be stored in 10-bit variables for both electrical and optical reading. The electrical recording may represent a potential measurement with values between, for example, about −100 mV and 100 mV. These values may be normalized to a floating-point value between [0, 1].

In the case of optical reading, light intensity emitted by the fluorescent substance may be measured. This reading may be correlated linearly with the voltage going through the neuron's membrane and may be represented as between, for example, about −100 mV and 100 mV. These values may also be normalized to a floating-point value between [0, 1].

Neuron Stimulation. In embodiments, stimulation commands may be encoded with 5-bit data. As a result, the implant device may be able to trigger a total of 32 different stimulation patterns. For example, the first bit may specify the type of stimulation (electrical or optical), and the last 4 bits may describe the actual patterns, resulting in 16 combinations for each type of stimulation. In the case of electrical stimulation, the patterns may vary in terms of applied electrical potential and timing. In the case of optical stimulation, the patterns may vary in terms of light wavelength, intensity, and timing.

Data Buffering. The compression module may process blocks of recorded data, hence, in embodiments, the recorded values may be buffered until an entire block is filled. The required size of the input buffer may be at least 100*10=1000 bits=125 bytes.

In embodiments, for the output, a second buffer may account for any potential problems in data transfer to the Gateway, such as packet loss over the Wi-Fi signal or unexpected transfer rate changes. Using a buffer for the output channel may also make the transfer process more robust, as sending data may be more efficient if data is first gathered in a data frame before being transferred to the recipient. In embodiments, the minimum required buffer size may be determined by the size of the largest Wi-Fi frame, for example, 2304 bytes.

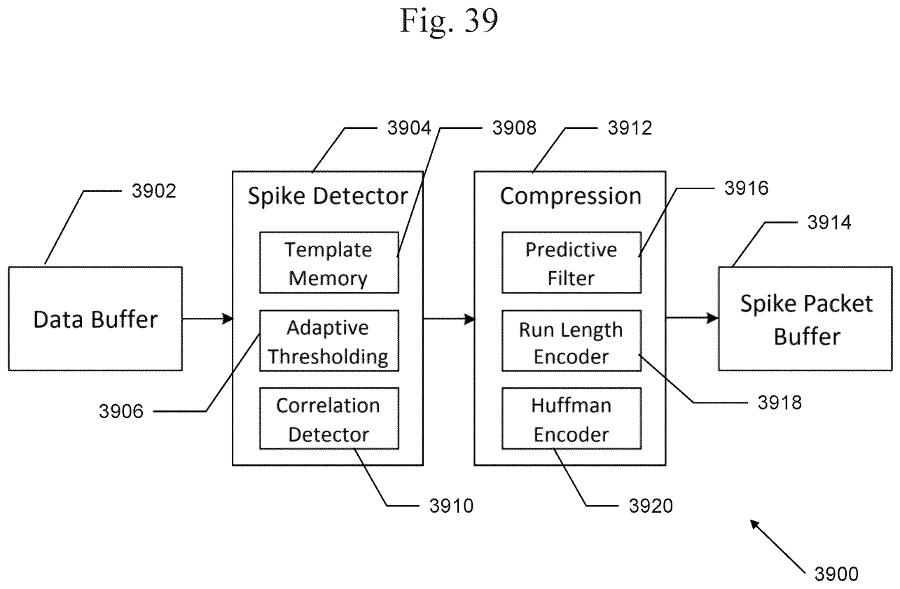

Spike Sorting. An exemplary data flow block diagram of a spike sorting technique 3900 is shown in FIG. 39. As shown in this example, when data arrives in a data buffer 3902, spike detection 3904 may be performed, using, for example, an adaptive threshold 3906 to recognize spiking events, template memory 3908 to identify neurons, and correlation detector 3910 to identify overlapping spikes.

The obtained spiking data may then be compressed 3912 so that it can be buffered 3914 and sent. In the spiking compression process predictive filters 3916 may be used to correct for potential erroneous measurements and Run Length Encoding 3918 and Huffman Coding 3920 may be used to compress the data encoded in zeroes (for when neurons are not spiking) and ones (when neurons are spiking).

In embodiments, the electrical potential data recorded from the CNTs may contain signals from multiple nearby neurons. Many neurons, however, have a distinctive spiking pattern, which enables their identification from these recordings. The neurons that are the closest (up to, for example, about 100 microns) to the CNT tip may be identified individually, while for neurons that are between, for example, about 100 and 150 microns, their spikes may be detected, but the background noise may be too strong for individual identification.

Noise Filtering. In embodiments, the first step in processing the data may be to apply a filter in order to remove noise. A band pass filter between 300 and 3000 Hz may be employed for electrical signals recorded from neurons.

Spike Detection. In embodiments, a spike may be detected when the electric field potential exceeds a given threshold. Because different neurons have different thresholds, the threshold value may be set through an adaptive method. For example,

$$T h r = 5 {σ}_{n}$$

$${σ}_{n} = median \{ \frac{\vert x \vert}{0.6745} \}$$

Where x is the bandpass filtered signal and σₙ is an estimate of the standard deviation of the background noise.

Feature Extraction. In embodiments, using wavelets to extract features from the raw waveforms may result in a better separation of the clusters for the templates. The wavelet coefficients may be selected so that they have a multimodal distribution, to be able to distinguish different spike shapes. This may be performed using, for example, a Kolmogorov-Smirnov test for Normality.

Clustering. In embodiments, in order associate the spikes to the neurons that produced them, clustering may be performed on the resulting data. For example, the Super-Paramagnetic Clustering (SPC) method may be used. SPC is a stochastic method that does not assume any particular distribution of the data and groups the spikes into clusters as a function of a single parameter, the temperature. In analogy with statistical mechanics, for low temperatures all the data may be grouped into a single cluster and for high temperatures the data may be split into many clusters with few members each. There is, however, a middle range of temperatures corresponding to the super-paramagnetic regime where the data may be split into relatively large size clusters, each one corresponding to an individual neuron that is recorded.

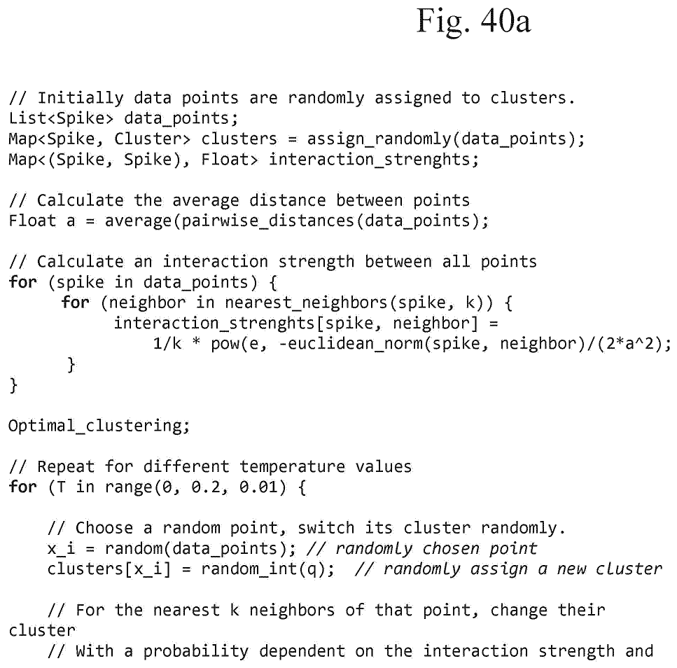

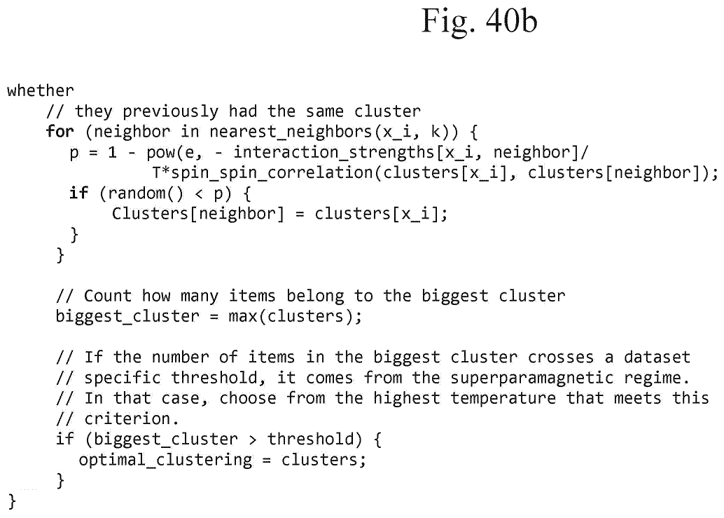

An example of pseudocode for performing an SPC method is shown in FIGS. 40a-b.

In embodiments, the clustering process describe above may be performed offline, for example, in the Cloud, and only the resulting neuron templates may be communicated to the implant, which may use them to detect new spikes in real time.

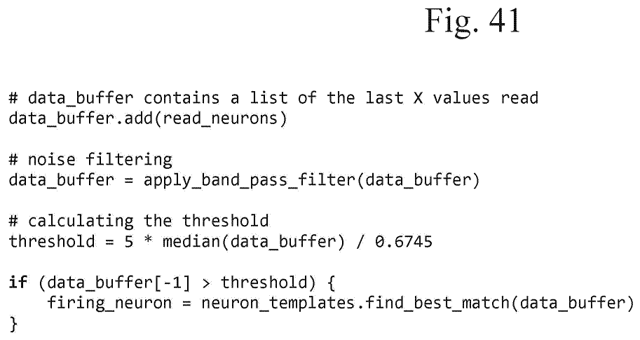

Potential challenges are represented by overlapping spikes, which happen when two close-by neurons fire at the same time. In this case, the two spikes might not be cleanly separable and a different method may be to solve this problem, such as looking for linear superpositions of other spike shapes. An example of pseudocode for such a Spike Sorting technique is shown in FIG. 41.

Data Compression. In embodiments, the implant device may generate up to 400 Mb/s of uncompressed data, which may exceed the bandwidth capabilities of low powered wireless transmission methods. Accordingly, in embodiments, the data may be compressed. Two major types of compression techniques may include lossy compression and lossless compression. The advantage of the lossless compression is that the raw data may be exactly reconstructed in the Cloud, but the compression ratio (around 2-3× at most) may not be as large as the one available with the lossy methods. With lossy compression, the original data cannot be reconstructed exactly. There is a tradeoff to be made between how much data is lost and how strong the compression is. Embodiments may use lossy compression, lossless compression, and/or a combination of the two techniques.

Optical and Electrochemical Data. In embodiments, Discrete Wavelet Transforms (DWT) with run-length encoding may be used to compress data in a lossy manner. In embodiments, using compression sensing with unsupervised dictionary learning may result in compression rates between 8× to 16×, with a signal to noise distortion ratio (SNDR) between 3.60 dB and 9.78 dB.

Electrical Recording Data. In embodiments, the methods described above may be used when the goal is to preserve the waveforms of the spikes. For a higher compression ratio, but at the cost of losing raw waveform information, embodiments may use spike detection and/or spike sorting. Examples of hardware implementations of spike detection may be as simple as a comparator with a pre-defined threshold. In this way, a compression ratio higher than 100×may be achieved with little power consumption.

In embodiments, bit encoding techniques may be applied to detected spikes. The activity wave may be segmented in X regions and then bit encoding may be used for each region. For example, if the activity range is split into 16 regions, the values may be encoded in just 4 bits instead of 10 bits. Then, the recording of each channel may be encoded in a fixed position in a block array. Each recording channel value (4 bits) may be a part of a time block container.

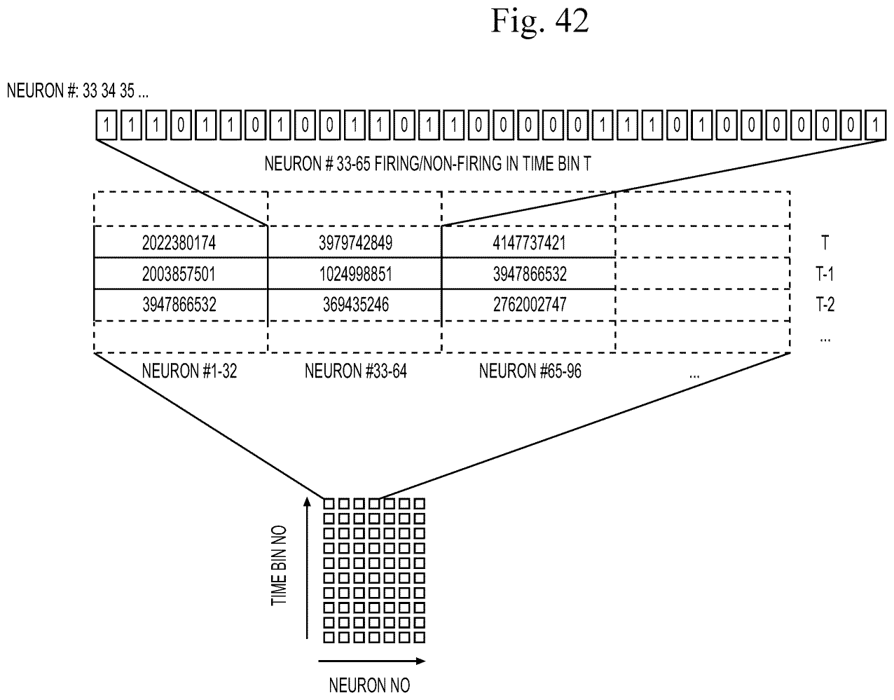

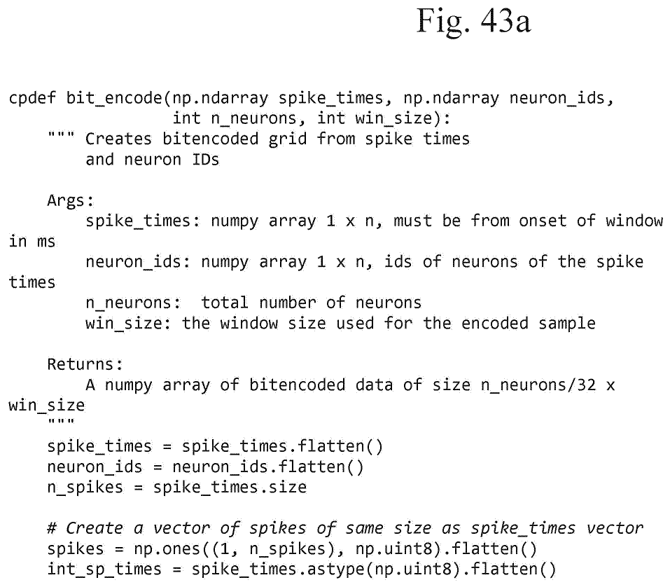

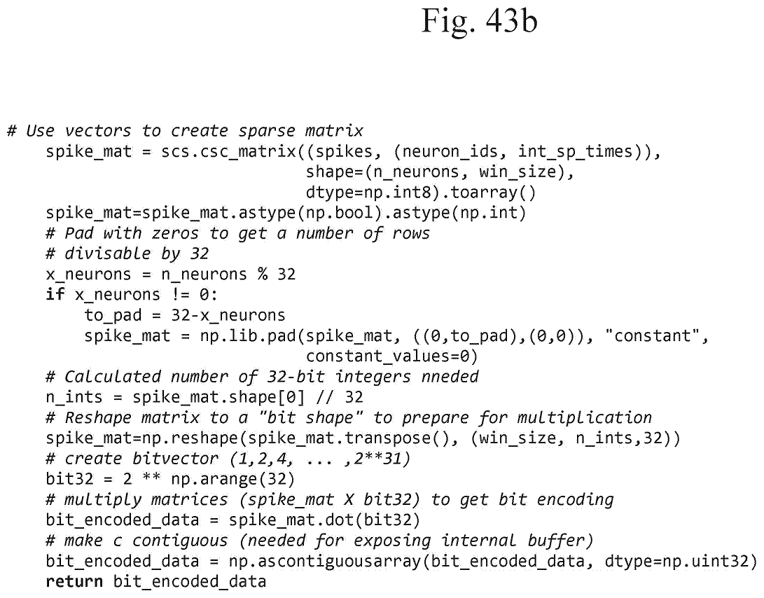

An example of bit encoding techniques for the case of 1 bit per channel is shown in FIG. 42. In embodiments, this technique may be extrapolated to, for example, 4 bits per reading channel. An example of a Python implementation of bit encoding techniques is shown in FIGS. 43a and 43b.

Example. For a better understanding, the following example is presented. In this example, there may be 1000 reading channels. In each channel, the recorded values may be encoded as 4 bits, with a sample rate of 10,000 samples per second. For sending data blocks, each taking 10 ms to transmit, a matrix may be generated wherein the bytes for each row is 1000 channels×4 bits/channel=4000 bits/8=500 bytes. The number of rows is 10 ms×20.000 sps/1000 channels =200 Rows. Thus, in total, the Header Information—containing the timestamp for time TO may include a Header Marker of 2 bytes, the Timestamp for T0 of 4 bytes, and a Data Size of 200 Rows×500 bytes=100,000 bytes.

In this specific example, 100,006 bytes, which include 1000 channels recorded at an interval of 10 ms, may be transmitted. If a compression of 100× is achieved, a data buffer of only 1 KB may be needed for each 10 ms span representing the recorded data from 1000 channels.

Running Modes. In embodiments, the Spike Sorting and Data Compression Modules may learn from recorded data in the first stage. Therefore, in embodiments, after being implanted, the implant device may run in a training mode for a period of time, at the end of which it may switch to an operating mode. If, in operation, the implant device configuration produces greater than some predefined level of errors, when evaluated by an evaluation model, than the implant device may switch back to training mode for re-configuration. The evaluation model may be configured depending on the specific task of the implant device. Such could be the case, for example when the implant device drifts, making the recorded data no longer match with the previously learned neuron spiking patterns.

For example, an evaluation metric that may be used to switch back to training mode may include determining if the number of spikes per minute, averaged over N hours or during a known supervised exercise, drops below X % of the initially recorded number of spikes per minute. If so, then the implant device may switch back to training mode to learn new dictionaries and new spike templates.

In embodiments, during the training phase, the implant device may collect the raw waveforms and send them to the Cloud for acquisition of the dictionaries for the data compression (if lossy compression is used) and for generation of the spike templates that may be used for the spike sorting process. Most of the lossy compression methods work by building a list of the most often repeated parts of the data, which may be stored in a dictionary. Then, the whole data may be scanned for parts that are very close to the entries of the dictionary and they may be replaced by a pointer to them. In this way, several bytes may be replaced with just a pointer into the dictionary. In embodiments, Cloud processing may be used create the dictionary that may be used for compression by the implant device. The lossy factor may be represented by how the similarity between the scanned data and the dictionary entries is modeled.

When enough data has been collected—meaning that the models perform to a specified task dependent accuracy threshold—the implant device may switch to the operating mode. In this mode, the implant device may perform the following actions: identifying active neurons and recording spiking activity, running the closed loop feedback controller module trained and computed on the Cloud, and compressing the recorded data and sending it to the Cloud.

In embodiments, besides the training and operating modes, the implant device may also be configured in terms of the data transfer ratio and compression method. In embodiments, examples of configurations that may be used include:

All the channels recording electrical signals. Data compression may be based on spike detection and bit encoding, transmitting only neural spike timings to the Cloud without any raw waveforms.

Only a number of N channels may be used in total for recording, in any of the three recording modes. Lossy compression may be performed on the waveforms and the resulting data may be sent to the Cloud. In this case, the maximum number of channels depends on the quantity of data that must be preserved during compression.

Less than 5% of the channels may be used in total for recording. The compression may be lossless and the full waveform may be reconstructable in the Cloud.

In embodiments, when the transfer rate is lower than the recording rate, the implant device may use appropriate techniques to filter the data to obtain a manageable data volume. The implant device may perform real time Nx compression of the recorded data. The N value may be defined depending on the hardware limitations and task goals. In embodiments, the implant device may have an input buffer for the neuron recordings of at least 125 bytes. In embodiments, the implant device may have an output buffer of at least 2304 bytes. In embodiments, the implant device may have two running modes: training mode and operating mode. In embodiments, the implant device may be able to classify spikes to identify which neuron they belong to. In embodiments, the implant device may test different lossy and lossless compression algorithms, with the goal of choosing the optimal method. In embodiments, the implant device may initially start in training mode. In embodiments, the implant device may be able to switch between running modes upon receiving a command from the Gateway.

Gateway Interface. In embodiments, the implant device may be wirelessly connected to a Gateway component by exposing an interface for the following processes: Transmitting neural recording streams, receiving control commands, and Receiving configuration commands. Embodiments may use wireless communications such as Wireless Data Communication Type—802.11ac, Wireless Frequency—5 GHz, and Radio Channel Size—80 MHz

In embodiments, Wi-Fi communications may be used, due to its high rate data transmission. However, embodiments may use alternatives to the 802.11ac Wi-Fi standard. For example, the Full-Duplex Wireless Integrated Transceiver for Implant-to-Air technology may be used. This technology includes a transmitter designed to support uplink neural recording applications with a data rate of up to 500 Mb/s and power consumption of 5.4 mW and 10.8 mW, respectively (10.8 pJ/b). This high-speed data transfer rate removes the need for compression in the implant device, which may reduce the overall power consumption and generated heat. Also, another advantage of this chipset is its size of just 0.8 mm×0.8 mm. Another example is the Thread Protocol, an IEEE 802.15.4 standard, which provides a data transfer rate of 250 Kbps. This technology may have advantages including great community support, low power consumption, supported by a large number of chipsets manufacturers, secured, and stable implementation.

In embodiments, the communication channel between the implant device and Gateway may include Bluetooth. This may be the case, for example, when the Gateway device is a smartphone. In order to accommodate this requirement, the implant device may be able to buffer the data transmitted to the Gateway in cases when the transfer speed is lower than the recording speed.

In embodiments, the recorded data may be encoded as a 10-bit floating point value. Given that most of the AI and Processing Tools on the Cloud Component are processing float data in 16 bits or 32 bits encoding, the input data may be converted in the Cloud to the corresponding data type.

In embodiments, a default version of software may be installed at the factory. In embodiments, the implant device may start with a provisioning procedure if the provisioning was not already done. In embodiments, the implant device may support over-the-air (OTA) updates. In embodiments, the software may be constantly updated with novel processing models to ensure its integrity and proper functionality for the specific performed task. These updates may be performed after implantation in the brain. In embodiments, the OTA update interface may be dependent on the hardware specifications. Embodiments may allow updates to be pushed over the wireless communication channel only from specific IP address. In embodiments, stimulation and recording operations may be paused during the OTA updates. In embodiments, the implant device may restart the recording and stimulation operations, after the OTA update is finished. In embodiments, the updates may preserve the integrity of implant device. In embodiments, if an OTA update fails for any technical reasons, the implant device may restart and continue to use its previous Software Version. In embodiments, OTA updates may be processed only when the battery level is higher than a threshold that guarantees a safe update and restart of the implant device. In embodiments, OTA updates may be accepted only from one or more specific configured Gateways. In embodiments, automatic OTA updates may be enabled/disabled through configuration parameters.

In embodiments, when the implant device is initially powered on, it may start a private WAN by initiating an AP (Access Point). The Gateway may connect to this AP, using a password that is specific to the target implant device, such as a serial number or other unique identifier. In embodiments, after a successful connection, the Gateway may initiate the Provisioning Phase. In embodiments, the Provisioning Phase may provide the default parameters for all the initial configurations of the target implant device. In embodiments, the initial configuration may include parameters such as predetermined MAC addresses of the accepted Gateways, power system configuration parameters, local WAN credentials, recording parameters, wireless charging parameters, configured blocks for reading, etc.

In embodiments, after the provisioning phase is finished, the implant device may execute a reset command. After the implant device has restarted, it may connect to the local WAN, being ready to receive new commands from the Gateway. In embodiments, if Wi-Fi/AP Provisioning is not supported, for example, with mobile devices, the implant device may use the Bluetooth channel for provisioning. In embodiments, when the implant device is initially powered on, it may start the Bluetooth Discovery process, to perform Bluetooth Low Energy (BLE) pairing with the Gateway. In embodiments, the implant device may use a fixed value pin for pairing that shall be linked to the target implant device. In embodiments, after the pairing operation is executed successfully, the same Wi-Fi provisioning steps mentioned above may be performed.

In embodiments, the Gateway may connect to the implant device using a secured configuration interface. In embodiments, the Gateway may have the rights to modify configuration parameters such as power system configuration parameters, wireless charging parameters, recording parameters, activated recording blocks, activated recording channels per block, etc. In embodiments, for security reasons, the MAC addresses of the valid gateways may not be changed via the Configuration Interface. Rather, they may be changed only via the Provisioning Configuration Process.

In embodiments, a Gateway may connect to one or multiple implant devices. In embodiments, a Gateway may save the data stream from all connected implant devices. In embodiments, the implant device may only accept as a Subscriber to its Published data the Gateway which has the MAC address that was configured during the Provisioning Phase. In embodiments, the communication channel between the implant device and Gateway may support continuous data streaming of, for example, up to 4 Mb/s.

In embodiments, the implant device may publish its recorded data when it is requested by the Gateway. In embodiments, the Gateway may receive the real time data from the implant device through a secured data streaming protocol. In embodiments, in the data streaming process, the Gateway may act as the receiver, while the implant device may be the publisher. In embodiments, the implant device may switch off the data transmission as long as there is no Gateway connected, for battery conservation. In embodiments, every sample time the implant device may apply a framing mechanism to create a data frame consisting of Header Marker and a Payload. In embodiments, the Header Marker may be used to mark the boundaries of the current frame. In embodiments, the Payload may be calculated as follows: A x N x CBS, where A is the number of activated reading blocks (up to, for example, 100), N is the number of activated recording channels per block (up to, for example, 10), and CBS is the size of the compressed reading per channel.

In embodiments, the implant device may have a Data Transfer Buffer placed between the PISO layer and the Communication Channel. In embodiments, the Data Transfer Buffer may be used in cases when the transfer rate falls below 4 Mb/second. In embodiments, the implant device may receive control commands from the Gateway. In embodiments, each individual stimulation command may be encoded in 2 bytes, containing, for example, a reference to the blocks that are closest to the targeted neurons (for example, 8 bits), the referenced channel inside the block (for example, 2 bits), and the desired encoded stimulation command from the, for example, 32 different stimulation patterns (for example, 5 bits).

In embodiments, individual stimulation commands may be grouped together to be executed simultaneously. In embodiments, for each tile block, for example, 1 up to 4 channels may be stimulated simultaneously. In embodiments, a stimulation command may have a size up to for example, 200×2 bytes.

In embodiments, the communication between the implant device and the Gateway may be secured. The implant device and Gateway Wi-Fi chipset may provide a hardware secure channel between these two devices. In embodiments, in order to react promptly to the recorded data, the implant device may use machine learning models for data processing. The models may be trained in the Cloud and then pushed to the implant device via the Gateway. In embodiments, the implant device Gateway Communication Module may request the machine learning models from the Gateway through a dedicated application programming interface (API). In embodiments, the control module/processing circuitry and the closed-loop control module may load the models and may use them for data processing. In embodiments, the machine learning models may be updated using the OTA updates. In embodiments, the implant device may receive activation and inactivation commands over the stimulation API. In embodiments, the implant device may receive status request commands, and may respond with information such as battery level, temperature level, software version, enabled reading/stimulation tiles, device state, etc.

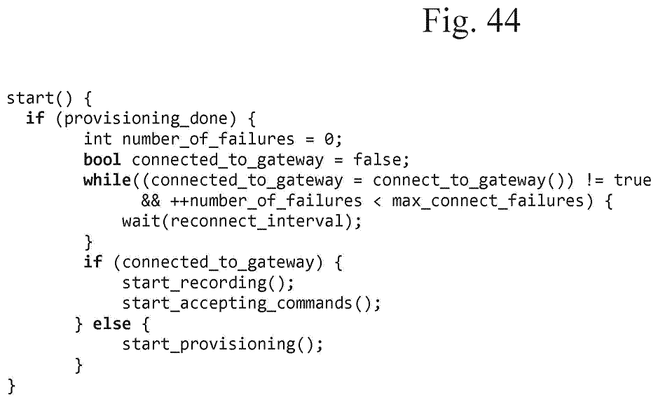

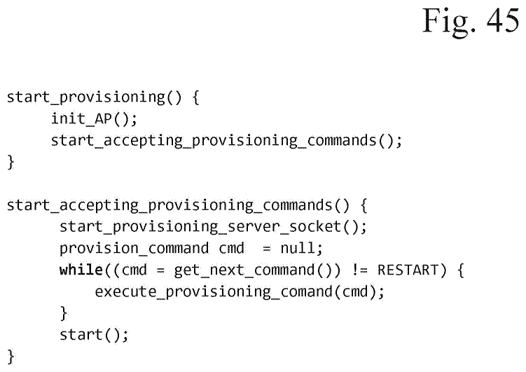

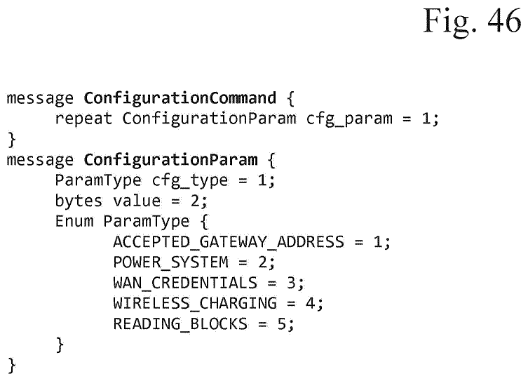

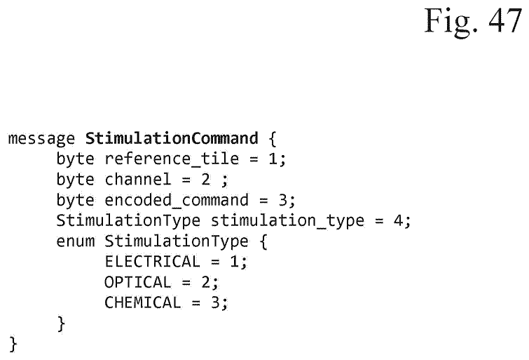

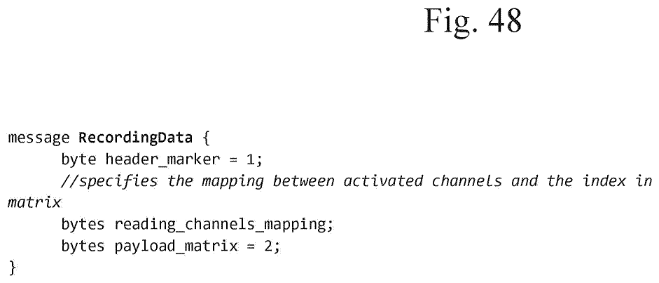

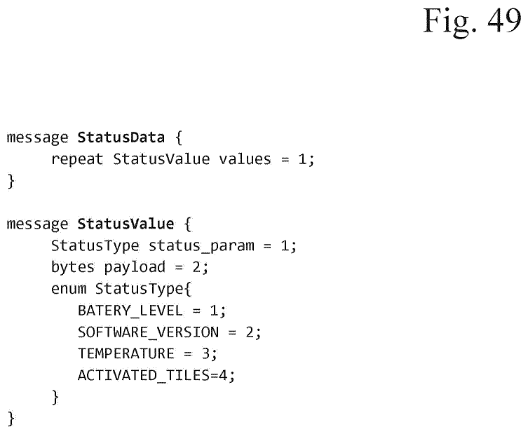

A pseudocode example of a Startup Procedure is shown in FIG. 44. A pseudocode example of a Provisioning Procedure is shown in FIG. 45. A pseudocode example of a Configuration Interface is shown in FIG. 46. A pseudocode example of a Stimulation Interface is shown in FIG. 47. A pseudocode example of a Recording Interface is shown in FIG. 48. A pseudocode example of a Status Interface is shown in FIG. 49.

In embodiments, circuitry of the implant device may meet certain specifications, for example, in terms of CPU, RAM and I/O characteristics.

CPU Speed. In embodiments, CPU speed may meet certain specifications. For example, the implant device may be able to record up to 20,000 samples per second. Each sample may be encoded in 10 bits. There may be 1000 channels per integrated circuit in the implant device. Accordingly, the transfer size per second may be calculated as 20,000 samples/second*10 bits/sample=200 Kbits/second/channel. 200 Kbits/second*1000 channels=2*10\hat{ }8 Bits/second =200 Mbits/second. Assuming that 100 operations (machine instructions) are needed for compressing one 32-bit integer, the number of operations needed for compressing one packet of 200 Mbs may be calculated as (2*10\hat{ }8 bits to process/32 bits per int)=6,250,000 Bits/operation; 6,250,000 Bits/operation*100 operations/int=625,000,000 operations. Assuming 1 operation per cycle, a CPU clock speed of at least 625 MHz may be needed.

For example, 1000 million integers may be compressed per second with Single instruction, multiple data (SIMD) acceleration. This results in approximately a 3× compression. Assuming a duration of one cycle per instruction, on a 3 GHz processor, compressing one integer would take 3*10\hat{ }9 /10\hat{ }9 =3 instructions. Given that SIMD instructions typically work on a 128-bit (16 bytes) architecture, with an 8-bit architecture, approximately 3*16=48 instructions may be needed to compress an integer. Taking into account the adjacent processes of copying data into memory and running the Closed Loop Module simultaneously, the 100 operations per integer are justified.

RAM Memory. In embodiments, RAM requirements may be estimated. Assuming a compression ratio of 10 and an Output Buffer of 2304 bytes (a limitation of the maximum packet frame supported by Wi-Fi. Therefore, the size of the Input Buffer that will store the data that needs to be compressed to the Output Buffer size will be ten times larger: 10*2304=23040 bytes. Further, the input and output buffers may be doubled to avoid synchronization issues between the reading and writing processes. In addition, in embodiments, a third intermediary buffer may be added of the same size as the Output Buffer, which may be used for storing other relevant data needed for the computation. Accordingly, an example of a formula for minimum total RAM size requirement is 3*(23040+2304)=76032 bytes=76 kB.

In embodiments, there may be a need for other RAM uses for example, running machine learning models, commands, and status communication with the Gateway, etc.

I/O Interface. In embodiments, the implant device may support 802.11ac Wi-Fi and Bluetooth Low Energy connectivity for transmitting data to the Gateway. In embodiments, to connect to the CNT layer, the implant device may also have at least 26 general purpose I/O pins. For example, 12 pins may be used for controlling the MUX Select Lines when recording data, 12 pins may be used for controlling the DEMUX Select Lines in stimulation commands, and two more pins may be used for the actual data transfer.

Device Size. In embodiments, the chipset size used for the implant device may be, for example, about 15 mm×15 mm. A number of currently available processor chips or chipsets meet this size, and some of them also provide the necessary CPU and RAM characteristics. some also support Wi-Fi/BLE, but there are small chips that could be used for this functionality.

Temperature & Power Management. In embodiments, the implant device may constantly monitor its temperature and power levels in order to make sure it doesn't damage brain tissue. When the implant device detects that temperature levels are starting to rise, it may throttle the neural recordings and stimulations. If the temperature increases by, for example, about 1° C., the implant device may stop all recording and stimulation activities and all processing until the temperature is back to normal.

In embodiments, when the implant device detects that the battery levels are getting low, it may enter a battery saving mode, where neural recordings and stimulations may be throttled. If the battery level reaches a critical threshold, for example, under about 10%, all recordings and stimulations may be stopped, to prevent the implant device from discharging completely.

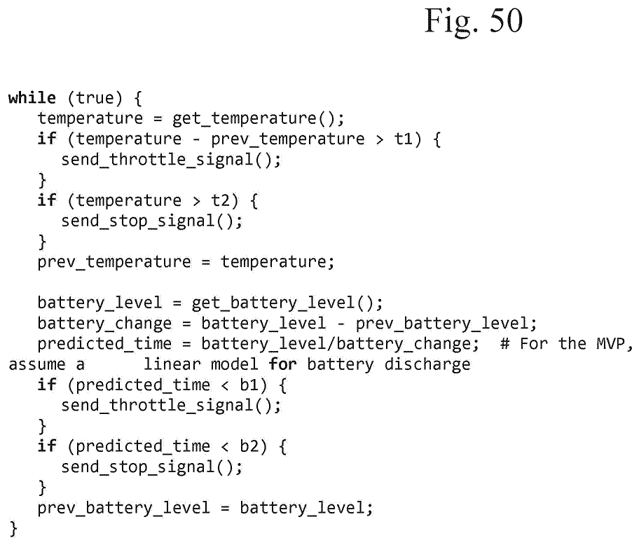

In embodiments, the implant device may also keep track of the total power output into the brain. Thermal limit requirements inside the brain may be <1 mW/mm². This limit may not be exceeded. As a safety threshold, throttling may start when power output is over 0.75 mw/mm². In embodiments, due to health and safety reasons, electrical stimulation potentials may be below the threshold of 700 mV at all times. An example of pseudocode for the temperature and power monitoring module is shown in FIG. 50.

Safety Thresholds. In embodiments, the implant device may limit its worst-case temperature rise (due to a local hot-spot) from 1° C. to 0.8° C. The typically accepted limit up to which a compact device may be allowed to heat up without damaging surrounding brain tissue is 1° C., so embodiments may provide additional safety margin.

The electrical stimulation potentials threshold for irreversible tissue damage is generally considered to be at 700 mV. Therefore, in embodiments, the implant device may limit electrical stimulation potentials to 700 mV. In order to stay below this threshold while still reaching the desired volume of tissue, embodiments may use multiple current release sites.

The Gateway. In embodiments, the implant device may be connected to the neurons, being able to read data and execute stimulation commands on them. Data received from the implant device may be analyzed by researchers and doctors. By using AI/ML models, the doctors may command different stimulation patterns for neurons from different brain areas in order to treat different brain related diseases.

In embodiments, the implant device may stream data up to 4 Mb/s. Pushing all these data directly to Cloud would require either a high band internet connection or a large buffer on the implant device. Both options may have disadvantages such as high costs, limited hardware resources, battery consumption etc. Also, the data content may be highly sensitive, which may require the data to be sent over a highly secured channel that may provide the consistent delivery and privacy of the data.

Responsibilities. Accordingly, in embodiments, the implant device may communicate directly with a Gateway component. The responsibilities of the Gateway may include receiving high speed data stream from the implant device, buffering the implant device recorded data, compressing the data, and streaming the data securely to the Cloud for processing and analysis, receiving complex control commands from the Cloud and delivering the commands to the implant device as neuron stimulation commands, sending configuration commands to the implant device, and requesting the implant device status information.

In embodiments, the implant device may be provisioned to stream data and to receive commands from only one single Gateway. In embodiments, the Gateway may have the capacity to receive data and send commands to multiple implant devices.

In embodiments, the Gateway may have sufficient processing power to handle the communication with multiple implant devices and to stream data to the Cloud and to receive commands from the Cloud. To reduce the complexity of the Gateway and to reduce the maintenance efforts, in embodiments, the Gateway may not contain complex logic or a complex User Interface. The only needed User Interface may be a Configuration/Maintenance Interface.

Examples of Types of Gateway. In embodiments, the software may run on gateway devices such as a mobile gateway, such as a smartphone, tablet, or wearable device, a home gateway, and a deep clinic (hospital) gateway. In embodiments, each of the gateway types may use the same data transfer and security protocols, but may allow for different data rates, buffering and analysis tools, and may have different associated implant device operation modes.

In embodiments, Gateway hardware may include, for example, a CPU/Main Board—for example, ready for operating system kernel installation, a Wireless Communication chipset, Wireless Card for connecting to a local Wi-Fi network, Internal Memory >2 GB, Internal Mass Storage >10 GB, etc.

In embodiments, a Gateway software configuration may include, for example, an operating system, Gateway Software, Web-Server software, that may, for example, be use for configuration purposes, etc.

In embodiments, a default version of the Gateway software may be installed on the Gateway from the factory. In embodiments, the Gateway may start with the provisioning procedure if the provisioning was not already done.

In embodiments, the Gateway software may be frequently updated with novel software versions to ensure data integrity and optimal functionality. In embodiments, the OTA updates may be triggered via Cloud commands. In embodiments, during the OTA updates, the Gateway may suspend the connection to implant devices and to the Cloud to be able to properly execute the OTA update. When the update is finished, the Gateway may restart and reconnect to implant devices and the Cloud. In embodiments, OTA updates may not alter the previously configured parameters. In embodiments, OTA updates may preserve the integrity of the Gateway. In embodiments, when the OTA update fails for any technical reasons, the Gateway Module may re-start and use the previous software version. In embodiments, OTA updates may be accepted only from a specific Cloud host and may be signed with a special OTA related key. In embodiments, automatic OTA updates may be enabled/disabled through the use of the configuration API.

In embodiments, during the initial power up, the Gateway may start its private WAN by initiating an AP(Access Point). In embodiments, in provisioning mode, Gateway may start a web-server that may be used to receive provisioning commands. In embodiments, while in the provisioning phase, a user connected to the AP initiated by Gateway may access the Gateway Configuration Interface via a browser. Example of provisioning parameters may include a connection address of the Cloud Host, Cloud connection credentials for the initial configuration cycle, Credentials needed to connect to a local Wi-Fi network, Gateway administration credentials, etc.

In embodiments, once the Cloud Host address and initial credentials are set correctly, the gateway may trigger a “pairing command”. As a result of the pairing command, the cloud may generate an 8-byte code. This code may be set using the Gateway Provisioning UI. The code may be transmitted to the Cloud to prove its identity. After a successful execution of this process, the Gateway may be ready to receive commands from the Cloud and to stream data to the Cloud.

In embodiments, a Local Configuration Interface may be available during the entire period that the Gateway is running for maintenance purposes. In case of malfunction, a technician may connect to this interface, analyze the status and configuration of the Gateway, and determine the cause of the problems. In embodiments, the technician may manually change the configuration parameters. Any manual changes of the configuration parameters may be synchronized with the Cloud.

In embodiments, the Gateway Configuration UI may be implemented as secured web application. In embodiments, the administration credentials may be set only during the provisioning phase or by a credential override command received from the Cloud. In embodiments, the Gateway may expose a configuration workspace without a user interface and the technician could connect for configuration using a mobile application.

In embodiments, after a successful provisioning, the Gateway may register itself as command executor, for the commands sent by the Cloud. Thus, the Gateway may receive any commands sent by a Cloud user for the purpose of commanding or configuring the implant device or the Gateway. In embodiments, once registered as a command executor, the Gateway may receive commands such as a Gateway configuration command, an implant device configuration command, an implant device state inactivation/activation command, an implant device stimulation command, an implant device status command, an implant device OTA command, an implant device control recording command, etc.

In embodiments, for each Gateway configuration command received from the Cloud, the Gateway may validate it and then change the configuration as requested. In embodiments, the data recording from the implant device modules may not be affected, by the execution of configuration commands on Gateway. In embodiments, for each implant device configuration command received from the Cloud, the Gateway may connect to the targeted implant device configuration API, and send the configuration command to that implant device. In embodiments, the implant device configuration commands received from Cloud may be translated to implant device configuration commands before being delivered to implant device over the implant device configuration API.

In embodiments, for each implant device activation/inactivation command received from the Cloud, the Gateway may connect to the targeted implant device stimulation API and then send the activation/inactivation command. In embodiments, the implant device activation commands received from Cloud may be translated into implant device activation commands before being delivered to implant device over the implant device stimulation API. In embodiments, for each implant device stimulation command received from the Cloud, the Gateway may connect to the targeted implant device stimulation API and then send the stimulation command. In embodiments, the implant device stimulation commands received from Cloud may be translated into implant device stimulation commands before being delivered to implant device over the implant device stimulation API. In embodiments, for each implant device status command received from the Cloud, the Gateway may connect to the targeted implant device status API, request the status, and send it back to the Cloud. In embodiments, the implant device status information may include information such as Battery Level, Recording State: on/off, Active Recording channels, Active Stimulation channels, Software version, etc.

In embodiments, for each implant device OTA command received from the Cloud, the Gateway may connect to the targeted implant device OTA API and deliver the software updates. In embodiments, for each implant device Control Recording command received from the Cloud, the Gateway may send to the target implant device the command for execution, for example, start or stop recording. In embodiments, the communication channel between implant device and Gateway may support continuous data streaming of up to 4 Mb/s. In embodiments in which each Gateway may be connected to multiple implant devices, parallel processing of the incoming data streams may be performed. In embodiments, the Gateway may be able to record multiple incoming data channels and to stream them separately to the Cloud.

In embodiments, the communication between the implant device and the Gateway may be secured. The implant device and Gateway Wi-Fi chipsets may ensure a hardware secure channel between these two devices.

In embodiments, the data recorded by implant device may be streamed at a speed up to 4 Mb/s. For such a high rate data transfer to the Cloud, embodiments may include a high-speed data connection. This may become a constraint in different clinics or facilities. Thus, in this scenario, the Gateway may need to handle a high-speed data publisher (the implant device) and a slower consumer—the upload stream to the Cloud. To solve this problem, in embodiments, the Gateway may buffer the data received from the implant device, package and compress it and only afterwards send it to the Cloud at the optimal provided transfer rate.

In embodiments, the Gateway may send to the Cloud data packets of similar sizes. In embodiments, the Gateway may start to send the data when the internal in-memory data buffer is full.

In embodiments, the data coming from the implant device may be compressed using an encoding algorithm. Still, the need to convert, for example, 10 bits float to 16 bits float, enlarges the data volume that needs to be transferred to the Cloud by 60%. To keep the transfer size low and to reduce the Cloud upload latency, the Gateway may compress these data before uploading it to Cloud.

Given that there could be multiple Implant devices connected to the same Gateway, in embodiments, the Gateway may be able to handle the incoming data in multiple parallel threads. The ongoing data transmission flow may not be affected by new incoming data streams. In embodiments, any incoming data channel for a specific implant device may be processed, compressed, and streamed to the Cloud independently of any other active data channels corresponding to other Implant devices.

In embodiments, when the Gateway is powered on, it may open the data incoming channels (server sockets) for all linked implant devices. It may be that for certain reason, for example, battery drain, implant device location changed, etc., the implant device may not be able to connect at that moment to the Gateway. Still, when the implant device enters the connection area and starts transmitting data, the Gateway may pair with the implant device and start receiving its data.

In embodiments, after the provisioning phase is finished, the Gateway may be paired with the Cloud, thus for each implant device that it controls it may, for example, register itself as a Commands Executor and initialize the Data Publisher Channel. In embodiments, any communication between Gateway and Cloud may be over a secure channel and may use an AES(128 bits) encryption key. In embodiments, execution/configuration commands received from Cloud may be encrypted with this key. In embodiments, the Gateway may encrypt all data pushed to the Cloud with the AES key. In embodiments, the AES keys may be periodically changed and may be transferred between Cloud and Gateway using, for example, the Diffie-Hellman Symmetric Key Exchange protocol.

In embodiments, the Gateway may ensure that any data recorded from the implant device may be transmitted to the Cloud. In embodiments, in case of communication failures between the Gateway and the Cloud, the Gateway may retry sending the data when the connection is restored. In embodiments, the Gateway may store locally (on persistent storage) the un-sent data in case the communication channel is broken for a longer period of time. In embodiments, the persistence buffer may have a pre-configured size. In embodiments, once this size is exceeded, the Gateway may apply a first-in-first-out (FIFO) eviction policy. Thus, the older entries may be deleted in order to make room for new incoming data. In embodiments, this may be the only configurable scenario in which the Gateway may lose data received from the implant device. In embodiments, once the connection is re-established the Gateway should automatically synchronize the data with the Cloud.

In embodiments, the data uploaded from Gateway to Cloud may not contain any private information about the patient. In embodiments, the link between the patient details and the recorded data may be stored and known only in the Cloud. In embodiments, each data incoming channel on the Cloud may be associated with a specific implant device. In embodiments, in the Cloud there may be a privacy information database, which may store the relations between the patient and the implant devices. In embodiments, no patient sensitive data may be transferred from Cloud to Gateway. In embodiments, the commands sent from the Cloud may address directly the implant device and may not contain any patient information.

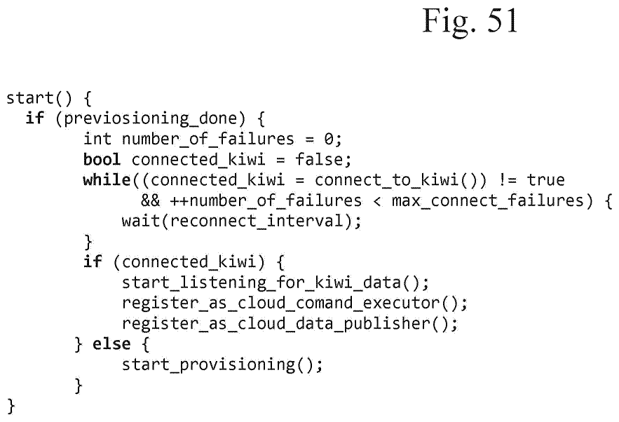

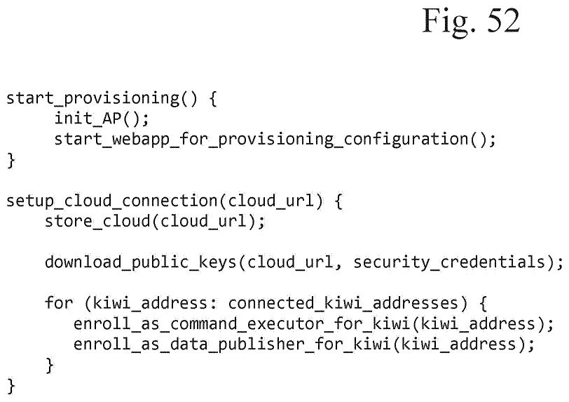

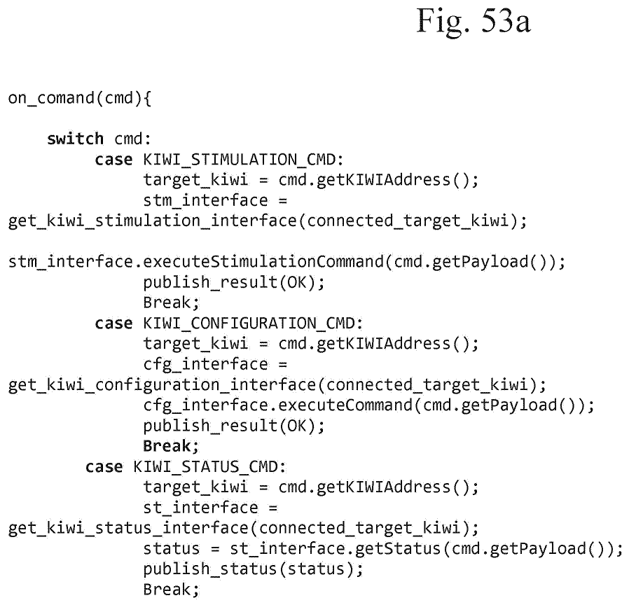

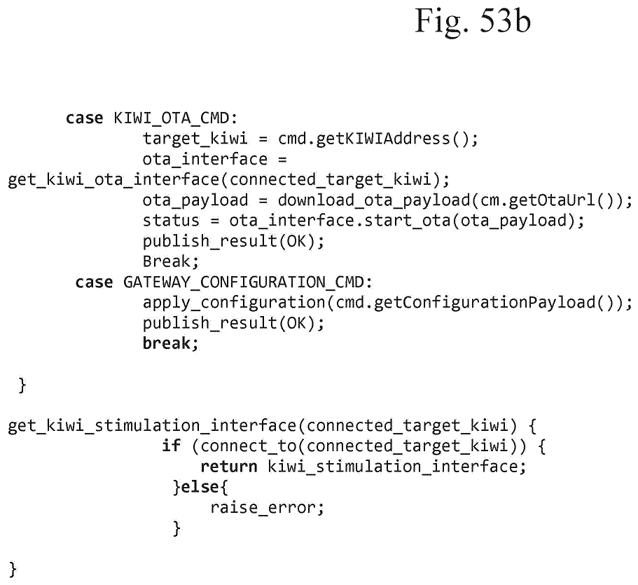

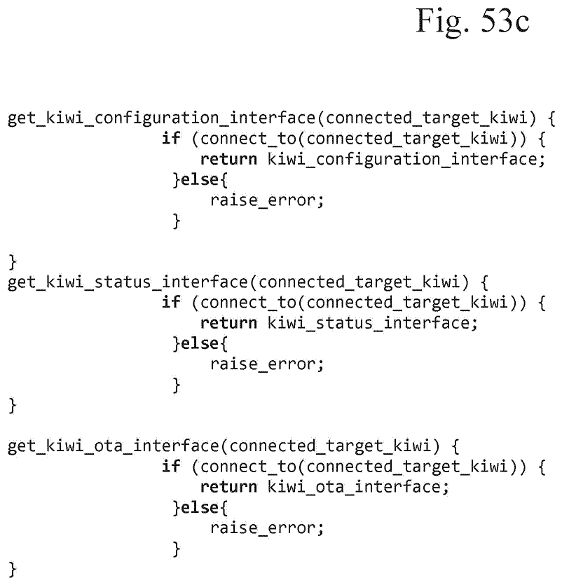

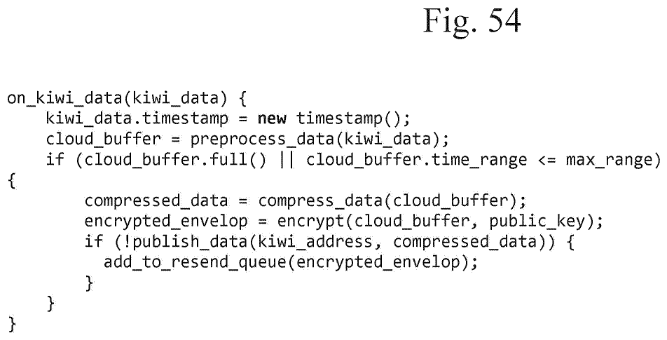

A pseudocode example of a startup procedure is shown in FIG. 51. A pseudocode example of a Provisioning procedure is shown in FIG. 52. A pseudocode example of a command execution procedure is shown in FIGS. 53a, 53b, and 53c. A pseudocode example of a data streaming procedure is shown in FIG. 54.

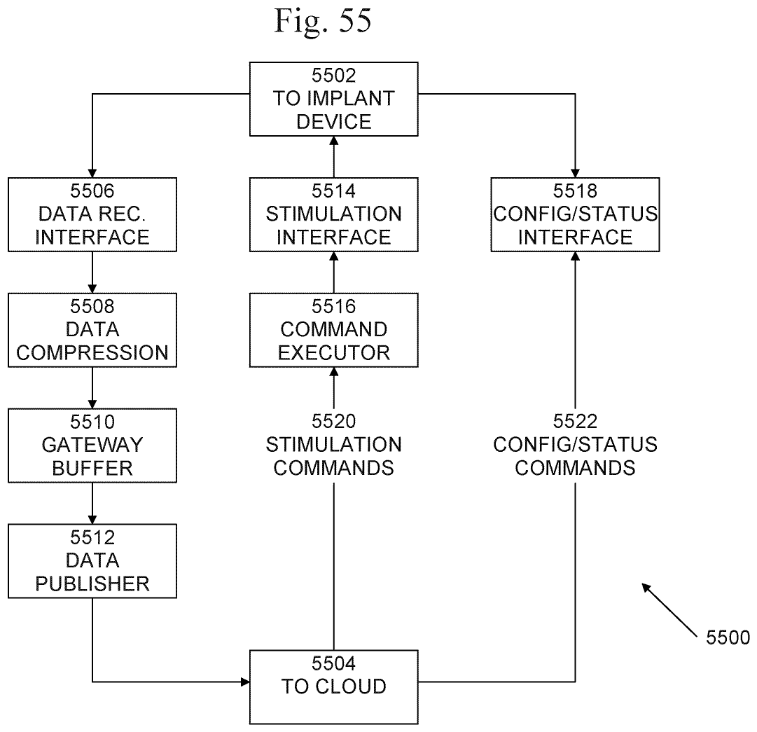

An exemplary block diagram of a Gateway 5500 is shown in FIG. 55. As shown in this example, Gateway 5500 may include communications with implant device 5502, communications with the Cloud 5504, a data recording interface 5506, data compression 5508, a buffer 5510, a data publisher 5512, a stimulation interface 5514, a command executor 5516, and a configuration/status interface 5518. Communications with implant device 5502 may include hardware and software to provide communications with the implant device. Communications with the Cloud 5504 may include hardware and software to provide communications with the Cloud. Data recording interface 5506 may include hardware and software to receive data from the implant device and process the data prior to data compression, as described above. Data compression 5508 may include hardware and software to provide compression of the processed data received from the implant device, as described above. Buffer 5510 may include hardware and software to provide temporary storage of compressed and/or uncompressed data, as described above. Data publisher 5512, may include hardware and software to publish and communicate data to the Cloud, as described above. Stimulation interface 5514, may include hardware and software to generate stimulation commands, and/or multiple or sequences of stimulation commands to be transmitted to the implant device, as described above. Command executor 5516, may include hardware and software to receive stimulation commands 5520 from the Cloud and execute those comments in conjunction with stimulation interface 5514 and the implant device, as described above. Configuration/status interface 5518, may include hardware and software to receive and process configuration/status commands from the Cloud, as described above.

The Cloud. Data recorded from the implant device may be processed and analyzed. Based on this data, the neuroscience researchers may build AI/ML models that may be used by practitioner doctors to treat different brain related maladies such as Parkinson, Alzheimer, etc.

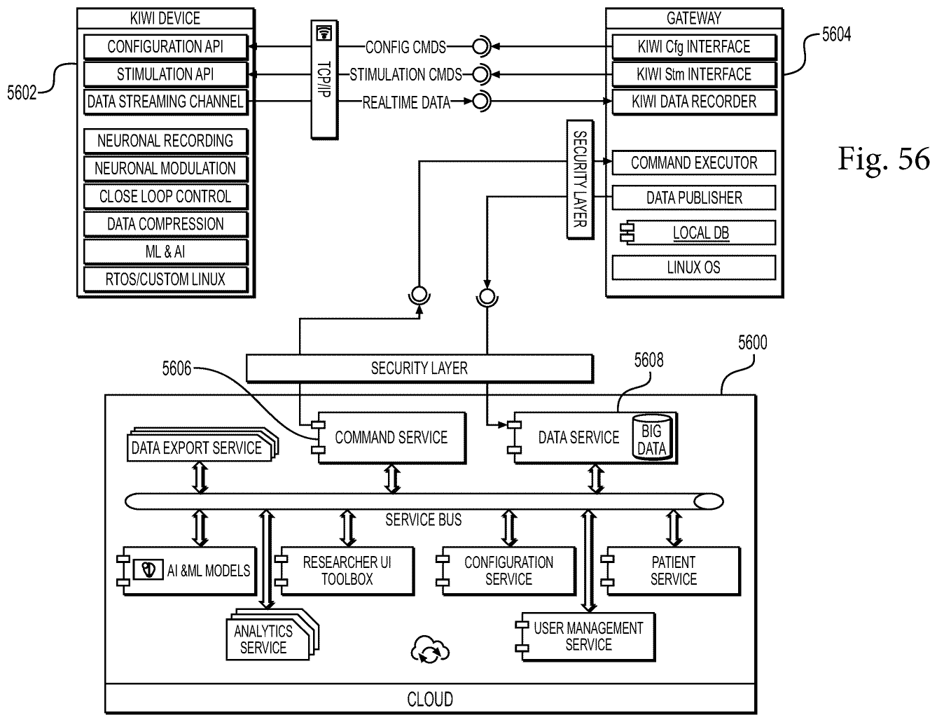

The Cloud may include of a cluster of nodes on which different microservices may be deployed. An exemplary high-level block diagram of the Cloud 5600 is shown in FIG. 56. Also shown in this example are implant device 5602 and Gateway 5604. As shown in this example, Cloud 5600 may include a command service 5606 and a data service 5608. Command Service 5606 may receive, for example, stimulation, activation, configuration, provisioning commands from the user via a User Interface and then may distribute them to the Gateways for execution. Command Service 5606 may also receive back the result of the command execution and present them to a user. Data Processing Service 5608 may take care of data ingestion coming from the implant device and the processing and storing of this data.

Command Service. In embodiments, Command Service 5606 may execute commands such as implant device OTA, implant device Configuration, Gateway Configuration, implant device stimulation, implant device activation/inactivation, implant device recording control, etc.

In embodiments, the commands may be transmitted from the Cloud as a request of a user (Medical Doctor, Researcher) and may reach an implant device which may be located in a local network behind a firewall. Accordingly, in embodiments, a Publish/Subscribe architecture may be used. In embodiments, the Cloud may publish commands for execution, while the Gateway may be registered as a subscriber for these commands. In embodiments, the Gateway may, in this case, play the role of commands executor.

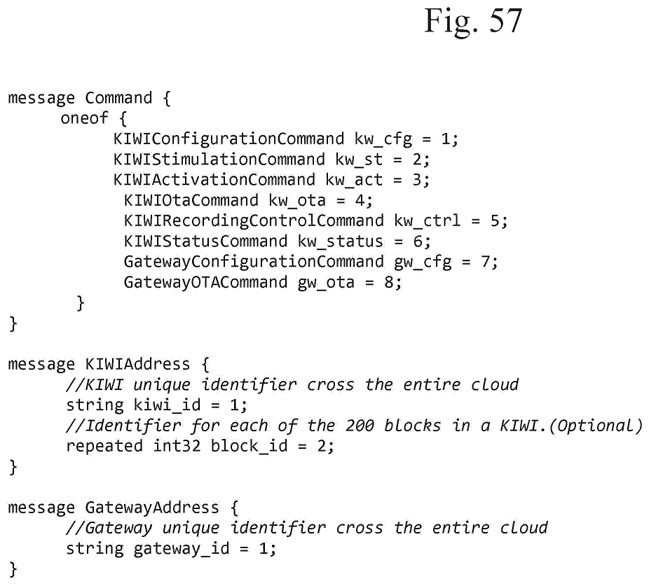

In embodiments, Command Service 5606 may be implemented as a microservice and may be deployed on multiple nodes in Cloud 5600. In embodiments, Command Service 5606 may expose an interface for command requests, which may be used by other services to send commands. In embodiments, each command may indicate the implant device or the Gateway to which it is addressed. In embodiments, when a user triggers a command from the user interface, the command may be created and then may be published on a commands Queue. The Command Executor which is registered for that implant device or Gateway Address may execute the command. A pseudocode example of a command message is shown in FIG. 57.

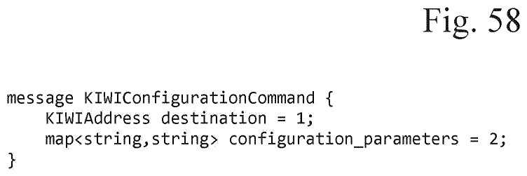

In embodiments, a Configuration Command may contain configuration changes which apply to the targeted implant device. In embodiments, the Configuration Command may include Configuration Parameters that may contain parameters that may be configured on an implant device. In embodiments, the Configuration Parameters may contain information such as Gateway IP/MAC addresses, Stimulation channels, recording channels, Recording reporting frequency, Scheduled start/stop, Stimulation methods—Optical, Electrical, Chemical, etc. A pseudocode example of a Configuration Command is shown in FIG. 58.

In embodiments, the Stimulation Command may include information about the stimulation of specific channels of the targeted implant device. A pseudocode example of a Stimulation Command is shown in FIG. 59. In embodiments, the Command Executor may apply the required stimulation command on the specified channels.

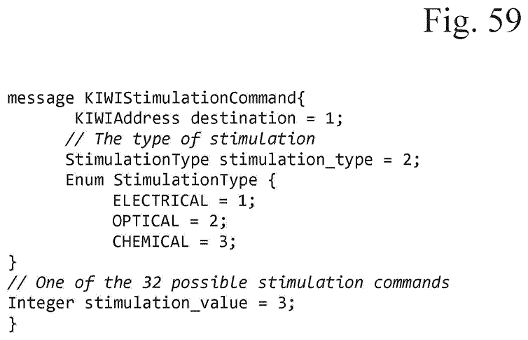

In embodiments, the Activation Command may include information about the activation/inactivation of certain channels of a targeted implant device. A pseudocode example of an Activation Command is shown in FIG. 60. In embodiments, the Command Executor may apply the required activation/inactivation on the specified channels.

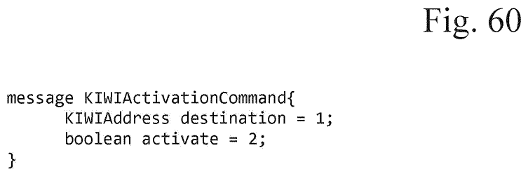

In embodiments, the OTA Command may include information about a new version of software that needs to be installed on the implant device. A pseudocode example of an OTA Command is shown in FIG. 61. In embodiments, when executing this command, the gateway to which the implant device is connected may download the OTA image data from a predetermined network address, verify it and then it will trigger the implant device OTA update by pushing the image data through the implant device OTA interface. In embodiments, after a successful OTA update installation, the implant device may restart and use the new software version.

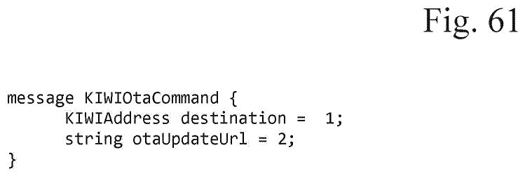

In embodiments, the Recording Control Command may be a request to start or suspend the recording on the implant device. A pseudocode example of a Recording Control Command is shown in FIG. 62. In embodiments, when executing this command, the Gateway may send the request to start or suspend recording or neuronal activity to the controlled implant device.

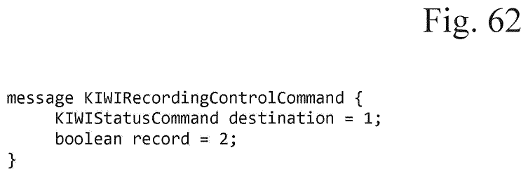

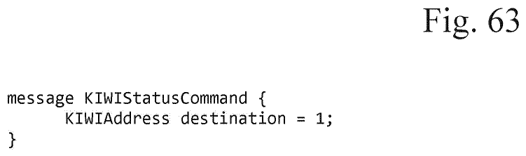

In embodiments, the Status Command may be a request to update the implant device Status on the Cloud. A pseudocode example of a Status Command is shown in FIG. 63. In embodiments, when executing this command, the Gateway may request the status information from the implant device and push the status information to the Cloud.

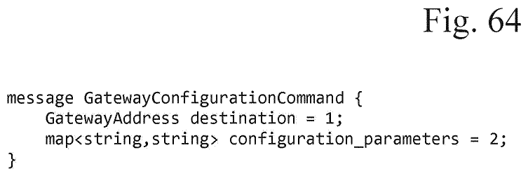

In embodiments, the Gateway Configuration Command may include information about the new configuration that needs to be set on the Gateway. A pseudocode example of a command message is shown in FIG. 64. In embodiments, the configuration parameters may include information such as Local Wi-Fi network credentials, Cloud host network address, local administration credentials, network addresses of connected implant devices, implant device heartbeat checking interval, etc.

In embodiments, the Gateway may have a predefined buffer for recording data from the implant device. In embodiments, when this buffer is full, the recordings may be pushed to the Cloud. If real time data recording and streaming to the Cloud is needed, this buffer may be disabled or it may have a smaller size.

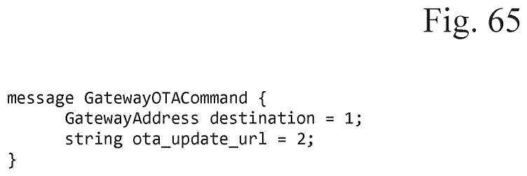

In embodiments, the Gateway OTA Command may include information about a new version of software to be installed on the Gateway. A pseudocode example of a command message is shown in FIG. 65. In embodiments, when executing this command, the Gateway may download the OTA image data from a predetermined network address, verify it, and then trigger the OTA update. In embodiments, after a successful OTA update installation, the Gateway may restart and use the new software version.

In embodiments, for each executed command, the Gateway may publish the status of execution back to the requestor of that command. In embodiments, when a command is added to the commands Queue, it will have an execution timestamp deadline. If the command is not taken from the Queue by any executor before the timestamp expires, the command may be marked with status “failed to execute” and the requestor may be informed about this failure. In embodiments, each command may be executed only once, irrespective of the result. The requester may decide to re-trigger the command in case of error, but this may be recognized as a new command. In embodiments, the commands may not contain any information related to the patient on which the implant device is applied. In embodiments, the commands may be executed only by the Gateway which controls the target implant device. In embodiments, the commands may be sent to Gateway over a secure channel. In embodiments, the system may guarantee the delivery of the commands to the Gateway component, where they may be executed. In case of error, the requestor of the command may be notified about the failure.

Data Service. In embodiments, Data Processing Service 5608 may be responsible for collecting the implant device data, decompressing the data (if need be), and storing the data for later use. In embodiments, there may be a large number of implant devices, which may send their data to the Cloud. Thus, on the Cloud, there may be a need for high scalability in recording this data and also there may be a demand to store a large amount of data. In embodiments, different technologies may support this. For example, the Publish/Subscribe Paradigm may enable the constant increase of implant devices and high parallelism of incoming data. In embodiments, the implant devices may act as data publishers while the Cloud that processes the data may act as a subscriber.

In embodiments, Data Service 5608 may be implemented as a microservice and may be deployed on multiple nodes on cloud. In embodiments, the Gateway may automatically upload the incoming data from the implant device to the Cloud. In embodiments, the Gateway may automatically register itself as a data publisher when one of the connected implant devices is starting to stream data. In embodiments, the communication channel between the Gateway and the Cloud may guarantee the delivery of the data. In case of connection errors, connection interruptions, lost packets, etc., the Gateway may be notified about the failure so that it can schedule a retry request. In embodiments, only a registered Gateway may stream data to the Cloud. Registered Gateways are those for which the provisioning step was executed and they have exchanged the encryption keys with the Cloud. In embodiments, the gateway and the Cloud may be connected over a secured channel. The messages transferred over this channel may be encrypted. The data streaming channel may be compliant with the existing medical standards.

In embodiments, for each channel, the implant device may record the specific value at a given time. The time of recording, reading value and recording type may be grouped together and may be streamed to the Cloud via the connected Gateway.

In embodiments, the data pushed from the Gateway to the Cloud may be time series data and may have a message structure similar to the example shown in FIG. 66. In this example, the message may include a plurality of floating point values, which may, for example, represent the data recorded from all active channels at a given timestamp, in which case, the order in the array may be fixed and may follow the physical tiles and channels numbering. As another example, the values may represent all data recorded from all active channels over a large interval of time. In embodiments, for each recorded channel the values may contain a timestamp=timestamp+blockIndex*readingInterval.

In embodiments, the data coming from the implant device may be encoded/compressed. Accordingly, when it arrives on the Cloud, the data may be reconstructed by applying a decoding/decompressing process. This process may include the entire pipeline of encoding/compression algorithms used at the implant device level while reading, processing, and sending data to the Gateway.

In embodiments, implant device data may be saved on the Cloud on a persistence layer in order to allow later-on batch processing and data retrieval. Any persistence technology may be used that provides the capability to handle the data volume. In embodiments, the data volume may be quite high. for example, an implant device may output up to 4 Mb/s. Assuming a full 24 hours recording, and 1000 implant devices, a data volume up to 432 TB per day may be produced.

Further, the persistence technology may provide the capability for data saving and retrieval to be as near to real time as possible. The high volume of data may generate big storage costs and also could increase the processing power needed for fast retrieval of the stored data.

In embodiments, to reduce the volume of data and to optimize the data retrieval speed, the persistence layer may support Backup Policies—based on predefined rules, the data that matches these rules may be backed up automatically, and Eviction policies—based on predefined rules, the data that matches these rules may be removed from the persistent storage.

In embodiments, Data Service 5608 may expose a data retrieval API that may be used by other Cloud services. This API may support data retrieval by using different filtering conditions. In embodiments, using this API and the filters, UI widgets, ML models, and data exporters may retrieve and use the data stored on the persistence layer. In embodiments, the interaction shall be performed through REST or QL filters.

In embodiments, after decoding and decompression, the implant device streamed data may be exposed to other components as a real time data stream, for example, for real time data visualization.

In embodiments, the incoming data from implant devices may not contain any information related to the patient. In embodiments, the Cloud may store the relation between the patients and implant device data, but this should be available only for Authorized User Roles and Authorized Operation Types. For example, researchers may have access only to anonymized data. In embodiments, practitioners may have access to patient private data only for the patients that are under their supervision.

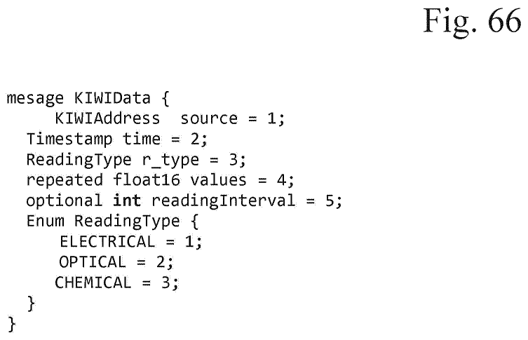

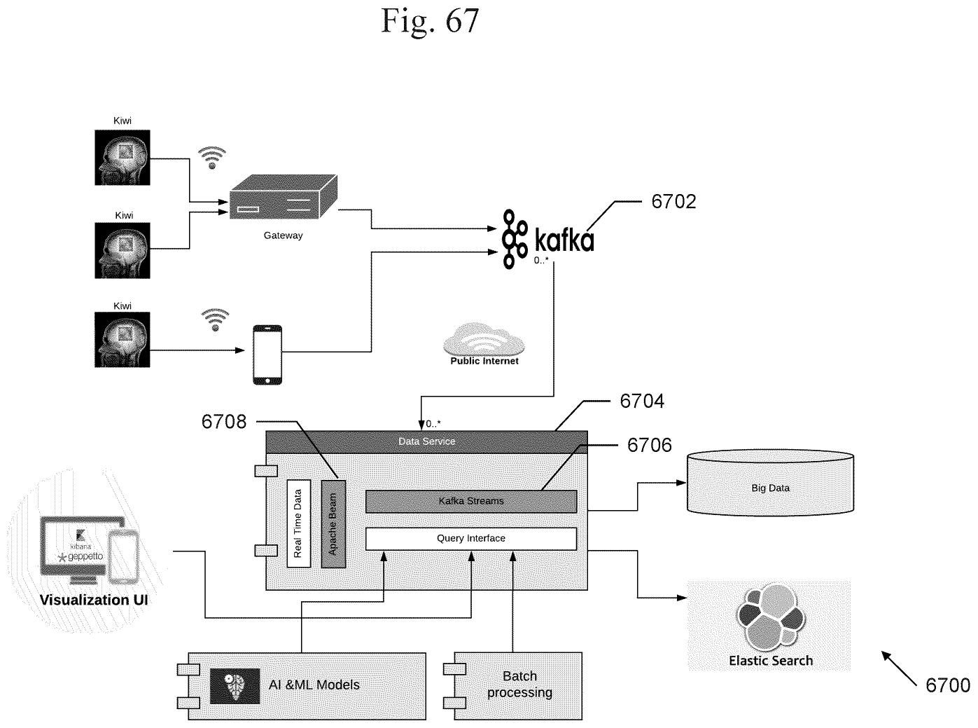

In embodiments, in order to support high scalability during data ingestion, the data processing service may be deployed in a cluster computing environment. Each data stream event may be processed by a single cluster node. An example of an architecture 6700 for data ingestion and data processing is shown in FIG. 67. In this example, technologies that may be included may ease the implementation of the functional and nonfunctional requirements of the Data Processing Service. It is to be noted that although specific technologies are described in this example, one of ordinary skill in the art would recognize that other technologies that provide similar or equivalent functionality may be used instead, or in addition to, the described technologies.

For example, APACHE KAFKA™ 6702 may be used for data streaming and ingestion. It may be used for building real-time data pipelines and streaming apps. KAFKA™ is horizontally scalable, fault-tolerant, and very fast, being used in production by large companies. In embodiments, the data coming from implant devices may be distributed for processing to Cloud Data Processing Service 6704, which may be deployed in several nodes on the Cloud. KAFKA™ may also provide an easy method for starting/stopping the KAFKA™ Processors (the Cloud Data Processing Service 6704). In embodiments, APACHE KAFKA™ Security with its flavors TLS™, KERBEROS®, and SASL™ may help in implementing a highly secure data transfer and consumption mechanism.

In embodiments, APACHE KAFKA™ Streams 6706 may ease the integration of Gateway and Data Processing Service in the KAFKA™ Ecosystem.

In embodiments, APACHE BEAM™ may unify the access for both streaming data and batch processed data. It may be used by the real time data integrators to visualize and process the real time data content.

In embodiments, a high volume of predicted data and data upload and retrieval may be handled by a Time Series database Examples of such technologies may include OPENTSDB™—A Distributed, Scalable Monitoring System, TIMESCALE™—an Open-Source Time-Series SQL Database Optimized for Fast Ingest, Complex Queries and Scale, BIGQUERY™—Analytics Data Warehouse, HBASE™, HDF5™, and ELASTICSEARCH™, which may be used as second index to retrieve data based on different filtering options.

In embodiments, add-on programs, such as GEPPETTO™ UI widgets may be used for visualizing neuronal activities. Further, KIBANAT™ is a charting library that may be used on top of ELASTICSEARCH™ for drawing all types of graphics: bar charts, pie charts, time series charts etc.

Processing Pipelines. In embodiments, to give doctors and researchers the ability to manipulate the data and apply various algorithms to classify patient data, recognize patterns, recommend treatment, and do any types of processing, the Cloud component may support pipelines. In embodiments, the pipelines may include separate blocks, which may determine what data to process and what code to run over it. Each block may be configured individually. for example, the configuration may be done via a Drag and Drop UI or via a coding interface.

In embodiments, there may be different kinds of pipelines, for different use cases. For example, a real-time processing pipeline may be used by doctors to treat patients. This pipeline may have low latency and may not need high throughput. Another example is a batch processing pipeline, which may be used by researchers who want to train new models. This pipeline may have very high throughput, but the latency requirements may not be high. Another example is an automatic pipeline based on a central schema, which may be used for aggregating and analyzing data from different sources, and for scheduling automatic training and processing in the entire system.

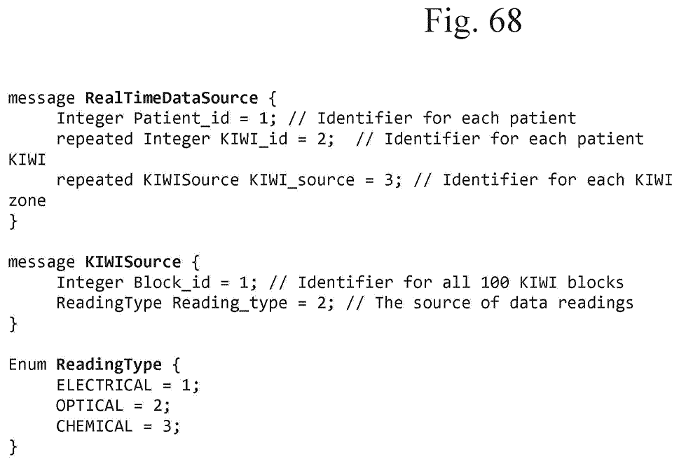

Real-time Processing. In embodiments, to enable the system to respond quickly to incoming data from the implant devices, real time processing may be provided. This means that each data point (for example, electrical measurement taken by the implant device) is processed as soon as it arrives into the cloud database. An example of an API that may be used to specify the input for real time processing is shown in FIG. 68.

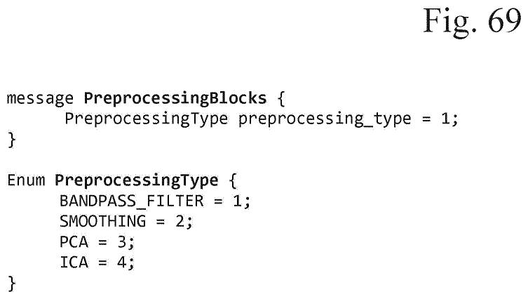

In embodiments, after specifying inputs, other kinds of operators may be applied to the data, element-wise, such as band pass filters, smoothing, and dimensionality reduction such as ICA or PCA. An example of an API that may be used to specify the pre-processing for real time processing is shown in FIG. 69.

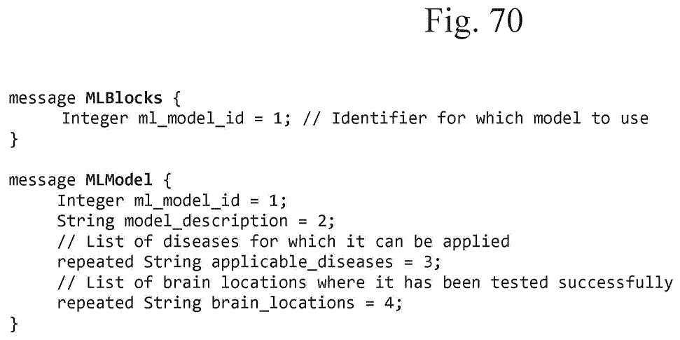

In embodiments, for real time processing, existing machine learning models may be applied to the data in order to obtain inferences about the patient. These machine learning models may exist in a central repository. These models may be annotated with information about what kind of diseases they apply to and what conditions have they been tested in (such as location of implant devices). An example of an API that may be used to specify the machine learning processing for real time processing is shown in FIG. 70.

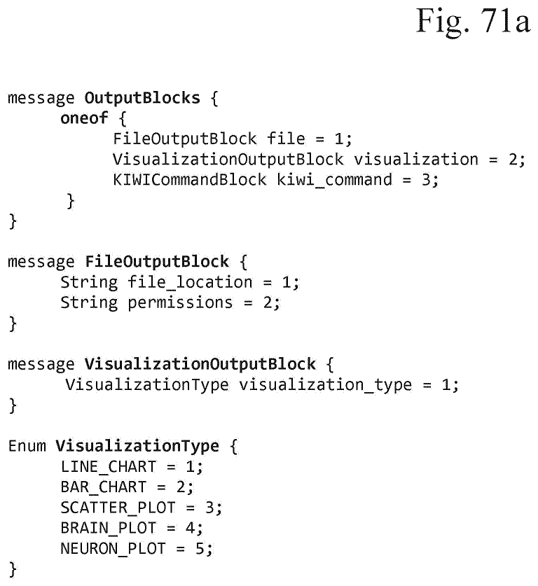

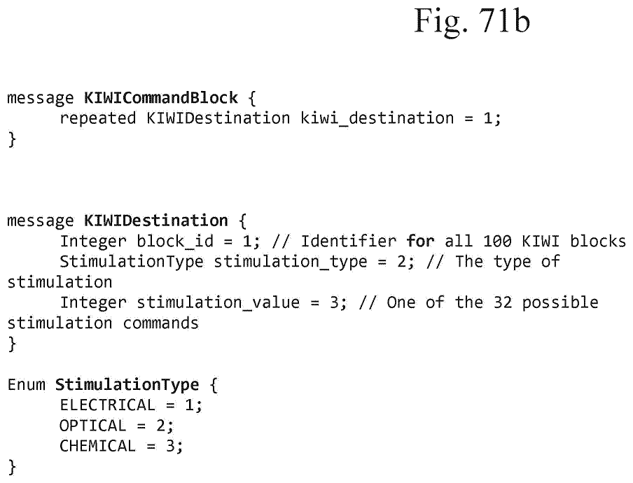

In embodiments, after all the processing has been done, the result may be output. This may mean either saving to disk, in a patient's file for example, or shown in a visualization, so that a user may understand what is going in the patient's brain in real time, or it may be used to send information to the implant device about what kind of neural stimulation commands to give. An example of an API that may be used to specify the output for real time processing is shown in FIGS. 71a and 71b.

Batch Processing. In embodiments, researchers may train algorithms over the data of many patients. These algorithms may take a long time to train, so there are few latency requirements in this case, but they need to be able to process a large amount of data, processing gigabytes of data every second.

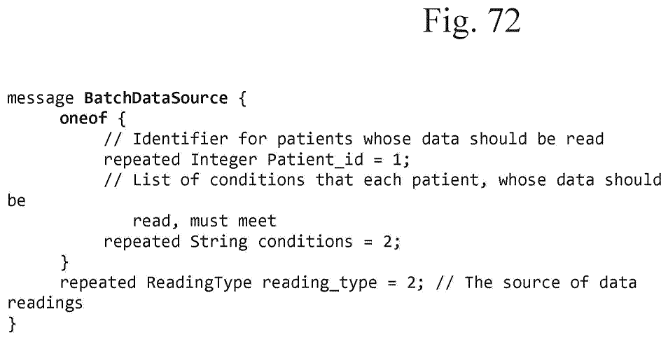

In embodiments, as input, the researchers may select data belonging to only some patients, according to various criteria (such as having a certain age, or a certain disease, etc.). The output of this pipeline may be the resulting trained models, along with statistics about how well they performed (accuracy, loss, etc.). An example of an API that may be used to specify the input for batch processing is shown in FIG. 72.

In embodiments, the preprocessing blocks for the batch pipelines may be similar to the Real Time Processing Blocks, and these functions may be accessed using a similar API.

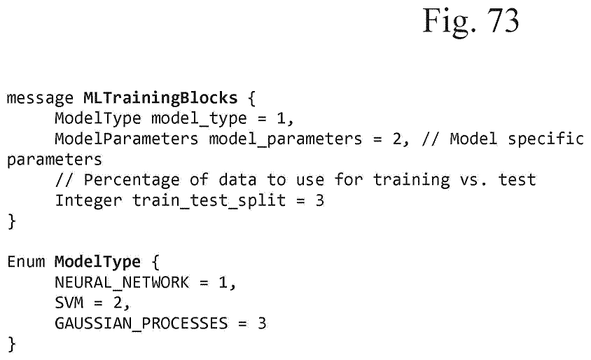

In embodiments, for batch processing, the researchers may have the option to use existing machine learning models or they may train new models which may then be saved into a central repository. These models may be annotated with information about what kind of diseases they apply to and where the data for them has been obtained (such as location of implant devices). For existing models, similar processing blocks and API may be used as for the Real Time Processing. For training new models, an example of an API that may be used to specify the machine learning for training new models for batch processing is shown in FIG. 73.

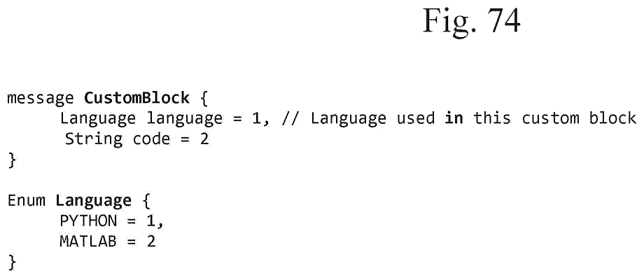

Custom Blocks. In embodiments, researchers may have the ability to run custom blocks where they can run any code they want. These custom blocks may have access to standard machine learning libraries and servers such as MATLAB™, TENSORFLOW™, SCIKIT-LEARN™ etc. An example of an API that may be used to specify the custom blocks for processing is shown in FIG. 74.

In embodiments, when the batch processing has been completed, the resulting model may be written to disk. At the same time, during training, a summary of the progress of the model training may be saved. An example of an API that may be used for output from batch processing is shown in FIG. 75.

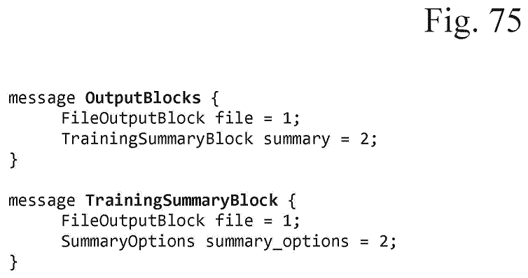

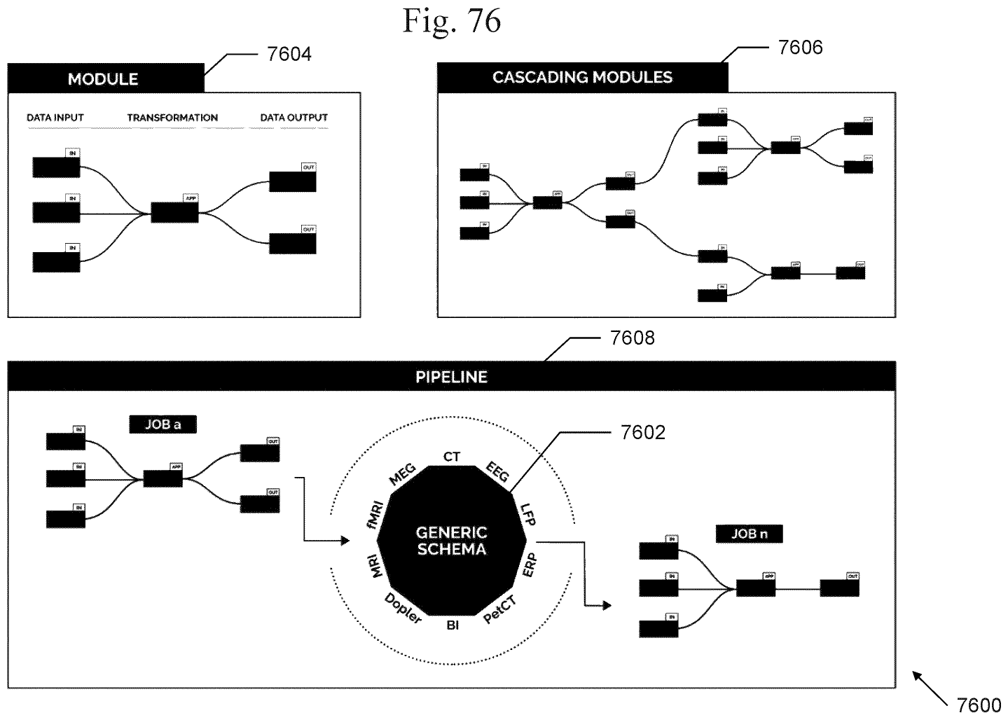

Automatic Pipeline. An exemplary block diagram of an automatic pipeline 7600, which may be used for aggregating and analyzing data from different sources, and for scheduling automatic training and processing in the entire system, is shown in FIG. 76. Pipeline 7600 may provide a way of joining different fields of expertise in a common collaboration environment. Each researcher may define his own experiments/tests that may be linked in a common workflow. The output of one Module (research) can trigger (automatically) a Module prepared by another researcher. All Modules may be versioned and may be easily reproduced by any team member.

Collaboration is only meaningful with a general understanding of each other, this applies also for any process run through the pipeline. In embodiments, the core of Pipeline 7600 may be the Generic Schema (GS) 7602 that may be used to map all the different data elements used by the different Modules. GS 7602 may be seen as the common language (describing data) used by each of the Modules even when using different programming languages. Furthermore GS 7602 may be heavily used by the Reporting layer that reports and analyses results across all modules.

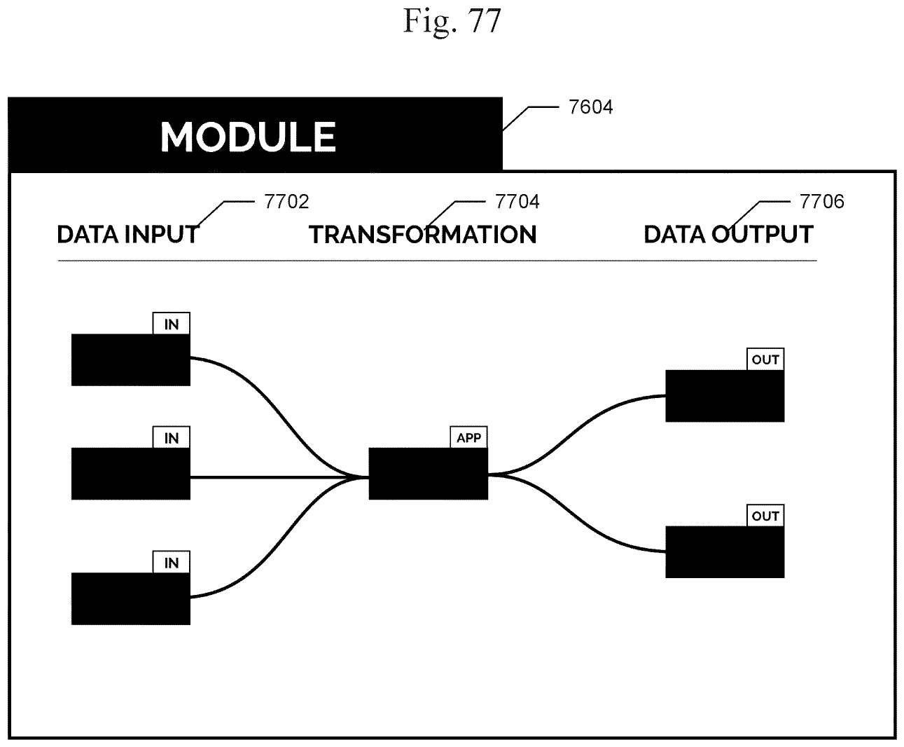

Modules 7604, also shown in FIG. 77. In embodiments, modules may be autonomous processes that may include Data Input 7702—one or more Data sets/sources, Transformation 7704 —code & scripts needed to do the transformation on the input, and Data Output 7706—one or more result sets. In embodiments, each module may be run in the cloud and may launch spot instances. In embodiments, each module may accept as input any data formats. In embodiments, code used in Transformation 7704 may be versioned using a version management system. In embodiments, rolling forward and backward may be possible with the same data sets.

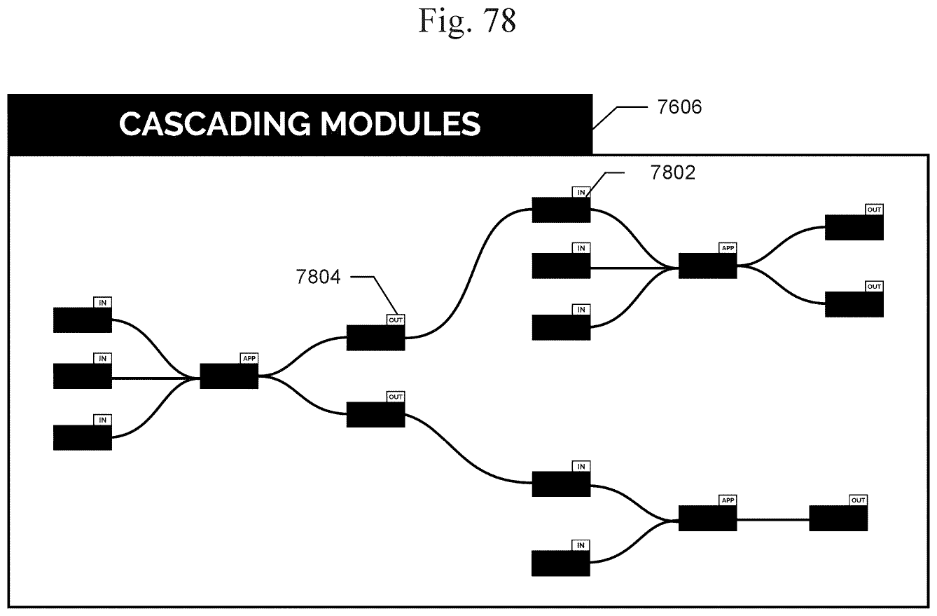

Cascading Modules—7606 in FIG. 76, also shown in FIG. 78. Each Module may have Data Inputs that may be of any commonly used file format or online stored data set. Alternatively, the Input 7802 of a Module may be defined as the Output 7804 from another Module. In embodiments, this feature may be used to define Cascading Modules 7606 (workflows) that perform their tasks based on other Modules. Monitoring of these flows may be done in a Console (start, end, duration).

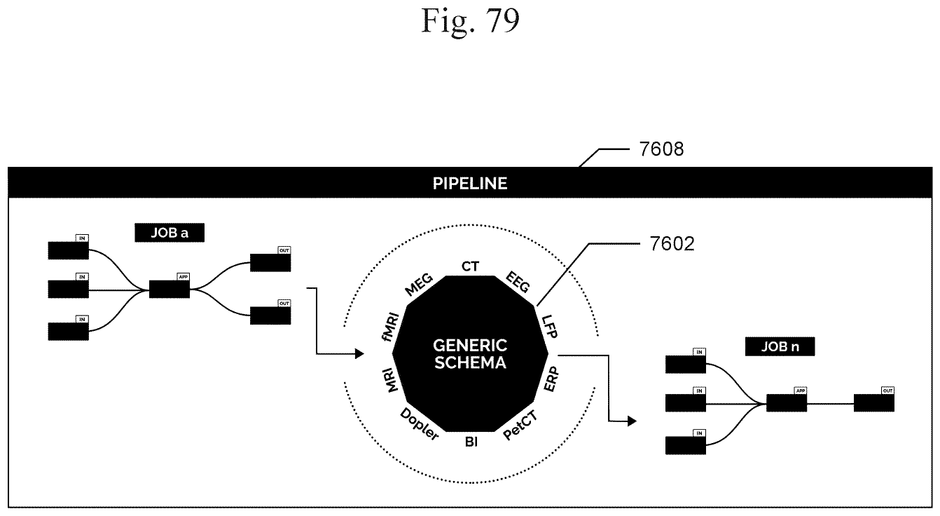

Pipeline—7608 in FIG. 76, also shown in FIG. 79. In embodiments, the orchestration of all modules may be done in Pipeline 7608. By configuring each pipeline, one may define flows that take results from each of the different fields (EEG, LFP, ERP, PetCT, MRI etc.) and make coherent analyses. The Generic Schema (7602 in FIG. 76) may ensure the results are easy to understand and correlate.

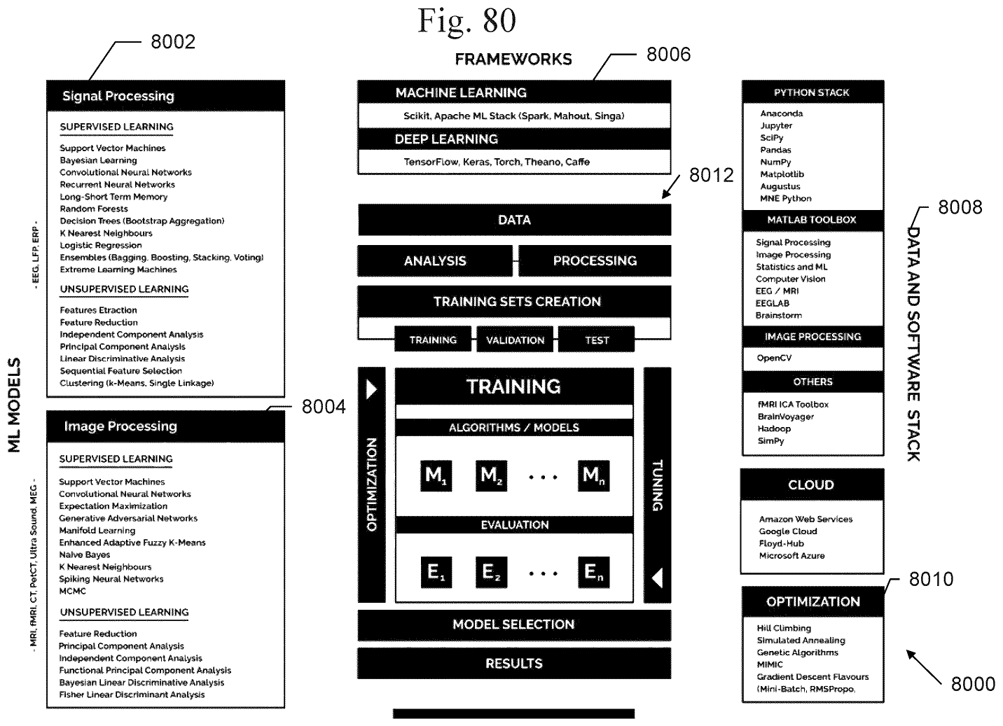

Machine Learning (ML) Toolbox 8000, shown in FIG. 80. In embodiments, the toolbox may include layers such as Machine Learning Models for Signal Processing 8002 and for Image Processing 8004, Machine Learning Frameworks 8006, Data and Software Stacks 8008 for Data Analysis, Data Processing, Cloud Computation, and Optimization Approaches 8010. Examples of Machine Learning Models for Signal Processing are shown in block 8002, and examples of Machine Learning Models for Image Processing are shown in block 8004. An example of a processing flow 8012 is also shown. Such processing flows may be customized depending on the needs of the task at hand.

In embodiments, some of the machine learning models may be general, applicable to all brain recording data. Examples of these may be Linear Discriminant Analysis and Sparse Logistic Regression. In embodiments, there may also be machine learning models which are targeted for a specific disease, such as Alzheimer's disease and Parkinson's disease.

In the case of Parkinson's disease, the machine learning models may be trained to recognize when the patient is having motor problems, either with bradykinesia or excessive tremors. When detecting these mental states, a signal would be sent to start activating neurons in the appropriate region, in order to help alleviate the symptoms.

In the case of Alzheimer's disease, the machine learning models may be used to recognize when a patient has problems recalling already learned concepts and stimulation may be applied to help in memory improvements.

The cloud system may also implement the Fundamental Code Unit framework to analyze and correlate all the data of a patient starting from low-level neurotransmitter levels and neural spiking data, to high level behavioral data such as language and gait analysis. The cloud system may further implement the BrainOS, which is a cloud platform that may harbor the parallel data flow and FCU analytic engine powered by neurocomputational algorithms and deep machine learning. EEG, ECG, and other physiological data (external and internal) may be uploaded to the cloud wirelessly from the Brain Code Collection System (BCCS) and KIWI. A suite of algorithms may analyze the aggregate data stream and formulate instructions for optimal electrical and/or optical neuromodulations in a closed loop feedback system. Integrated stimulation/control, recording/readout and modulated stimulation parameters will allow simultaneous optical and/or electrical recording and stimulation.

BrainOS may integrate methods and technologies such as Deep Cognitive Neural Networks (DCNNs), Spiking neural networks (SNNs), and Spatio-temporal firing unit localization, described below. BrainOS may also feature a no-code drag-and-drop AI user interface that will make it accessible for medical doctors and researchers to use the power of deep analytics without needing to spend time and resources learning to code.

Deep Cognitive Neural Networks (DCNNs) are a new type of neural network architecture exhibiting Highly Energy-Efficient Shallow Neural Networks on CMOS or PCMOS. For example, embodiments may be 226-300 times more efficient in terms of Energy Performance Product. (DCNNs) may provide Fast Decision-Making, for example, up to 80×faster decision-making in real-time, based on simulations, improved generalization for long-term learning in extreme environmental changes, improved performance without retraining, a mean squared error much lower compared with classic neural networks, and faster convergence behavior when integrated with reasoning algorithms.

Spiking neural networks (SNNs) are artificial neural networks that closely mimic biological neural networks. Based on the human species proof that biological neural networks work, spiking neurons are more faithful (while still simplified) representations of the biological neurons, when compared with DNN. Embodiments may utilize unsupervised learning algorithms based on Hebbian Learning (“cells that fire together wire together”) using retrograde signaling (strengthens the synaptic between the activated neuron and the excitatory neuron; neighbor neurons also become associated with the target-activated neuron). Spiking neural networks (probabilistic networks) may have predictable behavior at the network level. Small spiking networks are a valid approach, given the retina and optic nerve's capacity of using a tiny fraction of the cells in information processing at a given time. Embodiments may use Long-Term Potentiation (LTP) and Depression (LTD) as essential additions to the big picture of how the brain learns. Embodiments may use Neuro-Evolution Methods (Genetic Programming) for optimizing the models for specific tasks. Embodiments may use Actor-based (message-based) approach to concurrency, to ensure parallel and async “synaptic signaling”

Embodiments may provide:
Infinite Scalability—the “spiking brain” can live on a single computer, in a network or all over the internet.
Lightweight Computation—the neural level computation consists of simple addition and multiplication (there is no need for GPU devices or special hardware architecture—however, it is understandable that specially designed hardware can increase the performance of such networks).
Engram Interrogation—each neuron can be interrogated using specially designed messages (post and reply) even during the training period (without interrupting or perturbing the learning process). This feature enables us to understand the learning dynamics and even extract “incomplete memories” to further aid our understanding of the learning process in spiking networks context.
Recursive Data Structure Approach—each network can also act as a neuron, enabling us to experiment with a modular approach where some neurons are working together in isolation and provide a unique signal that can be sent to another network of neurons (exploring hypotheses like cortical columns).

Simulations show that the neural inhibition is especially relevant for the accuracy of the spiking network model when the same network is trained to perform/learn multiple tasks; but it has high-impact in reducing the required computation. Embodiments may successfully work with “low-resolution data” because of the double encoded nature of information: the information is both spatial and temporal. Embodiments may exhibit a behavior similar to supervised learning algorithms.

Spatio-temporal firing unit localization is a novel method that aims to detect when and where a neuronal unit fired. The method relies on knowing the geometry of the probes, the exact location of the recording electrodes, and a neuronal network model that can be tuned to reflect an actual biological neural network as recorded in vitro.

Embodiments of BrainOS may include a no-code drag-and-drop AI user interface that will provide different types of users (patients, doctors, neuroscientists) that are unfamiliar with AI tools to do AI data analytics without the pain of having to code or taking a deep dive in AI. BrainOS may integrate different data channels, such as KIWI action potential measurements, EEG, MRI, and IoT for monitoring Activities of Daily Living (ADL). Data display types and formats may include Tabular, Images, EEG, and integration with FCU.

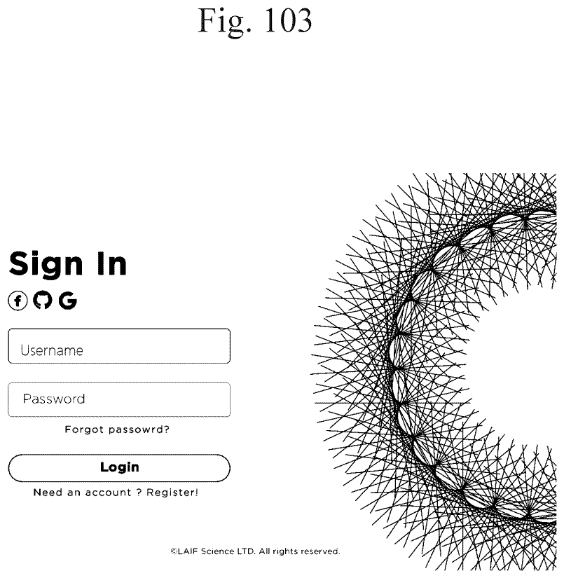

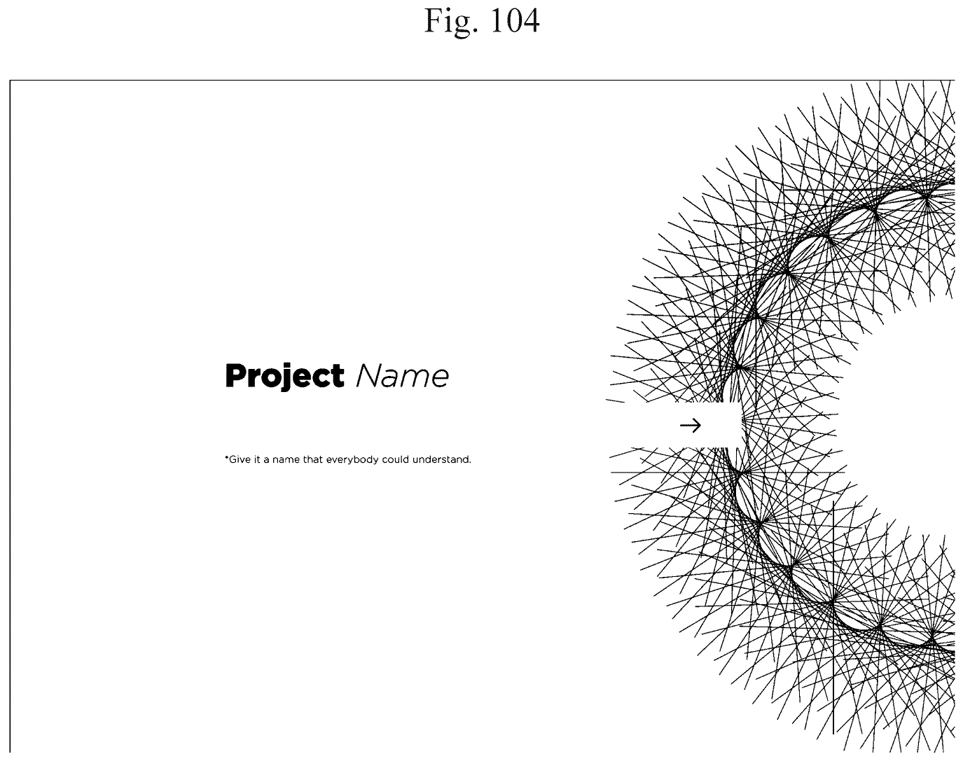

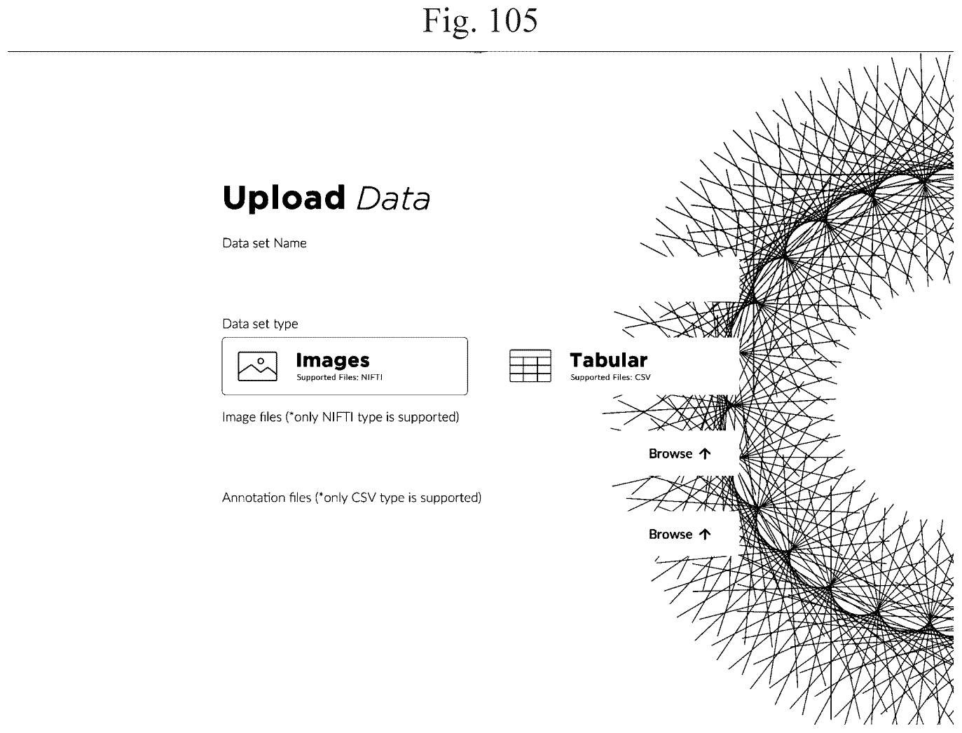

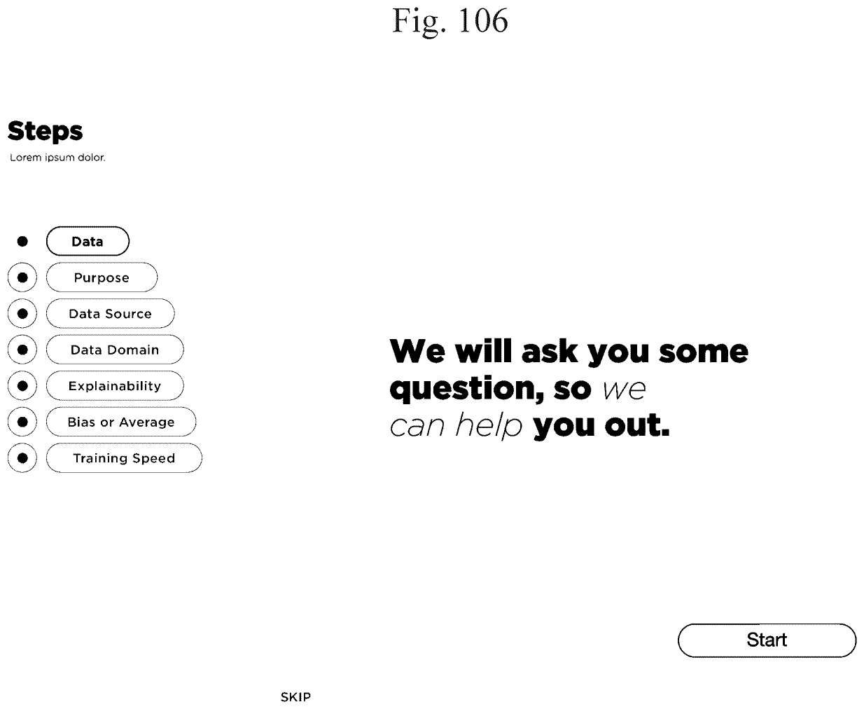

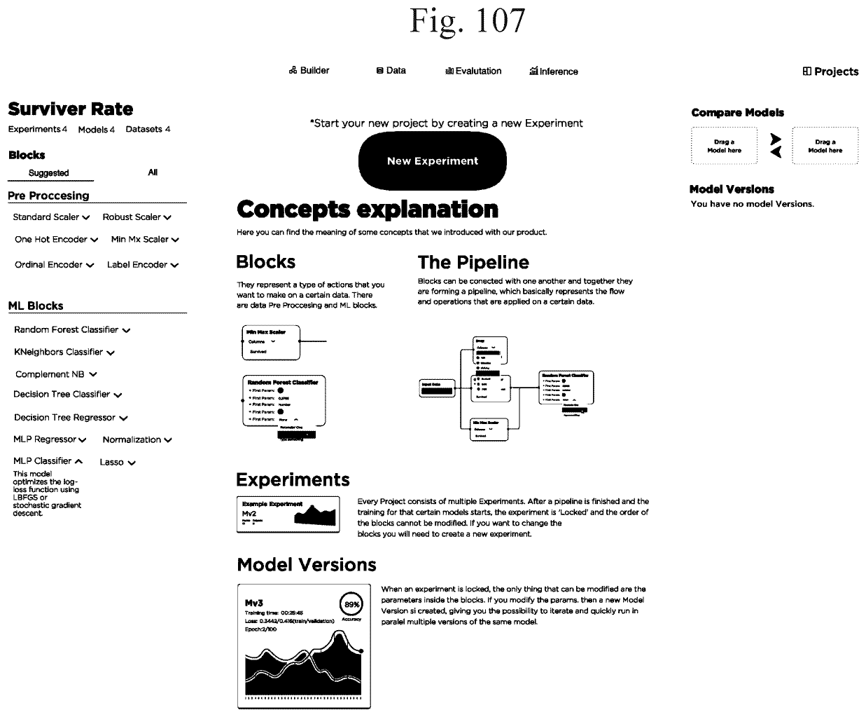

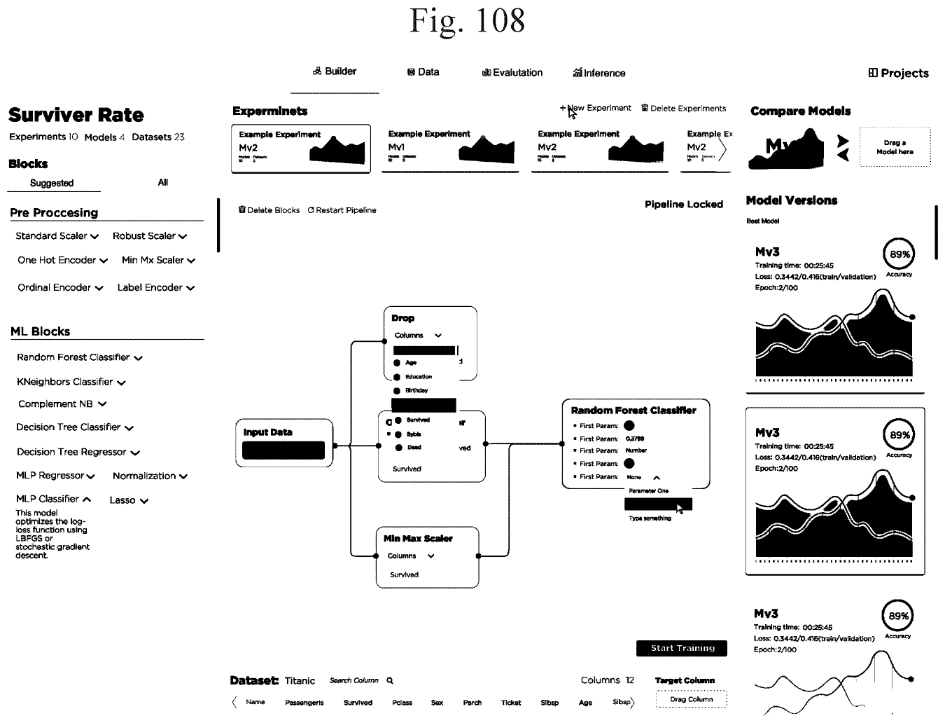

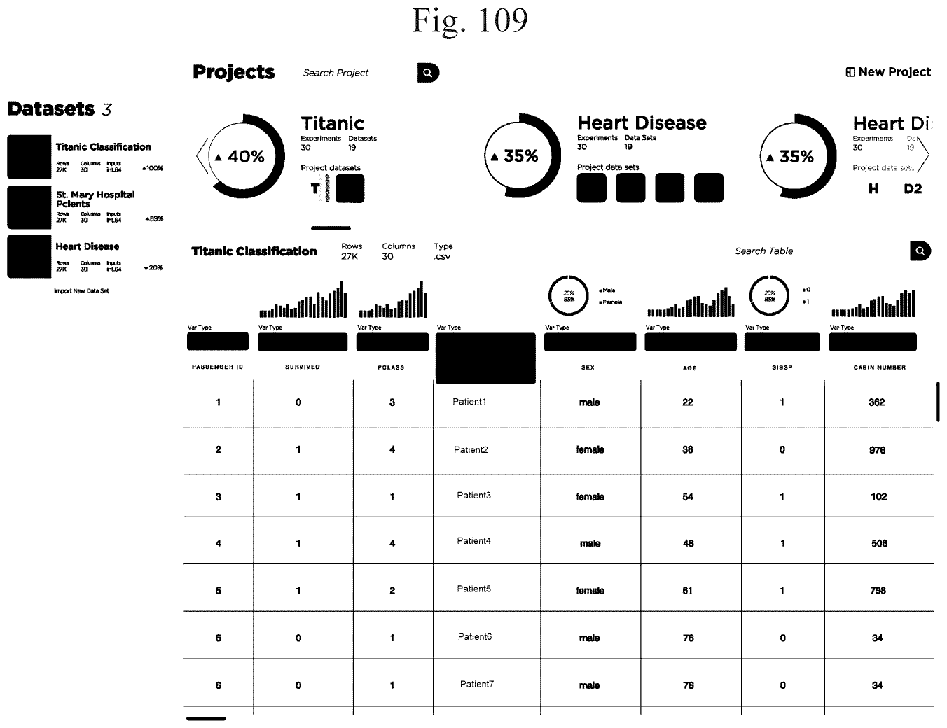

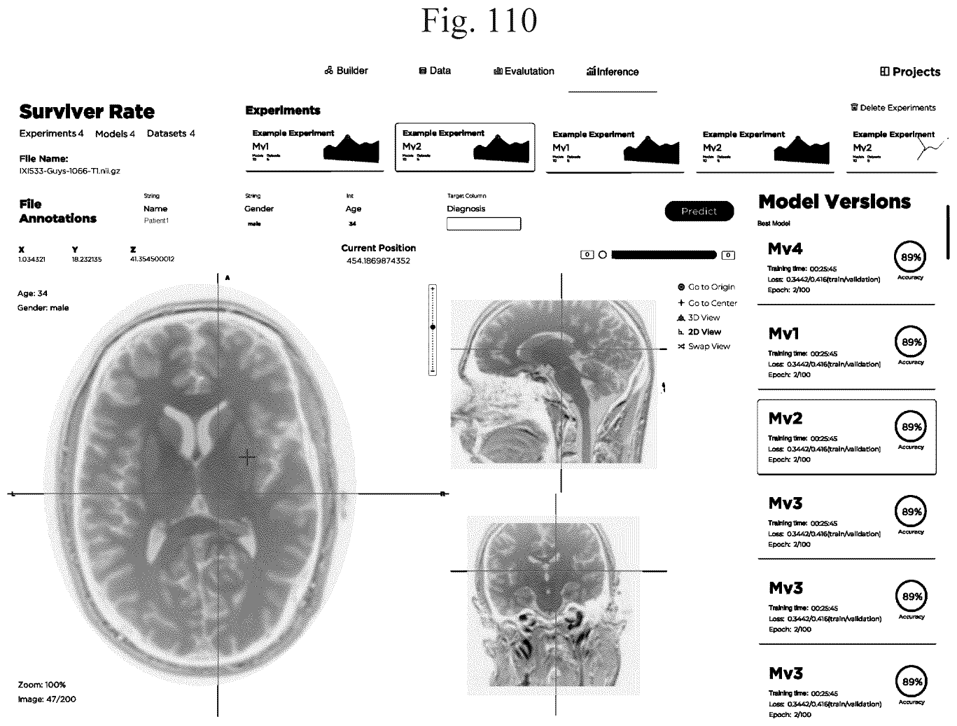

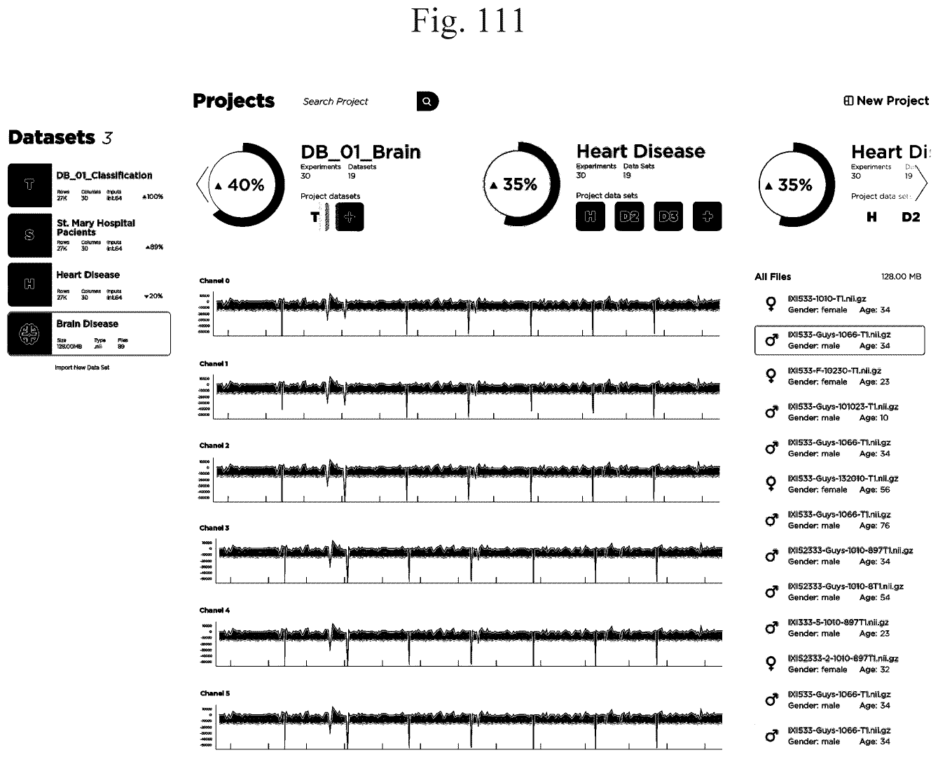

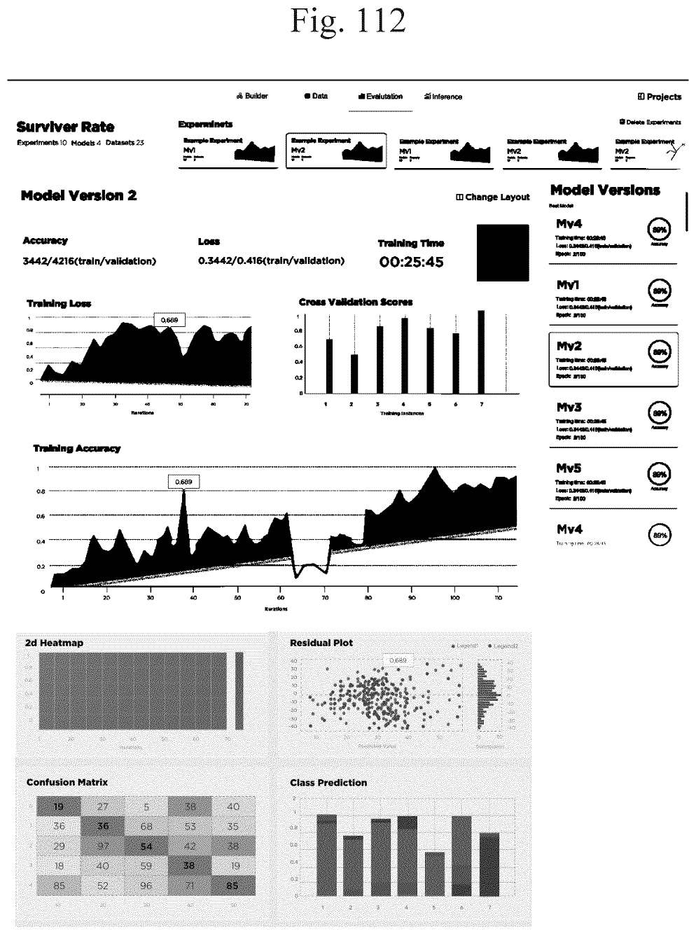

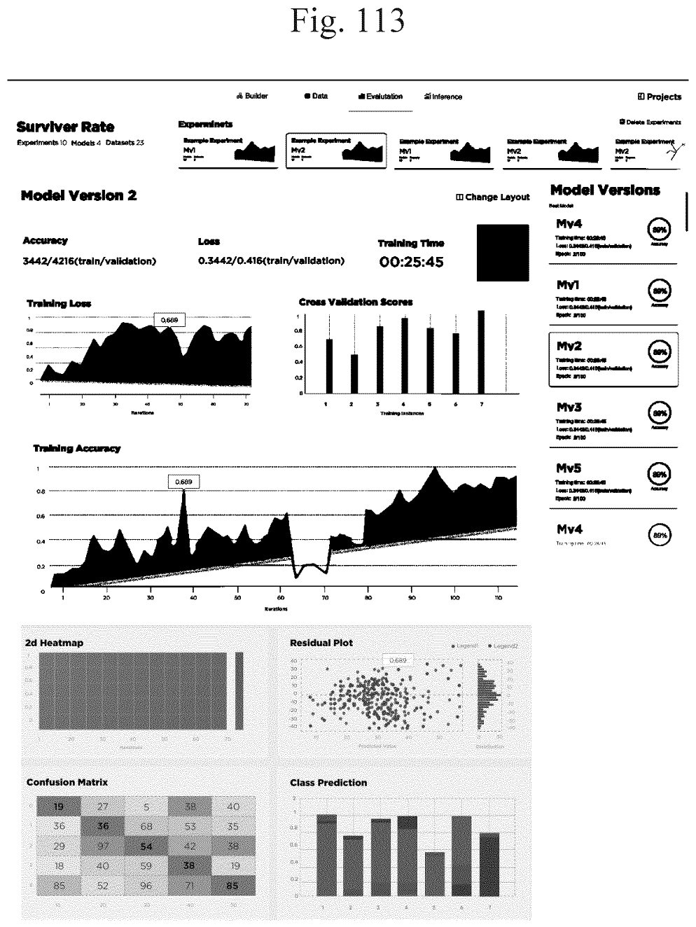

FIGS. 103-113 illustrate examples of a no-code drag-and-drop AI user interface. For example, FIG. 103 illustrates an example of a sign-in interface. FIG. 104 illustrates an example of a project name definition interface. FIG. 105 illustrates an example of a data upload interface, for example to upload Neuroimaging Informatics Technology Initiative (NIfTI) files that may include brain imaging data obtained using Magnetic Resonance Imaging methods, as well as tabular data, such as CSV files. FIG. 106 illustrates an example of a directed project definition interface, which may obtain information to define, for example a purpose of the AI project, a data source for the AI project, a data domain for the AI project, explainability parameters for the AI project, a bias or average for the AI project, a training speed for the AI project, etc. FIG. 107 illustrates an example of an experiment definition interface, which may provide the capability to set up and define experiments to be performed. FIG. 108 illustrates an example of an experiment data analysis interface, which may provide the capability to define data analysis and evaluation of dataset, such as data from defined experiments. FIG. 109 illustrates an example of a projects overview interface, which may provide information relating to and access to a plurality of projects. FIG. 110 illustrates an example of a data analysis interface, which may provide the capability to examine particular data, such as MRI imaging data, as well as other data. FIG. 111 illustrates an example of a data analysis interface, which may provide the capability to examine particular data, such as EEG data, as well as other data. FIG. 112 illustrates an example of a results analysis interface, which may provide the capability to examine results of AI training, such as accuracy, loss, time, etc. FIG. 113 illustrates an example of a results analysis interface, which may provide the capability to examine results of AI training, such as accuracy, loss, time, etc.

Data Processing. In embodiments, there may be many approaches for data processing and pre-processing. The methods used for this phase may depend on the type and state of the data that is to be processed and on the specifics of the task the system needs to solve. Examples of such processing may include Normalization, Standardization, Mean Removal, Filtering (ex. High/Low Pass), Artifact Rejection, Epoch Selection, Feature Extraction, Data Cleaning, Data Transformation, Image Segmentation, Image Augmentation, Image Enhancement etc.

Optimization Techniques. In embodiments, each model may have its own specific optimization aspects that may be handled. Examples of such optimization may include Optimizing Hyperparameters, such as Hill Climbing (Random Restart), Simulated Annealing, Genetic Algorithms, MIMIC, MCMC, Expectation Maximization, and Grid Search, as well as Gradient Descent Optimization, Stochastic Gradient Descent Optimization, Adaboost, Memento etc. In embodiments, these optimization techniques may be modified or customized. Likewise, other optimization techniques may be utilized.

User Interface. In embodiments, the Cloud User Interface (UI) may have, for example, three different types of users, each of which may have different capabilities.

Patients. In embodiments, the UI for the patients may be focused on data visualization. They may be able to see real time activity as it comes in from the implant device.

Patients may also be able to select from a list of stimulation commands that were prescribed by the doctor. These commands may be either based on their current activity (sleep, walk, etc.) or based on their physiological state (tremors, inability to focus, etc.). Patients may also be able to annotate certain time segments with activities they were involved in during that time span to indicate, for example, when they were doing physical activities, mental tasks, etc.

Doctors. In embodiments, doctors may be able to access individual patient data. For each patient, they may have the option to apply different predefined machine learning models —presented as software-based prescriptions—in order to determine the best treatment going forward. Doctors may be able to configure the implant device, based on the output of the previous models. They may be able to set different modes of operation for the implant device, and change its recording/stimulation parameters. They may also be able to visualize the data of the patient in different ways, and flag certain patients for detailed analysis from neuroscientists.

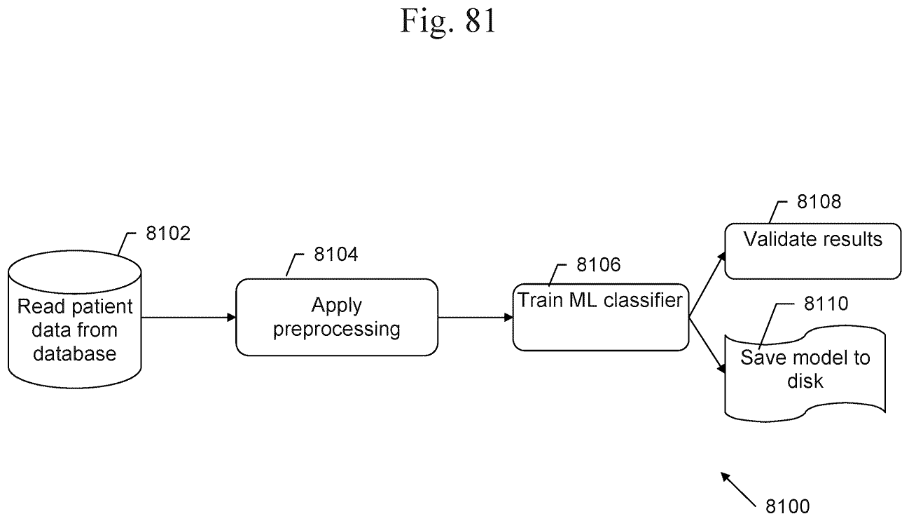

Researchers. In embodiments, researchers may compose pipelines to process the data from many patients. An example of a general description of such a pipeline 8100 is shown in FIG. 81. In this example, pipeline 8100 may include reading patient data from a database 8102, processing the data 8104, training a machine learning classifier model 8106, validating the results 8108, and saving the trained model to storage 8110, such as disk.

Visualization Interface. In embodiments, the system may interface with tools such as EEGLAB™, which is a widely used neuroscience package for MATLAB™ or GEPETTO™ which can be used to visualize neurons, in order to provide Visualization Interfaces with which researchers are already familiar. In embodiments, examples of visualization methods may include Scalp Maps, ERP Images, Line Charts, Neuron Visualizations, Data Statistics, etc.

Security. Given the medical nature of the data handled by the system, great care must be taken to avoid any unauthorized access to the data or any commands sent by unauthorized agents. Accordingly, embodiments may provide secure communications, secure streaming, secure access, and secure storage. For example, providing secure communications may include ensuring that all the RPCs (Remote Procedure Calls) issued between the various microservices that make up the system are encrypted using the latest SSL encryption standards. In embodiments, data that is streamed from the Gateway may also be encrypted, to prevent tampering and snooping. In embodiments, secure access may be provided by an Identity and Access Management layer, which may give permissions to each actor to access and execute only user specific data and commands. For example, patients should be able to view only their own data and send to the implant device commands that have been authorized by a doctor, doctors should be able to only view the full data of their patients, use pretrained models to prescribe new software-based treatments for their patients and send commands to their patients' implant devices. In embodiments, researchers should have access only to anonymized patient data that they can use for deriving new scientific insights using the AI Research Interface provided in the Cloud environment. In embodiments, to prevent unauthorized physical access in data centers and provide secure storage, the data may be stored with encryption.

Consistency & Durability Requirements. In embodiments, there are a variety of aspects that may be considered in terms of system availability, consistency, and fault tolerance. For example, issues such as location, data consistency, maintenance, and backups may be considered.

Location. In embodiments, the cloud servers may be placed in a single region or in multiple regions. Multiple regions may mean higher availability due to outages that take out a single region, but comes at higher cost and higher system architecture complexity.

Data consistency. In embodiments, data may be stored in multiple copies to reduce the chance of one outage leading to the deletion of all the data. In embodiments, the choice may be between consistent availability, meaning that all the data is the same all the time and everywhere, at the cost of higher latency, or eventual availability, which means that depending on where the data is read from, different information might be returned.

Maintenance and DevOps. In embodiments, there may be a tradeoff to be made between running the system on premises or on public cloud providers such as AMAZON WEB SERVICES™, GOOGLE CLOUD PLATFORM™ or AZURE™. This is because of different costs, maintenance work and infrastructure development. Considering the requirements for scaling up, public clouds may become cost-prohibitive, so they may be replaced with private hosted clouds, such as KUBERNETES™, or specialized clouds.

Backups. In embodiments, in order to ensure that data is not lost in case of system failure, regular backups may be done. They may happen at several levels. For example, data may be stored redundantly at the datacenter levels—to prevent loss due to individual machine failures. Likewise, data may be regularly copied to an offsite storage—to protect against geographic catastrophes.

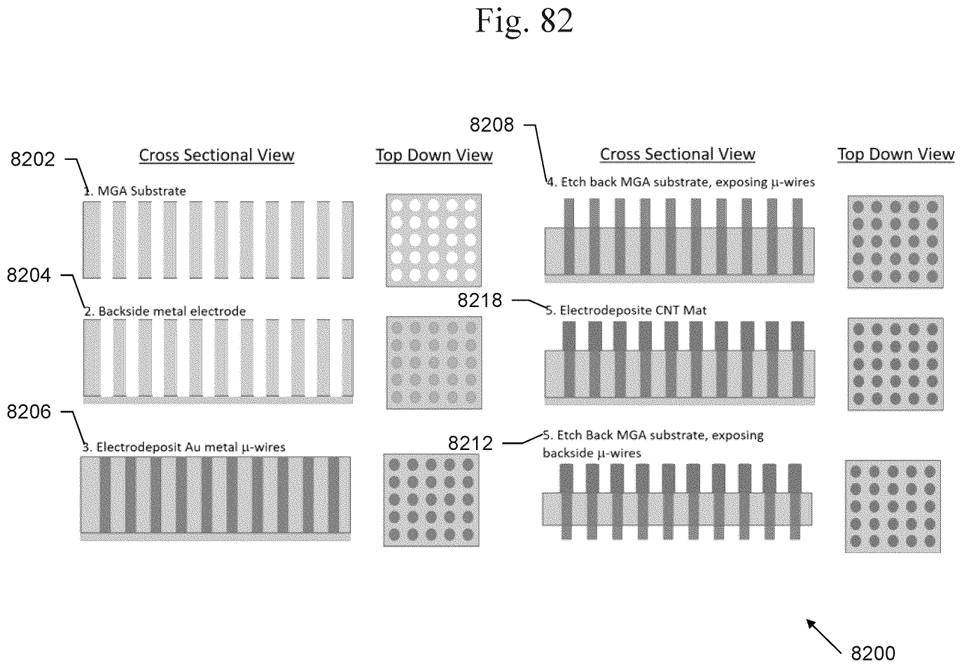

An example of a process 8200, which is of a portion of a process of fabrication of CNT implant devices, is shown in FIG. 82. In this example, a microelectrode array of connections between electronic readouts and in-vivo human neural tissue may be fabricated. Using electroplating as a deposition technique, a CNT-based microelectrode array may be formed through a 1-mm thick micro-channel glass array (MGA) substrate. In an embodiment, the electrode arrays may have CNT contacts on the front side, and metal contacts on the back. In an embodiment the electrode arrays may have metal contacts on both sides. Embodiments may include arrays with CNT contacts on the front side, and metal contacts on the back, and arrays with metal contacts on both sides.

Process 8200 may begin with 8202, in which an MGA substrate may be formed. At 8204, metal electrodes may be formed on the backside of the MGA substrate. At 8206, gold micro wires may be electrodeposited on the metal electrodes in the micro channels of the MGA substrate. At 8208, the topside of the MGA substrate may be etched to expose the gold micro wires. At 8210, the CNT material may be electrodeposited onto the exposed gold micro wires. At 8212, the backside of the MGA substrate may be etched to expose the backside gold micro wires.

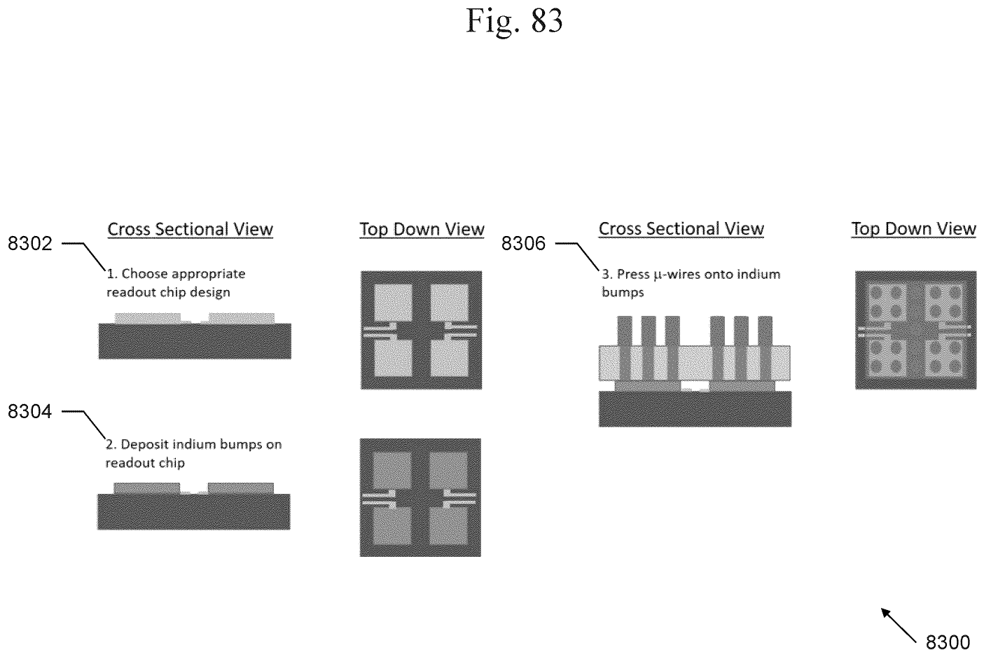

An example of a process 8300, which is of a portion of a process of fabrication of CNT implant devices, is shown in FIG. 83. In this example, the MGA/CNT-based microelectrodes may be hybridized to an electrical readout chip providing for a parallel neural-electronic interface to the brain. Process 8300 may begin with 8302, in which an appropriate readout chip design may be selected. At 8304, metal bumps, such as indium, may be deposited on the contacts of the readout chip. At 8306, the micro wires that were exposed on the backside at 8212 in FIG. 82 may be pressed onto the metal bumps, creating electrical contact with the readout chip.

Embodiments may be manufactured using, for example, a Silicon (Si) (1-1Ω) wafer. On the front of the Si wafer CNTs or graphenes may be grown, while on the back Aluminum may be deposited. The electrode contact may be achieved through the metallization of the wafer on its back side.

Graphenes can be grown directly on Si. The thickness (height) of the graphene film can start from several hundreds of nano-meters to 1-2 micron or more. CNTs may be grown on a thin film of titanium nitride (TiN). In this case, on the front of the silicon wafer a ˜50 nm TiN film may be deposited followed by the CNTs growth process. Embodiments may include CNTs of several microns in length. In embodiments, graphene or CNT electrodes may be as long as 100 microns in length, or longer. In embodiments, individual electrodes may have a variable opening between 1 um and 25 um. In embodiments, the silicon wafer may be processed to create dies with a size of 50 um×50 um or larger. For example, the wafer may be partitioned into a matrix of squares that has a side length of 1 mm, placing the electrodes structure in the center of each square, with a variable geometry. Given the metallization on the back of the wafer, in embodiments, the die may be directly glued on a conductive surface (PCB, Kapton ribbon with metallic deposition etc.) which may permit integration to any type of connector. The gluing can be done using, for example, Silver paste.

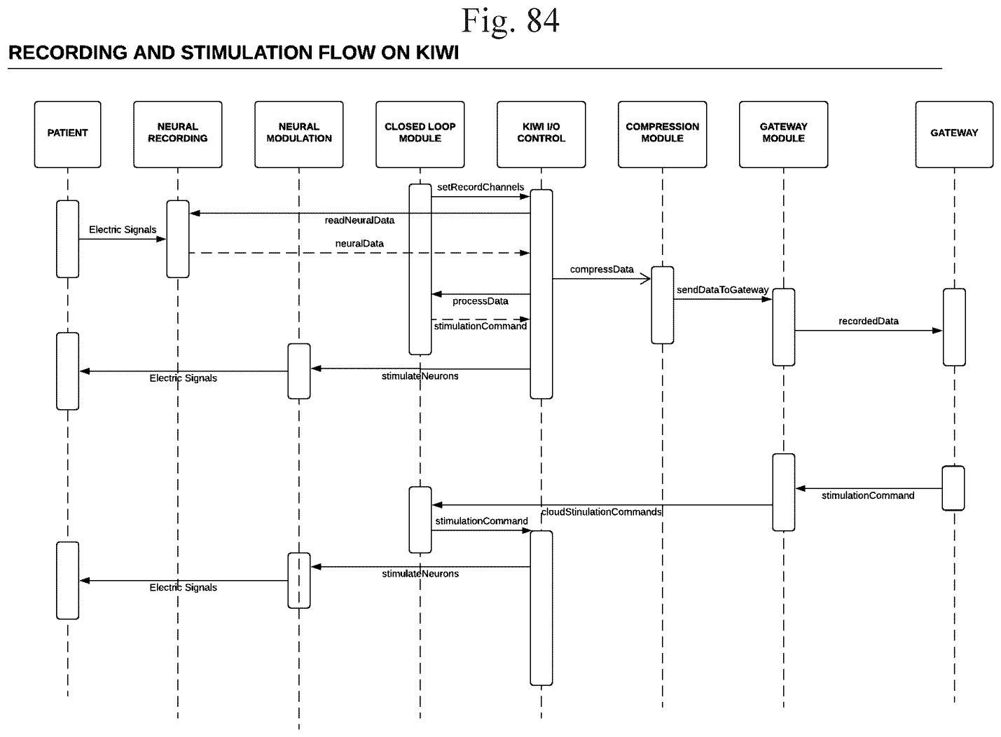

An example of a recording and stimulation signal and data flow on an implant device is shown in FIG. 84.

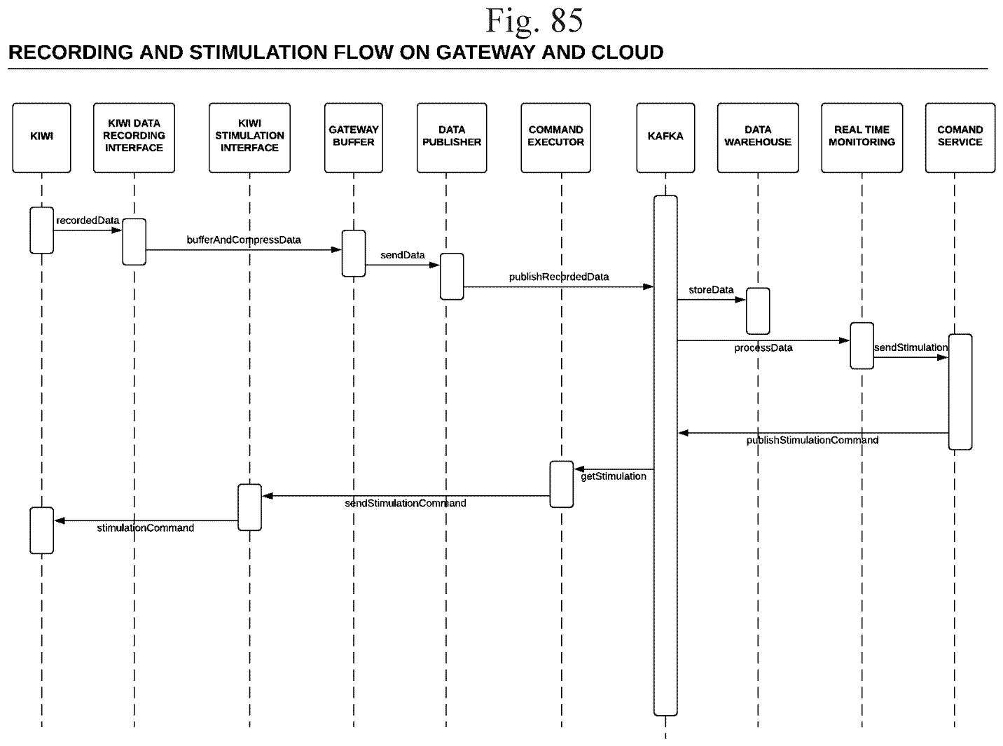

An example of a recording and stimulation signal and data flow on the Gateway and Cloud is shown in FIG. 85.

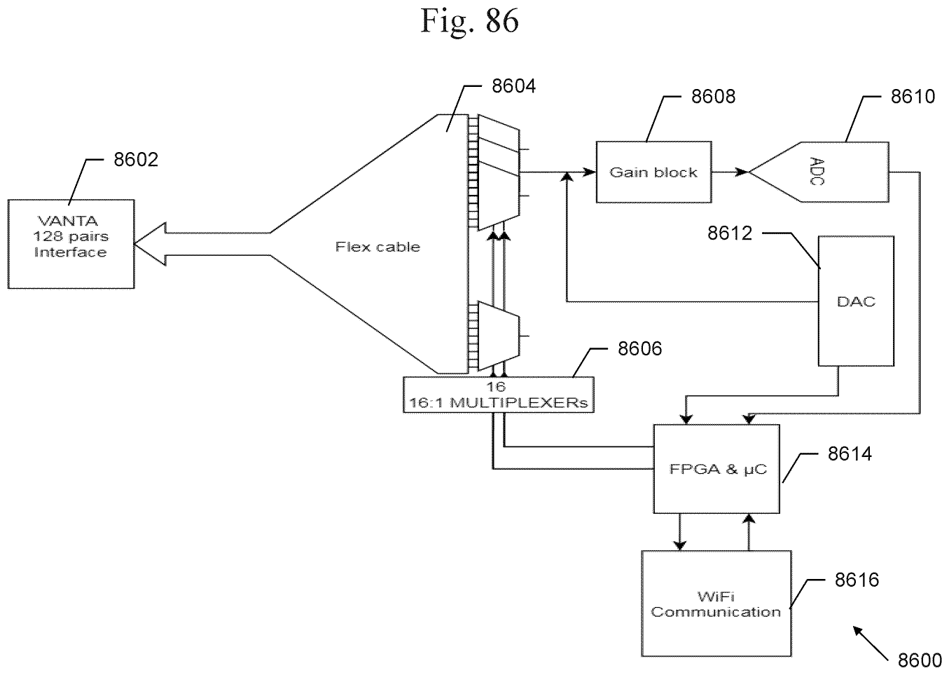

An exemplary block diagram of an embodiment of an implant device electrical system 8600 is shown in FIG. 86. In this example, system 8600 includes Vertically Aligned NanoTube Array (VANTA) 8602, cable 8604, analog multiplexers 8606, gain block 8608, ADC 8610, DAC 8612, control/processing circuitry 8614, and Wi-Fi communication circuitry 8616.

In embodiments, VANTA 8602 may include an array of vertically aligned nanotubes, as discussed above. Cable 8604 may connect VANTA 8602 to electronic circuitry, such as multiplexers 8606. In embodiments, cable 8604 may include a double layer flex cable, to connect the VANTA to the Analogue Front-end. Flex circuits offer the same advantages of a printed circuit board—repeatability, reliability, and high density—but with the added features of flexibility and vibration resistance.

In embodiments, the amplitudes of the analog signals may be adjusted by gain block 8608, which may include a plurality of amplifiers, one for each ADC. In embodiments, a plurality of ADCs 8610 may be multiplexed to a plurality of signals from VANTA 8602 by multiplexers 8606. The switching speed of multiplexers 8606 may be faster than the sampling frequency of ADCs 8610 by the number of the probes divided by the number of ADCs. Accordingly, in embodiments, the multiplexing frequency may be given by Fmux=CEIL(128 probes/16 ADCs)*3 kHz=24 kHz. The switching is fast enough so that the time taken to do a full scan of all the multiplexed channels would not significantly affect the measurement of the channels.

In embodiments, the ADC conversion may be triggered by the measured potential crossing a set threshold. As soon as the triggered ADC conversion starts, the adjacent ADCs may also be triggered.

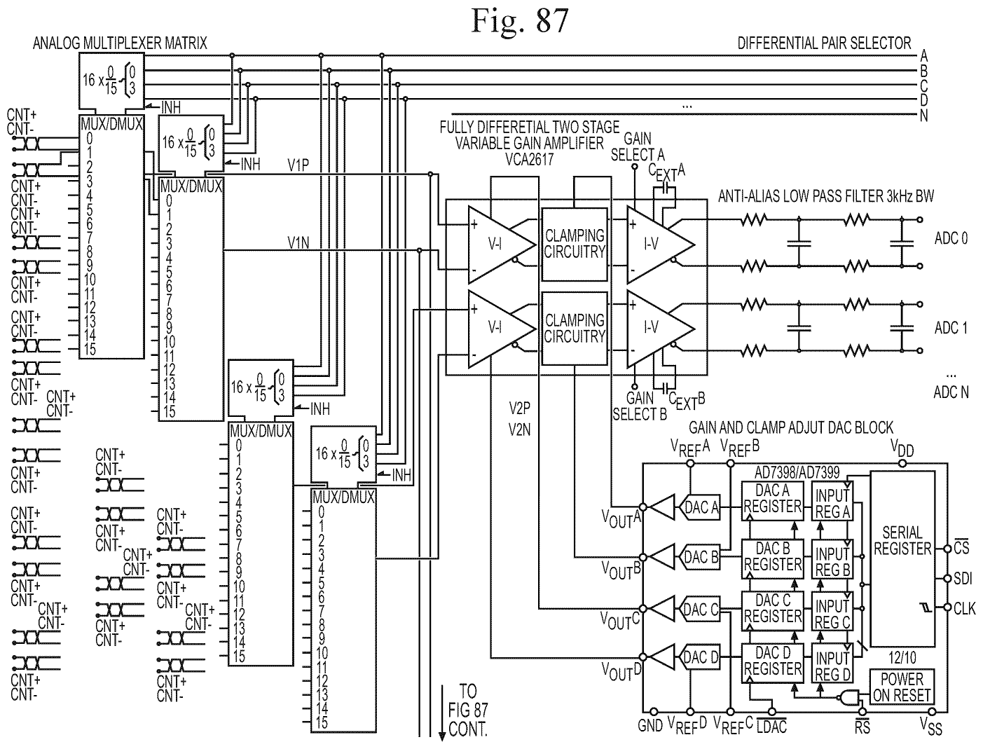

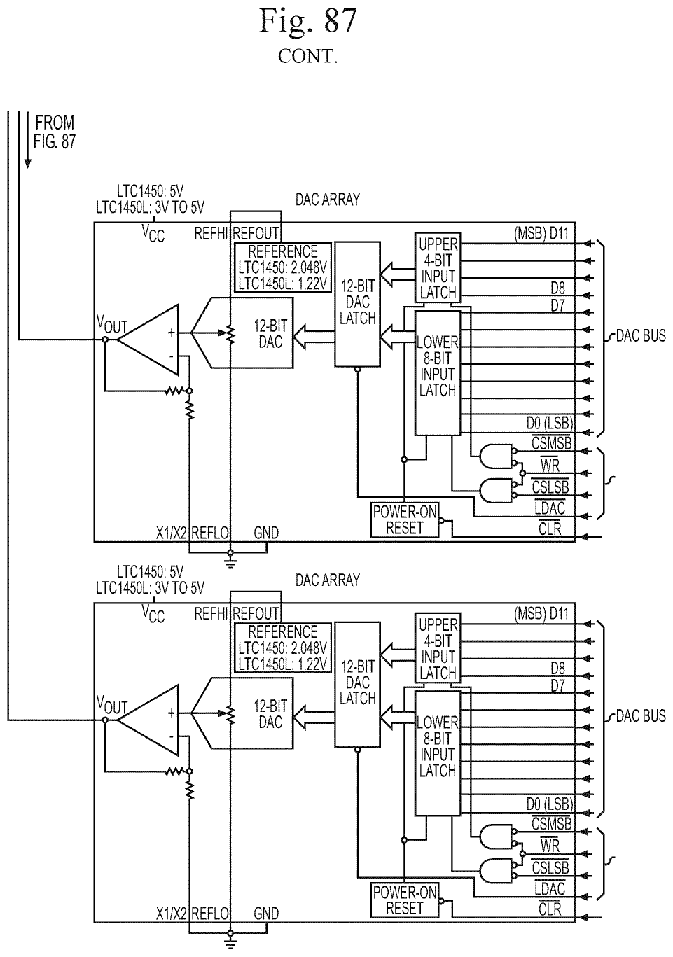

In embodiments, in order to increase the Signal to Noise Ratio (SNR) and acquire position data of action potential source, several ADC measurements may be taken simultaneously, in a grid formation. The grid dimensions may be dependent on probe spatial density. An example of a 4×4 probe multiplexer distribution is presented in FIG. 87. All the squares with the same number represent probes which share the same Amplifier and ADC through a multiplexer. The probes may be connected to multiplexers in such a way that, no matter which ADC is being triggered, no adjacent probe shall be multiplexed to the same channel.

After a 3×3 ADC grid is acquired (the grid containing the triggered channel and the surrounding 8 channels), the results may be processed by control/processing circuitry 8614. Control/processing circuitry 8614 may include a microcontroller or other computing device, as well as hardware processing functions, which may be implemented, for example, in an FPGA or ASIC. Such hardware processing may perform, for example, multiplication to increase SNR, weighting to accurately place the signal source, etc.

An exemplary embodiment of a portion of an implant device electrical system is shown in FIG. 87.

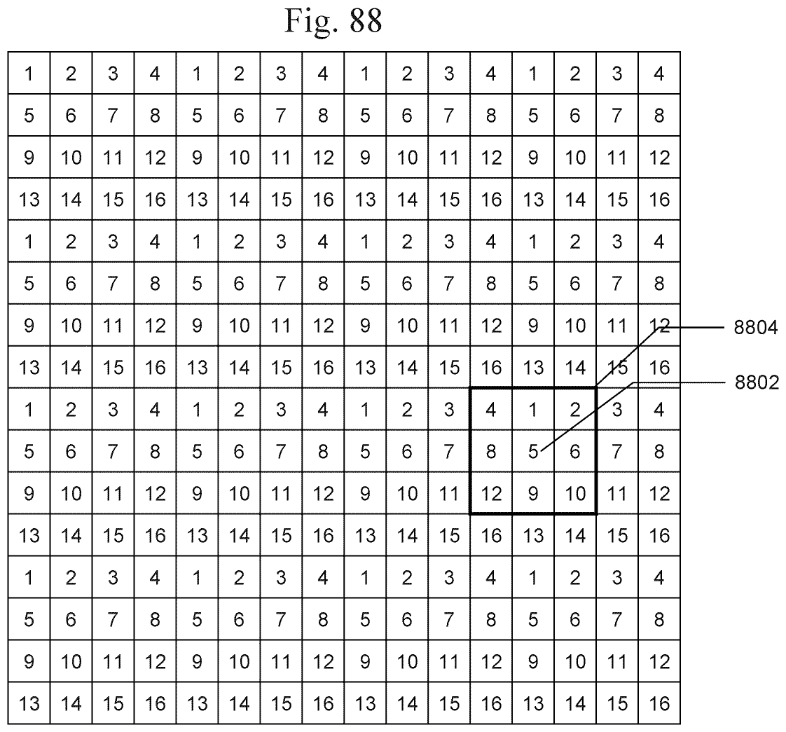

For example, as shown in FIG. 88, the action potential may fire in square 8802 with and may cross the set threshold. As a result, the corresponding ADC and all the adjacent ADCs 8804 may be triggered. Because the maximum length of an action potential is about 5 ms, all 9 ADCs may obtain samples for that time. The resulting data may be processed in control/processing circuitry 8614. For example, the signals may be multiplied to increase SNR. At the same time, based on the signal intensity, a point may be placed on the calculated position with the highest potential—spatial resolution depends on the number of channels sampled.

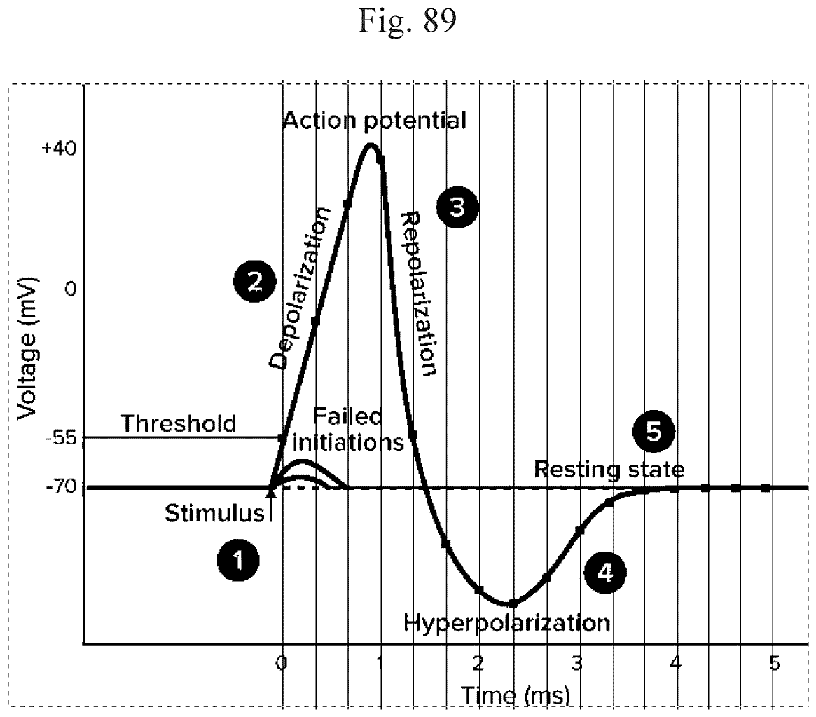

An example of the triggering of the first ADC and the quantization of the action potential is illustrated in FIG. 89 for a 3 kHz sampling rate. For a 5 ms long spike, the curve may be described by 16 points and model-based reconstruction of the signal may be used on the recorded data. In embodiments, the reading sampling rate may be increased, up to, for example, about 96 kHz, with increased power consumption.

An exemplary block diagram of multiplexer connections 9000 for two pairs of differential probes 9002, 9004 is shown in FIG. 90. Notice that the positive and negative probes are each connected to different multiplexers 9006, 9008 for simultaneous availability. As the DAC is enabled, the ADC is disabled for the same pair, allowing the reuse of the same multiplexer.

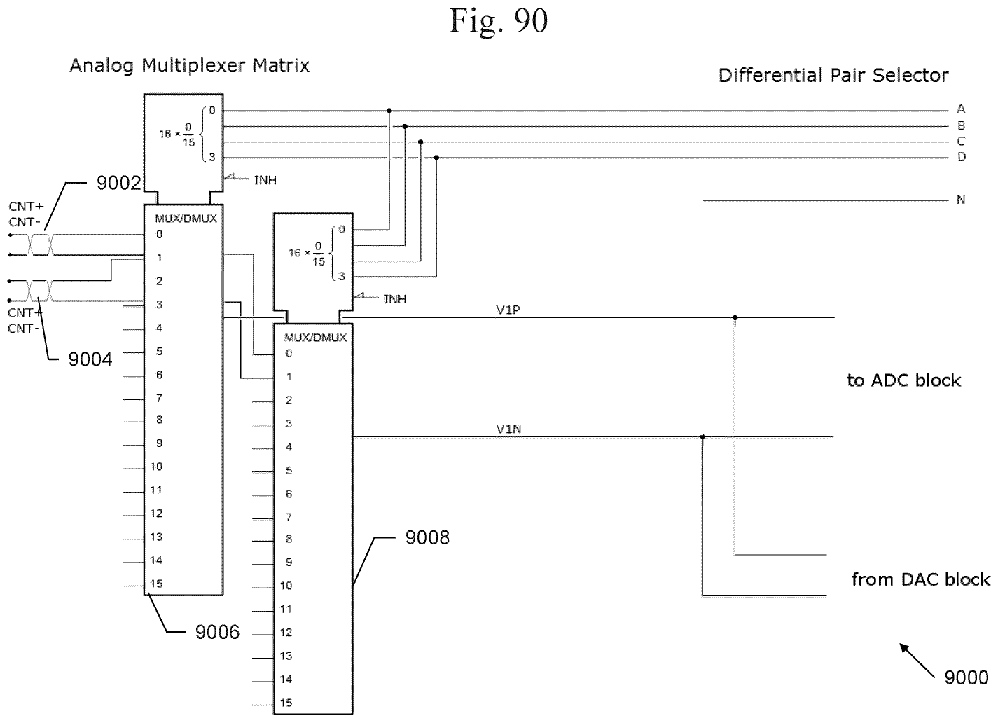

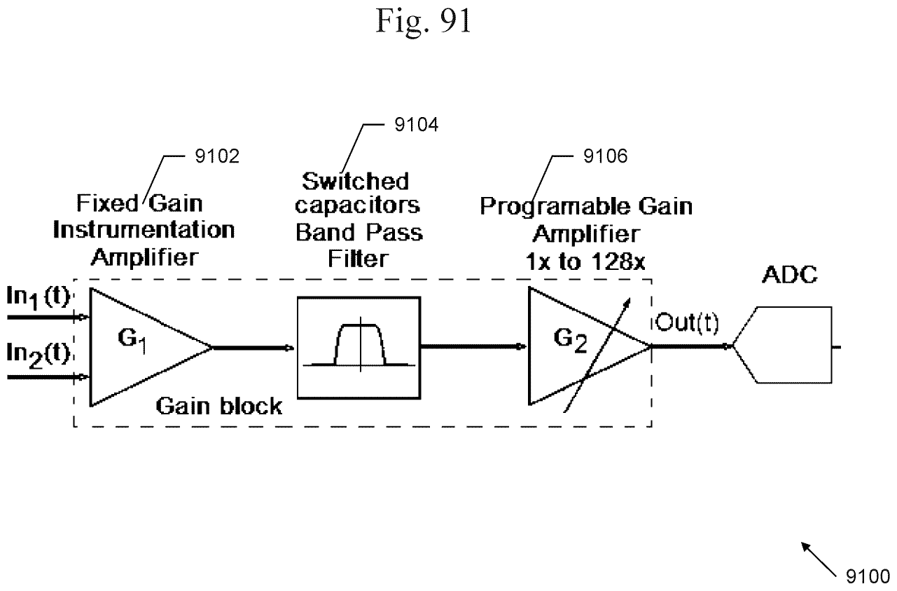

In embodiments, for recording, the signal from the multiplexer may be amplified using a Gain Block 9100, such as the example shown in FIG. 91, before being input to the ADC sampling unit. In embodiments, the First Amplifier Stage may include a differential input fixed gain instrumentation amplifier 9102. This design, while not adding much complexity, may be characterized by a low noise figure and a high common mode rejection ratio. It also doubles as an input driver with a very high input impedance, reducing load on the signal. In embodiments, amplifier stage 9102 may be followed by a switched capacitor bandpass filter of, for example, 3 kHz, to filter out the MUX switching noise. In embodiments, the Second Amplifier Stage may include a variable gain amplifier 9106 having a gain range of, for example, 1 to 128. The gain of amplifier 9106 may be programmable using, for example, a Gain and Clamp Adjust DAC Block, which may correct for clipping caused by probe-neuron distance variation.

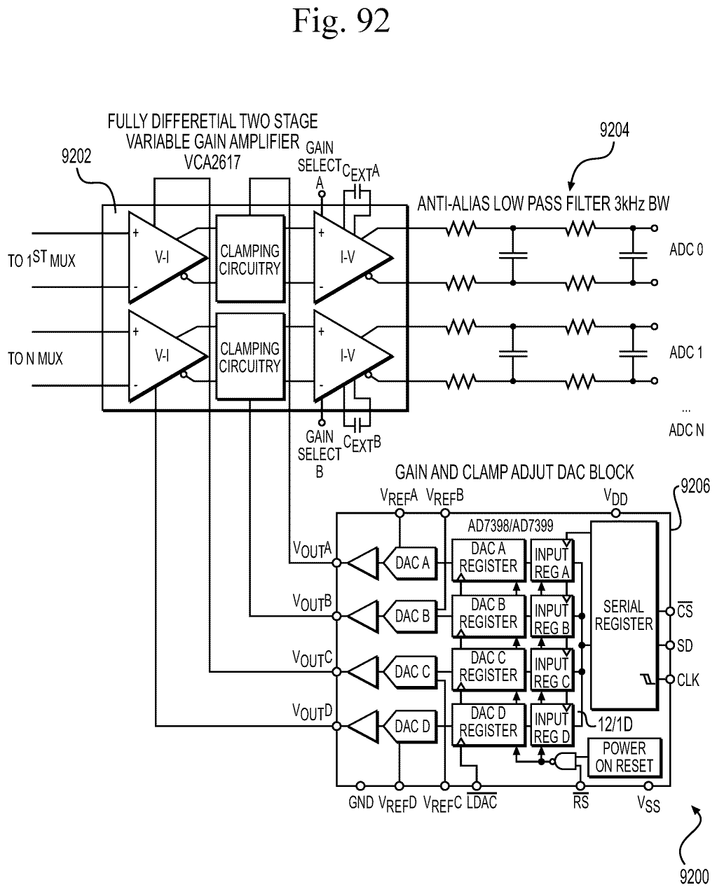

An exemplary block diagram of a Gain Block 9200 is shown in FIG. 92. In this example, Gain Block 9200 may include a differential two stage variable gain amplifier 9202, such as the VCA2617 from TEXAS INSTRUMENTS®, low pass anti-aliasing filter 9204 having a bandwidth of, for example, 3 kHz, and a gain and claim adjustment block 9206, such as the AD7398/AD7399 from ANALOG DEVICES®. In this example, amplifier 9202 may be continuously variable, voltage-controlled gain amplifier. Adjustment block 9206 may accept digital data to control DACs and output voltages to control the gain and clamping of amplifier 9202. Low pass filter 9204 may, for example, be implemented using passive components and may be used to restrict the bandwidth of signal before being sampled by the ADCs.

In embodiments, in order to measure a total of 128 differential probes, a compromise may be found between a high enough number of simultaneously sampled channels, for good signal characteristic, and a low number of ADCs, for space saving considerations. In embodiments, a 3×3 grid may be used, requiring a total of 9 triggered ADCs.

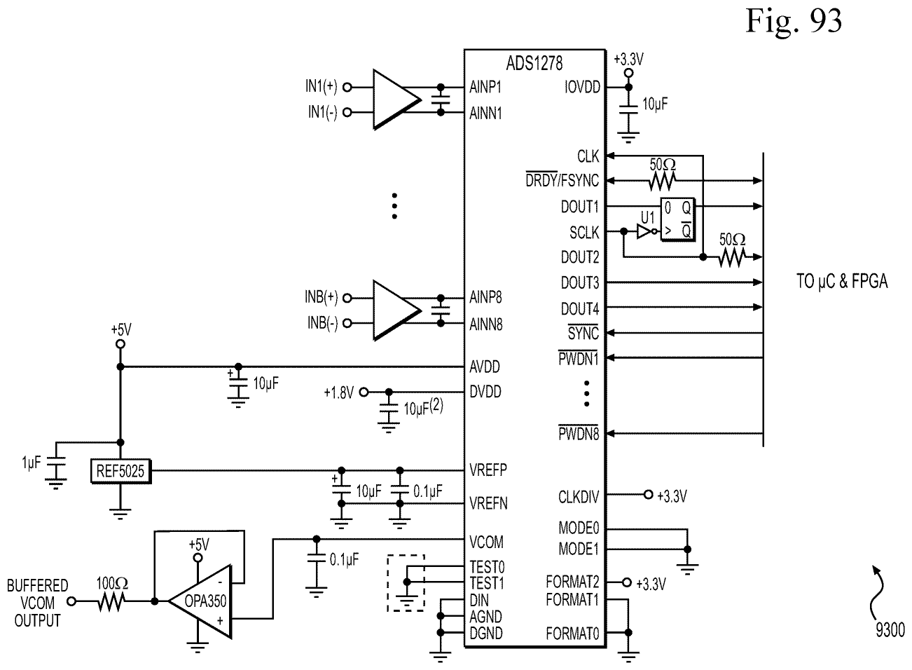

In embodiments, an ADC 9300, an example of which is shown in FIG. 93, such as the ADS1278 from TEXAS INSTRUMENTS®, may be used. In this embodiment, each ADC device may have 8 simultaneous sampling channels, thus, two ADS1278 devices may be used for a total of 16 simultaneous measurements. After multiplexing each ADC channel to 8 differential probes, the total 128 necessary measurement channels may be obtained. It is to be noted that the ADS1278 is a high precision 24-bit ADC with a high-power consumption. Given that the signals are repetitive in nature, embodiments may only need 10 bits of ADC precision for the encoding of the action potential signal. Accordingly, other ADCs having lower precision and lower power consumption may advantageously be utilized in embodiments.

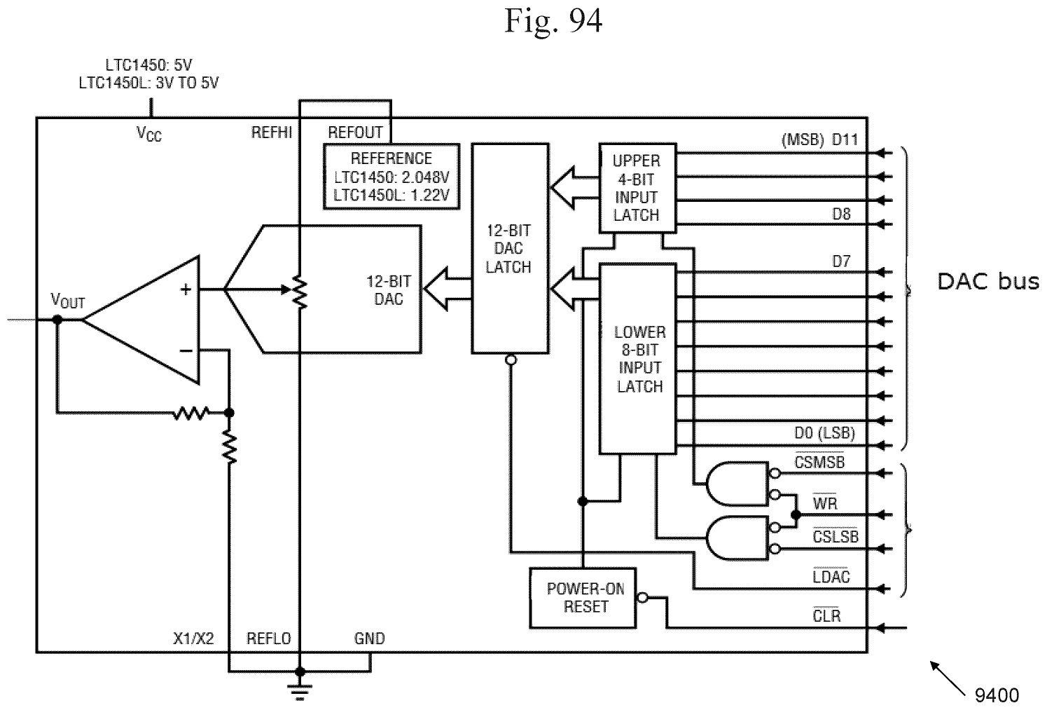

In embodiments, DAC Block circuitry 9400, an example of which is shown in FIG. 94, such as the LTC1450/LTC1450L from ANALOG DEVICES®, may be used for electrical stimulation of the neuronal tissue through the CNTs. DAC Block 9400 may include an array of high resolution DACs. The stimulation circuit may be able to generate multiple arbitrary waveforms. In embodiments, the DACs may interface with control/processing circuitry 8614 using a parallel or serial architecture in which all DACs are sharing the same data bus.

In embodiments, each DAC may have a Load Data Signal Line used for data output register update. The control/processing circuitry 8614 may load sample data into each DAC. After all the data has been uploaded, a single Load Data Line Toggle may set the analog output of the DAC at the desired values.

For example, consider 8 discrete signals having 256 samples stored as a matrix: stimulus_name[DAC_resolution][sample]. In this example, a write process may include loading a first sample of each stimulus into a corresponding DAC, toggling all Load Data Lines simultaneously and updating DAC output voltages, loading the next samples repeatedly until the stimulus signals have been generated, and setting the output channels to high impedance.

In embodiments, due to the quantization levels of the DAC, the output voltage may be affected by slight transitions. In order to clean up the signal, a low pass filter may be inserted at the DAC outputs.

In embodiments, operational modes for the Closed Loop Process may include Sequential Reading and Stimulation and Simultaneous Reading and Stimulation. The Sequential Reading and Stimulation mode may share the same Mux/Demux block between ADCs and DACs. This method may reduce design complexity, but cannot stimulate and read the neuronal activity in different locations of the tissue at precisely the same time.

The Simultaneous Reading and Stimulation mode may use a plurality of Mux/Demux blocks for ADCs and DACs. The high impedance of the ADC inputs and the Gain Block will not affect the stimulation. In embodiments, this architecture may stimulate the neuronal activity in a certain location and measure the response signal in an arbitrary location. There may be the need to set two different Mux/Demux addresses: one for stimulation and one for impulse response.

In embodiments, with use of the Multiplexing Pattern described above, the shortcomings of the first operation mode are alleviated, as there will be no two simultaneous writes in the same 4×4 cell.

In embodiments, control/processing circuitry 8614, shown in FIG. 86, may include a microcontroller or other computing device, as well as hardware processing functions, which may be implemented, for example, in an FPGA or ASIC. For example, in an FPGA implementation a SPARTAN-7® FPGA from XILINX® may be utilized. In another example, an IGLOO NANO® from MICROSEMI® may be used.

In embodiments, control/processing circuitry 8614 may perform data acquisition from the ADCs; separation of overlapped signals; action potential recognition and sorting including finding firing patterns, isolating signals from each other, and eliminating crosstalk temporally (time window cropping) and dimensionally (close signal multiplication); creating a perceived map of neurons based on signal strength and pattern recognition, thus further reducing necessary data throughput, and detecting higher-order features of the neural network.

In embodiments, control/processing circuitry 8614 may include a microcontroller or microprocessor for serialization, debugging, communication and control. For example, a single or multi-core CPU may be used. In embodiments, embedded memory, external memory, and peripherals may be located on the data bus and/or the instruction bus of these CPUs. An adequate address space, such as 4 GB, and functions such as DMA and built-in Wi-Fi may be utilized. Control/processing circuitry 8614 may be used for controlling the hardware components (MUX, ADC, DAC) and data transmission and acquisition rates.

Optical Recording & Stimulation. In embodiments, the range of radiation wavelengths for neuron stimulation may be between 380 nm and 470 nm, which may be obtained using one single LED by modulating the current characteristics. For example, a pixel density of 570 ppi (pixels per inch) for a 2×2 array (for color) will yield a pixel 22.3 microns wide. Depending on the pitch of the CNTs, the LEDs may be placed either in between the CNTs or right underneath them (the wires connected to the CNT may be run through the LED).

Optical Reading. In embodiments, if LEDs are used for optical stimulation, options for optical recording may include using the LEDs as radiation receptors to convert light into electric signals and using image sensors, such as CCD or CMOS image sensors. In embodiments, if LEDs are used as radiation receptors, the same device may be used both for optical stimulation and recording. In these embodiments, the recorded electric signal may be relatively weaker and noisier. This is an important drawback especially when the recorded signals have such small values. In embodiments, use of CCD or CMOS photodiodes may provide a stronger signal. In these embodiments, the optical reading and stimulation resolution may decrease due to the fact that these sensors have to be added in addition to the existing LEDs.

In embodiments, the circuitry may be in the form of a readout-integrated circuit (ROIC), which may be similar to or a modification of, for example, a solid-state imaging array. The ROIC may include a large array of “pixels”, each consisting of a photodiode, and small signal amplifier. In embodiments, the photodiode may be processed as a light emitting diode, and the input to the amplifier may be provided by the CNT connection to the neuron. In this manner, neurons may be stimulated optically, and interrogated electrically. The ROIC may include CCD or CMOS photodiodes or other imaging cells, to receive optical signals, electrical receiving circuitry, to receive electrical signals, light outputting circuitry, such as LED or lasers, to output optical signals, and electrical transmitting circuitry, to transmit electrical signals.

In embodiments, the light sources may be placed at the base of the CNTs, rather than using optic fibers. In these embodiments, the light does not have to be transported from the light sources to the recording site and back using an optical circuit. Exactly how many neurons may be optically reached depends on the distance between the neuronal tissue and the CNT board which in turn depends on the length of the CNTs. In these embodiments, a plastic magnifier on the LED may be used to focus the light emission. But considering the width of one LED is about 23 microns, this would be a challenging solution in terms of manufacturing.

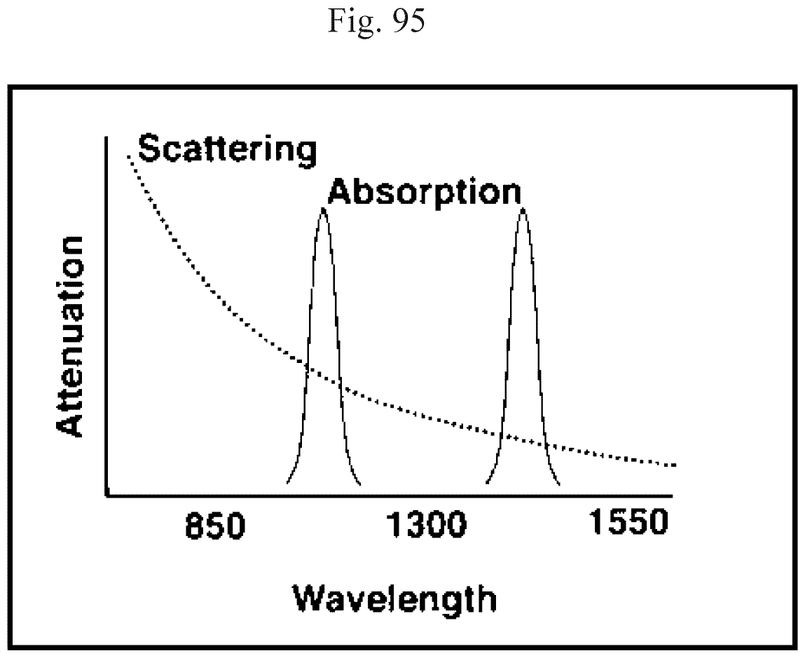

In embodiments, optical fibers may be used to take the emitted wave from the light source to the tissue. For example, for fiber optics with glass fibers, light may be used with wavelengths longer than visible light, typically around 850, 1300 and 1550 nm. The reason these wavelengths are preferred is that attenuation in the fibers is smaller for these wavelengths. As shown in FIG. 95, scattering effects are lower as the wavelength increases, and absorption occurs in in several specific wavelengths (called water bands), due to the absorption by minute amounts of water vapor in the glass. However, these wavelengths may be significantly larger than what it is needed for neural stimulation (380 to 470 nm). In embodiments, plastic optical fibers may be used.

An exemplary block diagram of a computing device 9600, which may be included in control/processing circuitry 8614, shown in FIG. 8, in which processes involved in the embodiments described herein may be implemented, is shown in FIG. 96. Computing device 9600 may be a programmed general-purpose computer system, such as an embedded processor, microcontroller, system on a chip, microprocessor, smartphone, tablet, or other mobile computing device, personal computer, workstation, server system, and minicomputer or mainframe computer. Computing device 9600 may include one or more processors (CPUs) 9602A-9602N, input/output circuitry 9604, network adapter 9606, and memory 9608. CPUs 9602A-9602N execute program instructions in order to carry out the functions of the present invention. Typically, CPUs 9602A-9602N are one or more microprocessors, such as an INTEL PENTIUM® processor. FIG. 96 illustrates an embodiment in which computing device 9600 is implemented as a single multi-processor computer system, in which multiple processors 9602A-9602N share system resources, such as memory 9608, input/output circuitry 9604, and network adapter 9606. However, the present invention also contemplates embodiments in which computing device 9600 is implemented as a plurality of networked computer systems, which may be single-processor computer systems, multi-processor computer systems, or a mix thereof.

Input/output circuitry 9604 provides the capability to input data to, or output data from, computing device 9600. For example, input/output circuitry may include input devices, such as keyboards, mice, touchpads, trackballs, scanners, etc., output devices, such as video adapters, monitors, printers, etc., and input/output devices, such as, modems, etc. Network adapter 9606 interfaces device 9600 with a network 9610. Network 9610 may be any public or proprietary LAN or WAN, including, but not limited to the Internet.

Memory 9608 stores program instructions that are executed by, and data that are used and processed by, CPU 9602 to perform the functions of computing device 9600. Memory 9608 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 9608 may vary depending upon the function that computing device 9600 is programmed to perform. For example, as shown in FIG. 1, computing devices may perform a variety of roles in the system, method, and computer program product described herein. For example, computing devices may perform one or more roles as end devices, gateways/base stations, application provider servers, and network servers. In the example shown in FIG. 96, exemplary memory contents are shown representing routines and data for all of these roles. However, one of skill in the art would recognize that these routines, along with the memory contents related to those routines, may not typically be included on one system or device, but rather are typically distributed among a plurality of systems or devices, based on well-known engineering considerations. The present invention contemplates any and all such arrangements.

In the example shown in FIG. 96, memory 9608 may include sensor data capture routines 9612, signal pre-processing routines 9614, signal processing routines 9616, machine learning routines 9618, output routines 9620, databases 9622, and operating system 9624. For example, sensor data capture routines 9612 may include routines that interact with one or more sensors, such as EEG sensors, and acquire data from the sensors for processing. Signal pre-processing routines 9614 may include routines to pre-process the received signal data, such as by performing band-pass filtering, artifact removal, finding common spatial patterns, segmentation, etc. Signal processing routines 9616 may include routines to process the pre-processed signal data, such as by performing time domain processing, such as spindle threshold processing, frequency domain processing, such as power spectrum processing, and time-frequency domain processing, such as wavelet analysis, etc. Machine learning routines 9618 may include routines to perform machine learning processing on the processed signal data. Databases 9622 may include databases that may be used by the processing routines. Operating system 9624 provides overall system functionality.

As shown in FIG. 96, the present invention contemplates implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry (such as that shown at 208 of FIG. 2) may include, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims. Further, it is to be noted that, as used in the claims, the term coupled may refer to electrical or optical connection and may include both direct connection between two or more devices and indirect connection of two or more devices through one or more intermediate devices.

What is claimed is:

1. A system to provide self-guided, self-directed diagnostics and treatment of neural conditions comprising:
a processor, memory accessible by the processor, and program instructions and data stored in the memory;
a plurality of stimulation devices connected to signal output circuitry interfacing the processor with the stimulation devices;
program instructions and data stored in the memory to configure the processor to control the stimulation devices to generate and transmit stimulation signals;
a plurality of sensing devices connected to signal input circuitry interfacing the processor with the sensing devices;
program instructions and data stored in the memory to configure the processor to receive sensed signals from the sensing devices;
a communication device adapted to wirelessly communicate with an external computer system;
wherein the processor, memory, plurality of stimulation devices, plurality of sensing devices, and communication device are contained in an implantable device adapted to be implanted in a body of a person; and
an external computer system comprising a processor, memory accessible by the processor, and program instructions and data stored in the memory, the program instructions and data stored in the memory to configure the processor to perform dynamic closed loop feedback of the stimulation signals based on the received sensed signals to provide self-guided, self-directed diagnostics and treatment of neural conditions using at least one recipe for a treatment strategy guided by artificial intelligence contained in the server computer system and communicated using the communication device;
wherein the external computer system is adapted to be attached to a body of the person.

2. The system of claim 1, wherein the plurality of stimulation devices comprises at least one of stimulation devices in the implantable device providing electrical stimulation, stimulation devices in the implantable device providing optical stimulation, and stimulation devices in the implantable device providing both electrical and optical stimulation.

3. The system of claim 2, wherein the plurality of stimulation devices further comprises stimulation devices not contained in the implantable device including at least one of a Transcranial Direct Current Stimulation (tDCS) device, a Transcranial Alternating Current Stimulation (tACS), and a Transcranial Photobiomodulation (tPBM).

4. The system of claim 2, wherein the plurality of sensing devices comprises at least one of sensing devices contained in the implantable device providing electrical sensing, sensing devices contained in the implantable device providing optical sensing, and sensing devices contained in the implantable device providing both electrical and optical sensing.

5. The system of claim 4, wherein the plurality of sensing devices further comprises sensing devices not contained in the implantable device including at least an Electro Encephalogram (EEG) device.

6. The system of claim 4, further comprising program instructions and data stored in the memory to configure the processor of the implant device to perform:
applying stimulation based on a first version of stimulation parameters,
sensing results of the applied stimulation to form patient treatment results data, transmitting the patient treatment results data to the external computer system,
receiving a second version of stimulation parameters from the external computer system, and
applying stimulation based on the second version of stimulation parameters; and further comprising program instructions and data stored in the memory of the external computer system to configure the processor of the external computer system to perform:
receiving the patient treatment results data,
modifying the first version of stimulation parameters using the patient treatment results data to form a second version of stimulation parameters, and
transmitting the second version of stimulation parameters to the implant device.

7. The system of claim 6, further comprising program instructions and data stored in the memory to configure the processors of the implant device and the external computer system to perform:
repeating the applying, sensing, transmitting, receiving, and modifying using each modified version of stimulation parameters.

8. The system of claim 1, wherein the communication device is adapted to communicate using WiFi wireless communications.

9. The system of claim 1, wherein the communication device is adapted to communicate using Ultra Wide-Band wireless communications.
